SEQUENCE LISTING (16 sequences)

SEQ ID NO: 1 - DNA, 20 bp, synthetic construct
  source 1..20: mol_type = other DNA
tatccggactcctccatctg

SEQ ID NO: 2 - DNA, 20 bp, synthetic construct
  source 1..20: mol_type = other DNA
tgttcgtgcgttcttacctg

SEQ ID NO: 3 - DNA, 20 bp, synthetic construct
  source 1..20: mol_type = other DNA
attcctgagaaggccaggat

SEQ ID NO: 4 - DNA, 20 bp, synthetic construct
  source 1..20: mol_type = other DNA
ctgccgacaagcattcctat

SEQ ID NO: 5 - DNA, 20 bp, synthetic construct
  source 1..20: mol_type = other DNA
accaaggttagccgcttttt

SEQ ID NO: 6 - DNA, 20 bp, synthetic construct
  source 1..20: mol_type = other DNA
cttgcccaacaaaatgacct

SEQ ID NO: 7 - DNA, 22 bp, synthetic construct
  source 1..22: mol_type = other DNA
gtaagctctgtttcggcagcac

SEQ ID NO: 8 - DNA, 20 bp, synthetic construct
  source 1..20: mol_type = other DNA
acattgcacgctttgccgac

SEQ ID NO: 9 - DNA, 20 bp, synthetic construct
  source 1..20: mol_type = other DNA
aattcaggttcggctgctgt

SEQ ID NO: 10 - DNA, 20 bp, synthetic construct
  source 1..20: mol_type = other DNA
caggcagacgttctgctact

SEQ ID NO: 11 - DNA, 20 bp, synthetic construct
  source 1..20: mol_type = other DNA
acgcgtctctttttgagcac

SEQ ID NO: 12 - DNA, 22 bp, synthetic construct
  source 1..22: mol_type = other DNA
ccaaattcaaaggacttgggct

SEQ ID NO: 13 - DNA, 22 bp, synthetic construct
  source 1..22: mol_type = other DNA
cgtagtccacttaagaaggccg

SEQ ID NO: 14 - DNA, 20 bp, synthetic construct
  source 1..20: mol_type = other DNA
gggctttcttcaaacctggc

SEQ ID NO: 15 - DNA, 408 bp, Akkermansia muciniphila
  source 1..408: mol_type = genomic DNA
tcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaag

SEQ ID NO: 16 - DNA, 408 bp, synthetic construct; identical to SEQ ID NO: 15
  source 1..408: mol_type = other DNA

(19) United States
(12) Patent Application Publication
(10) Pub. No.: US 2024/0183861 A1
(43) Pub. Date: Jun. 6, 2024

(54) PHARMACEUTICAL COMPOSITIONS AND USES THEREOF

(71) Applicant: Siolta Therapeutics, Inc., San Carlos, CA (US)

(72) Inventors: Nikole KIMES, San Francisco, CA (US); Ricardo VALLADARES, Brookhaven, GA (US); Jeanette GEHRIG, San Francisco, CA (US); Alison VRBANAC, San Francisco, CA (US)

(21) Appl. No.: 18/502,595

(22) Filed: Nov. 6, 2023

Related U.S. Application Data
(63) Continuation of application No. PCT/US2022/030847, filed on May 25, 2022.
(60) Provisional application No. 63/193,300, filed on May 26, 2021.
(60) Provisional application No. 63/240,264, filed on Sep. 2, 2021.

(51) Int. Cl. G01N 33/68; G01N 33/50; G01N 33/92

(52) U.S. Cl. CPC G01N 33/6869; G01N 33/5008; G01N 33/92; G01N 2333/5406; G01N 2333/723

(57) ABSTRACT

Provided herein are methods for administering pharmaceutical compositions comprising bacterial populations and methods for obtaining samples from subjects administered with the pharmaceutical compositions. Further provided are methods for assaying bacteria, compounds, markers, any derivatives thereof, or any combinations thereof. The reagents, kits, systems, or any combinations thereof for carrying out the same are also provided.

### CROSS REFERENCE

This application is a continuation of International Application No. PCT/US2022/030847, filed May 25, 2022, which claims. The benefit of U.S. Provisional Patent Application No. 63/193,300, filed on May 26, 2021, and U.S. Provisional Patent Application No. 63/240,264, filed on Sep. 2, 2021, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Dec. 1, 2023, is named 53206-711_301_SL.xml and is 15,884 bytes in size.

### BACKGROUND

Recent developments in the areas of microbiome and genome research provide evidence that the microbiome-host relationships influence human health or disease onset and progression. For example, they have been implicated in the inflammatory and metabolic diseases, playing key roles in the etiology of these disorders. The rising incidence of these diseases is concerning and represents a major public health challenge.

Restoring the microbiome-host can help treating these disorders. Effective treatments comprise administration of live biotherapeutics. Currently available pharmaceutical compositions, however, can lack effectiveness, scalability, reliability, or stability due to the unknown or unpredictable behaviors of the microbes subsequent to being administered into the host.

### BRIEF SUMMARY

Disclosed herein, in some embodiments, are methods. In an aspect, a method comprises: (a) administering to a subject a pharmaceutical composition comprising a bacterial population comprising at least two bacterial species selected from the group consisting of Akkermansia sp., Faecalibacterium sp., and Lactobacillus sp.; (b) assaying a microbial community in said subject relative to a control microbial community.

In an aspect, a method comprises: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a microbial community in said subject relative to a control microbial community.

Disclosed herein, in some embodiments, are methods. In an aspect, a method comprises: (a) obtaining a sample comprising a microbial community from a subject; (b) assaying said microbial community relative to a control microbial community, wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp.

In an aspect, a method comprises: (a) obtaining a sample comprising a microbial community from a subject; (b) assaying said microbial community relative to a control microbial community, wherein said sample is obtained from said subject subsequent to being administered with a pharmaceutical composition comprising a bacterial population comprising at least two bacterial species selected from the group consisting of Akkermansia sp., Faecalibacterium sp., and a species of the Lactobacillaceae family.

In some embodiments, the assaying of (b) comprises repeated measures of said microbial community. In some embodiments, the assaying of (b) comprises a linear mixed effect (LME) model. In some embodiments, the assaying of (b) measures a microbial diversity of said microbial community. In some embodiments, the microbial diversity comprises an alpha-diversity or a beta-diversity. In some embodiments, the microbial diversity comprises said alpha-diversity. In some embodiments, the microbial diversity comprises said beta-diversity. In some embodiments, the beta-diversity is measured by a weighted UniFrac distance or a Jaccard distance. In some embodiments, the beta-diversity is measured by a weighted UniFrac distance. In some embodiments, the beta-diversity is measured by a Jaccard distance.

In some embodiments, the assaying of (b) identifies an enrichment or a depletion of at least one bacterial species in said microbial community. In some embodiments, the assaying of (b) identifies said enrichment of at least one bacterial species in said microbial community. In some embodiments, the at least one enriched bacterial species in said microbial community synthesizes short chain fatty acids (SCFAs). In some embodiments, the assaying of (b) identifies said depletion of at least one bacterial species in said microbial community. In some embodiments, the at least one depleted bacterial species in said microbial community is associated with an inflammatory disease. In some embodiments, the inflammatory disease comprises an allergy or a dermatitis.

In some embodiments, the assaying of (b) identifies said enrichment or said depletion of at least one bacterial species in said microbial community using Statistical Framework Analysis of Composition of Microbiomes (ANCOM) or Applies Generalized Additive Models for Location, Scale and Shape (GAMLSS). In some embodiments, the enriched bacterial species in said microbial community comprises one strain selected from the group consisting of family Bacteroidaceae and family Christensenellaceae. In some embodiments, the enriched bacterial species in said microbial community comprises one strain selected from the group consisting of genus Bacteroides and genus Clostridiaceae SMB53. In some embodiments, the enriched bacterial species in said microbial community comprises one strain selected from the group consisting of Ruminococcus sp. and Streptococcus sp. In some embodiments, the enriched bacterial species in said microbial community comprises one strain selected from the group consisting of Roseburia faecis, Blautia producta, Bacteroides eggerthii, Bacteroides plebeius, and Coprococcus eutactus. In some embodiments, the depleted bacterial species in said microbial community comprises one strain selected from the group consisting of family Clostridiales, family Peptostreptococcaceae, and family Clostridiaceae. In some embodiments, the depleted bacterial species in said microbial community comprises one strain selected from the group consisting of genus Turicibacter, genus Oscillospira, genus Slackia, and genus Coprococcus. In some embodiments, the depleted bacterial species in said microbial community comprises one strain selected from the group consisting of Streptococcus sp., Haemophilus sp., and Enterococcus sp. In some embodiments, the depleted bacterial species in said microbial community comprises one strain selected from the group consisting of Bacteroides ovatus, Eggerthella lenta, Haemophilus parainfluenzae, and Veillonella dispar.

In some embodiments, the control microbial community comprises a microbial community in said subject without being administered to said pharmaceutical composition. In some embodiments, the control microbial community comprises a microbial community in said subject prior to being administered to said pharmaceutical composition.

In some embodiments, the subject is an adult, an adolescent, or an infant. In some embodiments, the subject has or is at risk of having an inflammatory disease, and wherein said inflammatory disease comprises an allergy or a dermatitis. In some embodiments, the allergy is allergic asthma, allergic pediatric asthma, or food allergy. In some embodiments, the pharmaceutical composition is stored in a plant-based capsule prior to being administered to said subject. In some embodiments, the bacterial population comprises said Akkermansia sp., said Faecalibacterium sp., and said Lactobacillus sp. In some embodiments, the bacterial population comprises said Akkermansia sp., said Faecalibacterium sp., and said species of the Lactobacillaceae family. In some embodiments, the bacterial species is selected from the strains listed in Table 1. In some embodiments, the bacterial population comprises A. muciniphila (DSM 33213), F. prausnitzii (DSM 33185), or L. crispatus (DSM 33187). In some embodiments, the bacterial population comprises A. muciniphila (DSM 33213), F. prausnitzii (DSM 33185), and L. crispatus (DSM 33187). In some embodiments, each bacterial species is present in an amount from about 10\hat{ }3 CFU/dose to about 10\hat{ }12 CFU/dose. In some embodiments, each bacterial species is present in an amount from about 10\hat{ }7 CFU/dose to about 10\hat{ }10 CFU/dose. In some embodiments, the Akkermansia sp. is present in an amount of about 10\hat{ }6 CFU/dose to about 2×10\hat{ }9 CFU/dose. In some embodiments, the Faecalibacterium sp. is present in an amount of about 10\hat{ }6 CFU/dose to about 2×10\hat{ }9 CFU/dose. In some embodiments, the Lactobacillus sp. is present in an amount of about 10\hat{ }7 CFU/dose to about 5×10\hat{ }9 CFU/dose. In some embodiments, the species of the Lactobacillaceae family is present in an amount of about 10\hat{ }7 CFU/dose to about 5×10\hat{ }9 CFU/dose. In some embodiments, the bacterial population is present in a total amount of about 10\hat{ }3 CFU/dose to about 10\hat{ }12 CFU/dose. In some embodiments, the bacterial population is present in a total amount of about 10\hat{ }7 CFU/dose to about 10\hat{ }10 CFU/dose.

Disclosed herein, are methods. In an aspect, a method comprises: (a) administering to a subject a pharmaceutical composition comprising a bacterial population comprising at least two bacterial species selected from the group consisting of Akkermansia sp., Faecalibacterium sp., and Lactobacillus sp.; (b) assaying at least one bacterial species of said bacterial population in said subject relative to said at least one species of a control bacterial population. In an aspect, a method comprises: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying at least one bacterial species of said bacterial population in said subject relative to said at least one species of a control bacterial population.

Disclosed herein, are methods. In an aspect, a method comprises: (a) obtaining a sample from a subject; (b) assaying at least one bacterial species of a bacterial population of said sample relative to said at least one species of a control bacterial population, wherein said sample is obtained from said subject subsequent to being administered with a pharmaceutical composition comprising said bacterial population comprising at least two bacterial species selected from the group consisting of Akkermansia sp., Faecalibacterium sp., and Lactobacillus sp. In an aspect, a method comprises: (a) obtaining a sample from a subject; (b) assaying at least one bacterial species of a bacterial population of said sample relative to said at least one species of a control bacterial population, wherein said sample is obtained from said subject subsequent to being administered with a pharmaceutical composition comprising said bacterial population comprising at least two bacterial species selected from the group consisting of Akkermansia sp., Faecalibacterium sp., and a species of the Lactobacillaceae family.

In some embodiments, the assaying of (b) comprises taking a mean abundance or a median abundance of said at least one bacterial species of said bacterial population and said control bacterial population. In some embodiments, the control bacterial population comprises said bacterial population in said subject without being administered to said pharmaceutical composition. In some embodiments, the control bacterial population comprises said bacterial population in said subject prior to being administered to said pharmaceutical composition. In some embodiments, the at least one bacterial species of said bacterial population is present in said sample during said subject is administered to said pharmaceutical composition. In some embodiments, the at least one bacterial species of said bacterial population is present in said sample in a washout period subsequent to said subject is administered to said pharmaceutical composition. In some embodiments, the assaying of (b) comprises a quantitative PCR.

In some embodiments, the subject is an adult, an adolescent, or an infant. In some embodiments, the subject has or is at risk of having an inflammatory disease, and wherein said inflammatory disease comprises an allergy or a dermatitis. In some embodiments, the allergy is allergic asthma, allergic pediatric asthma, or food allergy. In some embodiments, the pharmaceutical composition is stored in a plant-based capsule prior to being administered to said subject. In some embodiments, the bacterial population comprises said Akkermansia sp., said Faecalibacterium sp., and said Lactobacillus sp. In some embodiments, the bacterial population comprises said Akkermansia sp., said Faecalibacterium sp., and said species of the Lactobacillaceae family. In some embodiments, the bacterial species is selected from the strains listed in Table 1. In some embodiments, the bacterial population comprises A. muciniphila (DSM 33213), F. prausnitzii (DSM 33185), or L. crispatus (DSM 33187). In some embodiments, the bacterial population comprises A. muciniphila (DSM 33213), F. prausnitzii (DSM 33185), and L. crispatus (DSM 33187). In some embodiments, each bacterial species is present in an amount from about 10\hat{ }3 CFU/dose to about 10\hat{ }12 CFU/dose. In some embodiments, each bacterial species is present in an amount from about 10\hat{ }7 CFU/dose to about 10\hat{ }10 CFU/dose. In some embodiments, the Akkermansia sp. is present in an amount of about 10\hat{ }6 CFU/dose to about 2×10\hat{ }9 CFU/dose. In some embodiments, the Faecalibacterium sp. is present in an amount of about 10\hat{ }6 CFU/dose to about 2×10\hat{ }9 CFU/dose. In some embodiments, the Lactobacillus sp. is present in an amount of about 10\hat{ }7 CFU/dose to about 5×10\hat{ }9 CFU/dose. In some embodiments, the species of the Lactobacillaceae family is present in an amount of about 10\hat{ }7 CFU/dose to about 5×10\hat{ }9 CFU/dose. In some embodiments, the bacterial population is present in a total amount of about 10\hat{ }3 CFU/dose to about 10\hat{ }12 CFU/dose. In some embodiments, the bacterial population is present in a total amount of about 10\hat{ }7 CFU/dose to about 10\hat{ }10 CFU/dose.

Disclosed herein, are methods. In an aspect, a method comprises: (a) administering to a subject a pharmaceutical composition comprising a bacterial population comprising at least one strain of Akkermansia sp., at least one strain of Faecalibacterium sp., or at least one strain of Lactobacillus sp.; (b) assaying a steroid compound in said subject relative to said steroid compound in a control sample. In an aspect, a method comprises: (a) administering to a subject a pharmaceutical composition comprising a bacterial population comprising at least one strain of Akkermansia sp., at least one strain of Faecalibacterium sp., or at least one strain of a species of the Lactobacillaceae family; (b) assaying a steroid compound in said subject relative to said steroid compound in a control sample.

Disclosed herein, are methods. In an aspect, a method comprises: (a) obtaining a sample comprising a steroid compound from a subject; (b) assaying said steroid compound relative to said steroid compound in a control sample, wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp. In an aspect, a method comprises: (a) obtaining a sample comprising a steroid compound from a subject; (b) assaying said steroid compound relative to said steroid compound in a control sample, wherein said sample is obtained from said subject subsequent to being administered with a pharmaceutical composition comprising a bacterial population comprising at least two bacterial species selected from the group consisting of Akkermansia sp., Faecalibacterium sp., and a species of the Lactobacillaceae family.

In some embodiments, the steroid compound comprises an androgenic steroid compound. In some embodiments, the androgenic steroid compound is selected from the group consisting of androstenediol (3alpha, 17alpha) monosulfate, 11 beta-hydroxyandrosterone glucuronide, and androsterone glucuronide. In some embodiments, the steroid compound in a control sample comprises said steroid compound in a subject without being administered to said pharmaceutical composition. In some embodiments, the steroid compound in a control sample comprises said steroid compound in said subject prior to being administered to said pharmaceutical composition. In some embodiments, the steroid compound is present in said subject during said subject is administered to said pharmaceutical composition. In some embodiments, the steroid compound is present in said subject in a washout period subsequent to said subject is administered to said pharmaceutical composition. In some embodiments, the assaying of (b) comprises a liquid chromatography mass spectrometry (LC-MS).

In some embodiments, the subject is an adult, an adolescent, or an infant. In some embodiments, the subject has or is at risk of having an inflammatory disease, and wherein said inflammatory disease comprises an allergy or a dermatitis. In some embodiments, the allergy is allergic asthma, allergic pediatric asthma, or food allergy. In some embodiments, the pharmaceutical composition is stored in a plant-based capsule prior to being administered to said subject. In some embodiments, the bacterial population comprises said Akkermansia sp., said Faecalibacterium sp., and said Lactobacillus sp. In some embodiments, the bacterial population comprises said Akkermansia sp., said Faecalibacterium sp., and said species of the Lactobacillaceae family. In some embodiments, the bacterial species is selected from the strains listed in Table 1. In some embodiments, the bacterial population comprises A. muciniphila (DSM 33213), F. prausnitzii (DSM 33185), or L. crispatus (DSM 33187). In some embodiments, the bacterial population comprises A. muciniphila (DSM 33213), F. prausnitzii (DSM 33185), and L. crispatus (DSM 33187). In some embodiments, each bacterial species is present in an amount from about 10\hat{ }3 CFU/dose to about 10\hat{ }12 CFU/dose. In some embodiments, each bacterial species is present in an amount from about 10\hat{ }7 CFU/dose to about 10\hat{ }10 CFU/dose. In some embodiments, the Akkermansia sp. is present in an amount of about 10\hat{ }6 CFU/dose to about 2×10\hat{ }9 CFU/dose. In some embodiments, the Faecalibacterium sp. is present in an amount of about 10\hat{ }6 CFU/dose to about 2×10\hat{ }9 CFU/dose. In some embodiments, the Lactobacillus sp. is present in an amount of about 10\hat{ }7 CFU/dose to about 5×10\hat{ }9 CFU/dose. In some embodiments, the species of the Lactobacillaceae family is present in an amount of about 10\hat{ }7 CFU/dose to about 5×10\hat{ }9 CFU/dose. In some embodiments, the bacterial population is present in a total amount of about 10\hat{ }3 CFU/dose to about 10\hat{ }12 CFU/dose. In some embodiments, the bacterial population is present in a total amount of about 10\hat{ }7 CFU/dose to about 10\hat{ }10 CFU/dose.

Disclosed herein, are methods. In an aspect, a method comprises: (a) administering to a subject a pharmaceutical composition comprising a bacterial population comprising at least one strain of Akkermansia sp., at least one strain of Faecalibacterium sp., or at least one strain of Lactobacillus sp.; (b) assaying a microbially-derived metabolite in said subject relative to said microbially-derived metabolite in a control sample, wherein said microbially-derived metabolite is an amine oxide. In an aspect, a method comprises: (a) administering to a subject a pharmaceutical composition comprising a bacterial population comprising at least one strain of Akkermansia sp., at least one strain of Faecalibacterium sp., or at least one strain of a species of the Lactobacillaceae family; (b) assaying a microbially-derived metabolite in said subject relative to said microbially-derived metabolite in a control sample, wherein said microbially-derived metabolite is an amine oxide.

Disclosed herein, are methods. In an aspect, a method comprises: (a) obtaining a sample comprising a microbially-derived metabolite from a subject; (b) assaying said microbially-derived metabolite relative to said microbially-derived metabolite in a control sample, wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp., wherein said microbially-derived metabolite is an amine oxide. In an aspect, a method comprises: (a) obtaining a sample comprising a microbially-derived metabolite from a subject; (b) assaying said microbially-derived metabolite relative to said microbially-derived metabolite in a control sample, wherein said sample is obtained from said subject subsequent to being administered with a pharmaceutical composition comprising a bacterial population comprising at least two bacterial species selected from the group consisting of Akkermansia sp., Faecalibacterium sp., and a species of the Lactobacillaceae family, wherein said microbially-derived metabolite is an amine oxide.

In some embodiments, the microbially-derived metabolite comprises trimethylamine N-oxide. In some embodiments, the microbially-derived metabolite in a control sample comprises said microbially-derived metabolite in a subject without being administered to said pharmaceutical composition. In some embodiments, the microbially-derived metabolite in a control sample comprises said microbially-derived metabolite in said subject prior to being administered to said pharmaceutical composition. In some embodiments, the microbially-derived metabolite is present in said subject during said subject is administered to said pharmaceutical composition. In some embodiments, the microbially-derived metabolite is present in said subject in a washout period subsequent to said subject is administered to said pharmaceutical composition. In some embodiments, the assaying of (b) comprises a liquid chromatography mass spectrometry (LC-MS).

In some embodiments, the subject is an adult, an adolescent, or an infant. In some embodiments, the subject has or is at risk of having an inflammatory disease, and wherein said inflammatory disease comprises an allergy or a dermatitis. In some embodiments, the allergy is allergic asthma, allergic pediatric asthma, or food allergy. In some embodiments, the pharmaceutical composition is stored in a plant-based capsule prior to being administered to said subject. In some embodiments, the bacterial population comprises said Akkermansia sp., said Faecalibacterium sp., and said Lactobacillus sp. In some embodiments, the bacterial population comprises said Akkermansia sp., said Faecalibacterium sp., and said species of the Lactobacillaceae family. In some embodiments, the bacterial species is selected from the strains listed in Table 1. In some embodiments, the bacterial population comprises A. muciniphila (DSM 33213), F. prausnitzii (DSM 33185), or L. crispatus (DSM 33187). In some embodiments, the bacterial population comprises A. muciniphila (DSM 33213), F. prausnitzii (DSM 33185), and L. crispatus (DSM 33187). In some embodiments, each bacterial species is present in an amount from about 10\hat{ }3 CFU/dose to about 10\hat{ }12 CFU/dose. In some embodiments, each bacterial species is present in an amount from about 10\hat{ }7 CFU/dose to about 10\hat{ }10 CFU/dose. In some embodiments, the Akkermansia sp. is present in an amount of about 10\hat{ }6 CFU/dose to about 2×10\hat{ }9 CFU/dose. In some embodiments, the Faecalibacterium sp. is present in an amount of about 10\hat{ }6 CFU/dose to about 2×10\hat{ }9 CFU/dose. In some embodiments, the Lactobacillus sp. is present in an amount of about 10\hat{ }7 CFU/dose to about 5×10\hat{ }9 CFU/dose. In some embodiments, the species of the Lactobacillaceae family is present in an amount of about 10\hat{ }7 CFU/dose to about 5×10\hat{ }9 CFU/dose. In some embodiments, the bacterial population is present in a total amount of about 10\hat{ }3 CFU/dose to about 10\hat{ }12 CFU/dose. In some embodiments, the bacterial population is present in a total amount of about 10\hat{ }7 CFU/dose to about 10\hat{ }10 CFU/dose.

Disclosed herein, are methods. In an aspect, a method comprises: (a) administering to a subject a pharmaceutical composition comprising a bacterial population comprising at least one strain of Akkermansia sp., at least one strain of Faecalibacterium sp., or at least one strain of Lactobacillus sp.; (b) assaying a membrane lipid in said subject relative to said membrane lipid in a control sample, wherein said membrane lipid comprises phosphatidylinositol (PI) or phosphatidylethanolamine (PE). In an aspect, a method comprises: (a) administering to a subject a pharmaceutical composition comprising a bacterial population comprising at least one strain of Akkermansia sp., at least one strain of Faecalibacterium sp., or at least one strain of a species of the Lactobacillaceae family; (b) assaying a membrane lipid in said subject relative to said membrane lipid in a control sample, wherein said membrane lipid comprises phosphatidylinositol (PI) or phosphatidylethanolamine (PE).

Disclosed herein, are methods. In an aspect, a method comprises: (a) obtaining a sample comprising a membrane lipid from a subject; (b) assaying said membrane lipid relative to said membrane lipid in a control sample, wherein said membrane lipid comprises phosphatidylinositol (PI) or phosphatidylethanolamine (PE), and wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp. In an aspect, a method comprises: (a) obtaining a sample comprising a membrane lipid from a subject; (b) assaying said membrane lipid relative to said membrane lipid in a control sample, wherein said membrane lipid comprises phosphatidylinositol (PI) or phosphatidylethanolamine (PE), and wherein said sample is obtained from said subject subsequent to being administered with a pharmaceutical composition comprising a bacterial population comprising at least two bacterial species selected from the group consisting of Akkermansia sp., Faecalibacterium sp., and a species of the Lactobacillaceae family.

In some embodiments, the membrane lipid in a control sample comprises said membrane lipid in a subject without being administered to said pharmaceutical composition. In some embodiments, the membrane lipid in a control sample comprises said membrane lipid in said subject prior to being administered to said pharmaceutical composition. In some embodiments, the membrane lipid is present in said subject during said subject is administered to said pharmaceutical composition. In some embodiments, the membrane lipid is present in said subject in a washout period subsequent to said subject is administered to said pharmaceutical composition. In some embodiments, the assaying of (b) comprises a liquid chromatography mass spectrometry (LC-MS).

In some embodiments, the subject is an adult, an adolescent, or an infant. In some embodiments, the subject has or is at risk of having an inflammatory disease, and wherein said inflammatory disease comprises an allergy or a dermatitis. In some embodiments, the allergy is allergic asthma, allergic pediatric asthma, or food allergy. In some embodiments, the pharmaceutical composition is stored in a plant-based capsule prior to being administered to said subject. In some embodiments, the bacterial population comprises said Akkermansia sp., said Faecalibacterium sp., and said Lactobacillus sp. In some embodiments, the bacterial population comprises said Akkermansia sp., said Faecalibacterium sp., and said species of the Lactobacillaceae family. In some embodiments, the bacterial species is selected from the strains listed in Table 1. In some embodiments, the bacterial population comprises A. muciniphila (DSM 33213), F. prausnitzii (DSM 33185), or L. crispatus (DSM 33187). In some embodiments, the bacterial population comprises A. muciniphila (DSM 33213), F. prausnitzii (DSM 33185), and L. crispatus (DSM 33187). In some embodiments, each bacterial species is present in an amount from about 10\hat{ }3 CFU/dose to about 10\hat{ }12 CFU/dose. In some embodiments, each bacterial species is present in an amount from about 10\hat{ }7 CFU/dose to about 10\hat{ }10 CFU/dose. In some embodiments, the Akkermansia sp. is present in an amount of about 10\hat{ }6 CFU/dose to about 2×10\hat{ }9 CFU/dose. In some embodiments, the Faecalibacterium sp. is present in an amount of about 10\hat{ }6 CFU/dose to about 2×10\hat{ }9 CFU/dose. In some embodiments, the Lactobacillus sp. is present in an amount of about 10\hat{ }7 CFU/dose to about 5×10\hat{ }9 CFU/dose. In some embodiments, the species of the Lactobacillaceae family is present in an amount of about 10\hat{ }7 CFU/dose to about 5×10\hat{ }9 CFU/dose. In some embodiments, the bacterial population is present in a total amount of about 10\hat{ }3 CFU/dose to about 10\hat{ }12 CFU/dose. In some embodiments, the bacterial population is present in a total amount of about 10\hat{ }7 CFU/dose to about 10\hat{ }10 CFU/dose.

Disclosed herein, are methods. In an aspect, a method comprises: (a) administering to a subject a pharmaceutical composition comprising a bacterial population comprising at least one strain of Akkermansia sp., at least one strain of Faecalibacterium sp., or at least one strain of Lactobacillus sp.; (b) assaying a pro-inflammatory immune marker relative to said pro-inflammatory immune marker in a control sample, wherein said pro-inflammatory immune marker in present in a washout period subsequent to said administering of (a). In an aspect, a method comprises: (a) administering to a subject a pharmaceutical composition comprising a bacterial population comprising at least one strain of Akkermansia sp., at least one strain of Faecalibacterium sp., or at least one strain of a species of the Lactobacillaceae family; (b) assaying a pro-inflammatory immune marker relative to said pro-inflammatory immune marker in a control sample, wherein said pro-inflammatory immune marker in present in a washout period subsequent to said administering of (a).

Disclosed herein, are methods. In an aspect, a method comprises: (a) obtaining a sample comprising a pro-inflammatory immune marker from a subject; (b) assaying said pro-inflammatory immune marker relative to said pro-inflammatory immune marker in a control sample, wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp., and wherein said pro-inflammatory immune marker in present in a washout period subsequent to said subject is administered with said pharmaceutical composition. In an aspect, a method comprises: (a) obtaining a sample comprising a pro-inflammatory immune marker from a subject; (b) assaying said pro-inflammatory immune marker relative to said pro-inflammatory immune marker in a control sample, wherein said sample is obtained from said subject subsequent to being administered with a pharmaceutical composition comprising a bacterial population comprising at least two bacterial species selected from the group consisting of Akkermansia sp., Faecalibacterium sp., and a species of the Lactobacillaceae family, and wherein said pro-inflammatory immune marker in present in a washout period subsequent to said subject is administered with said pharmaceutical composition.

In some embodiments, the pro-inflammatory immune marker comprises basophil, eotaxin, histamine, IL-4, or a combination thereof. In some embodiments, the control pro-inflammatory immune marker comprises said pro-inflammatory immune marker in a subject without being administered to said pharmaceutical composition. In some embodiments, the control pro-inflammatory immune marker comprises said pro-inflammatory immune marker in said subject prior to being administered to said pharmaceutical composition. In some embodiments, the assaying of (b) comprises an enzyme-linked immunosorbent assay (ELISA) or cytometry.

In some embodiments, the subject is an adult, an adolescent, or an infant. In some embodiments, the subject has or is at risk of having an inflammatory disease, and wherein said inflammatory disease comprises an allergy or a dermatitis. In some embodiments, the allergy is allergic asthma, allergic pediatric asthma, or food allergy. In some embodiments, the pharmaceutical composition is stored in a plant-based capsule prior to being administered to said subject. In some embodiments, the bacterial population comprises said Akkermansia sp., said Faecalibacterium sp., and said Lactobacillus sp. In some embodiments, the bacterial population comprises said Akkermansia sp., said Faecalibacterium sp., and said species of the Lactobacillaceae family. In some embodiments, the bacterial species is selected from the strains listed in Table 1. In some embodiments, the bacterial population comprises A. muciniphila (DSM 33213), F. prausnitzii (DSM 33185), or L. crispatus (DSM 33187). In some embodiments, the bacterial population comprises A. muciniphila (DSM 33213), F. prausnitzii (DSM 33185), and L. crispatus (DSM 33187). In some embodiments, each bacterial species is present in an amount from about 10\hat{ }3 CFU/dose to about 10\hat{ }12 CFU/dose. In some embodiments, each bacterial species is present in an amount from about 10\hat{ }7 CFU/dose to about 10\hat{ }10 CFU/dose. In some embodiments, the Akkermansia sp. is present in an amount of about 10\hat{ }6 CFU/dose to about 2×10\hat{ }9 CFU/dose. In some embodiments, the Faecalibacterium sp. is present in an amount of about 10\hat{ }6 CFU/dose to about 2×10\hat{ }9 CFU/dose. In some embodiments, the Lactobacillus sp. is present in an amount of about 10\hat{ }7 CFU/dose to about 5×10\hat{ }9 CFU/dose. In some embodiments, the species of the Lactobacillaceae family is present in an amount of about 10\hat{ }7 CFU/dose to about 5×10\hat{ }9 CFU/dose. In some embodiments, the bacterial population is present in a total amount of about 10\hat{ }3 CFU/dose to about 10\hat{ }12 CFU/dose. In some embodiments, the bacterial population is present in a total amount of about 10\hat{ }7 CFU/dose to about 10\hat{ }10 CFU/dose.

Disclosed herein, in some embodiments, are pharmaceutical compositions. In an aspect, a pharmaceutical composition comprises: a purified bacterial population comprising at least one strain of Akkermansia sp., at least one strain of Faecalibacterium sp., or at least one strain of Lactobacillus sp. In an aspect, a pharmaceutical composition comprises: a purified bacterial population comprising at least one strain of Akkermansia sp., at least one strain of Faecalibacterium sp., or at least one strain of a species of the Lactobacillaceae family.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The features of the disclosure are set forth with particularity in the appended claims. A better understanding of the features and the disclosure will be obtained by reference to the following detailed description that sets forth illustrative embodiments, in which the principles of the invention are utilized, and the accompanying drawings (also “Figure” and “FIG.” herein), of which:

FIG. 1 shows a schematic of the three stages (screening period, treatment period, and washout period) of a human clinical study with an orally administered pharmaceutical composition A comprising Akkermansia muciniphila (DSM 33213), Faecalibacterium prausnitzii (DSM 33185), and Lactobacillus crispatus (DSM 33187).

FIGS. 2A-B show that the abundances of Akkermansia muciniphila (DSM 33213) and Faecalibacterium prausnitzii (DSM 33185) in the subjects are positively correlated during the treatment period of pharmaceutical composition A. FIG. 2A shows the numbers of Akkermansia muciniphila (DSM 33213) (X-axis) and Faecalibacterium prausnitzii (DSM 33185) (Y-axis) in the stool sample of the subject treated with pharmaceutical composition A. The abundances of the two strains had a correlation coefficient (R) of 0.414, which is statistically significant (p=0.018). FIG. 2B shows the numbers of Akkermansia muciniphila (DSM 33213) (X-axis) and Faecalibacterium prausnitzii (DSM 33185) (Y-axis) in the stool samples of the subject treated with the placebo control. The abundances of the two strains did not have any correlation (R=0.009, p=0.97). Data represent log-transformed colony-forming units (CFU) per gram of stool calculated using quantitative PCR and strain-specific primers. Grey shade represents error measurements. p-value represents Spearman's rank correlation coefficient.

FIGS. 3A-3F show that all three bacterial strains in pharmaceutical composition A engraft in the gastrointestinal tract of the subject during the treatment period while Akkermansia muciniphila (DSM 33213) maintains the engraftment in the washout period. FIG. 3A shows the median fold change from the baseline abundance level of Akkermansia muciniphila (DSM 33213) (Y-axis) during the treatment period of pharmaceutical composition A (Treatment) and after a 1-month washout period (Washout). The placebo control did not show a similar change. FIG. 3B shows the median fold change from the baseline abundance level of Faecalibacterium prausnitzii (DSM 33185) (Y-axis) during the treatment period of pharmaceutical composition A (Treatment) and after a 1-month washout period (Washout). The placebo control did not show a similar change. FIG. 3C shows the median fold change from the baseline abundance level of Lactobacillus crispatus (DSM 33187) (Y-axis) during the treatment period of pharmaceutical composition A (Treatment) and after a 1-month washout period (Washout). The placebo control did not show a similar change. FIG. 3D shows the mean fold change from the baseline abundance level of Akkermansia muciniphila (DSM 33213) (Y-axis) during the treatment period of pharmaceutical composition A (Treatment) and after a 1-month washout period (Washout). The placebo control did not show a similar change. FIG. 3E shows the mean fold change from the baseline abundance level of Faecalibacterium prausnitzii (DSM 33185) (Y-axis) during the treatment period of pharmaceutical composition A (Treatment) and after a 1-month washout period (Washout). The Placebo control did not show a similar change. FIG. 3F shows the mean fold change from the baseline abundance level of Lactobacillus crispatus (DSM 33187) (Y-axis) during the treatment period of pharmaceutical composition A (Treatment) and after a 1-month washout period (Washout). The Placebo control did not show a similar change.

FIGS. 4A-C show that sequencing depths in the microbiome analysis can capture the microbiome diversity in the subject administered with pharmaceutical composition A. FIG. 4A shows a rarefaction curve analysis of the sequences identified in the subjects of cohort 1. FIG. 4B shows a rarefaction curve analysis of the sequences identified in the subjects of cohort 2. FIG. 4C shows a rarefaction curve analysis of the sequences identified in the subjects of cohort 3. In FIGS. 4A-C, the Y-axis shows the number of unique bacterial taxa Observed amplicon sequence variant (ASVs), and the X-axis shows the sequencing depths (the number of 1000 reads). Individual curves represent individual subject in each cohort. Observed ASVs approached saturation with ˜5000 sequence reads.

FIGS. 5A-C show that subjects administered with pharmaceutical composition A have unique and stable gastrointestinal microbiomes. FIG. 5A shows a 3-dimensional Jaccard distance matrix plot of the gastrointestinal bacterial community in each subject in cohort 1. FIG. 5B shows a 3-dimensional Jaccard distance matrix plot of the bacterial community in each subject in cohort 2. FIG. 5C shows a 3-dimensional unweighted UniFrac distance matrix plot of the gastrointestinal bacterial community in each subject in all three cohorts. The color scale represents the age of the subject for each sample. The number of each axis represents the percent of variation explained by the respective principal coordinates.

FIGS. 6A-D show that the pharmaceutical composition A treatment results in a significant divergence of the gut microbiome. FIG. 6A shows that the alpha-diversity (Shannon, Y-axis) of the gastrointestinal microbiomes of subjects in cohort 2 did not differ significantly from each other during various time points of pharmaceutical composition A and placebo treatments. “V” in FIG. 6A stands for “visit” or the time point of the measurement FIG. 6B shows that the PERMONOVA analysis of variance with distance matrix (Y-axis) of gastrointestinal microbiome of subjects in the placebo or pharmaceutical composition A treatment group in cohort 2 did not differ significantly to that of the baseline level of the subjects administered with the composition. FIGS. 6C-D show that microbiome diversity in the subject treated with pharmaceutical composition A statistically significantly diverges from baseline. The gastrointestinal microbiome diversity was calculated by measuring the beta-diversity distance indexes of the microbial community (Y-axis) in the stool sample of the subject of the pharmaceutical composition treatment (Treatment) and placebo control (Placebo) in different days of the treatment and washout periods (X-axis). The p-value is calculated by a linear mixed effects model in Qiime2 using the q2-longitudinal plugin and the formula: Distance˜Day (Days treated)×Treatment (the pharmaceutical composition or placebo treatments). Grey shades represent error measurements. For the subjects treated with the pharmaceutical composition, the interaction between Day and Treatment has a p-value of 0.015, suggesting a statistically significant interaction between them, as opposed to the placebo treatment control. The distance index is Weighted UniFrac distance in FIG. 6C and Jaccard Distance in FIG. 6D.

FIG. 7A-Q show that the pharmaceutical composition A treatment results in a significant change of various species in the microbiome of the subjects. FIG. 7A shows an Analysis of Composition of Microbiomes (ANCOM) volcano plot of the microbiome of the subjects in all three cohorts combined. The X and Y-axis show the W statistic and the Centered Log Ratio (clr), respectively. Pharmaceutical composition A significantly altered the abundance of Akkermansia muciniphila, Adlercreutzia sp., and Enterorhabdus sp. in the subjects. FIG. 7B shows the relative abundance of Akkermansia muciniphila and Adlercreutzia sp. in various periods (baseline, treatment, and washout) of pharmaceutical composition A treatment (Drug product) and placebo group of the subjects of all three cohorts combined, as measured by quantitative PCR. FIG. 7C shows a sequence alignment of the Akkermansia muciniphila sequence identified in a subject treated with pharmaceutical composition A and the reference Akkermansia muciniphila sequence (SEQ ID NO: 15) of pharmaceutical composition A (SEQ ID NO: 16). FIG. 7D shows the relative abundance of Blautia sp. in the baseline and treatment period of the subjects of all three cohorts administered with pharmaceutical composition A, as measured by quantitative PCR. FIG. 7E shows the relative abundance of Ruminococcus sp. in the baseline and treatment period of the subjects of all three cohorts administered with pharmaceutical composition A, as measured by quantitative PCR. FIG. 7F shows the relative abundance of Coprococcus sp. in the baseline and treatment period of the subjects of all three cohorts administered with pharmaceutical composition A, as measured by quantitative PCR. FIG. 7G shows the relative abundance of Faecalibacterium prausnitzii strain #2 in the baseline and treatment period of the subjects in cohort 1 administered with pharmaceutical composition A, as measured by quantitative PCR. FIG. 7H shows the relative abundance of Faecalibacterium prausnitzii strain #3 in the baseline and treatment period of the subjects in cohort 1 administered with pharmaceutical composition A, as measured by quantitative PCR. Faecalibacterium prausnitzii strain #2 and Faecalibacterium prausnitzii strain #3 are different strains from Faecalibacterium prausnitzii in pharmaceutical composition A. FIG. 7I shows the relative abundance of Roseburia faecis in the baseline and treatment period of the subjects in cohort 1 administered with pharmaceutical composition A, as measured by quantitative PCR. FIG. 7J shows the relative abundance of Ruminococcus sp. in the baseline and treatment period of the subjects in cohort 1 administered with pharmaceutical composition A, as measured by quantitative PCR. FIG. 7K shows an ANCOM volcano plot of the microbiome of the subjects in cohort 3. The X and Y-axis show the W statistic and the Centered Log Ratio (clr), respectively. Pharmaceutical composition A significantly altered the abundance of the Clostridiales family XIII, the Mogibacteriaceae family, and Akkermansia muciniphila in the subjects. FIG. 7L shows the relative abundance of Bacteroides sp. in the baseline and treatment period of the subjects in cohort 3 administered with pharmaceutical composition A, as measured by quantitative PCR. FIG. 7M shows the relative abundance of Bacteroides ovatus in the baseline and treatment period of the subjects in cohort 3 administered with pharmaceutical composition A, as measured by quantitative PCR. FIG. 7N shows the relative abundance of Bacteroides uniformis in the baseline and treatment period of the subjects in cohort 3 administered with pharmaceutical composition A, as measured by quantitative PCR. FIG. 7O shows the relative abundance of Ruminococcus sp. in the baseline and treatment period of the subjects in cohort 3 administered with pharmaceutical composition A, as measured by quantitative PCR. FIG. 7P shows the relative abundance of Streptococcus sp. in the baseline and treatment period of the subjects in cohort 3 administered with pharmaceutical composition A, as measured by quantitative PCR. FIG. 7Q shows the relative abundance of Haemophilus sp. in the baseline and treatment period of the subjects in cohort 3 administered with pharmaceutical composition A, as measured by quantitative PCR. The number in FIGS. 7P-Q represent the fold-change of the relative abundance of the bacteria in the treatment period relative to that of the baseline period.

FIGS. 8A-G show that the pharmaceutical composition A treatment increases and decreases the abundance of anti-inflammatory and proinflammatory metabolites, respectively. FIG. 8A shows the relative abundance (scaled intensity; Y-axis) of plasma 9,10-DiHome in the subjects during various periods of the pharmaceutical composition A treatment. FIG. 8B shows the relative abundance (scaled intensity; Y-axis) of fecal 12, 13-DiHome in the subjects during various periods of the pharmaceutical composition A treatment. FIG. 8C shows the relative abundance (scaled intensity; Y-axis) of plasma acetoacetate in the subjects during various periods of the pharmaceutical composition A treatment. FIG. 8D shows the relative abundance (scaled intensity; Y-axis) of plasma 3-hydroxybutyrate (BHBA) in the subjects during various periods of the pharmaceutical composition A treatment. FIG. 8E shows the relative abundance (scaled intensity; Y-axis) of plasma and fecal Trimethylamine N-oxide (TMAO) in the subjects of cohort 1 and 2 during various periods of the pharmaceutical composition A treatment. FIG. 8F shows the relative abundance (scaled intensity; Y-axis) of plasma androstenediol (3alpha, 17alpha) monosulfate in the subjects during various periods of the pharmaceutical composition A treatment. FIG. 8G shows the relative abundance (scaled intensity; Y-axis) of plasma 11beta-hydroxyandrosterone glucuronide glucuronide the subjects during various periods of the pharmaceutical composition A treatment. In FIGS. 8A-G, metabolomes of the subjects were measured by untargeted mass spectrometry at the baseline level (BL), the treatment period (E/T), and the washout period (W/O). The levels of the metabolites are plotted as the scaled intensity from LC-MS outputs where values across all samples are scaled to median=1.

FIGS. 9A-C shows that the pharmaceutical composition A treatment reduces the level of the pro-inflammatory immune markers. FIG. 9A shows the level of eotaxin-1 in the blood (Y-axis) of subjects of cohort 1 treated with pharmaceutical composition A and placebo in the baseline level, the treatment period, and the washout period, as measured by an enzyme-linked immunosorbent assay (ELISA). FIG. 9B shows the level of basophils in the blood (Y-axis) of subjects of cohort 1 treated with pharmaceutical composition A and placebo in the baseline level, the treatment period, and the washout period, as measured by white blood cell (WBC) differential using flow cytometry. The numbers of subjects and p-value against the baseline level were shown on top of each comparison group. FIG. 9C shows the level of histamine in the blood (Y-axis) of subjects of cohort 1 treated with pharmaceutical composition A and placebo in the baseline level, the treatment period, and the washout period measured, as by enzyme-linked immunosorbent assay (ELISA). The numbers of subjects (N) and p-value against the baseline level were shown on top of each comparison group. One-way ANOVA was used to compare groups using non-parametric (no assumption of equal variability of differences) Dunnett's multiple comparison test. Correction was performed using Geisser-Greenhouse.

FIGS. 10A-C show limit of detection curves (the control threshold value was plotted against the number of bacterial cells) for quantifying the bacterial strains Akkermansia muciniphila (DSM 33213) (FIG. 10A), and Faecalibacterium prausnitzii (DSM 33185) (FIG. 10B), and Lactobacillus crispatus (DSM 33187) (FIG. 10C), respectively, in a fecal sample. The dotted lines connect the measured data points, the straight lines represent the fitted regression lines, and “R²” is the coefficient of determination. The data shown represent standard curves generated using pure bacterial DNA (10, 1, 0.1, 0.01, 0.001, 0.0001, and 0.00001 nanogram (ng) of 50 ng of total DNA (strain DNA+fecal DNA) of the three strains diluted in fecal DNA background. In instances where human samples are analyzed to determine the amount of strain (e.g., A. muciniphila (DSM 33213), F. prausnitzii (DSM 33185), and L. crispatus (DSM 33187)) DNA, the control samples can be run and analyzed in parallel to ensure the DNA primers used in the experiments are able to properly amplify the DNA of the bacterial strains.

### DETAILED DESCRIPTION

### Definitions

The term “androgenic steroid compound” as used herein refers to a steroid compound that has a similar chemical structure to a testosterone. In some cases, an androgenic steroid compound may also comprise a compound that has a similar biological effect to a testosterone when administered to a subject.

The term “capsule” as used herein refers a container comprising a sac covered by a membrane for containing a pharmaceutical composition. In some cases, a capsule may not be administered to a subject. In some cases, a capsule may not be administered to a subject alongside the pharmaceutical composition. In other cases, a capsule may be administered to a subject.

The term “depletion” or “depleted” as used herein when referring to a microorganism refers to a decrease in abundance of the microorganism in an environment or a sample. In some cases, the decrease in abundance may be a decrease in absolute abundance. In some cases, the decrease in abundance may be a decrease in relative abundance.

The term “enrichment” or “enriched” as used herein when referring to a microorganism refers to an increase in abundance of the microorganism in an environment or a sample. In some cases, the increase in abundance may be an increase in absolute abundance. In some cases, the increase in abundance may be an increase in relative abundance.

The term “inflammatory disease” as used therein refers to a type of diseases, symptoms, or conditions that when the immune system of a subject attacks the tissues or cells of the subject in which the tissues or cells are not infected, injured, or in a disease state. The tissues or cells may also be in a healthy state.

The term “membrane lipid” as used herein refers to a lipid compound that can form or can be found in the membrane layer of a cell.

The term “metabolic disease” as used therein refers to a type of diseases, symptoms, or conditions that disrupt or have a disrupted metabolic process. A disrupted metabolic process may have increased or decreased activity or efficiency relative to a comparable metabolic process that is not disrupted. A disrupted metabolic process may also be different frequency of activity or efficiency relative to a comparable metabolic process that is not disrupted.

The term “microbial community” as used herein refers to a complete set or a substantial portion of a complete set of microorganisms present in a subject. In some cases, a microbial community in a subject may also refer to a metagenome, a microbiota, or a microbiome.

The term “microbial diversity” as used herein refers to a composition or a make-up of a microbial community. In some cases, a microbial diversity may be represented by a qualitative measure or a quantitative measure.

The term “microbially-derived metabolite” as used herein refers to a compound or a substance formed or produced by a microorganism of a microbial community.

The term “pharmaceutical composition” or “composition,” when referring to a pharmaceutical product, refers to a composition that can elicit or induce at least one physiological effect to a subject when administered by the subject, preferably a human. Such physiological effect may positively contribute to the overall health of a subject. In some cases, such physiological effect may also curb, inhibit, reduce, or decrease a negative physiological phenomenon of the subject.

The term “plant-based” as used therein refers to a substance or material that is derived from plants. A plant-based substance or material may not comprise any animal or animal-derived meats, products or by-products. A plant-based substance or material may not comprise any meats, products, or by-products that originated in slaughtered animal.

The term “pro-inflammatory immune marker” as used herein refers to a compound or a protein in which its abundance positively correlates to the immune response of a subject that can be triggered by a foreign substance.

The term “repeated measures” as used herein refers to measuring schemes in which a measurement of a same variable is made on a same subject at least two times or multiple times.

The term “short chain fatty acids” or “SCFAs” refer to fatty acids with fewer than six carbon atoms produced by the metabolism of a substance by a microorganism. For example, such metabolism may comprise fermentation. In some cases, such metabolism may not comprise fermentation.

The term “strain” as used herein refers to a group of bacterial cells, isolates, progenies thereof, or derivatives thereof comprising at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in the genome sequences. For example, two bacterial cells, isolates, progenies thereof, derivatives thereof, or any combinations thereof may be the same strain if they share at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in the genome sequences. In some cases, a strain as used herein can also refer to a group of bacterial cells, isolates, progenies thereof, or derivatives thereof comprising at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in the gene sequence of a genetic locus. In some cases, a strain as used herein can also refer to a group of bacterial cells, isolates, progenies thereof, or derivatives thereof comprising at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in the 16S rRNA gene sequence. For example, two bacterial cells, isolates, progenies thereof, derivatives thereof, or any combinations thereof may be the same strain if they share at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in their 16S rRNA or rDNA sequence.

The term “washout” period as used herein refers to a period of time subsequent to (or after) a subject is administered with a pharmaceutical composition. In some cases, a washout period may also refer to a period of time in which a subject is not being administered with a pharmaceutical composition subsequent to or subsequent to being administered with the pharmaceutical composition.

The term “a” or “an” as used herein refers to one or to more than one (i.e., to at least one) of the grammatical object of the article. By way of example, “an element” means one element or more than one element.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Methods

Provided herein are methods. The methods may be used to generate, increase, or strengthen a beneficial effect(s) of a subject using a pharmaceutical composition comprising a bacterial population. The methods may be used to eliminate, decrease, or weaken a non-beneficial effect(s) of a subject using a pharmaceutical composition comprising a bacterial population. The desired properties that can be elicited by a pharmaceutical composition comprising a bacterial population, when administered by a subject, may be difficult to predict or determine (e.g., a change in the subject's microbiome and/or inflammatory responses), due to inability to accurately determine the properties. For example, a change of the microbiome may be difficult to measure due to the variations of the baseline properties of the microbiome in different subjects (e.g., age groups or administration methods). Provided here are methods to accurately measure any changes in the microbiome of the subject. Additionally, any desired properties elicited by the pharmaceutical composition may depend on the pharmacokinetics of the bacterial population after administered by a subject. Provided herein are methods to accurately measure the pharmacokinetics of the bacterial population. Furthermore, any desired properties elicited by the pharmaceutical composition may depend on measurements of relevant biological markers (e.g., pro-inflammatory or inflammatory metabolite or protein markers). The present disclosures describe various markers that can be used to measure whether or the extent that the desired properties are elicited by the pharmaceutical composition. Accurately determining any of the desired properties described herein can help predict and prepare proper make-up of the pharmaceutical compositions, such as the make-up of the bacterial population to be administered by a subject.

In some instances, a method may comprise administering a pharmaceutical composition. In some cases, a method may comprise obtaining a sample from a subject. In some cases, the sample is obtained from the subject subsequent to the subject being administered with a pharmaceutical composition. In some cases, the sample is obtained from the subject prior to the subject being administered with a pharmaceutical composition. In some cases, the sample is obtained from the subject during the subject is administered with a pharmaceutical composition. In some cases, a pharmaceutical composition may comprise any pharmaceutical composition described in this disclosure.

In some instances, a method may comprise assaying a microbial community, a bacterial population, an androgenic steroid compound, a microbially-derived metabolite, a membrane lipid, a pro-inflammatory immune marker, or a combination thereof of a subject.

In some cases, a method may comprise assaying a microorganism of a subject. In some cases, a method may comprise assaying a microbial strain, species, genus, family, order, class, phylum, kingdom, or a combination thereof. In some cases, a microorganism may comprise a bacterium, a fungus, a protozoon, a virus, an archaeon, or an alga. In some cases, a method may comprise assaying a bacterium of a subject. In some cases, a method may comprise assaying a fungus of a subject. In some cases, a method may comprise assaying a protozoon of a subject. In some cases, a method may comprise assaying a virus of a subject. In some cases, a method may comprise assaying an archaeon of a subject. In some cases, a method may comprise assaying an alga of a subject. In some cases, a method may comprise assaying a bacterial strain, species, genus, family, order, class, phylum, kingdom, or a combination thereof.

### Microbial Community

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying a microbial community in the subject relative to a control microbial community. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a microbial community in the subject relative to a control microbial community. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying a microbial community in the subject relative to a control microbial community. In some cases, a method may comprise the steps of: (a) administering to a subject a pharmaceutical composition comprising a bacterial population comprising at least two bacterial species selected from the group consisting of Akkermansia sp., Faecalibacterium sp., and a species of the Lactobacillus family; (b) assaying a microbial community in the subject relative to a control microbial community. In some cases, a method may comprise: (a) obtaining a sample comprising a microbial community from a subject; (b) assaying the microbial community relative to a control microbial community, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a microbial community from a subject; (b) assaying the microbial community relative to a control microbial community, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a microbial community from a subject; (b) assaying the microbial community relative to a control microbial community, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a microbial community from a subject; (b) assaying the microbial community relative to a control microbial community, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a microbial community from a subject; (3) assaying the microbial community in the subject relative to a control microbial community. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a microbial community from a subject; (3) assaying the microbial community in the subject relative to a control microbial community. In some cases, a method may comprise the steps of: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a microbial community from a subject; (3) assaying the microbial community in the subject relative to a control microbial community. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a microbial community from a subject; (3) assaying the microbial community in the subject relative to a control microbial community.

In some instances, a microbial community of a subject may comprise the microbial community in an organ or a tissue of a subject. In some cases, a microbial community of a subject may comprise the gastrointestinal community of the subject. In some cases, such organ or tissue may also comprise bile duct, ear, eye, gall bladder, gastrointestinal tract, liver, lung, mammary glands, mouth, ovary, saliva, seminal fluid, skin, uterus, vascular system, or any combination thereof.

In some instances, a microbial community may comprise at least about 1×10\hat{ }1 different numbers of microbial species, at least about 1×10\hat{ }2 different numbers of microbial species, at least about 1×10\hat{ }3 different numbers of microbial species, at least about 1×10\hat{ }4 different numbers of microbial species, at least about 1×10\hat{ }5 different numbers of microbial species, at least about 1×10\hat{ }6 different numbers of microbial species, at least about 1×10\hat{ }7 different numbers of microbial species, at least about 1×10\hat{ }8 different numbers of microbial species, at least about 1×10\hat{ }9 different numbers of microbial species, at least about 1×10\hat{ }10 different numbers of microbial species, at least about 1×10\hat{ }11 different numbers of microbial species, at least about 1×10\hat{ }12 different numbers of microbial species, at least about 1×10\hat{ }13 different numbers of microbial species, at least about 1×10\hat{ }14 different numbers of microbial species, at least about 1×10\hat{ }15 different numbers of microbial species, at least about 1×10\hat{ }16 different numbers of microbial species, at least about 1×10\hat{ }17 different numbers of microbial species, at least about 1×10\hat{ }18 different numbers of microbial species, at least about 1×10\hat{ }19 different numbers of microbial species, or at least about 1×10\hat{ }20 different numbers of microbial species. In some instances different numbers of microorganisms, a microbial community may comprise at least about 1×10\hat{ }1 different numbers of microorganisms, at least about 1×10\hat{ }2 different numbers of microorganisms, at least about 1×10\hat{ }5 different numbers of microorganisms, at least about 1×10\hat{ }10 different numbers of microorganisms, at least about 1×10\hat{ }20 different numbers of microorganisms, at least about 1×10\hat{ }50 different numbers of microorganisms, at least about 1×10\hat{ }100 different numbers of microorganisms, at least about 1×10\hat{ }200 different numbers of microorganisms, at least about 1×10\hat{ }500 different numbers of microorganisms, at least about 1×10\hat{ }1000 different numbers of microorganisms, at least about 1×10\hat{ }2000 different numbers of microorganisms, at least about 1×10\hat{ }5000 different numbers of microorganisms, at least about 1×10\hat{ }10000 different numbers of microorganisms, at least about 1×10\hat{ }20000 different numbers of microorganisms, or at least about 1×10\hat{ }50000 different numbers of microorganisms. In some instances, a microbial community may comprise at least about 5×10\hat{ }1 different numbers of genes, at least about 5×10\hat{ }2 different numbers of genes, at least about 5×10\hat{ }3 different numbers of genes, at least about 5×10\hat{ }4 different numbers of genes, at least about 5×10\hat{ }5 different numbers of genes, at least about 5×10\hat{ }6 different numbers of genes, at least about 5×10\hat{ }7 different numbers of genes, at least about 5×10\hat{ }8 different numbers of genes, at least about 5×10\hat{ }9 different numbers of genes, or at least about 5×10\hat{ }10 different numbers of genes. In some instances, a microbial community may comprise at least about 0.2 kilograms of the dry weight of nucleic acids, at least about 0.4 kilograms of the dry weight of nucleic acids, at least about 0.6 kilograms of the dry weight of nucleic acids, at least about 0.8 kilograms of the dry weight of nucleic acids, at least about 1 kilograms of the dry weight of nucleic acids, at least about 1.2 kilograms of the dry weight of nucleic acids, at least about 1.4 kilograms of the dry weight of nucleic acids, at least about 1.6 kilograms of the dry weight of nucleic acids, at least about 1.8 kilograms of the dry weight of nucleic acids, at least about 2 kilograms of the dry weight of nucleic acids, at least about 2.2 kilograms of the dry weight of nucleic acids, at least about 2.4 kilograms of the dry weight of nucleic acids, at least about 2.6 kilograms of the dry weight of nucleic acids, at least about 2.8 kilograms of the dry weight of nucleic acids, at least about 3 kilograms of the dry weight of nucleic acids, at least about 3.2 kilograms of the dry weight of nucleic acids, at least about 3.4 kilograms of the dry weight of nucleic acids, at least about 3.6 kilograms of the dry weight of nucleic acids, at least about 3.8 kilograms of the dry weight of nucleic acids, or at least about 4 kilograms of the dry weight of nucleic acids. In some instances, a microbial community of a subject may comprise a substantial portion of microbial genomic DNA. In some cases, a microbial community of a subject may comprise at least about 50%, 60%, 70%, 75%, 80%, 85%, 90%, 91%, 92%, 93%, 94%, 95%, 96%, 97%, 98%, 99% or 100% of microbial genomic DNA.

In some instances, measurements of the microbial community may comprise measuring the microbial diversity of the microbial community. In some cases, measuring the microbial diversity of a microbial community may comprise measuring the diversity index of the microbial community. In some cases, a diversity index may comprise alpha-diversity, a beta-diversity, or a combination thereof. In some cases, a diversity index may comprise alpha-diversity. In some cases, a diversity index may comprise beta-diversity. In some cases, a diversity index may comprise alpha-diversity and beta-diversity.

In some instances, measuring the alpha-diversity of a microbial community in a subject may comprise measuring the average species diversity of the microbial community in the subject. In some cases, measuring the alpha-diversity of a microbial community in a subject may comprise measuring the average species diversity of the microbial community in the gastrointestinal tract of the subject. In some cases, an alpha-diversity of a microbial community may comprise Shannon, Inverse Simpson, Simpson, Gini, Observed Operational taxonomic units (OTUs), Faith's, Chao1, or a combination thereof. In some cases, an alpha-diversity of a microbial community may comprise Shannon. In some cases, an alpha-diversity of a microbial community may comprise Inverse Simpson. In some cases, an alpha-diversity of a microbial community may comprise Simpson. In some cases, an alpha-diversity of a microbial community may comprise Gini. In some cases, an alpha-diversity of a microbial community may comprise OTUs. In some cases, an alpha-diversity of a microbial community may comprise Observed amplicon sequence variant (ASVs). In some cases, an alpha-diversity of a microbial community may comprise Faith's. In some cases, an alpha-diversity of a microbial community may comprise Chao1.

In some cases, Shannon (H) may be calculated by the following formula:

H=Σ[(pᵢ)×ln(pᵢ)]

where pᵢ is proportion of total microbial community represented by species i.

In some cases, Simpson (D) may be calculated by the following formula:

D=Σ(n/N²), or

D=Σn(n−1)/N(N−1)

where n is the total number of organisms of one species in a microbial community, and B is the total number of organisms of all species in the microbial community.

In some cases, Gini may be calculated by 1-D. In some cases, Inverse Simpson may be calculated by 1/D.

In some cases, observed OTUs may comprise the number of sequences that are observed for each taxonomic unit in a microbial community.

In some cases, Chao1 (Sₑₛₜ) may be calculated by the following formula:

Sₑₛₜ=S_{obs}+F1²/2F2

where S_{obs} is the number of species in the microbial community, F1 is the number of species with only a single occurrence in the microbial community, and F2 is the number of species with exactly two occurrences in the microbial community. In some cases, methods to calculate observed OTUs may also be used to calculate observed ASVs.

In some instances, Faith's (or Faith's phylogenetic diversity) may comprise the sum of the lengths of all the branches on a phylogenetic tree of a microbial community that span the members of the microbial community.

In some instances, measuring the beta-diversity of a microbial community in a subject may comprise measuring the change in the diversity of species of the microbial community in the subject across various measurements or time points. In some cases, measuring the beta-diversity of a microbial community in a subject may comprise measuring the change in the diversity of species of the microbial community in the gastrointestinal tract of the subject across various measurements or time points. In some cases, beta-diversity may comprise Jaccard distance, unweighted UniFrac distance, weighted UniFrac distance, Bray-Curtis distance, or a combination thereof. In some cases, beta-diversity may comprise Jaccard distance. In some cases, beta-diversity may comprise unweighted UniFrac distance. In some cases, beta-diversity may comprise weighted UniFrac distance. In some cases, beta-diversity may comprise Bray-Curtis distance.

In some cases, Jaccard distance (J_{d}) may be calculated by the following formula:

J_{d}=1−(|A∩B|/|A∪B|)

where A and B represent two different measurements.

In some cases, unweighted UniFrac distance (u) may be calculated by the following formula:

u=sum of unshared branch lengths/sum of total branch lengths

where all taxa found in a pair of measurements of a microbial community are placed on a phylogenetic tree, and a branch leading to taxa from only one measurement of the microbial community is an unshared branch.

In some cases, weighted UniFrac distance (W) may be calculated by the following formula:

W=NΣi=1Lᵢ|Aᵢ/A_{T}−Bᵢ/B_{T}|/NΣi=1Lⱼ

where N is the number of nodes in a phylogenetic tree, S is the number of taxas represented by the tree, lᵢ is the branch length between node i and its ancestor, Lⱼ is the total branch length from the root to the tip of the tree for taxa j, A and B are two measurements of a microbial community, Aᵢ and Bᵢ are the number of taxa from measurements A and B that descend from the node, and A_{T} and B_{T} are the total number of taxa from measurements A and B.

In some cases, Bray-Curtis distance (BC) may be calculated by the following formula:

BC_{AB}=1−2C_{AB}/S_{A}+S_{B}

where C_{AB} is the sum of the lesser species that exists in both measurements A and B, S_{A} and S_{B} are the sum of total number of species in measurement A and measurement B, respectively.

In some instances, assaying a microbial community of a subject may comprise repeated measures of the microbial community. In some cases, assaying a microbial community may comprise making measurements of the microbial community of the subject multiple times. In some cases, assaying a microbial community may comprise making measurements of the microbial community of the subject at least 2 times, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, at least 31 times, at least 32 times, at least 33 times, at least 34 times, at least 35 times, at least 36 times, at least 37 times, at least 38 times, at least 39 times, at least 40 times, at least 41 times, at least 42 times, at least 43 times, at least 44 times, at least 45 times, at least 46 times, at least 47 times, at least 48 times, at least 49 times, or at least 50 times. In some cases, assaying a microbial community may comprise making measurements of the microbial community of the subject from about 2 to about 3 times, from about 2 to about 4 times, from about 2 to about 5 times, from about 2 to about 6 times, from about 2 to about 7 times, from about 2 to about 8 times, from about 2 to about 9 times, from about 2 to about 10 times, from about 2 to about 20 times, from about 2 to about 30 times, from about 2 to about 40 times, or from about 2 to about 50 times. In some cases, the time between two measurements of the microbial community of the subject may be at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, or at least about 5 years. In some cases, the times between measurements in a series of measurements in a repeat measure may be the same. In some cases, the times between measurements in a series of measurements in a repeat measure may be different.

In some instances, assaying a microbial community of a subject may comprise making measurements of the microbial community prior to the subject is administered with a pharmaceutical composition. In some instances, assaying a microbial community of a subject may comprise making measurements of the microbial community during administration of the pharmaceutical composition. In some instances, assaying a microbial community of a subject may comprise making measurements of the microbial community subsequent to the administration of the pharmaceutical composition. In some instances, assaying a microbial community of a subject may comprise making measurements of the microbial community before, during, or subsequent to the administration of the pharmaceutical composition.

In some instances, assaying a microbial community of a subject may comprise making measurements of the microbial community at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year prior to the subject is administered with a pharmaceutical composition.

In some instances, assaying a microbial community of a subject may comprise making measurements of the microbial community at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year subsequent to the subject is administered with a pharmaceutical composition.

In some instances, assaying a microbial community of a subject may comprise comparing at least two measurements of the microbial community of the subject. The measurement being compared, in some cases, may comprise any measurements described in this disclosure. For example, in some cases, the measurement being compared may comprise a microbial diversity index. In some cases, the measurements being compared may comprise alpha-diversity. In some cases, the alpha-diversity being compared may comprise Shannon, Inverse Simpson, Simpson, Gini, Observed Operational taxonomic units (OTUs), Faith's, Chao1, or a combination thereof. In some cases, the measurements being compared may comprise beta-diversity. In some cases, the beta-diversity being compared may comprise Jaccard distance, unweighted UniFrac distance, weighted UniFrac distance, Bray-Curtis distance, or a combination thereof. In some cases, beta-diversity being compared may comprise Jaccard distance. In some cases, beta-diversity being compared may comprise unweighted UniFrac distance. In some cases, beta-diversity being compared may comprise weighted UniFrac distance. In some cases, beta-diversity being compared may comprise Bray-Curtis distance.

In some instances, assaying a microbial community of a subject may comprise testing the difference between at least two measurements of the microbial community of the subject. In some cases, testing the difference may comprise testing the difference is a statistically significant. In some cases, testing the difference between at least two measurements may comprise a model. In some cases, a model may comprise a linear mixed effects (LME) model. In some cases, a model may comprise a linear regression model. In other cases, a model may comprise an aggregation model.

In some instances, an LME model may test the relationship between the measurements (i.e., response variable) and one or at least one independent variables (e.g., variables of the pharmaceutical composition treatment such as doses or treatment time; or variables of the subject being treated-such as the age, sex, ethnicity, diet, weight, or other biological or physiological variables of the subject. In some cases, an LME model may incorporate the repeated measures sampling of a subject or subjects to test the difference between the measurements.

In some instances, assaying a microbial community in a subject may identify an enrichment or a depletion of at least one bacterial species in the microbial community. In some cases, assaying a microbial community in a subject relative to a control microbial community may identify an enrichment or a depletion of at least one bacterial species in the microbial community. In some cases, assaying a microbial community in a subject may identify an enrichment of at least one bacterial species in the microbial community. In some cases, assaying a microbial community in a subject relative to a control microbial community may identify an enrichment of at least one bacterial species in the microbial community. In some cases, assaying a microbial community in a subject may identify a depletion of at least one bacterial species in the microbial community. In some cases, assaying a microbial community in a subject relative to a control microbial community may identify a depletion of at least one bacterial species in the microbial community.

In some instances, assaying a microbial community in a subject may identify an enrichment or a depletion of at least about 1 bacterial species, at least about 2 bacterial species, at least about 3 bacterial species, at least about 4 bacterial species, at least about 5 bacterial species, at least about 6 bacterial species, at least about 7 bacterial species, at least about 8 bacterial species, at least about 9 bacterial species, at least about 10 bacterial species, at least about 11 bacterial species, at least about 12 bacterial species, at least about 13 bacterial species, at least about 14 bacterial species, at least about 15 bacterial species, at least about 16 bacterial species, at least about 17 bacterial species, at least about 18 bacterial species, at least about 19 bacterial species, at least about 20 bacterial species, at least about 30 bacterial species, at least about 40 bacterial species, at least about 50 bacterial species, at least about 60 bacterial species, at least about 70 bacterial species, at least about 80 bacterial species, at least about 90 bacterial species, at least about 100 bacterial species, at least about 200 bacterial species, at least about 500 bacterial species, at least about 1000 bacterial species, at least about 2000 bacterial species, at least about 1×10\hat{ }4 bacterial species, at least about 1×10\hat{ }5 bacterial species, at least about 1×10\hat{ }6 bacterial species, at least about 1×10\hat{ }7 bacterial species, at least about 1×10\hat{ }8 bacterial species, at least about 1×10\hat{ }9 bacterial species, at least about 1×10\hat{ }10 bacterial species in the microbial community. In some cases, assaying a microbial community in a subject may identify an enrichment or a depletion of at least about 1 bacterial species. In some cases, assaying a microbial community in a subject may identify an enrichment or a depletion of at least about 2 bacterial species. In some cases, assaying a microbial community in a subject may identify an enrichment or a depletion of at least about 3 bacterial species. In some cases, assaying a microbial community in a subject may identify an enrichment or a depletion of at least about 4 bacterial species. In some cases, assaying a microbial community in a subject may identify an enrichment or a depletion of at least about 5 bacterial species. In some cases, assaying a microbial community in a subject may identify an enrichment or a depletion of at least about 6 bacterial species. In some cases, assaying a microbial community in a subject may identify an enrichment or a depletion of at least about 7 bacterial species. In some cases, assaying a microbial community in a subject may identify an enrichment or a depletion of at least about 8 bacterial species. In some cases, assaying a microbial community in a subject may identify an enrichment or a depletion of at least about 9 bacterial species. In some cases, assaying a microbial community in a subject may identify an enrichment or a depletion of at least about 10 bacterial species. In some cases, assaying a microbial community in a subject may identify an enrichment or a depletion of at least about 11 bacterial species. In some cases, assaying a microbial community in a subject may identify an enrichment or a depletion of at least about 12 bacterial species. In some cases, assaying a microbial community in a subject may identify an enrichment or a depletion of at least about 13 bacterial species. In some cases, assaying a microbial community in a subject may identify an enrichment or a depletion of at least about 14 bacterial species. In some cases, assaying a microbial community in a subject may identify an enrichment or a depletion of at least about 15 bacterial species. In some cases, assaying a microbial community in a subject may identify an enrichment or a depletion of at least about 16 bacterial species. In some cases, assaying a microbial community in a subject may identify an enrichment or a depletion of at least about 17 bacterial species. In some cases, assaying a microbial community in a subject may identify an enrichment or a depletion of at least about 18 bacterial species. In some cases, assaying a microbial community in a subject may identify an enrichment or a depletion of at least about 19 bacterial species. In some cases, assaying a microbial community in a subject may identify an enrichment or a depletion of at least about 20 bacterial species.

In some instances, assaying a microbial community in a subject may identify an enrichment of at least about 1 bacterial species, at least about 2 bacterial species, at least about 3 bacterial species, at least about 4 bacterial species, at least about 5 bacterial species, at least about 6 bacterial species, at least about 7 bacterial species, at least about 8 bacterial species, at least about 9 bacterial species, at least about 10 bacterial species, at least about 11 bacterial species, at least about 12 bacterial species, at least about 13 bacterial species, at least about 14 bacterial species, at least about 15 bacterial species, at least about 16 bacterial species, at least about 17 bacterial species, at least about 18 bacterial species, at least about 19 bacterial species, at least about 20 bacterial species, at least about 30 bacterial species, at least about 40 bacterial species, at least about 50 bacterial species, at least about 60 bacterial species, at least about 70 bacterial species, at least about 80 bacterial species, at least about 90 bacterial species, at least about 100 bacterial species, at least about 200 bacterial species, at least about 500 bacterial species, at least about 1000 bacterial species, at least about 2000 bacterial species, at least about 1×10\hat{ }4 bacterial species, at least about 1×10\hat{ }5 bacterial species, at least about 1×10\hat{ }6 bacterial species, at least about 1×10\hat{ }7 bacterial species, at least about 1×10\hat{ }8 bacterial species, at least about 1×10\hat{ }9 bacterial species, at least about 1×10\hat{ }10 bacterial species in the microbial community. In some cases, assaying a microbial community in a subject may identify an enrichment of at least about 1 bacterial species. In some cases, assaying a microbial community in a subject may identify an enrichment of at least about 2 bacterial species. In some cases, assaying a microbial community in a subject may identify an enrichment of at least about 3 bacterial species. In some cases, assaying a microbial community in a subject may identify an enrichment of at least about 4 bacterial species. In some cases, assaying a microbial community in a subject may identify an enrichment of at least about 5 bacterial species. In some cases, assaying a microbial community in a subject may identify an enrichment of at least about 6 bacterial species. In some cases, assaying a microbial community in a subject may identify an enrichment of at least about 7 bacterial species. In some cases, assaying a microbial community in a subject may identify an enrichment of at least about 8 bacterial species. In some cases, assaying a microbial community in a subject may identify an enrichment of at least about 9 bacterial species. In some cases, assaying a microbial community in a subject may identify an enrichment of at least about 10 bacterial species. In some cases, assaying a microbial community in a subject may identify an enrichment of at least about 11 bacterial species. In some cases, assaying a microbial community in a subject may identify an enrichment of at least about 12 bacterial species. In some cases, assaying a microbial community in a subject may identify an enrichment of at least about 13 bacterial species. In some cases, assaying a microbial community in a subject may identify an enrichment of at least about 14 bacterial species. In some cases, assaying a microbial community in a subject may identify an enrichment of at least about 15 bacterial species. In some cases, assaying a microbial community in a subject may identify an enrichment of at least about 16 bacterial species. In some cases, assaying a microbial community in a subject may identify an enrichment of at least about 17 bacterial species. In some cases, assaying a microbial community in a subject may identify an enrichment of at least about 18 bacterial species. In some cases, assaying a microbial community in a subject may identify an enrichment of at least about 19 bacterial species. In some cases, assaying a microbial community in a subject may identify an enrichment of at least about 20 bacterial species.

In some instances, assaying a microbial community in a subject may identify a depletion of at least about 1 bacterial species, at least about 2 bacterial species, at least about 3 bacterial species, at least about 4 bacterial species, at least about 5 bacterial species, at least about 6 bacterial species, at least about 7 bacterial species, at least about 8 bacterial species, at least about 9 bacterial species, at least about 10 bacterial species, at least about 11 bacterial species, at least about 12 bacterial species, at least about 13 bacterial species, at least about 14 bacterial species, at least about 15 bacterial species, at least about 16 bacterial species, at least about 17 bacterial species, at least about 18 bacterial species, at least about 19 bacterial species, at least about 20 bacterial species, at least about 30 bacterial species, at least about 40 bacterial species, at least about 50 bacterial species, at least about 60 bacterial species, at least about 70 bacterial species, at least about 80 bacterial species, at least about 90 bacterial species, at least about 100 bacterial species, at least about 200 bacterial species, at least about 500 bacterial species, at least about 1000 bacterial species, at least about 2000 bacterial species, at least about 1×10\hat{ }4 bacterial species, at least about 1×10\hat{ }5 bacterial species, at least about 1×10\hat{ }6 bacterial species, at least about 1×10\hat{ }7 bacterial species, at least about 1×10\hat{ }8 bacterial species, at least about 1×10\hat{ }9 bacterial species, at least about 1×10\hat{ }10 bacterial species in the microbial community. In some cases, assaying a microbial community in a subject may identify a depletion of at least about 1 bacterial species. In some cases, assaying a microbial community in a subject may identify a depletion of at least about 2 bacterial species. In some cases, assaying a microbial community in a subject may identify a depletion of at least about 3 bacterial species. In some cases, assaying a microbial community in a subject may identify a depletion of at least about 4 bacterial species. In some cases, assaying a microbial community in a subject may identify a depletion of at least about 5 bacterial species. In some cases, assaying a microbial community in a subject may identify a depletion of at least about 6 bacterial species. In some cases, assaying a microbial community in a subject may identify a depletion of at least about 7 bacterial species. In some cases, assaying a microbial community in a subject may identify a depletion of at least about 8 bacterial species. In some cases, assaying a microbial community in a subject may identify a depletion of at least about 9 bacterial species. In some cases, assaying a microbial community in a subject may identify a depletion of at least about 10 bacterial species. In some cases, assaying a microbial community in a subject may identify a depletion of at least about 11 bacterial species. In some cases, assaying a microbial community in a subject may identify a depletion of at least about 12 bacterial species. In some cases, assaying a microbial community in a subject may identify a depletion of at least about 13 bacterial species. In some cases, assaying a microbial community in a subject may identify a depletion of at least about 14 bacterial species. In some cases, assaying a microbial community in a subject may identify a depletion of at least about 15 bacterial species. In some cases, assaying a microbial community in a subject may identify a depletion of at least about 16 bacterial species. In some cases, assaying a microbial community in a subject may identify a depletion of at least about 17 bacterial species. In some cases, assaying a microbial community in a subject may identify a depletion of at least about 18 bacterial species. In some cases, assaying a microbial community in a subject may identify a depletion of at least about 19 bacterial species. In some cases, assaying a microbial community in a subject may identify a depletion of at least about 20 bacterial species.

In some instances, an enrichment of a bacterial species may comprise an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase in the amount of the bacterial species relative to the amount of the bacterial species before the enrichment. In some instances, a depletion of a bacterial species may comprise an at least about 1% decrease, an at least about 5% decrease, an at least about 10% decrease, an at least about 15% decrease, an at least about 20% decrease, an at least about 25% decrease, an at least about 30% decrease, an at least about 35% decrease, an at least about 40% decrease, an at least about 45% decrease, an at least about 50% decrease, an at least about 55% decrease, an at least about 60% decrease, an at least about 65% decrease, an at least about 70% decrease, an at least about 75% decrease, an at least about 80% decrease, an at least about 85% decrease, an at least about 90% decrease, an at least about 91% decrease, an at least about 92% decrease, an at least about 93% decrease, an at least about 94% decrease, an at least about 95% decrease, an at least about 96% decrease, an at least about 97% decrease, an at least about 98% decrease, an at least about 99% decrease, or a 100% decrease in the amount of the bacterial species relative to the amount of the bacterial species before the depletion.

In some cases the amount of the bacterial species may be represented by the number of the bacterial species; the number of the microorganisms of the bacterial species; the amount of the nucleic acid representing the number of the bacterial species or the number of the microorganisms of the bacterial species; the dry weight of nucleic acids representing the number of the bacterial species or the number of the microorganisms of the bacterial species; the dry weight of the bacterial species or the microorganisms of the bacterial species; or a combination thereof.

In some instances, assaying a microbial community in a subject may identify an enrichment or a depletion of at least about 1 bacterial strain, at least about 2 bacterial strains, at least about 3 bacterial strains, at least about 4 bacterial strains, at least about 5 bacterial strains, at least about 6 bacterial strains, at least about 7 bacterial strains, at least about 8 bacterial strains, at least about 9 bacterial strains, at least about 10 bacterial strains, at least about 11 bacterial strains, at least about 12 bacterial strains, at least about 13 bacterial strains, at least about 14 bacterial strains, at least about 15 bacterial strains, at least about 16 bacterial strains, at least about 17 bacterial strains, at least about 18 bacterial strains, at least about 19 bacterial strains, at least about 20 bacterial strains, at least about 30 bacterial strains, at least about 40 bacterial strains, at least about 50 bacterial strains, at least about 60 bacterial strains, at least about 70 bacterial strains, at least about 80 bacterial strains, at least about 90 bacterial strains, at least about 100 bacterial strains, at least about 200 bacterial strains, at least about 500 bacterial strains, at least about 1000 bacterial strains, at least about 2000 bacterial strains, at least about 1×10\hat{ }4 bacterial strains, at least about 1×10\hat{ }5 bacterial strains, at least about 1×10\hat{ }6 bacterial strains, at least about 1×10\hat{ }7 bacterial strains, at least about 1×10\hat{ }8 bacterial strains, at least about 1×10\hat{ }9 bacterial strains, at least about 1×10\hat{ }10 bacterial strains in the microbial community. In some cases, assaying a microbial community in a subject may identify an enrichment or a depletion of at least about 1 bacterial strain. In some cases, assaying a microbial community in a subject may identify an enrichment or a depletion of at least about 2 bacterial strains. In some cases, assaying a microbial community in a subject may identify an enrichment or a depletion of at least about 3 bacterial strains. In some cases, assaying a microbial community in a subject may identify an enrichment or a depletion of at least about 4 bacterial strains. In some cases, assaying a microbial community in a subject may identify an enrichment or a depletion of at least about 5 bacterial strains. In some cases, assaying a microbial community in a subject may identify an enrichment or a depletion of at least about 6 bacterial strains. In some cases, assaying a microbial community in a subject may identify an enrichment or a depletion of at least about 7 bacterial strains. In some cases, assaying a microbial community in a subject may identify an enrichment or a depletion of at least about 8 bacterial strains. In some cases, assaying a microbial community in a subject may identify an enrichment or a depletion of at least about 9 bacterial strains. In some cases, assaying a microbial community in a subject may identify an enrichment or a depletion of at least about 10 bacterial strains. In some cases, assaying a microbial community in a subject may identify an enrichment or a depletion of at least about 11 bacterial strains. In some cases, assaying a microbial community in a subject may identify an enrichment or a depletion of at least about 12 bacterial strains. In some cases, assaying a microbial community in a subject may identify an enrichment or a depletion of at least about 13 bacterial strains. In some cases, assaying a microbial community in a subject may identify an enrichment or a depletion of at least about 14 bacterial strains. In some cases, assaying a microbial community in a subject may identify an enrichment or a depletion of at least about 15 bacterial strains. In some cases, assaying a microbial community in a subject may identify an enrichment or a depletion of at least about 16 bacterial strains. In some cases, assaying a microbial community in a subject may identify an enrichment or a depletion of at least about 17 bacterial strains. In some cases, assaying a microbial community in a subject may identify an enrichment or a depletion of at least about 18 bacterial strains. In some cases, assaying a microbial community in a subject may identify an enrichment or a depletion of at least about 19 bacterial strains. In some cases, assaying a microbial community in a subject may identify an enrichment or a depletion of at least about 20 bacterial strains.

In some instances, assaying a microbial community in a subject may identify an enrichment of at least about 1 bacterial strain, at least about 2 bacterial strains, at least about 3 bacterial strains, at least about 4 bacterial strains, at least about 5 bacterial strains, at least about 6 bacterial strains, at least about 7 bacterial strains, at least about 8 bacterial strains, at least about 9 bacterial strains, at least about 10 bacterial strains, at least about 11 bacterial strains, at least about 12 bacterial strains, at least about 13 bacterial strains, at least about 14 bacterial strains, at least about 15 bacterial strains, at least about 16 bacterial strains, at least about 17 bacterial strains, at least about 18 bacterial strains, at least about 19 bacterial strains, at least about 20 bacterial strains, at least about 30 bacterial strains, at least about 40 bacterial strains, at least about 50 bacterial strains, at least about 60 bacterial strains, at least about 70 bacterial strains, at least about 80 bacterial strains, at least about 90 bacterial strains, at least about 100 bacterial strains, at least about 200 bacterial strains, at least about 500 bacterial strains, at least about 1000 bacterial strains, at least about 2000 bacterial strains, at least about 1×10\hat{ }4 bacterial strains, at least about 1×10\hat{ }5 bacterial strains, at least about 1×10\hat{ }6 bacterial strains, at least about 1×10\hat{ }7 bacterial strains, at least about 1×10\hat{ }8 bacterial strains, at least about 1×10\hat{ }9 bacterial strains, at least about 1×10\hat{ }10 bacterial strains in the microbial community. In some cases, assaying a microbial community in a subject may identify an enrichment of at least about 1 bacterial strain. In some cases, assaying a microbial community in a subject may identify an enrichment of at least about 2 bacterial strains. In some cases, assaying a microbial community in a subject may identify an enrichment of at least about 3 bacterial strains. In some cases, assaying a microbial community in a subject may identify an enrichment of at least about 4 bacterial strains. In some cases, assaying a microbial community in a subject may identify an enrichment of at least about 5 bacterial strains. In some cases, assaying a microbial community in a subject may identify an enrichment of at least about 6 bacterial strains. In some cases, assaying a microbial community in a subject may identify an enrichment of at least about 7 bacterial strains. In some cases, assaying a microbial community in a subject may identify an enrichment of at least about 8 bacterial strains. In some cases, assaying a microbial community in a subject may identify an enrichment of at least about 9 bacterial strains. In some cases, assaying a microbial community in a subject may identify an enrichment of at least about 10 bacterial strains. In some cases, assaying a microbial community in a subject may identify an enrichment of at least about 11 bacterial strains. In some cases, assaying a microbial community in a subject may identify an enrichment of at least about 12 bacterial strains. In some cases, assaying a microbial community in a subject may identify an enrichment of at least about 13 bacterial strains. In some cases, assaying a microbial community in a subject may identify an enrichment of at least about 14 bacterial strains. In some cases, assaying a microbial community in a subject may identify an enrichment of at least about 15 bacterial strains. In some cases, assaying a microbial community in a subject may identify an enrichment of at least about 16 bacterial strains. In some cases, assaying a microbial community in a subject may identify an enrichment of at least about 17 bacterial strains. In some cases, assaying a microbial community in a subject may identify an enrichment of at least about 18 bacterial strains. In some cases, assaying a microbial community in a subject may identify an enrichment of at least about 19 bacterial strains. In some cases, assaying a microbial community in a subject may identify an enrichment of at least about 20 bacterial strains.

In some instances, assaying a microbial community in a subject may identify a depletion of at least about 1 bacterial strain, at least about 2 bacterial strains, at least about 3 bacterial strains, at least about 4 bacterial strains, at least about 5 bacterial strains, at least about 6 bacterial strains, at least about 7 bacterial strains, at least about 8 bacterial strains, at least about 9 bacterial strains, at least about 10 bacterial strains, at least about 11 bacterial strains, at least about 12 bacterial strains, at least about 13 bacterial strains, at least about 14 bacterial strains, at least about 15 bacterial strains, at least about 16 bacterial strains, at least about 17 bacterial strains, at least about 18 bacterial strains, at least about 19 bacterial strains, at least about 20 bacterial strains, at least about 30 bacterial strains, at least about 40 bacterial strains, at least about 50 bacterial strains, at least about 60 bacterial strains, at least about 70 bacterial strains, at least about 80 bacterial strains, at least about 90 bacterial strains, at least about 100 bacterial strains, at least about 200 bacterial strains, at least about 500 bacterial strains, at least about 1000 bacterial strains, at least about 2000 bacterial strains, at least about 1×10\hat{ }4 bacterial strains, at least about 1×10\hat{ }5 bacterial strains, at least about 1×10\hat{ }6 bacterial strains, at least about 1×10\hat{ }7 bacterial strains, at least about 1×10\hat{ }8 bacterial strains, at least about 1×10\hat{ }9 bacterial strains, at least about 1×10\hat{ }10 bacterial strains in the microbial community. In some cases, assaying a microbial community in a subject may identify a depletion of at least about 1 bacterial strain. In some cases, assaying a microbial community in a subject may identify a depletion of at least about 2 bacterial strains. In some cases, assaying a microbial community in a subject may identify a depletion of at least about 3 bacterial strains. In some cases, assaying a microbial community in a subject may identify a depletion of at least about 4 bacterial strains. In some cases, assaying a microbial community in a subject may identify a depletion of at least about 5 bacterial strains. In some cases, assaying a microbial community in a subject may identify a depletion of at least about 6 bacterial strains. In some cases, assaying a microbial community in a subject may identify a depletion of at least about 7 bacterial strains. In some cases, assaying a microbial community in a subject may identify a depletion of at least about 8 bacterial strains. In some cases, assaying a microbial community in a subject may identify a depletion of at least about 9 bacterial strains. In some cases, assaying a microbial community in a subject may identify a depletion of at least about 10 bacterial strains. In some cases, assaying a microbial community in a subject may identify a depletion of at least about 11 bacterial strains. In some cases, assaying a microbial community in a subject may identify a depletion of at least about 12 bacterial strains. In some cases, assaying a microbial community in a subject may identify a depletion of at least about 13 bacterial strains. In some cases, assaying a microbial community in a subject may identify a depletion of at least about 14 bacterial strains. In some cases, assaying a microbial community in a subject may identify a depletion of at least about 15 bacterial strains. In some cases, assaying a microbial community in a subject may identify a depletion of at least about 16 bacterial strains. In some cases, assaying a microbial community in a subject may identify a depletion of at least about 17 bacterial strains. In some cases, assaying a microbial community in a subject may identify a depletion of at least about 18 bacterial strains. In some cases, assaying a microbial community in a subject may identify a depletion of at least about 19 bacterial strains. In some cases, assaying a microbial community in a subject may identify a depletion of at least about 20 bacterial strains.

In some instances, an enrichment of a bacterial strain may comprise an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase in the amount of the bacterial strains relative to the amount of the bacterial strains before the enrichment. In some instances, a depletion of a bacterial strain may comprise an at least about 1% decrease, an at least about 5% decrease, an at least about 10% decrease, an at least about 15% decrease, an at least about 20% decrease, an at least about 25% decrease, an at least about 30% decrease, an at least about 35% decrease, an at least about 40% decrease, an at least about 45% decrease, an at least about 50% decrease, an at least about 55% decrease, an at least about 60% decrease, an at least about 65% decrease, an at least about 70% decrease, an at least about 75% decrease, an at least about 80% decrease, an at least about 85% decrease, an at least about 90% decrease, an at least about 91% decrease, an at least about 92% decrease, an at least about 93% decrease, an at least about 94% decrease, an at least about 95% decrease, an at least about 96% decrease, an at least about 97% decrease, an at least about 98% decrease, an at least about 99% decrease, or a 100% decrease in the amount of the bacterial strains relative to the amount of the bacterial strains before the depletion.

In some cases the amount of the bacterial strains may be represented by the number of the bacterial strains; the number of the microorganisms of the bacterial strains; the amount of the nucleic acid representing the number of the bacterial strains or the number of the microorganisms of the bacterial strains; the dry weight of nucleic acids representing the number of the bacterial strains or the number of the microorganisms of the bacterial strains; the dry weight of the bacterial strains or the microorganisms of the bacterial strains; or a combination thereof.

In some instances, assaying a microbial community in a subject may identify an enrichment or a depletion of at least about 1 bacterial family, genus, order, class, or phylum; at least about 2 bacterial families, genera, orders, classes, or phyla; at least about 3 bacterial families, genera, orders, classes, or phyla; at least about 4 bacterial families, genera, orders, classes, or phyla; at least about 5 bacterial families, genera, orders, classes, or phyla; at least about 6 bacterial families, genera, orders, classes, or phyla; at least about 7 bacterial families, genera, orders, classes, or phyla; at least about 8 bacterial families, genera, orders, classes, or phyla; at least about 9 bacterial families, genera, orders, classes, or phyla; at least about 10 bacterial families, genera, orders, classes, or phyla; at least about 11 bacterial families, genera, orders, classes, or phyla; at least about 12 bacterial families, genera, orders, classes, or phyla; at least about 13 bacterial families, genera, orders, classes, or phyla; at least about 14 bacterial families, genera, orders, classes, or phyla; at least about 15 bacterial families, genera, orders, classes, or phyla; at least about 16 bacterial families, genera, orders, classes, or phyla; at least about 17 bacterial families, genera, orders, classes, or phyla; at least about 18 bacterial families, genera, orders, classes, or phyla; at least about 19 bacterial families, genera, orders, classes, or phyla; at least about 20 bacterial families, genera, orders, classes, or phyla; at least about 30 bacterial families, genera, orders, classes, or phyla; at least about 40 bacterial families, genera, orders, classes, or phyla; at least about 50 bacterial families, genera, orders, classes, or phyla; at least about 60 bacterial families, genera, orders, classes, or phyla; at least about 70 bacterial families, genera, orders, classes, or phyla; at least about 80 bacterial families, genera, orders, classes, or phyla; at least about 90 bacterial families, genera, orders, classes, or phyla; at least about 100 bacterial families, genera, orders, classes, or phyla; at least about 200 bacterial families, genera, orders, classes, or phyla; at least about 500 bacterial families, genera, orders, classes, or phyla; at least about 1000 bacterial families, genera, orders, classes, or phyla; at least about 2000 bacterial families, genera, orders, classes, or phyla; at least about 1×10\hat{ }4 bacterial families, genera, orders, classes, or phyla; at least about 1×10\hat{ }5 bacterial families, genera, orders, classes, or phyla; at least about 1×10\hat{ }6 bacterial families, genera, orders, classes, or phyla; at least about 1×10\hat{ }7 bacterial families, genera, orders, classes, or phyla; at least about 1×10\hat{ }8 bacterial families, genera, orders, classes, or phyla; at least about 1×10\hat{ }9 bacterial families, genera, orders, classes, or phyla; at least about 1×10\hat{ }10 bacterial strains in the microbial community. In some cases, assaying a microbial community in a subject may identify an enrichment or a depletion of at least about 1 bacterial strain. In some cases, assaying a microbial community in a subject may identify an enrichment or a depletion of at least about 2 bacterial strains. In some cases, assaying a microbial community in a subject may identify an enrichment or a depletion of at least about 3 bacterial strains. In some cases, assaying a microbial community in a subject may identify an enrichment or a depletion of at least about 4 bacterial strains. In some cases, assaying a microbial community in a subject may identify an enrichment or a depletion of at least about 5 bacterial strains. In some cases, assaying a microbial community in a subject may identify an enrichment or a depletion of at least about 6 bacterial strains. In some cases, assaying a microbial community in a subject may identify an enrichment or a depletion of at least about 7 bacterial strains. In some cases, assaying a microbial community in a subject may identify an enrichment or a depletion of at least about 8 bacterial strains. In some cases, assaying a microbial community in a subject may identify an enrichment or a depletion of at least about 9 bacterial strains. In some cases, assaying a microbial community in a subject may identify an enrichment or a depletion of at least about 10 bacterial strains. In some cases, assaying a microbial community in a subject may identify an enrichment or a depletion of at least about 11 bacterial strains. In some cases, assaying a microbial community in a subject may identify an enrichment or a depletion of at least about 12 bacterial strains. In some cases, assaying a microbial community in a subject may identify an enrichment or a depletion of at least about 13 bacterial strains. In some cases, assaying a microbial community in a subject may identify an enrichment or a depletion of at least about 14 bacterial strains. In some cases, assaying a microbial community in a subject may identify an enrichment or a depletion of at least about 15 bacterial strains. In some cases, assaying a microbial community in a subject may identify an enrichment or a depletion of at least about 16 bacterial strains. In some cases, assaying a microbial community in a subject may identify an enrichment or a depletion of at least about 17 bacterial strains. In some cases, assaying a microbial community in a subject may identify an enrichment or a depletion of at least about 18 bacterial strains. In some cases, assaying a microbial community in a subject may identify an enrichment or a depletion of at least about 19 bacterial strains. In some cases, assaying a microbial community in a subject may identify an enrichment or a depletion of at least about 20 bacterial strains.

In some instances, assaying a microbial community in a subject may identify an enrichment of at least about 1 bacterial family, genus, order, class, or phylum; at least about 2 bacterial families, genera, orders, classes, or phyla; at least about 3 bacterial families, genera, orders, classes, or phyla; at least about 4 bacterial families, genera, orders, classes, or phyla; at least about 5 bacterial families, genera, orders, classes, or phyla; at least about 6 bacterial families, genera, orders, classes, or phyla; at least about 7 bacterial families, genera, orders, classes, or phyla; at least about 8 bacterial families, genera, orders, classes, or phyla; at least about 9 bacterial families, genera, orders, classes, or phyla; at least about 10 bacterial families, genera, orders, classes, or phyla; at least about 11 bacterial families, genera, orders, classes, or phyla; at least about 12 bacterial families, genera, orders, classes, or phyla; at least about 13 bacterial families, genera, orders, classes, or phyla; at least about 14 bacterial families, genera, orders, classes, or phyla; at least about 15 bacterial families, genera, orders, classes, or phyla; at least about 16 bacterial families, genera, orders, classes, or phyla; at least about 17 bacterial families, genera, orders, classes, or phyla; at least about 18 bacterial families, genera, orders, classes, or phyla; at least about 19 bacterial families, genera, orders, classes, or phyla; at least about 20 bacterial families, genera, orders, classes, or phyla; at least about 30 bacterial families, genera, orders, classes, or phyla; at least about 40 bacterial families, genera, orders, classes, or phyla; at least about 50 bacterial families, genera, orders, classes, or phyla; at least about 60 bacterial families, genera, orders, classes, or phyla; at least about 70 bacterial families, genera, orders, classes, or phyla; at least about 80 bacterial families, genera, orders, classes, or phyla; at least about 90 bacterial families, genera, orders, classes, or phyla; at least about 100 bacterial families, genera, orders, classes, or phyla; at least about 200 bacterial families, genera, orders, classes, or phyla; at least about 500 bacterial families, genera, orders, classes, or phyla; at least about 1000 bacterial families, genera, orders, classes, or phyla; at least about 2000 bacterial families, genera, orders, classes, or phyla; at least about 1×10\hat{ }4 bacterial families, genera, orders, classes, or phyla; at least about 1×10\hat{ }5 bacterial families, genera, orders, classes, or phyla; at least about 1×10\hat{ }6 bacterial families, genera, orders, classes, or phyla; at least about 1×10\hat{ }7 bacterial families, genera, orders, classes, or phyla; at least about 1×10\hat{ }8 bacterial families, genera, orders, classes, or phyla; at least about 1×10\hat{ }9 bacterial families, genera, orders, classes, or phyla; at least about 1×10\hat{ }10 bacterial strains in the microbial community. In some cases, assaying a microbial community in a subject may identify an enrichment of at least about 1 bacterial strain. In some cases, assaying a microbial community in a subject may identify an enrichment of at least about 2 bacterial strains. In some cases, assaying a microbial community in a subject may identify an enrichment of at least about 3 bacterial strains. In some cases, assaying a microbial community in a subject may identify an enrichment of at least about 4 bacterial strains. In some cases, assaying a microbial community in a subject may identify an enrichment of at least about 5 bacterial strains. In some cases, assaying a microbial community in a subject may identify an enrichment of at least about 6 bacterial strains. In some cases, assaying a microbial community in a subject may identify an enrichment of at least about 7 bacterial strains. In some cases, assaying a microbial community in a subject may identify an enrichment of at least about 8 bacterial strains. In some cases, assaying a microbial community in a subject may identify an enrichment of at least about 9 bacterial strains. In some cases, assaying a microbial community in a subject may identify an enrichment of at least about 10 bacterial strains. In some cases, assaying a microbial community in a subject may identify an enrichment of at least about 11 bacterial strains. In some cases, assaying a microbial community in a subject may identify an enrichment of at least about 12 bacterial strains. In some cases, assaying a microbial community in a subject may identify an enrichment of at least about 13 bacterial strains. In some cases, assaying a microbial community in a subject may identify an enrichment of at least about 14 bacterial strains. In some cases, assaying a microbial community in a subject may identify an enrichment of at least about 15 bacterial strains. In some cases, assaying a microbial community in a subject may identify an enrichment of at least about 16 bacterial strains. In some cases, assaying a microbial community in a subject may identify an enrichment of at least about 17 bacterial strains. In some cases, assaying a microbial community in a subject may identify an enrichment of at least about 18 bacterial strains. In some cases, assaying a microbial community in a subject may identify an enrichment of at least about 19 bacterial strains. In some cases, assaying a microbial community in a subject may identify an enrichment of at least about 20 bacterial strains.

In some instances, assaying a microbial community in a subject may identify a depletion of at least about 1 bacterial family, genus, order, class, or phylum; at least about 2 bacterial families, genera, orders, classes, or phyla; at least about 3 bacterial families, genera, orders, classes, or phyla; at least about 4 bacterial families, genera, orders, classes, or phyla; at least about 5 bacterial families, genera, orders, classes, or phyla; at least about 6 bacterial families, genera, orders, classes, or phyla; at least about 7 bacterial families, genera, orders, classes, or phyla; at least about 8 bacterial families, genera, orders, classes, or phyla; at least about 9 bacterial families, genera, orders, classes, or phyla; at least about 10 bacterial families, genera, orders, classes, or phyla; at least about 11 bacterial families, genera, orders, classes, or phyla; at least about 12 bacterial families, genera, orders, classes, or phyla; at least about 13 bacterial families, genera, orders, classes, or phyla; at least about 14 bacterial families, genera, orders, classes, or phyla; at least about 15 bacterial families, genera, orders, classes, or phyla; at least about 16 bacterial families, genera, orders, classes, or phyla; at least about 17 bacterial families, genera, orders, classes, or phyla; at least about 18 bacterial families, genera, orders, classes, or phyla; at least about 19 bacterial families, genera, orders, classes, or phyla; at least about 20 bacterial families, genera, orders, classes, or phyla; at least about 30 bacterial families, genera, orders, classes, or phyla; at least about 40 bacterial families, genera, orders, classes, or phyla; at least about 50 bacterial families, genera, orders, classes, or phyla; at least about 60 bacterial families, genera, orders, classes, or phyla; at least about 70 bacterial families, genera, orders, classes, or phyla; at least about 80 bacterial families, genera, orders, classes, or phyla; at least about 90 bacterial families, genera, orders, classes, or phyla; at least about 100 bacterial families, genera, orders, classes, or phyla; at least about 200 bacterial families, genera, orders, classes, or phyla; at least about 500 bacterial families, genera, orders, classes, or phyla; at least about 1000 bacterial families, genera, orders, classes, or phyla; at least about 2000 bacterial families, genera, orders, classes, or phyla; at least about 1×10\hat{ }4 bacterial families, genera, orders, classes, or phyla; at least about 1×10\hat{ }5 bacterial families, genera, orders, classes, or phyla; at least about 1×10\hat{ }6 bacterial families, genera, orders, classes, or phyla; at least about 1×10\hat{ }7 bacterial families, genera, orders, classes, or phyla; at least about 1×10\hat{ }8 bacterial families, genera, orders, classes, or phyla; at least about 1×10\hat{ }9 bacterial families, genera, orders, classes, or phyla; at least about 1×10\hat{ }10 bacterial strains in the microbial community.

In some cases, assaying a microbial community in a subject may identify a depletion of at least about 1 bacterial family, genus, order, class, or phylum. In some cases, assaying a microbial community in a subject may identify a depletion of at least about 2 bacterial families, genera, orders, classes, or phyla. In some cases, assaying a microbial community in a subject may identify a depletion of at least about 3 bacterial families, genera, orders, classes, or phyla. In some cases, assaying a microbial community in a subject may identify a depletion of at least about 4 bacterial families, genera, orders, classes, or phyla. In some cases, assaying a microbial community in a subject may identify a depletion of at least about 5 bacterial families, genera, orders, classes, or phyla. In some cases, assaying a microbial community in a subject may identify a depletion of at least about 6 bacterial families, genera, orders, classes, or phyla. In some cases, assaying a microbial community in a subject may identify a depletion of at least about 7 bacterial families, genera, orders, classes, or phyla. In some cases, assaying a microbial community in a subject may identify a depletion of at least about 8 bacterial families, genera, orders, classes, or phyla. In some cases, assaying a microbial community in a subject may identify a depletion of at least about 9 bacterial families, genera, orders, classes, or phyla. In some cases, assaying a microbial community in a subject may identify a depletion of at least about 10 bacterial families, genera, orders, classes, or phyla. In some cases, assaying a microbial community in a subject may identify a depletion of at least about 11 bacterial families, genera, orders, classes, or phyla. In some cases, assaying a microbial community in a subject may identify a depletion of at least about 12 bacterial families, genera, orders, classes, or phyla. In some cases, assaying a microbial community in a subject may identify a depletion of at least about 13 bacterial families, genera, orders, classes, or phyla. In some cases, assaying a microbial community in a subject may identify a depletion of at least about 14 bacterial families, genera, orders, classes, or phyla. In some cases, assaying a microbial community in a subject may identify a depletion of at least about 15 bacterial families, genera, orders, classes, or phyla. In some cases, assaying a microbial community in a subject may identify a depletion of at least about 16 bacterial families, genera, orders, classes, or phyla. In some cases, assaying a microbial community in a subject may identify a depletion of at least about 17 bacterial families, genera, orders, classes, or phyla. In some cases, assaying a microbial community in a subject may identify a depletion of at least about 18 bacterial families, genera, orders, classes, or phyla. In some cases, assaying a microbial community in a subject may identify a depletion of at least about 19 bacterial families, genera, orders, classes, or phyla. In some cases, assaying a microbial community in a subject may identify a depletion of at least about 20 bacterial families, genera, orders, classes, or phyla.

In some instances, an enrichment of a bacterial family, genus, order, class, or phylum may comprise an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase in the amount of the bacterial strains relative to the amount of the bacterial strains before the enrichment. In some instances, a depletion of a bacterial strain may comprise an at least about 1% decrease, an at least about 5% decrease, an at least about 10% decrease, an at least about 15% decrease, an at least about 20% decrease, an at least about 25% decrease, an at least about 30% decrease, an at least about 35% decrease, an at least about 40% decrease, an at least about 45% decrease, an at least about 50% decrease, an at least about 55% decrease, an at least about 60% decrease, an at least about 65% decrease, an at least about 70% decrease, an at least about 75% decrease, an at least about 80% decrease, an at least about 85% decrease, an at least about 90% decrease, an at least about 91% decrease, an at least about 92% decrease, an at least about 93% decrease, an at least about 94% decrease, an at least about 95% decrease, an at least about 96% decrease, an at least about 97% decrease, an at least about 98% decrease, an at least about 99% decrease, or a 100% decrease in the amount of the bacterial family, genus, order, class, or phylum relative to the amount of the bacterial family, genus, order, class, or phylum before the depletion.

In some cases, the amount of the bacterial family, genus, order, class, or phylum may be represented by the number of the bacterial families, genera, orders, classes, or phyla; the number of the microorganisms of the bacterial families, genera, orders, classes, or phyla; the amount of the nucleic acid representing the number of the bacterial strains or the number of the microorganisms of the bacterial families, genera, orders, classes, or phyla; the dry weight of nucleic acids representing the number of the bacterial strains or the number of the microorganisms of the bacterial families, genera, orders, classes, or phyla; the dry weight of the bacterial strains or the microorganisms of the bacterial families, genera, orders, classes, or phyla; or a combination thereof.

In some instances, a microorganism may be enriched or depleted in a microbial community of a subject subsequent to, during, or prior to the subject is administered with a pharmaceutical composition. In some cases, a microorganism may be enriched or depleted in a microbial community of a subject subsequent to the subject is administered with a pharmaceutical composition. In some cases, a microorganism may be enriched or depleted in a microbial community of a subject during when the subject is administered with a pharmaceutical composition. In some cases, a microorganism may be enriched or depleted in a microbial community of a subject prior to the subject is administered with a pharmaceutical composition. In some cases, a microorganism may be enriched or depleted in a microbial community of a subject in response to a pharmaceutical composition administered to a subject.

In some cases, a bacterial species may be enriched or depleted in a microbial community of a subject subsequent to, during, or prior to the subject is administered with a pharmaceutical composition. In some cases, a bacterial species may be enriched or depleted in a microbial community of a subject subsequent to the subject is administered with a pharmaceutical composition. In some cases, a bacterial species may be enriched or depleted in a microbial community of a subject during when the subject is administered with a pharmaceutical composition. In some cases, a bacterial species may be enriched or depleted in a microbial community of a subject prior to the subject is administered with a pharmaceutical composition. In some cases, a bacterial species may be enriched or depleted in a microbial community of a subject in response to a pharmaceutical composition administered to a subject.

In some cases, a bacterial strain may be enriched or depleted in a microbial community of a subject subsequent to, during, or prior to the subject is administered with a pharmaceutical composition. In some cases, a bacterial strain may be enriched or depleted in a microbial community of a subject subsequent to the subject is administered with a pharmaceutical composition. In some cases, a bacterial strain may be enriched or depleted in a microbial community of a subject during when the subject is administered with a pharmaceutical composition. In some cases, a bacterial strain may be enriched or depleted in a microbial community of a subject prior to the subject is administered with a pharmaceutical composition. In some cases, a bacterial strain may be enriched or depleted in a microbial community of a subject in response to a pharmaceutical composition administered to a subject.

In some cases, a bacterial family, genus, order, class, or phylum may be enriched or depleted in a microbial community of a subject subsequent to, during, or prior to the subject is administered with a pharmaceutical composition. In some cases, a bacterial family, genus, order, class, or phylum may be enriched or depleted in a microbial community of a subject subsequent to the subject is administered with a pharmaceutical composition. In some cases, a bacterial family, genus, order, class, or phylum may be enriched or depleted in a microbial community of a subject during when the subject is administered with a pharmaceutical composition. In some cases, a bacterial family, genus, order, class, or phylum may be enriched or depleted in a microbial community of a subject prior to the subject is administered with a pharmaceutical composition. In some cases, a bacterial family, genus, order, class, or phylum may be enriched or depleted in a microbial community of a subject in response to a pharmaceutical composition administered to a subject.

In some instances, a bacterial species that can synthesize short chain fatty acids (SCFAs) may be enriched in the microbial community. In some cases, a bacterial species may metabolize at least one species of SCFAs may be enriched in the microbial community. In some instances, a bacterial species that can synthesize short chain fatty acids SCFAs may be enriched in the microbial community. In some cases, a bacterial species may metabolize at least one species of SCFAs may be enriched in the microbial community. In some cases, a bacterial strain that can synthesize SCFAs may be enriched in the microbial community. In some cases, a bacterial strain may metabolize at least one strain of SCFAs may be enriched in the microbial community. In some instances, a bacterial strain that can synthesize short chain fatty acids SCFAs may be enriched in the microbial community. In some cases, a bacterial strain may metabolize at least one strain of SCFAs may be enriched in the microbial community. In some cases, a microorganism that can synthesize SCFAs may be enriched in the microbial community. In some cases, a microorganism may metabolize at least one species of SCFAs may be enriched in the microbial community. In some instances, a microorganism that can synthesize SCFAs may be enriched in the microbial community. In some cases, a microorganism may metabolize at least one species of SCFAs may be enriched in the microbial community.

In some instances, a bacterial strain associated with an inflammatory disease may be depleted in the microbial community. In some cases, a bacterial strain related to an inflammatory disease may be depleted in the microbial community. In some cases, a bacterial strain that can cause an inflammatory disease may be depleted in the microbial community. In some instances, a bacterial species associated with an inflammatory disease may be depleted in the microbial community. In some cases, a bacterial species related to an inflammatory disease may be depleted in the microbial community. In some cases, a bacterial species that can cause an inflammatory disease may be depleted in the microbial community. In some instances, a microorganism associated with an inflammatory disease may be depleted in the microbial community. In some cases, a microorganism related to an inflammatory disease may be depleted in the microbial community. In some cases, a microorganism that can cause an inflammatory disease may be depleted in the microbial community. In some cases, the inflammatory disease can comprise an allergy or a dermatitis.

In some instances, identifying an enrichment or a depletion of a microorganism may comprise using Statistical Framework Analysis of Composition of Microbiomes (ANCOM) or Applies Generalized Additive Models for Location, Scale and Shape (GAMLSS). In some cases, identifying an enrichment or a depletion of a microorganism may comprise using ANCOM. In some cases, identifying an enrichment or a depletion of a microorganism may comprise using GAMLSS. In some cases, identifying an enrichment or a depletion of a microorganism may comprise using ANCOM and GAMLSS. In some instances, identifying an enrichment or a depletion of a bacterial species may comprise using ANCOM or GAMLSS. In some cases, identifying an enrichment or a depletion of a bacterial species may comprise using ANCOM. In some cases, identifying an enrichment or a depletion of a bacterial species may comprise using GAMLSS. In some cases, identifying an enrichment or a depletion of a bacterial species may comprise using ANCOM and GAMLSS. In some instances, identifying an enrichment or a depletion of a bacterial strain may comprise using ANCOM or GAMLSS. In some cases, identifying an enrichment or a depletion of a bacterial strain may comprise using ANCOM. In some cases, identifying an enrichment or a depletion of a bacterial strain may comprise using GAMLSS. In some cases, identifying an enrichment or a depletion of a bacterial strain may comprise using ANCOM and GAMLSS. In some instances, identifying an enrichment or a depletion of a bacterial family, genus, order, class, or phylum may comprise using ANCOM or GAMLSS. In some cases, identifying an enrichment or a depletion of a bacterial family, genus, order, class, or phylum may comprise using ANCOM. In some cases, identifying an enrichment or a depletion of a bacterial family, genus, order, class, or phylum may comprise using GAMLSS. In some cases, identifying an enrichment or a depletion of a bacterial family, genus, order, class, or phylum may comprise using ANCOM and GAMLSS.

In some instances, an enriched bacterial species in a microbial community may comprise one species of family Bacteroidaceae or family Christensenellaceae. In some cases, an enriched bacterial species in a microbial community may comprise one species of family Bacteroidaceae. In some cases, an enriched bacterial species in a microbial community may comprise one species of family Christensenellaceae.

In some cases, an enriched bacterial species in a microbial community may comprise one species of genus Bacteroides or genus Clostridiaceae SMB53. In some cases, an enriched bacterial species in a microbial community may comprise one species of genus Bacteroides. In some cases, an enriched bacterial species in a microbial community may comprise one species of genus Clostridiaceae SMB53.

In some cases, an enriched bacterial species in a microbial community may comprise one species of Ruminococcus sp., Adlercreutzia sp., Enterorhabdus sp., Blautia sp., or Streptococcus sp. In some cases, an enriched bacterial species in a microbial community may be selected from at least two of Ruminococcus sp., Adlercreutzia sp., Enterorhabdus sp., Blautia sp., or Streptococcus sp. In some cases, an enriched bacterial species in a microbial community may be selected from at least three of Ruminococcus sp., Adlercreutzia sp., Enterorhabdus sp., Blautia sp., or Streptococcus sp. In some cases, an enriched bacterial species in a microbial community may be selected from at least four of Ruminococcus sp., Adlercreutzia sp., Enterorhabdus sp., Blautia sp., or Streptococcus sp. In some cases, an enriched bacterial species in a microbial community may comprise one species of Adlercreutzia sp. In some cases, an enriched bacterial species in a microbial community may comprise one species of Enterorhabdus sp. In some cases, an enriched bacterial species in a microbial community may comprise one species of Blautia sp. In some cases, an enriched bacterial species in a microbial community may comprise one species of Ruminococcus sp. In some cases, an enriched bacterial species in a microbial community may comprise one species of Streptococcus sp.

In some instances, an enriched bacterial species in a microbial community may comprise one species of Roseburia faecis, Blautia producta, Bacteroides eggerthii, Bacteroides plebeius, or Coprococcus eutactus. In some cases, an enriched bacterial species in a microbial community may comprise one species selected from at least two of Roseburia faecis, Blautia producta, Bacteroides eggerthii, Bacteroides plebeius, or Coprococcus eutactus. In some cases, an enriched bacterial species in a microbial community may comprise one species selected from at least three of Roseburia faecis, Blautia producta, Bacteroides eggerthii, Bacteroides plebeius, or Coprococcus eutactus. In some cases, an enriched bacterial species in a microbial community may comprise one species selected from at least four of Roseburia faecis, Blautia producta, Bacteroides eggerthii, Bacteroides plebeius, or Coprococcus eutactus.

In some instances, an enriched bacterial species in a microbial community may comprise one species of Roseburia faecis, Blautia producta, Bacteroides eggerthii, Bacteroides plebeius, Faecalibacterium prausnitzii, or Coprococcus eutactus. In some cases, an enriched bacterial species in a microbial community may comprise one species selected from at least two of Roseburia faecis, Blautia producta, Bacteroides eggerthii, Bacteroides plebeius, Faecalibacterium prausnitzii, or Coprococcus eutactus. In some cases, an enriched bacterial species in a microbial community may comprise one species selected from at least three of Roseburia faecis, Blautia producta, Bacteroides eggerthii, Bacteroides plebeius, Faecalibacterium prausnitzii, or Coprococcus eutactus. In some cases, an enriched bacterial species in a microbial community may comprise one species selected from at least four of Roseburia faecis, Blautia producta, Bacteroides eggerthii, Bacteroides plebeius, Faecalibacterium prausnitzii, or Coprococcus eutactus. In some cases, an enriched bacterial species in a microbial community may comprise one species selected from at least five of Roseburia faecis, Blautia producta, Bacteroides eggerthii, Bacteroides plebeius, Faecalibacterium prausnitzii, or Coprococcus eutactus.

In some cases, an enriched bacterial species in a microbial community may comprise one species of Roseburia faecis. In some cases, an enriched bacterial species in a microbial community may comprise one species of Blautia producta. In some cases, an enriched bacterial species in a microbial community may comprise one species of Bacteroides eggerthii. In some cases, an enriched bacterial species in a microbial community may comprise one species of Bacteroides plebeius. In some cases, an enriched bacterial species in a microbial community may comprise one species of Coprococcus eutactus. In some cases, an enriched bacterial species in a microbial community may comprise one species of Faecalibacterium prausnitzii. In some cases, the enriched Faecalibacterium prausnitzii in the microbial community may be different from the Faecalibacterium prausnitzii in a pharmaceutical composition administered to a subject.

In some cases, a depleted bacterial species in a microbial community may comprise one species of family Clostridiales, family Peptostreptococcaceae, or family Clostridiaceae. In some cases, a depleted bacterial species in a microbial community may comprise one species of family Clostridiales. In some cases, a depleted bacterial species in a microbial community may comprise one species of family Peptostreptococcaceae. In some cases, a depleted bacterial species in a microbial community may comprise one species of family Clostridiaceae.

In some cases, a depleted bacterial species in a microbial community may comprise one species of genus Turicibacter, genus Oscillospira, genus Slackia, or genus Coprococcus.

In some cases, a depleted bacterial species in a microbial community may comprise one species selected from at least three of genus Turicibacter, genus Oscillospira, genus Slackia, or genus Coprococcus. In some cases, a depleted bacterial species in a microbial community may comprise one species of genus Turicibacter. In some cases, a depleted bacterial species in a microbial community may comprise one species of genus Oscillospira. In some cases, a depleted bacterial species in a microbial community may comprise one species of genus Slackia. In some cases, a depleted bacterial species in a microbial community may comprise one species of genus Coprococcus.

In some cases, a depleted bacterial species in a microbial community may comprise one species of Streptococcus sp., Haemophilus sp., or Enterococcus sp. In some cases, a depleted bacterial species in a microbial community may comprise one species selected from at least two of Streptococcus sp., Haemophilus sp., or Enterococcus sp. In some cases, a depleted bacterial species in a microbial community may comprise one species of Streptococcus sp. In some cases, a depleted bacterial species in a microbial community may comprise one species of Haemophilus sp. In some cases, a depleted bacterial species in a microbial community may comprise one species of Enterococcus sp.

In some cases, a depleted bacterial species in a microbial community may comprise one species of Bacteroides ovatus, Eggerthella lenta, Haemophilus parainfluenzae, or Veillonella dispar. In some cases, a depleted bacterial species in a microbial community may comprise one species selected from at least two of Bacteroides ovatus, Eggerthella lenta, Haemophilus parainfluenzae, or Veillonella dispar. In some cases, a depleted bacterial species in a microbial community may comprise one species selected from at least three of Bacteroides ovatus, Eggerthella lenta, Haemophilus parainfluenzae, or Veillonella dispar. In some cases, a depleted bacterial species in a microbial community may comprise one species selected from at least four of Bacteroides ovatus, Eggerthella lenta, Haemophilus parainfluenzae, or Veillonella dispar. In some cases, a depleted bacterial species in a microbial community may comprise one species of Bacteroides ovatus. In some cases, a depleted bacterial species in a microbial community may comprise one species of Eggerthella lenta. In some cases, a depleted bacterial species in a microbial community may comprise one species of Haemophilus parainfluenzae. In some cases, a depleted bacterial species in a microbial community may comprise one species of Veillonella dispar.

In some instances, an enriched bacterial strain in a microbial community may comprise one strain of family Bacteroidaceae or family Christensenellaceae. In some cases, an enriched bacterial strain in a microbial community may comprise one strain of family Bacteroidaceae. In some cases, an enriched bacterial strain in a microbial community may comprise one strain of family Christensenellaceae.

In some cases, an enriched bacterial strain in a microbial community may comprise one strain of genus Bacteroides or genus Clostridiaceae SMB53. In some cases, an enriched bacterial strain in a microbial community may comprise one strain of genus Bacteroides. In some cases, an enriched bacterial strain in a microbial community may comprise one strain of genus Clostridiaceae SMB53.

In some cases, an enriched bacterial strain in a microbial community may comprise one strain of Ruminococcus sp. Adlercreutzia sp., Enterorhabdus sp., Blautia sp., or Streptococcus sp. In some cases, an enriched bacterial strain in a microbial community may be selected from at least two of Ruminococcus sp. Adlercreutzia sp., Enterorhabdus sp., Blautia sp., or Streptococcus sp. In some cases, an enriched bacterial strain in a microbial community may be selected from at least three of Ruminococcus sp. Adlercreutzia sp., Enterorhabdus sp., Blautia sp., or Streptococcus sp. In some cases, an enriched bacterial strain in a microbial community may be selected from at least four of Ruminococcus sp. Adlercreutzia sp., Enterorhabdus sp., Blautia sp., or Streptococcus sp. In some cases, an enriched bacterial strain in a microbial community may comprise one strain of Adlercreutzia sp. In some cases, an enriched bacterial strain in a microbial community may comprise one strain of Enterorhabdus sp. In some cases, an enriched bacterial strain in a microbial community may comprise one strain of Blautia sp. In some cases, an enriched bacterial strain in a microbial community may comprise one strain of Ruminococcus sp. In some cases, an enriched bacterial strain in a microbial community may comprise one strain of Streptococcus sp.

In some instances, an enriched bacterial strain in a microbial community may comprise one strain of Roseburia faecis, Blautia producta, Bacteroides eggerthii, Bacteroides plebeius, or Coprococcus eutactus. In some cases, an enriched bacterial strain in a microbial community may comprise one strain selected from at least two of Roseburia faecis, Blautia producta, Bacteroides eggerthii, Bacteroides plebeius, or Coprococcus eutactus. In some cases, an enriched bacterial strain in a microbial community may comprise one strain selected from at least three of Roseburia faecis, Blautia producta, Bacteroides eggerthii, Bacteroides plebeius, or Coprococcus eutactus. In some cases, an enriched bacterial strain in a microbial community may comprise one strain selected from at least four of Roseburia faecis, Blautia producta, Bacteroides eggerthii, Bacteroides plebeius, or Coprococcus eutactus.

In some instances, an enriched bacterial strain in a microbial community may comprise one strain of Roseburia faecis, Blautia producta, Bacteroides eggerthii, Bacteroides plebeius, Faecalibacterium prausnitzii, or Coprococcus eutactus. In some cases, an enriched bacterial strain in a microbial community may comprise one strain selected from at least two of Roseburia faecis, Blautia producta, Bacteroides eggerthii, Bacteroides plebeius, Faecalibacterium prausnitzii, or Coprococcus eutactus. In some cases, an enriched bacterial strain in a microbial community may comprise one strain selected from at least three of Roseburia faecis, Blautia producta, Bacteroides eggerthii, Bacteroides plebeius, Faecalibacterium prausnitzii, or Coprococcus eutactus. In some cases, an enriched bacterial strain in a microbial community may comprise one strain selected from at least four of Roseburia faecis, Blautia producta, Bacteroides eggerthii, Bacteroides plebeius, Faecalibacterium prausnitzii, or Coprococcus eutactus. In some cases, an enriched bacterial strain in a microbial community may comprise one strain selected from at least five of Roseburia faecis, Blautia producta, Bacteroides eggerthii, Bacteroides plebeius, Faecalibacterium prausnitzii, or Coprococcus eutactus.

In some cases, an enriched bacterial strain in a microbial community may comprise one strain of Roseburia faecis. In some cases, an enriched bacterial strain in a microbial community may comprise one strain of Blautia producta. In some cases, an enriched bacterial strain in a microbial community may comprise one strain of Bacteroides eggerthii. In some cases, an enriched bacterial strain in a microbial community may comprise one strain of Bacteroides plebeius. In some cases, an enriched bacterial strain in a microbial community may comprise one strain of Coprococcus eutactus. In some cases, an enriched bacterial strain in a microbial community may comprise one strain of Faecalibacterium prausnitzii. In some cases, the enriched Faecalibacterium prausnitzii in the microbial community may be different from the Faecalibacterium prausnitzii in a pharmaceutical composition administered to a subject.

In some cases, a depleted bacterial strain in a microbial community may comprise one strain of family Clostridiales, family Peptostreptococcaceae, or family Clostridiaceae. In some cases, a depleted bacterial strain in a microbial community may comprise one strain of family Clostridiales. In some cases, a depleted bacterial strain in a microbial community may comprise one strain of family Peptostreptococcaceae. In some cases, a depleted bacterial strain in a microbial community may comprise one strain of family Clostridiaceae.

In some cases, a depleted bacterial strain in a microbial community may comprise one strain of genus Turicibacter, genus Oscillospira, genus Slackia, or genus Coprococcus.

In some cases, a depleted bacterial strain in a microbial community may comprise one strain selected from at least three of genus Turicibacter, genus Oscillospira, genus Slackia, or genus Coprococcus. In some cases, a depleted bacterial strain in a microbial community may comprise one strain of genus Turicibacter. In some cases, a depleted bacterial strain in a microbial community may comprise one strain of genus Oscillospira. In some cases, a depleted bacterial strain in a microbial community may comprise one strain of genus Slackia. In some cases, a depleted bacterial strain in a microbial community may comprise one strain of genus Coprococcus.

In some cases, a depleted bacterial strain in a microbial community may comprise one strain of Streptococcus sp., Haemophilus sp., or Enterococcus sp. In some cases, a depleted bacterial strain in a microbial community may comprise one strain selected from at least two of Streptococcus sp., Haemophilus sp., or Enterococcus sp. In some cases, a depleted bacterial strain in a microbial community may comprise one strain of Streptococcus sp. In some cases, a depleted bacterial strain in a microbial community may comprise one strain of Haemophilus sp. In some cases, a depleted bacterial strain in a microbial community may comprise one strain of Enterococcus sp.

In some cases, a depleted bacterial strain in a microbial community may comprise one strain of Bacteroides ovatus, Eggerthella lenta, Haemophilus parainfluenzae, or Veillonella dispar. In some cases, a depleted bacterial strain in a microbial community may comprise one strain selected from at least two of Bacteroides ovatus, Eggerthella lenta, Haemophilus parainfluenzae, or Veillonella dispar. In some cases, a depleted bacterial strain in a microbial community may comprise one strain selected from at least three of Bacteroides ovatus, Eggerthella lenta, Haemophilus parainfluenzae, or Veillonella dispar. In some cases, a depleted bacterial strain in a microbial community may comprise one strain selected from at least four of Bacteroides ovatus, Eggerthella lenta, Haemophilus parainfluenzae, or Veillonella dispar. In some cases, a depleted bacterial strain in a microbial community may comprise one strain of Bacteroides ovatus. In some cases, a depleted bacterial strain in a microbial community may comprise one strain of Eggerthella lenta. In some cases, a depleted bacterial strain in a microbial community may comprise one strain of Haemophilus parainfluenzae. In some cases, a depleted bacterial strain in a microbial community may comprise one strain of Veillonella dispar.

In some instances, a control microbial community may comprise a microbial community in a subject without being administered to the pharmaceutical composition. In some cases, a control microbial community may comprise a microbial community in a subject prior to being administered to the pharmaceutical composition. In some cases, a control microbial community may comprise a microbial community in a subject without being administered to the pharmaceutical composition or in a subject prior to being administered to the pharmaceutical composition. In some cases, a control microbial community may comprise a microbial community in a second subject, wherein the second subject may not be administered to the pharmaceutical composition. In some cases, a control microbial community may comprise a microbial community in a second subject prior to being administered to the pharmaceutical composition.

### Pharmacokinetics of Pharmaceutical Compositions

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying the bacterial population in the subject relative to a control bacterial population. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying the bacterial population in the subject relative to a control bacterial population. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying the bacterial population in the subject relative to a control bacterial population. In some cases, a method may comprise the steps of: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying the bacterial population in the subject relative to a control bacterial population. In some cases, a method may comprise: (a) obtaining a sample comprising a bacterial population from a subject; (b) assaying the bacterial population relative to a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a bacterial population from a subject; (b) assaying the bacterial population relative to a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a bacterial population from a subject; (b) assaying the bacterial population relative to a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a bacterial population from a subject; (b) assaying the bacterial population relative to a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising the bacterial population from a subject; (3) assaying the bacterial population in the subject relative to a control bacterial population. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising the bacterial population from a subject; (3) assaying the bacterial population in the subject relative to a control bacterial population. In some cases, a method may comprise the steps of: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising the bacterial population from a subject; (3) assaying the bacterial population in the subject relative to a control bacterial population. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising the bacterial population from a subject; (3) assaying the bacterial population in the subject relative to a control bacterial population.

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying at least one bacterial species of the bacterial population in the subject relative to the at least one bacterial species of a control bacterial population. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying at least one bacterial species of the bacterial population in the subject relative to the at least one bacterial species of a control bacterial population. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying at least one bacterial species of the bacterial population in the subject relative to the at least one bacterial species of a control bacterial population. In some cases, a method may comprise the steps of: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying at least one bacterial species of the bacterial population in the subject relative to the at least one bacterial species of a control bacterial population. In some cases, a method may comprise: (a) obtaining a sample comprising a bacterial population from a subject; (b) assaying at least one bacterial species of the bacterial population relative to the at least one bacterial species of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a bacterial population from a subject; (b) assaying at least one bacterial species of the bacterial population relative to the at least one bacterial species of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a bacterial population from a subject; (b) assaying at least one bacterial species of the bacterial population relative to the at least one bacterial species of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a bacterial population from a subject; (b) assaying at least one bacterial species of the bacterial population relative to the at least one bacterial species of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising the bacterial population from a subject; (3) assaying at least one bacterial species of the bacterial population in the subject relative to the at least one bacterial species of a control bacterial population. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising the bacterial population from a subject; (3) assaying at least one bacterial species of the bacterial population in the subject relative to the at least one bacterial species of a control bacterial population. In some cases, a method may comprise the steps of: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising the bacterial population from a subject; (3) assaying at least one bacterial species of the bacterial population in the subject relative to the at least one bacterial species of a control bacterial population. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising the bacterial population from a subject; (3) assaying at least one bacterial species of the bacterial population in the subject relative to the at least one bacterial species of a control bacterial population.

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying at least one bacterial strain of the bacterial population in the subject relative to the at least one bacterial strain of a control bacterial population. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying at least one bacterial strain of the bacterial population in the subject relative to the at least one bacterial strain of a control bacterial population. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying at least one bacterial strain of the bacterial population in the subject relative to the at least one bacterial strain of a control bacterial population. In some cases, a method may comprise the steps of: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying at least one bacterial strain of the bacterial population in the subject relative to the at least one bacterial strain of a control bacterial population. In some cases, a method may comprise: (a) obtaining a sample comprising a bacterial population from a subject; (b) assaying at least one bacterial strain of the bacterial population relative to the at least one bacterial strain of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a bacterial population from a subject; (b) assaying at least one bacterial strain of the bacterial population relative to the at least one bacterial strain of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a bacterial population from a subject; (b) assaying at least one bacterial strain of the bacterial population relative to the at least one bacterial strain of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a bacterial population from a subject; (b) assaying at least one bacterial strain of the bacterial population relative to the at least one bacterial strain of a control bacterial population, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising the bacterial population from a subject; (3) assaying at least one bacterial strain of the bacterial population in the subject relative to the at least one bacterial strain of a control bacterial population. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising the bacterial population from a subject; (3) assaying at least one bacterial strain of the bacterial population in the subject relative to the at least one bacterial strain of a control bacterial population. In some cases, a method may comprise the steps of: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising the bacterial population from a subject; (3) assaying at least one bacterial strain of the bacterial population in the subject relative to the at least one bacterial strain of a control bacterial population. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising the bacterial population from a subject; (3) assaying at least one bacterial strain of the bacterial population in the subject relative to the at least one bacterial strain of a control bacterial population.

In some instances, assaying a bacterial population may comprise taking a mean amount, a median amount, a mode amount, a range amount, a first quartile amount, a second quartile amount, a third quartile amount, or a fourth quartile amount of at least one bacterial strain, species, genus, family, order, class, phylum, or kingdom of the bacterial population and a control bacterial population. In some cases, assaying a bacterial population may comprise taking a mean amount of at least one bacterial strain, species, genus, family, order, class, phylum, or kingdom of the bacterial population and a control bacterial population. In some cases, assaying a bacterial population may comprise taking a median amount of at least one bacterial strain, species, genus, family, order, class, phylum, or kingdom of the bacterial population and a control bacterial population.

In some instances, assaying a bacterial species of a bacterial population may comprise taking a mean amount, a median amount, a mode amount, a range amount, a first quartile amount, a second quartile amount, a third quartile amount, or a fourth quartile amount of the bacterial species of the bacterial population and a control bacterial population. In some cases, assaying a bacterial species of a bacterial population may comprise taking a mean amount of the bacterial species of the bacterial population and a control bacterial population. In some cases, assaying a bacterial species of a bacterial population may comprise taking a median amount of the bacterial species of the bacterial population and a control bacterial population. In some cases, assaying a bacterial species of a bacterial population may comprise taking a mode amount of the bacterial species of the bacterial population and a control bacterial population. In some cases, assaying a bacterial species of a bacterial population may comprise taking a range amount of the bacterial species of the bacterial population and a control bacterial population. In some cases, assaying a bacterial species of a bacterial population may comprise taking a first quartile amount of the bacterial species of the bacterial population and a control bacterial population. In some cases, assaying a bacterial species of a bacterial population may comprise taking a second quartile amount of the bacterial species of the bacterial population and a control bacterial population. In some cases, assaying a bacterial species of a bacterial population may comprise taking a third quartile amount of the bacterial species of the bacterial population and a control bacterial population. In some cases, assaying a bacterial species of a bacterial population may comprise taking a fourth quartile amount of the bacterial species of the bacterial population and a control bacterial population.

In some instances, assaying a bacterial strain of a bacterial population may comprise taking a mean amount, a median amount, a mode amount, a range amount, a first quartile amount, a second quartile amount, a third quartile amount, or a fourth quartile amount of the bacterial strain of the bacterial population and a control bacterial population. In some cases, assaying a bacterial strain of a bacterial population may comprise taking a mean amount of the bacterial strain of the bacterial population and a control bacterial population. In some cases, assaying a bacterial strain of a bacterial population may comprise taking a median amount of the bacterial strain of the bacterial population and a control bacterial population. In some cases, assaying a bacterial strain of a bacterial population may comprise taking a mode amount of the bacterial strain of the bacterial population and a control bacterial population. In some cases, assaying a bacterial strain of a bacterial population may comprise taking a range amount of the bacterial strain of the bacterial population and a control bacterial population. In some cases, assaying a bacterial strain of a bacterial population may comprise taking a first quartile amount of the bacterial strain of the bacterial population and a control bacterial population. In some cases, assaying a bacterial strain of a bacterial population may comprise taking a second quartile amount of the bacterial strain of the bacterial population and a control bacterial population. In some cases, assaying a bacterial strain of a bacterial population may comprise taking a third quartile amount of the bacterial strain of the bacterial population and a control bacterial population. In some cases, assaying a bacterial strain of a bacterial population may comprise taking a fourth quartile amount of the bacterial strain of the bacterial population and a control bacterial population.

In some instances, the amount of a bacteria strain in a subject may have an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase relative to the amount of the bacteria strain of a control bacterial population. In some cases, the amount of a bacteria species in a subject may have an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase relative to the amount of the bacteria species of a control bacterial population.

In some cases, the amount of the bacterial species or strains may be represented by the number of the bacterial species or strains; the number of the microorganisms of the bacterial species or strains; the abundance of the nucleic acid representing the number of the bacterial species or strains or the number of the microorganisms of the bacterial species or strains; the dry weight of nucleic acids representing the number of the bacterial species or strains or the number of the microorganisms of the bacterial species or strains, the dry weight of the bacterial species, bacterial strains, or the microorganisms; or a combination thereof.

In some instances, a control bacterial population may comprise a bacterial population in a subject without being administered to the pharmaceutical composition. In some cases, a control bacterial population may comprise a bacterial population in a subject prior to being administered to the pharmaceutical composition. In some cases, a control bacterial population may comprise a bacterial population in a subject without being administered to the pharmaceutical composition or in a subject prior to being administered to the pharmaceutical composition. In some cases, a control bacterial population may comprise a bacterial population in a second subject, wherein the second subject may not be administered to the pharmaceutical composition. In some cases, a control bacterial population may comprise a bacterial population in a second subject prior to being administered to the pharmaceutical composition.

In some instances, a bacterial species of a bacterial population in a pharmaceutical composition may be present in a subject subsequent to, during, or prior to the subject is administered to the pharmaceutical composition. In some cases, a bacterial species of a bacterial population in a pharmaceutical composition may be present in a subject subsequent to the subject is administered to the pharmaceutical composition. In some cases, a bacterial species of a bacterial population in a pharmaceutical composition may be present in a subject in a washout period subsequent to the subject is administered to the pharmaceutical composition. In some cases, a bacterial species of a bacterial population in a pharmaceutical composition may be present in a subject during the subject is administered to the pharmaceutical composition. In some cases, a bacterial species of a bacterial population in a pharmaceutical composition may be present in a subject prior to the subject is administered to the pharmaceutical composition.

### Steroids

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying a steroid compound in the subject relative to the steroid compound of a control sample. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a steroid compound in the subject relative to the steroid compound of a control sample. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying a steroid compound in the subject relative to the steroid compound of a control sample. In some cases, a method may comprise the steps of: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a steroid compound in the subject relative to the steroid compound of a control sample. In some cases, a method may comprise: (a) obtaining a sample comprising a steroid compound from a subject; (b) assaying the steroid compound relative to the steroid compound of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a steroid compound from a subject; (b) assaying the steroid compound relative to the steroid compound of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a steroid compound from a subject; (b) assaying the steroid compound relative to the steroid compound of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a steroid compound from a subject; (b) assaying the steroid compound relative to the steroid compound of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a steroid compound from a subject; (3) assaying the steroid compound in the subject relative to the steroid compound of a control sample. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a steroid compound from a subject; (3) assaying the steroid compound in the subject relative to the steroid compound of a control sample. In some cases, a method may comprise the steps of: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a steroid compound from a subject; (3) assaying the steroid compound in the subject relative to the steroid compound of a control sample. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a steroid compound from a subject; (3) assaying the steroid compound in the subject relative to the steroid compound of a control sample.

In some instances, the abundance of a steroid compound in a subject may have an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase relative to the abundance of the steroid compound of a control sample.

In some instances, a steroid compound may comprise a progestogenic steroid compound, a corticosteroid steroid compound, an androgenic steroid compound, an estrogenic steroid compound, or a combination thereof. In some cases, a steroid compound may be selected from at least two of a progestogenic steroid compound, a corticosteroid steroid compound, an androgenic steroid compound, or an estrogenic steroid compound. In some cases, a steroid compound may be selected from at least three of a progestogenic steroid compound, a corticosteroid steroid compound, an androgenic steroid compound, or an estrogenic steroid compound. In some cases, a steroid compound may comprise a progestogenic steroid compound. In some cases, a steroid compound may comprise a corticosteroid steroid compound. In some cases, a steroid compound may comprise an androgenic steroid compound. In some cases, a steroid compound may comprise an estrogenic steroid compound.

In some instances, an androgenic steroid compound may comprise androstenediol (3alpha, 17alpha) monosulfate, 11beta-hydroxyandrosterone glucuronide, androsterone glucuronide, a derivative thereof, or a combination thereof. In some cases, an androgenic steroid compound may be selected from at least two of androstenediol (3alpha, 17alpha) monosulfate or a derivative thereof; 11beta-hydroxyandrosterone glucuronide or a derivative thereof; or androsterone glucuronide or a derivative thereof. In some cases, an androgenic steroid compound may comprise androstenediol (3alpha, 17alpha) monosulfate or a derivative thereof. In some cases, an androgenic steroid compound may comprise 11 beta-hydroxyandrosterone glucuronide or a derivative thereof. In some cases, an androgenic steroid compound may comprise androsterone glucuronide or a derivative thereof. In some cases, an androgenic steroid compound may comprise androstenediol (3alpha, 17alpha) monosulfate. In some cases, an androgenic steroid compound may comprise 11beta-hydroxyandrosterone glucuronide. In some cases, an androgenic steroid compound may comprise androsterone glucuronide.

In some instances, a steroid compound in a control sample may comprise a steroid compound in a subject without being administered to a pharmaceutical composition. In some cases, a steroid compound in a control sample may comprise a steroid compound in a subject prior to being administered to a pharmaceutical composition. In some cases, a steroid compound in a control sample may comprise a steroid compound in a subject without being administered to a pharmaceutical composition or in a subject prior to being administered to a pharmaceutical composition. In some cases, a steroid compound in a control sample may comprise a steroid compound in a second subject, wherein the second subject may not be administered to a pharmaceutical composition. In some cases, a steroid compound in a control sample may comprise a steroid compound in a second subject prior to being administered to a pharmaceutical composition.

In some instances, a steroid compound may be present in a subject subsequent to, during, or prior to the subject is administered to a pharmaceutical composition. In some cases, a steroid compound may be present in a subject subsequent to the subject is administered to a pharmaceutical composition. In some cases, a steroid compound may be present in a subject in a washout period subsequent to the subject is administered to a pharmaceutical composition. In some cases, a steroid compound may be present in a subject during the subject is administered to a pharmaceutical composition. In some cases, a steroid compound may be present in a subject prior to the subject is administered to a pharmaceutical composition.

### Microbially-Derived Metabolites

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying a microbially-derived metabolite in the subject relative to the microbially-derived metabolite of a control sample. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a microbially-derived metabolite in the subject relative to the microbially-derived metabolite of a control sample. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying a microbially-derived metabolite in the subject relative to the microbially-derived metabolite of a control sample. In some cases, a method may comprise the steps of: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a microbially-derived metabolite in the subject relative to the microbially-derived metabolite of a control sample. In some cases, a method may comprise: (a) obtaining a sample comprising a microbially-derived metabolite from a subject; (b) assaying the microbially-derived metabolite relative to the microbially-derived metabolite of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a microbially-derived metabolite from a subject; (b) assaying the microbially-derived metabolite relative to the microbially-derived metabolite of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a microbially-derived metabolite from a subject; (b) assaying the microbially-derived metabolite relative to the microbially-derived metabolite of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a microbially-derived metabolite from a subject; (b) assaying the microbially-derived metabolite relative to the microbially-derived metabolite of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a microbially-derived metabolite from a subject; (3) assaying the microbially-derived metabolite in the subject relative to the microbially-derived metabolite of a control sample. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a microbially-derived metabolite from a subject; (3) assaying the microbially-derived metabolite in the subject relative to the microbially-derived metabolite of a control sample. In some cases, a method may comprise the steps of: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a microbially-derived metabolite from a subject; (3) assaying the microbially-derived metabolite in the subject relative to the microbially-derived metabolite of a control sample. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a microbially-derived metabolite from a subject; (3) assaying the microbially-derived metabolite in the subject relative to the microbially-derived metabolite of a control sample.

In some instances, a microbially-derived metabolite may comprise a bile acid, a SCFA, an amino acid, a branched-chain of amino acids, a linear-chain of amino acids, a trimethylamine N-oxide, an indole, or a derivative thereof, or a combination thereof. In some cases, a microbially-derived metabolite may be selected from at least two of a bile acid, a SCFA, an amino acid, a branched-chain of amino acids, a linear-chain of amino acids, a trimethylamine N-oxide, an indole, or a derivative thereof, or a combination thereof. In some cases, a microbially-derived metabolite may be selected from at least three of a bile acid, a SCFA, an amino acid, a branched-chain of amino acids, a linear-chain of amino acids, a trimethylamine N-oxide, an indole, or a derivative thereof, or a combination thereof. In some cases, a microbially-derived metabolite may be selected from at least four of a bile acid, a SCFA, an amino acid, a branched-chain of amino acids, a linear-chain of amino acids, a trimethylamine N-oxide, an indole, or a derivative thereof, or a combination thereof. In some cases, a microbially-derived metabolite may be selected from at least five of a bile acid, a SCFA, an amino acid, a branched-chain of amino acids, a linear-chain of amino acids, a trimethylamine N-oxide, an indole, or a derivative thereof, or a combination thereof. In some cases, a microbially-derived metabolite may be selected from at least six of a bile acid, a SCFA, an amino acid, a branched-chain of amino acids, a linear-chain of amino acids, a trimethylamine N-oxide, an indole, or a derivative thereof, or a combination thereof. In some cases, a microbially-derived metabolite may comprise a tryptophan or a derivative thereof. In some cases, a microbially-derived metabolite may comprise a bile acid or a derivative thereof. In some cases, a microbially-derived metabolite may comprise a SCFA or a derivative thereof. In some cases, a microbially-derived metabolite may comprise an amino acid or a derivative thereof. In some cases, a microbially-derived metabolite may comprise a branched-chain of amino acids or a derivative thereof. In some cases, a microbially-derived metabolite may comprise a linear-chain of amino acids or a derivative thereof. In some cases, a microbially-derived metabolite may comprise a trimethylamine N-oxide or a derivative thereof. In some cases, a microbially-derived metabolite may comprise an indole or a derivative thereof. In some cases, a microbially-derived metabolite may comprise a tryptophan. In some cases, a microbially-derived metabolite may comprise a bile acid. In some cases, a microbially-derived metabolite may comprise a SCFA. In some cases, a microbially-derived metabolite may comprise an amino acid. In some cases, a microbially-derived metabolite may comprise a branched-chain of amino acids. In some cases, a microbially-derived metabolite may comprise a linear-chain of amino acids. In some cases, a microbially-derived metabolite may comprise a trimethylamine N-oxide. In some cases, a microbially-derived metabolite may comprise an indole.

In some instances, the abundance of a microbially-derived metabolite in a subject may have an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase relative to the abundance of the microbially-derived metabolite of a control sample.

In some instances, a microbially-derived metabolite in a control sample may comprise a microbially-derived metabolite in a subject without being administered to a pharmaceutical composition. In some cases, a microbially-derived metabolite in a control sample may comprise a microbially-derived metabolite in a subject prior to being administered to a pharmaceutical composition. In some cases, a microbially-derived metabolite in a control sample may comprise a microbially-derived metabolite in a subject without being administered to a pharmaceutical composition or in a subject prior to being administered to a pharmaceutical composition. In some cases, a microbially-derived metabolite in a control sample may comprise a microbially-derived metabolite in a second subject, wherein the second subject may not be administered to a pharmaceutical composition. In some cases, a microbially-derived metabolite in a control sample may comprise a microbially-derived metabolite in a second subject prior to being administered to a pharmaceutical composition.

In some instances, a microbially-derived metabolite may be present in a subject subsequent to, during, or prior to the subject is administered to a pharmaceutical composition. In some cases, a microbially-derived metabolite may be present in a subject subsequent to the subject is administered to a pharmaceutical composition. In some cases, a microbially-derived metabolite may be present in a subject in a washout period subsequent to the subject is administered to a pharmaceutical composition. In some cases, a microbially-derived metabolite may be present in a subject during the subject is administered to a pharmaceutical composition. In some cases, a microbially-derived metabolite may be present in a subject prior to the subject is administered to a pharmaceutical composition.

### Membrane Lipids

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying a membrane lipid in the subject relative to the membrane lipid of a control sample. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a membrane lipid in the subject relative to the membrane lipid of a control sample. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying a membrane lipid in the subject relative to the membrane lipid of a control sample. In some cases, a method may comprise the steps of: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a membrane lipid in the subject relative to the membrane lipid of a control sample. In some cases, a method may comprise: (a) obtaining a sample comprising a membrane lipid from a subject; (b) assaying the membrane lipid relative to the membrane lipid of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a membrane lipid from a subject; (b) assaying the membrane lipid relative to the membrane lipid of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a membrane lipid from a subject; (b) assaying the membrane lipid relative to the membrane lipid of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a membrane lipid from a subject; (b) assaying the membrane lipid relative to the membrane lipid of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a membrane lipid from a subject; (3) assaying the membrane lipid in the subject relative to the membrane lipid of a control sample. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a membrane lipid from a subject; (3) assaying the membrane lipid in the subject relative to the membrane lipid of a control sample. In some cases, a method may comprise the steps of: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a membrane lipid from a subject; (3) assaying the membrane lipid in the subject relative to the membrane lipid of a control sample. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a membrane lipid from a subject; (3) assaying the membrane lipid in the subject relative to the membrane lipid of a control sample.

In some instances, a membrane lipid may comprise a phospholipid, a glycolipid, a cholesterol, or a combination thereof. In some cases, a membrane lipid may be selected from at least two of a phospholipid, a glycolipid, or a cholesterol. In some cases, a membrane lipid may comprise a phospholipid. In some cases, a membrane lipid may comprise a glycolipid. In some cases, a membrane lipid may comprise a cholesterol. In some instances, a phospholipid may comprise a phosphatidylcholine (PC), a phosphatidylethanolamine (PE), a phosphatidylinositol (PI) or a combination thereof. In some cases, a membrane lipid may be selected from at least two of a PC, a PE, or a PI. In some cases, a membrane lipid may comprise a PE. In some cases, a membrane lipid may comprise a PC. In some cases, a membrane lipid may comprise a PI.

In some instances, a PC may comprise 1,2-dipalmitoyl-GPC (16:0/16:0), 1-palmitoyl-2-palmitoleoyl-GPC (16:0/16:1), 1-myristoyl-2-palmitoyl-GPC (14:0/16:0), 1-palmitoyl-2-oleoyl-GPC (16:0/18:1) or a combination thereof. In some cases, a PC may be selected from at least two of 1,2-dipalmitoyl-GPC (16:0/16:0), 1-palmitoyl-2-palmitoleoyl-GPC (16:0/16:1), 1-myristoyl-2-palmitoyl-GPC (14:0/16:0), or 1-palmitoyl-2-oleoyl-GPC (16:0/18:1). In some cases, a PC may be selected from at least three of 1,2-dipalmitoyl-GPC (16:0/16:0), 1-palmitoyl-2-palmitoleoyl-GPC (16:0/16:1), 1-myristoyl-2-palmitoyl-GPC (14:0/16:0), or 1-palmitoyl-2-oleoyl-GPC (16:0/18:1).

In some cases, a PC may comprise 1,2-dipalmitoyl-GPC (16:0/16:0). In some cases, a PC may comprise 1-palmitoyl-2-palmitoleoyl-GPC (16:0/16:1). In some cases, a PC may comprise 1-myristoyl-2-palmitoyl-GPC (14:0/16:0). In some cases, a PC may comprise 1-palmitoyl-2-oleoyl-GPC (16:0/18:1).

In some instances, a PE may comprise 1-palmitoyl-2-arachidonoyl-GPE (16:0/20:4), 1-palmitoyl-2-linoleoyl-GPE (16:0/18:2), or a combination thereof. In some cases, a PE may comprise 1-palmitoyl-2-arachidonoyl-GPE (16:0/20:4). In some cases, a PE may comprise 1-palmitoyl-2-linoleoyl-GPE (16:0/18:2).

In some instances, a PI may comprise 1-palmitoyl-2-linoleoyl-GPI (16:0/18:2).

In some instances, the abundance of a membrane lipid in a subject may have an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase relative to the abundance of the membrane lipid of a control sample.

In some instances, a membrane lipid in a control sample may comprise a membrane lipid in a subject without being administered to a pharmaceutical composition. In some cases, a membrane lipid in a control sample may comprise a membrane lipid in a subject prior to being administered to a pharmaceutical composition. In some cases, a membrane lipid in a control sample may comprise a membrane lipid in a subject without being administered to a pharmaceutical composition or in a subject prior to being administered to a pharmaceutical composition. In some cases, a membrane lipid in a control sample may comprise a membrane lipid in a second subject, wherein the second subject may not be administered to a pharmaceutical composition. In some cases, a membrane lipid in a control sample may comprise a membrane lipid in a second subject prior to being administered to a pharmaceutical composition.

In some instances, a membrane lipid may be present in a subject subsequent to, during, or prior to the subject is administered to a pharmaceutical composition. In some cases, a membrane lipid may be present in a subject subsequent to the subject is administered to a pharmaceutical composition. In some cases, a membrane lipid may be present in a subject in a washout period subsequent to the subject is administered to a pharmaceutical composition. In some cases, a membrane lipid may be present in a subject during the subject is administered to a pharmaceutical composition. In some cases, a membrane lipid may be present in a subject prior to the subject is administered to a pharmaceutical composition.

### Pro-Inflammatory Immune Markers

In some instances, a method may comprise: (a) administering to a subject a pharmaceutical composition comprising a bacterial population comprising at least two bacterial species selected from the group consisting of Akkermansia sp., Faecalibacterium sp., and Lactobacillus sp.; (b) assaying a pro-inflammatory immune marker in the subject relative to the pro-inflammatory immune marker of a control sample. In some instances, a method may comprise: (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a pro-inflammatory immune marker in the subject relative to the pro-inflammatory immune marker of a control sample. In some cases, a method may comprise the steps of: (a) administering to a subject a pharmaceutical composition comprising a bacterial population comprising at least two bacterial species selected from the group consisting of Akkermansia sp., Faecalibacterium sp., and Lactobacillus sp.; (b) assaying a pro-inflammatory immune marker in the subject relative to the pro-inflammatory immune marker of a control sample. In some cases, a method may comprise the steps of (a) administering to a subject a pharmaceutical composition comprising a bacterial population comprising at least two bacterial species selected from the group consisting of Akkermansia sp., Faecalibacterium sp., and a species of the Lactobacillaceae family; (b) assaying a pro-inflammatory immune marker in the subject relative to the pro-inflammatory immune marker of a control sample. In some cases, a method may comprise: (a) obtaining a sample comprising a pro-inflammatory immune marker from a subject; (b) assaying the pro-inflammatory immune marker relative to the pro-inflammatory immune marker of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise: (a) obtaining a sample comprising a pro-inflammatory immune marker from a subject; (b) assaying the pro-inflammatory immune marker relative to the pro-inflammatory immune marker of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise the steps of: (a) obtaining a sample comprising a pro-inflammatory immune marker from a subject; (b) assaying the pro-inflammatory immune marker relative to the pro-inflammatory immune marker of a control sample, wherein the sample is obtained from the subject subsequent to being administered with a pharmaceutical composition comprising a bacterial population comprising at least two bacterial species selected from the group consisting of Akkermansia sp., Faecalibacterium sp., and Lactobacillus sp. In some cases, a method may comprise the steps of: (a) obtaining a sample comprising a pro-inflammatory immune marker from a subject; (b) assaying the pro-inflammatory immune marker relative to the pro-inflammatory immune marker of a control sample, wherein the sample is obtained from the subject subsequent to being administered with a pharmaceutical composition comprising a bacterial population comprising at least two bacterial species selected from the group consisting of Akkermansia sp., Faecalibacterium sp., and a species of the Lactobacillaceae family. In some cases, a method may comprise: (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a pro-inflammatory immune marker from a subject; (3) assaying the pro-inflammatory immune marker in the subject relative to the pro-inflammatory immune marker of a control sample. In some cases, a method may comprise: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a pro-inflammatory immune marker from a subject; (3) assaying the pro-inflammatory immune marker in the subject relative to the pro-inflammatory immune marker of a control sample. In some cases, a method may comprise the steps of (1) administering to a subject a pharmaceutical composition comprising a bacterial population comprising at least two bacterial species selected from the group consisting of Akkermansia sp., Faecalibacterium sp., and Lactobacillus sp.; (2) obtaining a sample comprising a pro-inflammatory immune marker from a subject; (3) assaying the pro-inflammatory immune marker in the subject relative to the pro-inflammatory immune marker of a control sample. In some cases, a method may comprise the steps of: (1) administering to a subject a pharmaceutical composition comprising a bacterial population comprising at least two bacterial species selected from the group consisting of Akkermansia sp., Faecalibacterium sp., and a species of the Lactobacillaceae family; (2) obtaining a sample comprising a pro-inflammatory immune marker from a subject; (3) assaying the pro-inflammatory immune marker in the subject relative to the pro-inflammatory immune marker of a control sample.

In some instances, a pro-inflammatory immune marker may comprise a nucleotide, a polynucleotide, a protein, an amino acid, a peptide, a lipid, a cell, a chemical compound, a saccharide, or a polysaccharide. In some cases, a protein pro-inflammatory immune marker may comprise an antibody. In some cases, a protein pro-inflammatory immune marker may comprise a cytokine. In some cases, a protein pro-inflammatory immune marker may comprise an interleukin. In some cases, a chemical compound pro-inflammatory immune marker may comprise a histamine. In some cases, a cell pro-inflammatory immune marker may comprise a white blood cell. Such a white blood cell, in some cases, may comprise a granulocyte, a monocyte, or a lymphocyte. In some cases, a granulocyte pro-inflammatory immune marker may comprise a neutrophil, an eosinophil, or a basophil. In some cases, a granulocyte pro-inflammatory immune marker may comprise a neutrophil. In some cases, a granulocyte pro-inflammatory immune marker may comprise an eosinophil. In some cases, a granulocyte pro-inflammatory immune marker may comprise a basophil. In some cases, a lymphocyte pro-inflammatory immune marker may comprise a T cell or a B cell. In some cases, a lymphocyte pro-inflammatory immune marker may comprise a T cell. In some cases, a lymphocyte pro-inflammatory immune marker may comprise a B cell.

In some instances, a pro-inflammatory immune marker may comprise a basophil, an eotaxin, a histamine, an interleukin-4 (IL-4), or a combination thereof. In some cases, a pro-inflammatory immune marker may be selected from at least two of a basophil, an eotaxin, a histamine, or an IL-4. In some cases, a pro-inflammatory immune marker may be selected from at least three of a basophil, an eotaxin, a histamine, or an IL-4. In some cases, a pro-inflammatory immune marker may comprise a basophil. In some cases, a pro-inflammatory immune marker may comprise an eotaxin. In some cases, a pro-inflammatory immune marker may comprise a histamine. In some cases, a pro-inflammatory immune marker may comprise an IL-4.

In some instances, the abundance of a pro-inflammatory immune marker may have an at least about 1% decrease, an at least about 5% decrease, an at least about 10% decrease, an at least about 15% decrease, an at least about 20% decrease, an at least about 25% decrease, an at least about 30% decrease, an at least about 35% decrease, an at least about 40% decrease, an at least about 45% decrease, an at least about 50% decrease, an at least about 55% decrease, an at least about 60% decrease, an at least about 65% decrease, an at least about 70% decrease, an at least about 75% decrease, an at least about 80% decrease, an at least about 85% decrease, an at least about 90% decrease, an at least about 91% decrease, an at least about 92% decrease, an at least about 93% decrease, an at least about 94% decrease, an at least about 95% decrease, an at least about 96% decrease, an at least about 97% decrease, an at least about 98% decrease, an at least about 99% decrease, or a 100% decrease relative to the abundance of the pro-inflammatory immune marker in a control sample.

In some instances, a pro-inflammatory immune marker in a control sample may comprise a pro-inflammatory immune marker in a subject without being administered to a pharmaceutical composition. In some cases, a pro-inflammatory immune marker in a control sample may comprise a pro-inflammatory immune marker in a subject prior to being administered to a pharmaceutical composition. In some cases, a pro-inflammatory immune marker in a control sample may comprise a pro-inflammatory immune marker in a subject without being administered to a pharmaceutical composition or in a subject prior to being administered to a pharmaceutical composition. In some cases, a pro-inflammatory immune marker in a control sample may comprise a pro-inflammatory immune marker in a second subject, wherein the second subject may not be administered to a pharmaceutical composition. In some cases, a pro-inflammatory immune marker in a control sample may comprise a pro-inflammatory immune marker in a second subject prior to being administered to a pharmaceutical composition.

In some instances, a pro-inflammatory immune marker may be present in a subject subsequent to, during, or prior to the subject is administered to a pharmaceutical composition. In some cases, a pro-inflammatory immune marker may be present in a subject subsequent to the subject is administered to a pharmaceutical composition. In some cases, a pro-inflammatory immune marker may be present in a subject in a washout period subsequent to the subject is administered to a pharmaceutical composition. In some cases, a pro-inflammatory immune marker may be present in a subject during the subject is administered to a pharmaceutical composition. In some cases, a pro-inflammatory immune marker may be present in a subject prior to the subject is administered to a pharmaceutical composition.

In some cases, other inflammatory markers or immune markers be also be assayed. In some cases, anti-inflammatory immune markers may be assayed. In the case of an anti-inflammatory immune marker, the pharmaceutical composition provided herein may increase the abundance of the anti-inflammatory immune marker. In some instances, the abundance of an anti-inflammatory immune marker in a subject may have an at least about 5% increase, an at least about 10% increase, an at least about 20% increase, an at least about 50% increase, an at least about 100% increase, an at least about 200% increase, an at least about 500% increase, an at least about 1000% increase, an at least about 2000% increase, an at least about 5000% increase, an at least about 10000% increase, an at least about 20000% increase, an at least about 50000% increase, an at least about 100000% increase, an at least about 200000% increase, or an at least about 500000% increase relative to the abundance of the anti-inflammatory immune marker of a control sample.

### Assaying Methods

Provided herein are methods for assaying.

In some cases, identification of a microorganism, a bacterial strain, a bacterial species, or bacterial isolate may be performed by sequencing of the full-length 16S rRNA gene. Such a method may use one or more amplification primer followed by nucleic acid sequencing. Full-length 16S rRNA gene sequence reads can be aligned in the Ribosomal Database Project (RDP), manually curated using nucleic acid analysis and sequencing programs (e.g., ARB, mother, etc.) to classify reads to operational taxonomic units (OTUs) amplicon sequence variants (ASVs). The full-length 16S rRNA gene sequence of each species-level OUT or ASV can then be compared to the RDP reference database to assign taxonomic designations to the genus and/or strain level followed by a BLASTn search to either a characterized or candidate novel species. In some cases, the microorganism, bacterial strain, bacterial species, or bacterial isolate may be present in a bacterial population, a microbial community a pharmaceutical composition, or a subject.

In some instances, assaying a microorganism, a bacterial strain, a bacterial species, or a bacterial isolate may comprise a quantitative PCR. In some cases, a microorganism, a bacterial strain, or a bacterial species may be identified or quantified by at least one primer in a quantitative PCR. In some cases, at least one primer in a quantitative PCR may be configured to prime to a locus of a microorganism, a bacterial strain, or a bacterial species in a quantitative PCR. The locus being primed may comprise a 16S rRNA gene sequence. The locus being primed may also comprise any gene sequence that can distinguish a microorganism, a bacterial strain, a bacterial species, or a bacterial isolate from other microorganisms, bacterial strains, bacterial species, or bacterial isolates.

In some instances, assaying a microorganism, a bacterial strain, a bacterial species, or a bacterial isolate may also comprise a sequencing reaction. In some cases, such a sequencing reaction may comprise a high-throughput sequencing reaction. In some cases, assaying a microorganism, a bacterial strain, a bacterial species, or a bacterial isolate by a sequencing reaction may also comprise identifying sequence reads of the microorganism, bacterial strain, bacterial species, or bacterial isolate. In some cases, the sequence reads that can identify a microorganism, a bacterial strain, a bacterial species, or a bacterial isolate may comprise the 16S rRNA gene sequence of the microorganism, bacterial strain, bacterial species, or bacterial isolate. The sequence read may also comprise any gene sequence that can distinguish a microorganism, a bacterial strain, a bacterial species, or a bacterial isolate from other microorganisms, bacterial strains, bacterial species, or bacterial isolates. In some cases, sequencing may be substituted by a microarray or other PCR-/sequencing-based analytic methods. In some cases, using sequencing to analyze bacterial species in a bacterial community is described in EXAMPLE 3.

In some cases, identification of a bacterial species or strain of Akkermansia sp., Lactobacillus sp., or Faecalibacterium sp. may be carried by method described in EXAMPLE 5 or EXAMPLE 6. In some cases, identification of a bacterial species or strain of Akkermansia sp., a species of the Lactobacillaceae family, or Faecalibacterium sp. may be carried by method described in EXAMPLE 5 or EXAMPLE 6. In some cases, methods described in EXAMPLE 5 or EXAMPLE 6 may be modified to identify any microorganism.

In some cases, assaying a protein or peptide may comprise a Western blot, a mass spectrometry (MS), a cytometry, an ELISA (enzyme-linked immunosorbent assay), or a combination thereof. In some cases, assaying a protein pro-inflammatory immune marker may comprise a Western blot, a mass spectrometry (MS), a cytometry, an ELISA (enzyme-linked immunosorbent assay). In some cases, assaying a protein pro-inflammatory immune marker may comprise a MS. In some cases, assaying a protein pro-inflammatory immune marker may comprise a cytometry. In some cases, assaying a protein pro-inflammatory immune marker may comprise an ELISA. In some cases, for protein/peptide-based pro-inflammatory immune marker, quantitative PCR, microarray, or sequencing may also be used for assaying the nucleic acid encoding the pro-inflammatory immune marker.

In some cases, assaying a chemical compound may comprise a MS. In some cases, assaying a chemical compound may comprise a liquid chromatography-mass spectrometry (LC-MS). In some cases, assaying a steroid compound may comprise a LC-MS. In some cases, assaying a microbially-derived metabolite may comprise a LC-MS. In some cases, assaying a membrane lipid may comprise a LC-MS. In some cases, assaying a pro-inflammatory immune marker may comprise a LC-MS.

In some cases—when assaying a nucleotide, a polynucleotide, a protein, an amino acid, a peptide, a lipid, a cell, a chemical compound, a saccharide, or a polysaccharide—the abundance therewith may comprise copy number, weight, dry weight, mole, volume, mass, or any combinations thereof of the substances being assayed.

In some instances, assaying a microbial community, a bacterial population, an androgenic steroid compound, a microbially-derived metabolite, a membrane lipid, a pro-inflammatory immune marker, or a combination thereof of a subject may be carried out prior to, during, or subsequent to the subject is administered with a pharmaceutical composition. In some instances, assaying a microbial community, a bacterial population, an androgenic steroid compound, a microbially-derived metabolite, a membrane lipid, a pro-inflammatory immune marker, or a combination thereof of a subject may be carried out prior to the subject is administered with a pharmaceutical composition. In some instances, assaying a microbial community, a bacterial population, an androgenic steroid compound, a microbially-derived metabolite, a membrane lipid, a pro-inflammatory immune marker, or a combination thereof of a subject may be carried out during the subject is administered with a pharmaceutical composition. In some instances, assaying a microbial community, a bacterial population, an androgenic steroid compound, a microbially-derived metabolite, a membrane lipid, a pro-inflammatory immune marker, or a combination thereof of a subject may be carried out subsequent to the subject is administered with a pharmaceutical composition. In some instances, assaying a microbial community, a bacterial population, an androgenic steroid compound, a microbially-derived metabolite, a membrane lipid, a pro-inflammatory immune marker, or a combination thereof of a subject may be carried out prior to, during, and subsequent to the subject is administered with a pharmaceutical composition.

In some cases, the assaying may be carried out at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, or at least about 5 years prior to a subject is administered with a pharmaceutical composition.

In some cases, the assaying may be carried out in a treatment period or during a pharmaceutical composition is being administered to a subject. In some cases, a treatment period or a period during a treatment may comprise a period of time between two different doses of pharmaceutical composition being administered.

In some cases, the assaying may be carried out in a washout period when it is carried out subsequent to a subject is administered with a pharmaceutical composition. In some instances, a washout period may comprise at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, or at least about 5 years subsequent to a subject is administered with a pharmaceutical composition. In some instances, a washout period may comprise any periods thereof without any administration of the pharmaceutical composition subsequent to the last administration of the pharmaceutical composition.

### Pharmaceutical Composition and Bacterial Populations

Provided herein are pharmaceutical compositions that can comprise one or more bacterial populations.

In some instances, a bacterial population may comprise at least one strain of Lactobacillus sp., at least one strain of Akkermansia sp., or at least one strain of Faecalibacterium sp. In some instances, a bacterial population may comprise at least one strain of a species of the Lactobacillaceae family, at least one strain of Akkermansia sp., or at least one strain of Faecalibacterium sp. In some instances, a bacterial population may comprise at least two of: at least one strain of Lactobacillus sp., at least one strain of Akkermansia sp., or at least one strain of Faecalibacterium sp. In some instances, a bacterial population may comprise at least two of: at least one strain of a species of the Lactobacillaceae family, at least one strain of Akkermansia sp., or at least one strain of Faecalibacterium sp. In some cases, a bacterial population may comprise at least one strain of Lactobacillus sp., at least one strain of Akkermansia sp., or at least one strain of Faecalibacterium sp. In some cases, a bacterial population may comprise at least one strain of a species of the Lactobacillaceae family, at least one strain of Akkermansia sp., or at least one strain of Faecalibacterium sp. In some cases, a bacterial population may comprise at least one strain of Lactobacillus sp., at least one strain of Akkermansia sp., and at least one strain of Faecalibacterium sp. In some cases, a bacterial population may comprise at least one strain of a species of the Lactobacillaceae family, at least one strain of Akkermansia sp., and at least one strain of Faecalibacterium sp.

In some instances, a bacterial population may comprise at least one strain of Lactobacillus sp. sp. or at least one strain of Akkermansia sp. In some instances, a bacterial population may comprise at least one strain of a species of Lactobacillaceae family or at least one strain of Akkermansia sp. In some cases, a bacterial population may comprise at least one strain of Lactobacillus sp. or at least one strain of Faecalibacterium sp. In some cases, a bacterial population may comprise at least one strain of a species of the Lactobacillaceae family or at least one strain of Faecalibacterium sp. In some cases, a bacterial population may comprise at least one strain of Akkermansia sp. or at least one strain of Faecalibacterium sp. In some cases, a bacterial population may comprise at least one strain of Lactobacillus sp. and at least one strain of Akkermansia sp. In some cases, a bacterial population may comprise at least one strain of a species of the Lactobacillaceae family and at least one strain of Akkermansia sp. In some cases, a bacterial population may comprise at least one strain of Lactobacillus sp. and at least one strain of Faecalibacterium sp. In some cases, a bacterial population may comprise at least one strain of a species of the Lactobacillaceae family and at least one strain of Faecalibacterium sp. In some cases, a bacterial population may comprise at least one strain of Akkermansia sp. and at least one strain of Faecalibacterium sp.

In some instances, a bacterial population may comprise at least two strains of Lactobacillus sp., at least two strains of Akkermansia sp., or at least two strains of Faecalibacterium sp. In some instances, a bacterial population may comprise at least two strains of species of the Lactobacillus family, at least two strains of Akkermansia sp., or at least two strains of Faecalibacterium sp. In some cases, a bacterial population may comprise at least three strains of Lactobacillus sp., at least three strains of Akkermansia sp., or at least three strains of Faecalibacterium sp. In some cases, a bacterial population may comprise at least three strains of species of the Lactobacillus family, at least three strains of Akkermansia sp., or at least three strains of Faecalibacterium sp. In some cases, a bacterial population may comprise at least more than three strains of Lactobacillus sp., at least more than three strains of Akkermansia sp., or at least more than three strains of Faecalibacterium sp. In some cases, a bacterial population may comprise at least more than three strains of species of the Lactobacillaceae family, at least more than three strains of Akkermansia sp., or at least more than three strains of Faecalibacterium sp. In some cases, a bacterial population may comprise at least one strain of Lactobacillus sp. In some cases, a bacterial population may comprise at least one strain of a species of the Lactobacillaceae family. In some cases, a bacterial population may comprise at least one strain of Akkermansia sp. In some cases, a bacterial population may comprise at least one strain of Faecalibacterium sp. In some cases, a bacterial population may comprise at least two strains of Lactobacillus sp. In some cases, a bacterial population may comprise at least two strains of species of the Lactobacillaceae family. In some cases, a bacterial population may comprise at least two strains of Akkermansia sp. In some cases, a bacterial population may comprise at least two strains of Faecalibacterium sp. In some cases, a bacterial population may comprise at least three strains of Lactobacillus sp. In some cases, a bacterial population may comprise at least three strains of species of the Lactobacillaceae family. In some cases, a bacterial population may comprise at least three strains of Akkermansia sp. In some cases, a bacterial population may comprise at least three strains of Faecalibacterium sp. In some cases, a bacterial population may comprise at least more than three strains of Lactobacillus sp. In some cases, a bacterial population may comprise at least more than three strains of species of the Lactobacillaceae family. In some cases, a bacterial population may comprise at least more than three strains of Akkermansia sp. In some cases, a bacterial population may comprise at least more than three strains of Faecalibacterium sp.

In some instances, a pharmaceutical composition that can comprise a bacterial population. Such bacterial population can comprise one or more different bacterial species and/or strains. Such bacterial species and/or strains can belong to one or more different bacterial phyla. Such bacterial phyla may include Verrucomicrobia, Firmicutes, or a combination thereof. Such bacterial phyla may also include Verrucomicrobia, Firmicutes, Proteobacteria, Actinobacteria, and/or Bacteroidetes, or a combination thereof.

In some instances, a species of Lactobacillus family may comprise a species of the Lactobacillus genus proposed in 1901, which is described in Zheng, J., et. al. Int. J Syst. Evol. Microbiol. 2020; 70:2782-2858 and is entirely incorporated herein by reference. The Lactobacillus genus may comprise Gram-positive, fermentative, facultatively anaerobic, and/or non-spore forming microorganisms. In some cases, the number of microorganisms that can be classified as Lactobacillus genus may increase, compared to those classified in 1901, with the broad definition of the 1901 classification. Lactobacillus genus may comprise about 261 species that comprise distinctive phenotypic, ecological, and/or genotypic characteristics. The number of species in the genus and/or the level of diversity within the Lactobacillus genus may exceed those of other bacterial genera and/or bacterial families. In this case, Lactobacillus can be reclassified. For example, the average nucleotide identity (ANI), average amino acid identity (AAI), core-gene average amino acid identity (cAAI), core genome phylogeny, signature genes, and metabolic, and/or ecological criteria of the bacterial species in the Lactobacillus genus and its sister taxa in the Lactobacillaceae and Leuconostocacae families are used to reclassify the Lactobacillus genus classified using the definition of 1901.

In some cases, under the reclassification system, the species of the Lactobacillaceae family may comprise about 26 different genera (Lactobacillus, Paralactobacillus, Pediococcus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus, and Lentilactobacillus), as well as merging the Leuconostocacae family into the Lactobacillaceae family. A comparison of the reclassified Lactobacillus species can be found using the Lactotax database, which can be found in the link: http://lactobacillus.ualberta.ca/ and is entirely incorporated herein by reference. The classification of Lactobacillus described herein, is also provided in Parks, D H et. al. Nat Biotechnol. 2018 November; 36(10):996-1004; Salvetti, E, et. al. Appl Environ Microbio. 2018 Aug. 17; 84(17). Print 2018 Sep. 1 Erratum in: Appl Environ Microbio. 2018 Oct. 1; 84(20); Markets and Markets: https://www.marketsandmarkets.com/Market-Reports/probiotic-market-advanced-technologies-and-global-market-69.html); Parker, C T, et. al. Int. J. Syst. Evol. Microbiol. 68:1825-1829; Duar, D M, et. al. FEMS Microbiol Rev. 2017 Aug. 1; 41(Supp_1):S27-S48; or Pane and Vinot 2019: https://www.microbiometimes.com/the-lactobacillus-taxonomy-change-is-coming-why-and-how-to-make-the-most-of-it/, each of which is entirely incorporated herein by reference.

In some instances, a bacterial population described herein may comprise one or more Lactobacillus sp. The one or more Lactobacillus sp. may include Lactobacillus johnsonii, Lactobacillus rhamnosus, Lactobacillus zeae, Lactobacillus acidipiscis, Lactobacillus acidophilus, Lactobacillus agilis, Lactobacillus aviarius, Lactobacillus brevis, Lactobacillus coleohominis, Lactobacillus crispatus, Lactobacillus crustorum, Lactobacillus curvatus, Lactobacillus diolivorans, Lactobacillus farraginis, Lactobacillus fermentum, Lactobacillus fuchuensis, Lactobacillus harbinensis, Lactobacillus helveticus, Lactobacillus hilgardii, Lactobacillus intestinalis, Lactobacillus jensenii, Lactobacillus kefiranofaciens, Lactobacillus kefiri, Lactobacillus lindneri, Lactobacillus mali, Lactobacillus manihotivorans, Lactobacillus mucosae, Lactobacillus oeni, Lactobacillus oligofermentans, Lactobacillus panis, Lactobacillus pantheris, Lactobacillus parabrevis, Lactobacillus paracollinoides, Lactobacillus parakefiri, Lactobacillus paraplantarum, Lactobacillus pentosus, Lactobacillus pontis, Lactobacillus reuteri, Lactobacillus rossiae, Lactobacillus salivarius, Lactobacillus siliginis, Lactobacillus sucicola, Lactobacillus vaccinostercus, Lactobacillus vaginalis, Lactobacillus vini, Lactococcus garvieae, or Lactococcus lactis, or a combination thereof. In some embodiments, the Lactobacillus sp. may comprise Lactobacillus johnsonii or Lactobacillus crispatus. In such instances, a bacterial population herein may comprise one or more Lactobacillus johnsonii or Lactobacillus crispatus strains. Such one or more Lactobacillus crispatus strain(s) may include Lactobacillus crispatus (DSM 33187) (i.e., L. crispatus (DSM 33187)). In various instances, a bacterial population herein comprises Lactobacillus crispatus (DSM 33187).

In some instances, a bacterial population described herein may comprise one or more Lactobacillus sp. The one or more Lactobacillus sp. may comprise Lactobacillus acidophilus, Lactobacillus crispatus, Lactobacillus helveticus, Lactobacillus intestinalis, Lactobacillus jensenii, Lactobacillus johnsonii, or Lactobacillus kefiranofaciens. In some cases, the bacterial population of the pharmaceutical composition may comprise at least two or a combination of Lactobacillus sp. The at least two or the combination of Lactobacillus sp. may comprise any one of Lactobacillus acidophilus, Lactobacillus crispatus, Lactobacillus helveticus, Lactobacillus intestinalis, Lactobacillus jensenii, Lactobacillus johnsonii, or Lactobacillus kefiranofaciens

In some instances, a bacterial population described herein may comprise one or more species of Lactobacillaceae family. The one or more species of the Lactobacillaceae family may include Lactobacillus johnsonii, Lactocaseibacillus rhamnosus, Lactocaseibacillus zeae, Ligilactobacillus acidipiscis, Lactobacillus acidophilus, Ligilactobacillus agilis, Ligilactobacillus aviarius, Levilactobacillus brevis, Limosilactobacillus coleohominis, Lactobacillus crispatus, Companilactobacillus crustorum, Latilactobacillus curvatus, Lentilactobacillus diolivorans, Lentilactobacillus farraginis, Limosilactobacillus fermentum, Latilactobacillus fuchuensis, Schleiferilactobacillus harbinensis, Lactobacillus helveticus, Lentilactobacillus hilgardii, Lactobacillus intestinalis, Lactobacillus jensenii, Lactobacillus kefiranofaciens, Lentilactobacillus kefiri, Fructilactobacillus lindneri, Liquorilactobacillus mali, Lactocaseibacillus manihotivorans, Limosilactobacillus mucosae, Liquorilactobacillus oeni, Paucilactobacillus oligofermentans, Limosilactobacillus panis, Lactocaseibacillus pantheris, Levilactobacillus parabrevis, Secundilactobacillus paracollinoides, Lentilactobacillus parakefiri, Lactoplantibacillus paraplantarum, Lactoplantibacillus pentosus, Limosiactobacillus pontis, Limosilactobacillus reuteri, Furfurilactobacillus rossiae, Ligilactobacillus salivarius, Furfurilactobacillus siliginis, Liquorilactobacillus sucicola, Paucilactobacillus vaccinostercus, Limosilactobacillus vaginalis, Liquorilactobacillus vini, Laclococcus garvieae, or Lactococcus lactis, or a combination thereof. In some embodiments, the species of the Lactobacillaceae family. is Lactobacillus johnsonii or Lactobacillus crispatus. In such instances, a bacterial population herein may comprise one Lactobacillus johnsonii or Lactobacillus crispatus strains. Such one or more Lactobacillus crispatus strain(s) may include Lactobacillus crispatus (DSM 33187) (i.e., L. crispatus (DSM 33187)). In various instances, a bacterial population herein comprises Lactobacillus crispatus (DSM 33187).

In some instances, a bacterial population described herein may comprise one or more species of the Lactobacillaceae family. The one or more species of the Lactobacillaceae family may include Lactobacillus acidophilus, Lactobacillus crispatus, Lactobacillus helveticus Lactobacillus intestinalis, Lactobacillus jensenii, Lactobacillus johnsonii, and Lactobacillus kefiranofaciens. TABLE 5 below shows the names of various Lactobacillus sp. under the 1901 classification and the reclassification.

TABLE 5
Lactobacillus sp. names before and after reclassification

| Name of Lactobacillus species | Name of Lactobacillus species |
|---|---|
| classified in 1901 | under reclassification |
| Lactobacillus acidipiscis | Ligilactobacillus acidipiscis |
| Lactobacillus acidophilus | Lactobacillus acidophilus |
| Lactobacillus agilis | Ligilactobacillus agilis |
| Lactobacillus aviarius | Ligilactobacillus aviarius |
| Lactobacillus brevis | Levilactobacillus brevis |
| Lactobacillus coleohominis | Limosilactobacillus coleohominis |
| Lactobacillus crispatus | Lactobacillus crispatus |
| Lactobacillus crustorum | Companilactobacillus crustorum |
| Lactobacillus curvatus | Latilactobacillus curvatus |
| Lactobacillus diolivorans | Lentilactobacillus diolivorans |
| Lactobacillus farraginis | Lentilactobacillus farraginis |
| Lactobacillus fermentum | Limosilactobacillus fermentum |
| Lactobacillus fuchuensis | Latilactobacillus fuchuensis |
| Lactobacillus harbinensis | Schleiferilactobacillus harbinensis |
| Lactobacillus helveticus | Lactobacillus helveticus |
| Lactobacillus hilgardii | Lentilactobacillus hilgardii |
| Lactobacillus intestinalis | Lactobacillus intestinalis |
| Lactobacillus jensenii | Lactobacillus jensenii |
| Lactobacillus johnsonii | Lactobacillus johnsonii |
| Lactobacillus kefiranofaciens | Lactobacillus kefiranofaciens |
| Lactobacillus kefiri | Lentilactobacillus kefiri |
| Lactobacillus lindneri | Fructilactobacillus lindneri |
| Lactobacillus mali | Liquorilactobacillus mali |
| Lactobacillus manihotivorans | Lacticaseibacillus manihotivorans |
| Lactobacillus mucosae | Limosilactobacillus mucosae |
| Lactobacillus oeni | Liquorilactobacillus oeni |
| Lactobacillus oligofermentans | Paucilactobacillus oligofermentans |
| Lactobacillus panis | Limosilactobacillus panis |
| Lactobacillus pantheris | Lacticaseibacillus pantheris |
| Lactobacillus parabrevis | Levilactobacillus parabrevis |
| Lactobacillus paracollinoides | Secundilactobacillus paracollinoides |
| Lactobacillus parakefiri | Lentilactobacillus parakefiri |
| Lactobacillus paraplantarum | Lactiplantibacillus paraplantarum |
| Lactobacillus pentosus | Lactiplantibacillus pentosus |
| Lactobacillus pontis | Limosilactobacillus pontis |
| Lactobacillus reuteri | Limosilactobacillus reuteri |
| Lactobacillus rhamnosus | Lacticaseibacillus rhamnosus |
| Lactobacillus rossiae | Furfurilactobacillus rossiae |
| Lactobacillus salivarius | Ligilactobacillus salivarius |
| Lactobacillus siliginis | Furfurilactobacillus siliginis |
| Lactobacillus sucicola | Liquorilactobacillus sucicola |
| Lactobacillus vaccinostercus | Paucilactobacillus vaccinostercus |
| Lactobacillus vaginalis | Limosilactobacillus vaginalis |
| Lactobacillus vini | Liquorilactobacillus vini |
| Lactobacillus zeae | Lacticaseibacillus zeae |

In some instances, a bacterial population herein may comprise one or more Akkermansia sp. Such one or more Akkermansia sp. may include Akkermansia muciniphila, Akkermansia glycaniphila, or a combination thereof. In some instances, the one or more Akkermansia sp. Is Akkermansia muciniphila. In such instances, a bacterial population herein may comprise one or more Akkermansia muciniphila strains. Such one or more Akkermansia muciniphila strains may include Akkermansia muciniphila (DSM 33213). In various instances, a bacterial population herein comprises Akkermansia muciniphila (DSM 33213).

In some instances, a bacterial population may comprise one or more Faecalibacterium sp. The one or more Faecalibacterium sp. may include Faecalibacterium prausnitzii. In such instances, a bacterial population herein may comprise one or more Faecalibacterium prausnitzii strains. Such one or more Faecalibacterium prausnitzii strains may include Faecalibacterium prausnitzii (DSM 33185), Faecalibacterium prausnitzii (DSM 33191), Faecalibacterium prausnitzii (DSM 33186), or Faecalibacterium prausnitzii (DSM 33190), or a combination thereof. In various instances, a bacterial population herein comprises Faecalibacterium prausnitzii (DSM 33185).

Further provided herein are bacterial populations that may comprise one or more strains of any one or more of Bacteroides sp., Blautia sp., Bifidobacterium sp., Coprococcus sp., or Dorea sp. In such instances, a bacterial population herein may comprise any one or more of Bacteroides faecis (DSM 33177), Bacteroides thetaiotaomicron (DSM 33178), Blautia producta (DSM 33180), Bifidobacterium longum (DSM 33179), Coprococcus comes (DSM 33176), or Dorea longicatena (DSM 33188). Exemplary strains for inclusion in a bacterial population described herein are listed in TABLE 1.

TABLE 1
Exemplary Bacterial Strains

| | Deposit | | Deposit |
|---|---|---|---|
| Bacterial Strain | ID # | Bacterial Strain | ID # |
| A. muciniphila | DSM 33213 | F. prausnitzii | DSM 33191 |
| B. longum | DSM 33179 | F. prausnitzii | DSM 33186 |
| B. producta | DSM 33180 | F. prausnitzi | DSM 33190 |
| B. thetaiotaomicron | DSM 33178 | L. crispatus | DSM 33187 |
| C. comes | DSM 33176 | B. faecis | DSM 33177 |
| F. prausnitzii | DSM 33185 | Dorea longicatena | DSM 33188 |

In some instances, a bacterial population may comprise one strain of Lactobacillus sp., one strain of Akkermansia sp., or one strain of Faecalibacterium sp. In some instances, a bacterial population may comprise one strain of a species of the Lactobacillaceae family, one strain of Akkermansia sp., or one strain of Faecalibacterium sp. In some cases, a bacterial population may comprise one strain of Lactobacillus sp., one strain of Akkermansia sp., and one strain of Faecalibacterium sp. In some cases, a bacterial population may comprise one strain of a species of the Lactobacillaceae family, one strain of Akkermansia sp., and one strain of Faecalibacterium sp. In some cases, a bacterial population may comprise one strain of Lactobacillus sp. or one strain of Akkermansia sp. In some cases, a bacterial population may comprise one strain of a species of the Lactobacillaceae family or one strain of Akkermansia sp. In some cases, a bacterial population may comprise one strain of Lactobacillus sp. or one strain of Faecalibacterium sp. In some cases, a bacterial population may comprise one strain of a species of the Lactobacillaceae family or one strain of Faecalibacterium sp. In some cases, a bacterial population may comprise one strain of Akkermansia sp. or one strain of Faecalibacterium sp. In some cases, a bacterial population may comprise one strain of Lactobacillus sp. and one strain of Akkermansia sp. In some cases, a bacterial population may comprise one strain of a species of the Lactobacillaceae family and one strain of Akkermansia sp. In some cases, a bacterial population may comprise one strain of Lactobacillus sp. and one strain of Faecalibacterium sp. In some cases, a bacterial population may comprise one strain of a species of the Lactobacillaceae family and one strain of Faecalibacterium sp. In some cases, a bacterial population may comprise one strain of Akkermansia sp. and one strain of Faecalibacterium sp.

In some instances, a bacterial population may comprise Lactobacillus crispatus (DSM 33187), Akkermansia muciniphila (DSM 33213), or Faecalibacterium prausnitzii (DSM 33185). In some cases, a bacterial population may comprise at least two of Lactobacillus crispatus (DSM 33187), Akkermansia muciniphila (DSM 33213), or Faecalibacterium prausnitzii (DSM 33185). In some cases, a bacterial population may comprise Lactobacillus crispatus (DSM 33187), Akkermansia muciniphila (DSM 33213), and Faecalibacterium prausnitzii (DSM 33185). In some cases, a bacterial population may comprise Lactobacillus crispatus (DSM 33187). In some cases, a bacterial population may comprise Akkermansia sp. In some cases, a bacterial population may comprise Faecalibacterium prausnitzii (DSM 33185). In some cases, a bacterial population may comprise at least two strains of Lactobacillus crispatus (DSM 33187).

In some instances, a bacterial population may comprise, consist essentially of, or consist of 20, 19, 18, 17, 16, 15, 14, 13, 12, 11, 10, 9, 8, 7, 6, 5, 4, 3, 2 or 1 bacterial species and/or strain(s). In some instances, such bacterial populations can comprise at least one bacterial strain selected from TABLE 1. In some embodiments, a bacterial population can consist of up to 3 different bacterial strains. In some embodiments, a bacterial population described herein comprises at least one, at least two, or all three bacterial strains listed in TABLE 2. In some instances, a bacterial population comprises or consists of the bacterial strains L. crispatus (DSM 33187), A. muciniphila (DSM 33213), and F. prausnitzii (DSM 33185).

TABLE 2
A Subset of Bacterial Strains

| Bacterial strain | Deposit ID # |
|---|---|
| Lactobacillus crispatus | DSM 33187 |
| Akkermansia muciniphila | DSM 33213 |
| Faecalibacterium prausnitzii | DSM 33185 |

In some cases, a purified or substantially purified bacterial strain or a mixture of more than one bacterial strain, refers to the bacterial strain or bacterial strains that are substantially enriched in a sample. In some cases, a purified or substantially purified bacterial strain(s) in sample may comprise at least about 50%, 60%, 70%, 80%, 85%, 90%, 95%, 99% or greater of the bacterial strain(s) in the sample. In some cases, a purified or substantially purified bacterial strain(s) in sample may also comprise less than about 40%, 30%, 20%, 15%, 14%, 13%, 12%, 11%, 10%, 9%, 8%, 7%, 6%, 5%, 4%, 3%, 2%, 1% or less of the strains other than the bacterial strain(s) present in the sample. Such strain may comprise a bacterial strain. Such strain may also comprise any non-bacterial strains such as strains from other organisms.

In some cases, a pharmaceutical composition may comprise pharmaceutical composition A, as outlined in EXAMPLE 1.

In some instances, a bacterial population may comprise a varying number of colony-forming units (CFU/dose) of each of the bacterial species and/or strain it contains. In some cases, a bacterial population may comprise from about 1×10\hat{ }3 CFU/dose to about 1×10\hat{ }12 CFU/dose of a bacterial species or strain. In some cases, a bacterial population may comprise from about 1×10\hat{ }3 CFU/dose to about 1×10\hat{ }11 CFU/dose of a bacterial species or strain. In some cases, a bacterial population may comprise from about 1×10\hat{ }3 CFU/dose to about 1×10\hat{ }10 CFU/dose of a bacterial species or strain. In some cases, a bacterial population may comprise from about 1×10\hat{ }3 CFU/dose to about 1×10\hat{ }9 CFU/dose of a bacterial species or strain. In some cases, a bacterial population may comprise from about 1×10\hat{ }3 CFU/dose to about 1×10\hat{ }8 CFU/dose of a bacterial species or strain. In some cases, a bacterial population may comprise from about 1×10\hat{ }4 CFU/dose to about 1×10\hat{ }12 CFU/dose of a bacterial species or strain. In some cases, a bacterial population may comprise from about 1×10\hat{ }4 CFU/dose to about 1×10\hat{ }11 CFU/dose of a bacterial species or strain. In some cases, a bacterial population may comprise from about 1×10\hat{ }4 CFU/dose to about 1×10\hat{ }10 CFU/dose of a bacterial species or strain. In some cases, a bacterial population may comprise from about 1×10\hat{ }4 CFU/dose to about 1×10\hat{ }9 CFU/dose of a bacterial species or strain. In some cases, a bacterial population may comprise from about 1×10\hat{ }4 CFU/dose to about 1×10\hat{ }8 CFU/dose of a bacterial species or strain. In some cases, a bacterial population may comprise from about 1×10\hat{ }5 CFU/dose to about 1×10\hat{ }12 CFU/dose of a bacterial species or strain. In some cases, a bacterial population may comprise from about 1×10\hat{ }5 CFU/dose to about 1×10\hat{ }11 CFU/dose of a bacterial species or strain. In some cases, a bacterial population may comprise from about 1×10\hat{ }5 CFU/dose to about 1×10\hat{ }10 CFU/dose of a bacterial species or strain. In some cases, a bacterial population may comprise from about 1×10\hat{ }5 CFU/dose to about 1×10\hat{ }9 CFU/dose of a bacterial species or strain. In some cases, a bacterial population may comprise from about 1×10\hat{ }5 CFU/dose to about 1×10\hat{ }8 CFU/dose of a bacterial species or strain. In some cases, a bacterial population may comprise from about 1×10\hat{ }6 CFU/dose to about 1×10\hat{ }12 CFU/dose of a bacterial species or strain. In some cases, a bacterial population may comprise from about 1×10\hat{ }6 CFU/dose to about 1×10\hat{ }11 CFU/dose of a bacterial species or strain. In some cases, a bacterial population may comprise from about 1×10\hat{ }6 CFU/dose to about 1×10\hat{ }10 CFU/dose of a bacterial species or strain. In some cases, a bacterial population may comprise from about 1×10\hat{ }6 CFU/dose to about 1×10\hat{ }9 CFU/dose of a bacterial species or strain. In some cases, a bacterial population may comprise from about 1×10\hat{ }6 CFU/dose to about 1×10\hat{ }8 CFU/dose of a bacterial species or strain. In some cases, a bacterial population may comprise from about 1×10\hat{ }7 CFU/dose to about 1×10\hat{ }12 CFU/dose of a bacterial species or strain. In some cases, a bacterial population may comprise from about 1×10\hat{ }7 CFU/dose to about 1×10\hat{ }11 CFU/dose of a bacterial species or strain. In some cases, a bacterial population may comprise from about 1×10\hat{ }7 CFU/dose to about 1×10\hat{ }10 CFU/dose of a bacterial species or strain. In some cases, a bacterial population may comprise from about 1×10\hat{ }7 CFU/dose to about 1×10\hat{ }9 CFU/dose of a bacterial species or strain. In some cases, a bacterial population may comprise from about 1×10\hat{ }7 CFU/dose to about 1×10\hat{ }8 CFU/dose of a bacterial species or strain. In some cases, a bacterial population may comprise from about 1×10\hat{ }8 CFU/dose to about 1×10\hat{ }12 CFU/dose of a bacterial species or strain. In some cases, a bacterial population may comprise from about 1×10\hat{ }8 CFU/dose to about 1×10\hat{ }11 CFU/dose of a bacterial species or strain. In some cases, a bacterial population may comprise from about 1×10\hat{ }8 CFU/dose to about 1×10\hat{ }10 CFU/dose of a bacterial species or strain. In some cases, a bacterial population may comprise from about 1×10\hat{ }8 CFU/dose to about 1×10\hat{ }9 CFU/dose of a bacterial species or strain. In some cases, a bacterial population may comprise from about 1×10\hat{ }9 CFU/dose to about 1×10\hat{ }12 CFU/dose of a bacterial species or strain. In some cases, a bacterial population may comprise from about 1×10\hat{ }9 CFU/dose to about 1×10\hat{ }11 CFU/dose of a bacterial species or strain. In some cases, a bacterial population may comprise from about 1×10\hat{ }9 CFU/dose to about 1×10\hat{ }10 CFU/dose of a bacterial species or strain. In some cases, such bacterial population may also comprise from about 1×10\hat{ }7 CFU/dose to about 1×10\hat{ }10 CFU/dose of a bacterial species or strain. In some instances, a bacterial population may comprise at least about 1×10\hat{ }3 CFU/dose, 5×10\hat{ }3 CFU/dose, 1×10\hat{ }4 CFU/dose, 5×10\hat{ }4 CFU/dose, 1×10\hat{ }5 CFU/dose, 5×10\hat{ }5 CFU/dose, 1×10\hat{ }6 CFU/dose, 5×10\hat{ }6 CFU/dose, 1×10\hat{ }7 CFU/dose, 5×10\hat{ }7 CFU/dose, 1×10\hat{ }8 CFU/dose, 5×10\hat{ }8 CFU/dose, 1×10\hat{ }9 CFU/dose, 5×10\hat{ }9 CFU/dose, 1×10\hat{ }10 CFU/dose, 5×10\hat{ }10 CFU/dose, 1×10\hat{ }11 CFU/dose, 5×10\hat{ }11 CFU/dose, or 1×10\hat{ }12 CFU/dose, but no more than about 5×10\hat{ }12 CFU/dose of a bacterial species or strain. The bacterial populations may also comprise from about 1×10\hat{ }6 to about 1×10\hat{ }11 CFU/dose per bacterial species or strain. In some cases, the bacterial populations may comprise from about 1×10\hat{ }3 to about 1×10\hat{ }12 CFU/dose per bacterial species or strain. In some instances, the bacterial populations may comprise from about 1×10\hat{ }8 to about 5×10\hat{ }10 CFU/dose per bacterial species or strain. In some instances, the bacterial populations may comprise from about 1×10\hat{ }7 to about 5×10\hat{ }10 CFU/dose per bacterial species or strain. In various cases, a bacterial population may comprise about 5×10\hat{ }8 CFU/dose per bacterial species or strain.

In some instances, a bacterial population may comprise a varying number of colony-forming units (CFU/dose) of bacterial cells. In some cases, a bacterial population may comprise from about 1×10\hat{ }3 CFU/dose to about 1×10\hat{ }12 CFU/dose of bacterial cells. In some cases, a bacterial population may comprise from about 1×10\hat{ }3 CFU/dose to about 1×10\hat{ }11 CFU/dose of bacterial cells. In some cases, a bacterial population may comprise from about 1×10\hat{ }3 CFU/dose to about 1×10\hat{ }10 CFU/dose of bacterial cells. In some cases, a bacterial population may comprise from about 1×10\hat{ }3 CFU/dose to about 1×10\hat{ }9 CFU/dose of bacterial cells. In some cases, a bacterial population may comprise from about 1×10\hat{ }3 CFU/dose to about 1×10\hat{ }8 CFU/dose of bacterial cells. In some cases, a bacterial population may comprise from about 1×10\hat{ }4 CFU/dose to about 1×10\hat{ }12 CFU/dose of bacterial cells. In some cases, a bacterial population may comprise from about 1×10\hat{ }4 CFU/dose to about 1×10\hat{ }11 CFU/dose of bacterial cells. In some cases, a bacterial population may comprise from about 1×10\hat{ }4 CFU/dose to about 1×10\hat{ }10 CFU/dose of bacterial cells. In some cases, a bacterial population may comprise from about 1×10\hat{ }4 CFU/dose to about 1×10\hat{ }9 CFU/dose of bacterial cells. In some cases, a bacterial population may comprise from about 1×10\hat{ }4 CFU/dose to about 1×10\hat{ }8 CFU/dose of bacterial cells. In some cases, a bacterial population may comprise from about 1×10\hat{ }5 CFU/dose to about 1×10\hat{ }12 CFU/dose of bacterial cells. In some cases, a bacterial population may comprise from about 1×10\hat{ }5 CFU/dose to about 1×10\hat{ }11 CFU/dose of bacterial cells. In some cases, a bacterial population may comprise from about 1×10\hat{ }5 CFU/dose to about 1×10\hat{ }10 CFU/dose of bacterial cells. In some cases, a bacterial population may comprise from about 1×10\hat{ }5 CFU/dose to about 1×10\hat{ }9 CFU/dose of bacterial cells. In some cases, a bacterial population may comprise from about 1×10\hat{ }5 CFU/dose to about 1×10\hat{ }8 CFU/dose of bacterial cells. In some cases, a bacterial population may comprise from about 1×10\hat{ }6 CFU/dose to about 1×10\hat{ }12 CFU/dose of bacterial cells. In some cases, a bacterial population may comprise from about 1×10\hat{ }6 CFU/dose to about 1×10\hat{ }11 CFU/dose of bacterial cells. In some cases, a bacterial population may comprise from about 1×10\hat{ }6 CFU/dose to about 1×10\hat{ }10 CFU/dose of bacterial cells. In some cases, a bacterial population may comprise from about 1×10\hat{ }6 CFU/dose to about 1×10\hat{ }9 CFU/dose of bacterial cells. In some cases, a bacterial population may comprise from about 1×10\hat{ }6 CFU/dose to about 1×10\hat{ }8 CFU/dose of bacterial cells. In some cases, a bacterial population may comprise from about 1×10\hat{ }7 CFU/dose to about 1×10\hat{ }12 CFU/dose of bacterial cells. In some cases, a bacterial population may comprise from about 1×10\hat{ }7 CFU/dose to about 1×10\hat{ }11 CFU/dose of bacterial cells. In some cases, a bacterial population may comprise from about 1×10\hat{ }7 CFU/dose to about 1×10\hat{ }10 CFU/dose of bacterial cells. In some cases, a bacterial population may comprise from about 1×10\hat{ }7 CFU/dose to about 1×10\hat{ }9 CFU/dose of bacterial cells. In some cases, a bacterial population may comprise from about 1×10\hat{ }7 CFU/dose to about 1×10\hat{ }8 CFU/dose of bacterial cells. In some cases, a bacterial population may comprise from about 1×10\hat{ }8 CFU/dose to about 1×10\hat{ }12 CFU/dose of bacterial cells. In some cases, a bacterial population may comprise from about 1×10\hat{ }8 CFU/dose to about 1×10\hat{ }11 CFU/dose of bacterial cells. In some cases, a bacterial population may comprise from about 1×10\hat{ }8 CFU/dose to about 1×10\hat{ }10 CFU/dose of bacterial cells. In some cases, a bacterial population may comprise from about 1×10\hat{ }8 CFU/dose to about 1×10\hat{ }9 CFU/dose of bacterial cells. In some cases, a bacterial population may comprise from about 1×10\hat{ }9 CFU/dose to about 1×10\hat{ }12 CFU/dose of bacterial cells. In some cases, a bacterial population may comprise from about 1×10\hat{ }9 CFU/dose to about 1×10\hat{ }11 CFU/dose of bacterial cells. In some cases, a bacterial population may comprise from about 1×10\hat{ }9 CFU/dose to about 1×10\hat{ }10 CFU/dose of bacterial cells. In some cases, such bacterial population may also comprise from about 1×10\hat{ }7 CFU/dose to about 1×10\hat{ }10 CFU/dose of bacterial cells. In some instances, a bacterial population may comprise at least about 1×10\hat{ }3 CFU/dose, 5×10\hat{ }3 CFU/dose, 1×10\hat{ }4 CFU/dose, 5×10\hat{ }4 CFU/dose, 1×10\hat{ }5 CFU/dose, 5×10\hat{ }5 CFU/dose, 1×10\hat{ }6 CFU/dose, 5×10\hat{ }6 CFU/dose, 1×10\hat{ }7 CFU/dose, 5×10\hat{ }7 CFU/dose, 1×10\hat{ }8 CFU/dose, 5×10\hat{ }8 CFU/dose, 1×10\hat{ }9 CFU/dose, 5×10\hat{ }9 CFU/dose, 1×10\hat{ }10 CFU/dose, 5×10\hat{ }10 CFU/dose, 1×10\hat{ }11 CFU/dose, 5×10\hat{ }11 CFU/dose, or 1×10\hat{ }12 CFU/dose, but no more than about 5×10\hat{ }12 CFU/dose of bacterial cells.

In some cases, at least one strain of Akkermansia sp. of a pharmaceutical composition may comprise from about 1×10\hat{ }6 CFU/dose to about 5×10\hat{ }9 CFU/dose. In some cases, at least one strain of Akkermansia sp. of a pharmaceutical composition may comprise from about 1×10\hat{ }6 CFU/dose to about 4×10\hat{ }9 CFU/dose. In some cases, at least one strain of Akkermansia sp. of a pharmaceutical composition may comprise from about 1×10\hat{ }6 CFU/dose to about 3×10\hat{ }9 CFU/dose. In some cases, at least one strain of Akkermansia sp. of a pharmaceutical composition may comprise from about 1×10\hat{ }6 CFU/dose to about 2×10\hat{ }9 CFU/dose. In some cases, at least one strain of Akkermansia sp. of a pharmaceutical composition may comprise from about 1×10\hat{ }6 CFU/dose to about 1×10\hat{ }9 CFU/dose. In some cases, at least one strain of Akkermansia sp. of a pharmaceutical composition may comprise from about 1×10\hat{ }6 CFU/dose to about 9×10\hat{ }8 CFU/dose. In some cases, at least one strain of Akkermansia sp. of a pharmaceutical composition may comprise from about 1×10\hat{ }6 CFU/dose to about 8×10\hat{ }8 CFU/dose. In some cases, at least one strain of Akkermansia sp. of a pharmaceutical composition may comprise from about 1×10\hat{ }6 CFU/dose to about 7×10\hat{ }8 CFU/dose. In some cases, at least one strain of Akkermansia sp. of a pharmaceutical composition may comprise from about 1×10\hat{ }6 CFU/dose to about 6×10\hat{ }8 CFU/dose. In some cases, at least one strain of Akkermansia sp. of a pharmaceutical composition may comprise from about 1×10\hat{ }6 CFU/dose to about 5×10\hat{ }8 CFU/dose. In some cases, at least one strain of Akkermansia sp. of a pharmaceutical composition may comprise from about 1×10\hat{ }6 CFU/dose to about 4×10\hat{ }8 CFU/dose. In some cases, at least one strain of Akkermansia sp. of a pharmaceutical composition may comprise from about 1×10\hat{ }6 CFU/dose to about 3×10\hat{ }8 CFU/dose. In some cases, at least one strain of Akkermansia sp. of a pharmaceutical composition may comprise from about 1×10\hat{ }6 CFU/dose to about 2×10\hat{ }8 CFU/dose. In some cases, at least one strain of Akkermansia sp. of a pharmaceutical composition may comprise from about 1×10\hat{ }6 CFU/dose to about 1×10\hat{ }8 CFU/dose. In some cases, at least one strain of Akkermansia sp. of a pharmaceutical composition may comprise from about 1×10\hat{ }6 CFU/dose to about 9×10\hat{ }7 CFU/dose. In some cases, at least one strain of Akkermansia sp. of a pharmaceutical composition may comprise from about 1×10\hat{ }6 CFU/dose to about 8×10\hat{ }7 CFU/dose. In some cases, at least one strain of Akkermansia sp. of a pharmaceutical composition may comprise from about 1×10\hat{ }6 CFU/dose to about 7×10\hat{ }7 CFU/dose. In some cases, at least one strain of Akkermansia sp. of a pharmaceutical composition may comprise from about 1×10\hat{ }6 CFU/dose to about 6×10\hat{ }7 CFU/dose. In some cases, at least one strain of Akkermansia sp. of a pharmaceutical composition may comprise from about 1×10\hat{ }6 CFU/dose to about 5×10\hat{ }7 CFU/dose. In some cases, at least one strain of Akkermansia sp. of a pharmaceutical composition may comprise from about 1×10\hat{ }6 CFU/dose to about 4×10\hat{ }7 CFU/dose. In some cases, at least one strain of Akkermansia sp. of a pharmaceutical composition may comprise from about 1×10\hat{ }6 CFU/dose to about 3×10\hat{ }7 CFU/dose. In some cases, at least one strain of Akkermansia sp. of a pharmaceutical composition may comprise from about 1×10\hat{ }6 CFU/dose to about 2×10\hat{ }7 CFU/dose. In some cases, at least one strain of Akkermansia sp. of a pharmaceutical composition may comprise from about 1×10\hat{ }6 CFU/dose to about 1×10\hat{ }7 CFU/dose. In some cases, at least one strain of Akkermansia sp. may comprise about 5×10\hat{ }9 CFU/dose. In some cases, at least one strain of Akkermansia sp. may comprise about 4×10\hat{ }9 CFU/dose. In some cases, at least one strain of Akkermansia sp. may comprise about 3×10\hat{ }9 CFU/dose. In some cases, at least one strain of Akkermansia sp. may comprise about 2×10\hat{ }9 CFU/dose. In some cases, at least one strain of Akkermansia sp. may comprise about 1×10\hat{ }9 CFU/dose. In some cases, at least one strain of Akkermansia sp. may comprise about 9×10\hat{ }8 CFU/dose. In some cases, at least one strain of Akkermansia sp. may comprise about 8×10\hat{ }8 CFU/dose. In some cases, at least one strain of Akkermansia sp. may comprise about 7×10\hat{ }8 CFU/dose. In some cases, at least one strain of Akkermansia sp. may comprise about 6×10\hat{ }8 CFU/dose. In some cases, at least one strain of Akkermansia sp. may comprise about 5×10\hat{ }8 CFU/dose. In some cases, at least one strain of Akkermansia sp. may comprise about 4×10\hat{ }8 CFU/dose. In some cases, at least one strain of Akkermansia sp. may comprise about 3×10\hat{ }8 CFU/dose. In some cases, at least one strain of Akkermansia sp. may comprise about 2×10\hat{ }8 CFU/dose. In some cases, at least one strain of Akkermansia sp. may comprise about 1×10\hat{ }8 CFU/dose. In some cases, at least one strain of Akkermansia sp. may comprise about 9×10\hat{ }7 CFU/dose. In some cases, at least one strain of Akkermansia sp. may comprise about 8×10\hat{ }7 CFU/dose. In some cases, at least one strain of Akkermansia sp. may comprise about 7×10\hat{ }7 CFU/dose. In some cases, at least one strain of Akkermansia sp. may comprise about 6×10\hat{ }7 CFU/dose. In some cases, at least one strain of Akkermansia sp. may comprise about 5×10\hat{ }7 CFU/dose. In some cases, at least one strain of Akkermansia sp. may comprise about 4×10\hat{ }7 CFU/dose. In some cases, at least one strain of Akkermansia sp. may comprise about 3×10\hat{ }7 CFU/dose. In some cases, at least one strain of Akkermansia sp. may comprise about 2×10\hat{ }7 CFU/dose. In some cases, at least one strain of Akkermansia sp. may comprise about 1×10\hat{ }7 CFU/dose.

In some cases, A. muciniphila (DSM 33213) of a pharmaceutical composition may comprise from about 1×10\hat{ }6 CFU/dose to about 5×10\hat{ }9 CFU/dose. In some cases, A. muciniphila (DSM 33213) of a pharmaceutical composition may comprise from about 1×10\hat{ }6 CFU/dose to about 4×10\hat{ }9 CFU/dose. In some cases, A. muciniphila (DSM 33213) of a pharmaceutical composition may comprise from about 1×10\hat{ }6 CFU/dose to about 3×10\hat{ }9 CFU/dose. In some cases, A. muciniphila (DSM 33213) of a pharmaceutical composition may comprise from about 1×10\hat{ }6 CFU/dose to about 2×10\hat{ }9 CFU/dose. In some cases, A. muciniphila (DSM 33213) of a pharmaceutical composition may comprise from about 1×10\hat{ }6 CFU/dose to about 1×10\hat{ }9 CFU/dose. In some cases, A. muciniphila (DSM 33213) of a pharmaceutical composition may comprise from about 1×10\hat{ }6 CFU/dose to about 9×10\hat{ }8 CFU/dose. In some cases, A. muciniphila (DSM 33213) of a pharmaceutical composition may comprise from about 1×10\hat{ }6 CFU/dose to about 8×10\hat{ }8 CFU/dose. In some cases, A. muciniphila (DSM 33213) of a pharmaceutical composition may comprise from about 1×10\hat{ }6 CFU/dose to about 7×10\hat{ }8 CFU/dose. In some cases, A. muciniphila (DSM 33213) of a pharmaceutical composition may comprise from about 1×10\hat{ }6 CFU/dose to about 6×10\hat{ }8 CFU/dose. In some cases, A. muciniphila (DSM 33213) of a pharmaceutical composition may comprise from about 1×10\hat{ }6 CFU/dose to about 5×10\hat{ }8 CFU/dose. In some cases, A. muciniphila (DSM 33213) of a pharmaceutical composition may comprise from about 1×10\hat{ }6 CFU/dose to about 4×10\hat{ }8 CFU/dose. In some cases, A. muciniphila (DSM 33213) of a pharmaceutical composition may comprise from about 1×10\hat{ }6 CFU/dose to about 3×10\hat{ }8 CFU/dose. In some cases, A. muciniphila (DSM 33213) of a pharmaceutical composition may comprise from about 1×10\hat{ }6 CFU/dose to about 2×10\hat{ }8 CFU/dose. In some cases, A. muciniphila (DSM 33213) of a pharmaceutical composition may comprise from about 1×10\hat{ }6 CFU/dose to about 1×10\hat{ }8 CFU/dose. In some cases, A. muciniphila (DSM 33213) of a pharmaceutical composition may comprise from about 1×10\hat{ }6 CFU/dose to about 9×10\hat{ }7 CFU/dose. In some cases, A. muciniphila (DSM 33213) of a pharmaceutical composition may comprise from about 1×10\hat{ }6 CFU/dose to about 8×10\hat{ }7 CFU/dose. In some cases, A. muciniphila (DSM 33213) of a pharmaceutical composition may comprise from about 1×10\hat{ }6 CFU/dose to about 7×10\hat{ }7 CFU/dose. In some cases, A. muciniphila (DSM 33213) of a pharmaceutical composition may comprise from about 1×10\hat{ }6 CFU/dose to about 6×10\hat{ }7 CFU/dose. In some cases, A. muciniphila (DSM 33213) of a pharmaceutical composition may comprise from about 1×10\hat{ }6 CFU/dose to about 5×10\hat{ }7 CFU/dose. In some cases, A. muciniphila (DSM 33213) of a pharmaceutical composition may comprise from about 1×10\hat{ }6 CFU/dose to about 4×10\hat{ }7 CFU/dose. In some cases, A. muciniphila (DSM 33213) of a pharmaceutical composition may comprise from about 1×10\hat{ }6 CFU/dose to about 3×10\hat{ }7 CFU/dose. In some cases, A. muciniphila (DSM 33213) of a pharmaceutical composition may comprise from about 1×10\hat{ }6 CFU/dose to about 2×10\hat{ }7 CFU/dose. In some cases, A. muciniphila (DSM 33213) of a pharmaceutical composition may comprise from about 1×10\hat{ }6 CFU/dose to about 1×10\hat{ }7 CFU/dose. In some cases, A. muciniphila (DSM 33213) may comprise about 5×10\hat{ }9 CFU/dose. In some cases, A. muciniphila (DSM 33213) may comprise about 4×10\hat{ }9 CFU/dose. In some cases, A. muciniphila (DSM 33213) may comprise about 3×10\hat{ }9 CFU/dose. In some cases, A. muciniphila (DSM 33213) may comprise about 2×10\hat{ }9 CFU/dose. In some cases, A. muciniphila (DSM 33213) may comprise about 1×10\hat{ }9 CFU/dose. In some cases, A. muciniphila (DSM 33213) may comprise about 9×10\hat{ }8 CFU/dose. In some cases, A. muciniphila (DSM 33213) may comprise about 8×10\hat{ }8 CFU/dose. In some cases, A. muciniphila (DSM 33213) may comprise about 7×10\hat{ }8 CFU/dose. In some cases, A. muciniphila (DSM 33213) may comprise about 6×10\hat{ }8 CFU/dose. In some cases, A. muciniphila (DSM 33213) may comprise about 5×10\hat{ }8 CFU/dose. In some cases, A. muciniphila (DSM 33213) may comprise about 4×10\hat{ }8 CFU/dose. In some cases, A. muciniphila (DSM 33213) may comprise about 3×10\hat{ }8 CFU/dose. In some cases, A. muciniphila (DSM 33213) may comprise about 2×10\hat{ }8 CFU/dose. In some cases, A. muciniphila (DSM 33213) may comprise about 1×10\hat{ }8 CFU/dose. In some cases, A. muciniphila (DSM 33213) may comprise about 9×10\hat{ }7 CFU/dose. In some cases, A. muciniphila (DSM 33213) may comprise about 8×10\hat{ }7 CFU/dose. In some cases, A. muciniphila (DSM 33213) may comprise about 7×10\hat{ }7 CFU/dose. In some cases, A. muciniphila (DSM 33213) may comprise about 6×10\hat{ }7 CFU/dose. In some cases, A. muciniphila (DSM 33213) may comprise about 5×10\hat{ }7 CFU/dose. In some cases, A. muciniphila (DSM 33213) may comprise about 4×10\hat{ }7 CFU/dose. In some cases, A. muciniphila (DSM 33213) may comprise about 3×10\hat{ }7 CFU/dose. In some cases, A. muciniphila (DSM 33213) may comprise about 2×10\hat{ }7 CFU/dose. In some cases, A. muciniphila (DSM 33213) may comprise about 1×10\hat{ }7 CFU/dose.

In some cases, at least one strain of Faecalibacterium sp. of a pharmaceutical composition may comprise from about 1×10\hat{ }6 CFU/dose to about 5×10\hat{ }9 CFU/dose. In some cases, at least one strain of Faecalibacterium sp. of a pharmaceutical composition may comprise from about 1×10\hat{ }6 CFU/dose to about 4×10\hat{ }9 CFU/dose. In some cases, at least one strain of Faecalibacterium sp. of a pharmaceutical composition may comprise from about 1×10\hat{ }6 CFU/dose to about 3×10\hat{ }9 CFU/dose. In some cases, at least one strain of Faecalibacterium sp. of a pharmaceutical composition may comprise from about 1×10\hat{ }6 CFU/dose to about 2×10\hat{ }9 CFU/dose. In some cases, at least one strain of Faecalibacterium sp. of a pharmaceutical composition may comprise from about 1×10\hat{ }6 CFU/dose to about 1×10\hat{ }9 CFU/dose. In some cases, at least one strain of Faecalibacterium sp. of a pharmaceutical composition may comprise from about 1×10\hat{ }6 CFU/dose to about 9×10\hat{ }8 CFU/dose. In some cases, at least one strain of Faecalibacterium sp. of a pharmaceutical composition may comprise from about 1×10\hat{ }6 CFU/dose to about 8×10\hat{ }8 CFU/dose. In some cases, at least one strain of Faecalibacterium sp. of a pharmaceutical composition may comprise from about 1×10\hat{ }6 CFU/dose to about 7×10\hat{ }8 CFU/dose. In some cases, at least one strain of Faecalibacterium sp. of a pharmaceutical composition may comprise from about 1×10\hat{ }6 CFU/dose to about 6×10\hat{ }8 CFU/dose. In some cases, at least one strain of Faecalibacterium sp. of a pharmaceutical composition may comprise from about 1×10\hat{ }6 CFU/dose to about 5×10\hat{ }8 CFU/dose. In some cases, at least one strain of Faecalibacterium sp. of a pharmaceutical composition may comprise from about 1×10\hat{ }6 CFU/dose to about 4×10\hat{ }8 CFU/dose. In some cases, at least one strain of Faecalibacterium sp. of a pharmaceutical composition may comprise from about 1×10\hat{ }6 CFU/dose to about 3×10\hat{ }8 CFU/dose. In some cases, at least one strain of Faecalibacterium sp. of a pharmaceutical composition may comprise from about 1×10\hat{ }6 CFU/dose to about 2×10\hat{ }8 CFU/dose. In some cases, at least one strain of Faecalibacterium sp. of a pharmaceutical composition may comprise from about 1×10\hat{ }6 CFU/dose to about 1×10\hat{ }8 CFU/dose. In some cases, at least one strain of Faecalibacterium sp. of a pharmaceutical composition may comprise from about 1×10\hat{ }6 CFU/dose to about 9×10\hat{ }7 CFU/dose. In some cases, at least one strain of Faecalibacterium sp. of a pharmaceutical composition may comprise from about 1×10\hat{ }6 CFU/dose to about 8×10\hat{ }7 CFU/dose. In some cases, at least one strain of Faecalibacterium sp. of a pharmaceutical composition may comprise from about 1×10\hat{ }6 CFU/dose to about 7×10\hat{ }7 CFU/dose. In some cases, at least one strain of Faecalibacterium sp. of a pharmaceutical composition may comprise from about 1×10\hat{ }6 CFU/dose to about 6×10\hat{ }7 CFU/dose. In some cases, at least one strain of Faecalibacterium sp. of a pharmaceutical composition may comprise from about 1×10\hat{ }6 CFU/dose to about 5×10\hat{ }7 CFU/dose. In some cases, at least one strain of Faecalibacterium sp. of a pharmaceutical composition may comprise from about 1×10\hat{ }6 CFU/dose to about 4×10\hat{ }7 CFU/dose. In some cases, at least one strain of Faecalibacterium sp. of a pharmaceutical composition may comprise from about 1×10\hat{ }6 CFU/dose to about 3×10\hat{ }7 CFU/dose. In some cases, at least one strain of Faecalibacterium sp. of a pharmaceutical composition may comprise from about 1×10\hat{ }6 CFU/dose to about 2×10\hat{ }7 CFU/dose. In some cases, at least one strain of Faecalibacterium sp. of a pharmaceutical composition may comprise from about 1×10\hat{ }6 CFU/dose to about 1×10\hat{ }7 CFU/dose. In some cases, at least one strain of Faecalibacterium sp. may comprise about 5×10\hat{ }9 CFU/dose. In some cases, at least one strain of Faecalibacterium sp. may comprise about 4×10\hat{ }9 CFU/dose. In some cases, at least one strain of Faecalibacterium sp. may comprise about 3×10\hat{ }9 CFU/dose. In some cases, at least one strain of Faecalibacterium sp. may comprise about 2×10\hat{ }9 CFU/dose. In some cases, at least one strain of Faecalibacterium sp. may comprise about 1×10\hat{ }9 CFU/dose. In some cases, at least one strain of Faecalibacterium sp. may comprise about 9×10\hat{ }8 CFU/dose. In some cases, at least one strain of Faecalibacterium sp. may comprise about 8×10\hat{ }8 CFU/dose. In some cases, at least one strain of Faecalibacterium sp. may comprise about 7×10\hat{ }8 CFU/dose. In some cases, at least one strain of Faecalibacterium sp. may comprise about 6×10\hat{ }8 CFU/dose. In some cases, at least one strain of Faecalibacterium sp. may comprise about 5×10\hat{ }8 CFU/dose. In some cases, at least one strain of Faecalibacterium sp. may comprise about 4×10\hat{ }8 CFU/dose. In some cases, at least one strain of Faecalibacterium sp. may comprise about 3×10\hat{ }8 CFU/dose. In some cases, at least one strain of Faecalibacterium sp. may comprise about 2×10\hat{ }8 CFU/dose. In some cases, at least one strain of Faecalibacterium sp. may comprise about 1×10\hat{ }8 CFU/dose. In some cases, at least one strain of Faecalibacterium sp. may comprise about 9×10\hat{ }7 CFU/dose. In some cases, at least one strain of Faecalibacterium sp. may comprise about 8×10\hat{ }7 CFU/dose. In some cases, at least one strain of Faecalibacterium sp. may comprise about 7×10\hat{ }7 CFU/dose. In some cases, at least one strain of Faecalibacterium sp. may comprise about 6×10\hat{ }7 CFU/dose. In some cases, at least one strain of Faecalibacterium sp. may comprise about 5×10\hat{ }7 CFU/dose. In some cases, at least one strain of Faecalibacterium sp. may comprise about 4×10\hat{ }7 CFU/dose. In some cases, at least one strain of Faecalibacterium sp. may comprise about 3×10\hat{ }7 CFU/dose. In some cases, at least one strain of Faecalibacterium sp. may comprise about 2×10\hat{ }7 CFU/dose. In some cases, at least one strain of Faecalibacterium sp. may comprise about 1×10\hat{ }7 CFU/dose.

In some cases, F. prausnitzii (DSM 33185) of a pharmaceutical composition may comprise from about 1×10\hat{ }6 CFU/dose to about 5×10\hat{ }9 CFU/dose. In some cases, F. prausnitzii (DSM 33185) of a pharmaceutical composition may comprise from about 1×10\hat{ }6 CFU/dose to about 4×10\hat{ }9 CFU/dose. In some cases, F. prausnitzii (DSM 33185) of a pharmaceutical composition may comprise from about 1×10\hat{ }6 CFU/dose to about 3×10\hat{ }9 CFU/dose. In some cases, F. prausnitzii (DSM 33185) of a pharmaceutical composition may comprise from about 1×10\hat{ }6 CFU/dose to about 2×10\hat{ }9 CFU/dose. In some cases, F. prausnitzii (DSM 33185) of a pharmaceutical composition may comprise from about 1×10\hat{ }6 CFU/dose to about 1×10\hat{ }9 CFU/dose. In some cases, F. prausnitzii (DSM 33185) of a pharmaceutical composition may comprise from about 1×10\hat{ }6 CFU/dose to about 9×10\hat{ }8 CFU/dose. In some cases, F. prausnitzii (DSM 33185) of a pharmaceutical composition may comprise from about 1×10\hat{ }6 CFU/dose to about 8×10\hat{ }8 CFU/dose. In some cases, F. prausnitzii (DSM 33185) of a pharmaceutical composition may comprise from about 1×10\hat{ }6 CFU/dose to about 7×10\hat{ }8 CFU/dose. In some cases, F. prausnitzii (DSM 33185) of a pharmaceutical composition may comprise from about 1×10\hat{ }6 CFU/dose to about 6×10\hat{ }8 CFU/dose. In some cases, F. prausnitzii (DSM 33185) of a pharmaceutical composition may comprise from about 1×10\hat{ }6 CFU/dose to about 5×10\hat{ }8 CFU/dose. In some cases, F. prausnitzii (DSM 33185) of a pharmaceutical composition may comprise from about 1×10\hat{ }6 CFU/dose to about 4×10\hat{ }8 CFU/dose. In some cases, F. prausnitzii (DSM 33185) of a pharmaceutical composition may comprise from about 1×10\hat{ }6 CFU/dose to about 3×10\hat{ }8 CFU/dose. In some cases, F. prausnitzii (DSM 33185) of a pharmaceutical composition may comprise from about 1×10\hat{ }6 CFU/dose to about 2×10\hat{ }8 CFU/dose. In some cases, F. prausnitzii (DSM 33185) of a pharmaceutical composition may comprise from about 1×10\hat{ }6 CFU/dose to about 1×10\hat{ }8 CFU/dose. In some cases, F. prausnitzii (DSM 33185) of a pharmaceutical composition may comprise from about 1×10\hat{ }6 CFU/dose to about 9×10\hat{ }7 CFU/dose. In some cases, F. prausnitzii (DSM 33185) of a pharmaceutical composition may comprise from about 1×10\hat{ }6 CFU/dose to about 8×10\hat{ }7 CFU/dose. In some cases, F. prausnitzii (DSM 33185) of a pharmaceutical composition may comprise from about 1×10\hat{ }6 CFU/dose to about 7×10\hat{ }7 CFU/dose. In some cases, F. prausnitzii (DSM 33185) of a pharmaceutical composition may comprise from about 1×10\hat{ }6 CFU/dose to about 6×10\hat{ }7 CFU/dose. In some cases, F. prausnitzii (DSM 33185) of a pharmaceutical composition may comprise from about 1×10\hat{ }6 CFU/dose to about 5×10\hat{ }7 CFU/dose. In some cases, F. prausnitzii (DSM 33185) of a pharmaceutical composition may comprise from about 1×10\hat{ }6 CFU/dose to about 4×10\hat{ }7 CFU/dose. In some cases, F. prausnitzii (DSM 33185) of a pharmaceutical composition may comprise from about 1×10\hat{ }6 CFU/dose to about 3×10\hat{ }7 CFU/dose. In some cases, F. prausnitzii (DSM 33185) of a pharmaceutical composition may comprise from about 1×10\hat{ }6 CFU/dose to about 2×10\hat{ }7 CFU/dose. In some cases, F. prausnitzii (DSM 33185) of a pharmaceutical composition may comprise from about 1×10\hat{ }6 CFU/dose to about 1×10\hat{ }7 CFU/dose. In some cases, F. prausnitzii (DSM 33185) may comprise about 5×10\hat{ }9 CFU/dose. In some cases, F. prausnitzii (DSM 33185) may comprise about 4×10\hat{ }9 CFU/dose. In some cases, F. prausnitzii (DSM 33185) may comprise about 3×10\hat{ }9 CFU/dose. In some cases, F. prausnitzii (DSM 33185) may comprise about 2×10\hat{ }9 CFU/dose. In some cases, F. prausnitzii (DSM 33185) may comprise about 1×10\hat{ }9 CFU/dose. In some cases, F. prausnitzii (DSM 33185) may comprise about 9×10\hat{ }8 CFU/dose. In some cases, F. prausnitzii (DSM 33185) may comprise about 8×10\hat{ }8 CFU/dose. In some cases, F. prausnitzii (DSM 33185) may comprise about 7×10\hat{ }8 CFU/dose. In some cases, F. prausnitzii (DSM 33185) may comprise about 6×10\hat{ }8 CFU/dose. In some cases, F. prausnitzii (DSM 33185) may comprise about 5×10\hat{ }8 CFU/dose. In some cases, F. prausnitzii (DSM 33185) may comprise about 4×10\hat{ }8 CFU/dose. In some cases, F. prausnitzii (DSM 33185) may comprise about 3×10\hat{ }8 CFU/dose. In some cases, F. prausnitzii (DSM 33185) may comprise about 2×10\hat{ }8 CFU/dose. In some cases, F. prausnitzii (DSM 33185) may comprise about 1×10\hat{ }8 CFU/dose. In some cases, F. prausnitzii (DSM 33185) may comprise about 9×10\hat{ }7 CFU/dose. In some cases, F. prausnitzii (DSM 33185) may comprise about 8×10\hat{ }7 CFU/dose. In some cases, F. prausnitzii (DSM 33185) may comprise about 7×10\hat{ }7 CFU/dose. In some cases, F. prausnitzii (DSM 33185) may comprise about 6×10\hat{ }7 CFU/dose. In some cases, F. prausnitzii (DSM 33185) may comprise about 5×10\hat{ }7 CFU/dose. In some cases, F. prausnitzii (DSM 33185) may comprise about 4×10\hat{ }7 CFU/dose. In some cases, F. prausnitzii (DSM 33185) may comprise about 3×10\hat{ }7 CFU/dose. In some cases, F. prausnitzii (DSM 33185) may comprise about 2×10\hat{ }7 CFU/dose. In some cases, F. prausnitzii (DSM 33185) may comprise about 1×10\hat{ }7 CFU/dose.

In some cases, at least one strain of Lactobacillus sp. of a pharmaceutical composition may comprise from about 1×10\hat{ }7 CFU/dose to about 5×10\hat{ }10 CFU/dose. In some cases, at least one strain of a species of the Lactobacillaceae family of a pharmaceutical composition may comprise from about 1×10\hat{ }7 CFU/dose to about 5×10\hat{ }10 CFU/dose. In some cases, at least one strain of Lactobacillus sp. of a pharmaceutical composition may comprise from about 1×10\hat{ }7 CFU/dose to about 4×10\hat{ }10 CFU/dose. In some cases, at least one strain of a species of the Lactobacillaceae family of a pharmaceutical composition may comprise from about 1×10\hat{ }7 CFU/dose to about 4×10\hat{ }10 CFU/dose. In some cases, at least one strain of Lactobacillus sp. of a pharmaceutical composition may comprise from about 1×10\hat{ }7 CFU/dose to about 3×10\hat{ }10 CFU/dose. In some cases, at least one strain of a species of the Lactobacillaceae family of a pharmaceutical composition may comprise from about 1×10\hat{ }7 CFU/dose to about 3×10\hat{ }10 CFU/dose. In some cases, at least one strain of Lactobacillus sp. of a pharmaceutical composition may comprise from about 1×10\hat{ }7 CFU/dose to about 2×10\hat{ }10 CFU/dose. In some cases, at least one strain of a species of the Lactobacillaceae family of a pharmaceutical composition may comprise from about 1×10\hat{ }7 CFU/dose to about 2×10\hat{ }10 CFU/dose. In some cases, at least one strain of Lactobacillus sp. of a pharmaceutical composition may comprise from about 1×10\hat{ }7 CFU/dose to about 1×10\hat{ }\hat{ }10 CFU/dose. In some cases, at least one strain of a species of the Lactobacillaceae family of a pharmaceutical composition may comprise from about 1×10\hat{ }7 CFU/dose to about 1×10\hat{ }\hat{ }10 CFU/dose. In some cases, at least one strain of Lactobacillus sp. of a pharmaceutical composition may comprise from about 1×10\hat{ }7 CFU/dose to about 9×10\hat{ }9 CFU/dose. In some cases, at least one strain of a species of the Lactobacillaceae family of a pharmaceutical composition may comprise from about 1×10\hat{ }7 CFU/dose to about 9×10\hat{ }9 CFU/dose. In some cases, at least one strain of Lactobacillus sp. of a pharmaceutical composition may comprise from about 1×10\hat{ }7 CFU/dose to about 8×10\hat{ }9 CFU/dose. In some cases, at least one strain of a species of the Lactobacillaceae family of a pharmaceutical composition may comprise from about 1×10\hat{ }7 CFU/dose to about 8×10\hat{ }9 CFU/dose. In some cases, at least one strain of Lactobacillus sp. of a pharmaceutical composition may comprise from about 1×10\hat{ }7 CFU/dose to about 7×10\hat{ }9 CFU/dose. In some cases, at least one strain of a species of the Lactobacillaceae family of a pharmaceutical composition may comprise from about 1×10\hat{ }7 CFU/dose to about 7×10\hat{ }9 CFU/dose. In some cases, at least one strain of Lactobacillus sp. of a pharmaceutical composition may comprise from about 1×10\hat{ }7 CFU/dose to about 6×10\hat{ }9 CFU/dose. In some cases, at least one strain of a species of the Lactobacillaceae family of a pharmaceutical composition may comprise from about 1×10\hat{ }7 CFU/dose to about 6×10\hat{ }9 CFU/dose. In some cases, at least one strain of Lactobacillus sp. of a pharmaceutical composition may comprise from about 1×10\hat{ }7 CFU/dose to about 5×10\hat{ }9 CFU/dose. In some cases, at least one strain of a species of the Lactobacillaceae family of a pharmaceutical composition may comprise from about 1×10\hat{ }7 CFU/dose to about 5×10\hat{ }9 CFU/dose. In some cases, at least one strain of Lactobacillus sp. of a pharmaceutical composition may comprise from about 1×10\hat{ }7 CFU/dose to about 4×10\hat{ }9 CFU/dose. In some cases, at least one strain of a species of the Lactobacillaceae family of a pharmaceutical composition may comprise from about 1×10\hat{ }7 CFU/dose to about 4×10\hat{ }9 CFU/dose. In some cases, at least one strain of Lactobacillus sp. of a pharmaceutical composition may comprise from about 1×10\hat{ }7 CFU/dose to about 3×10\hat{ }9 CFU/dose. In some cases, at least one strain of a species of the Lactobacillaceae family of a pharmaceutical composition may comprise from about 1×10\hat{ }7 CFU/dose to about 3×10\hat{ }9 CFU/dose. In some cases, at least one strain of Lactobacillus sp. of a pharmaceutical composition may comprise from about 1×10\hat{ }7 CFU/dose to about 2×10\hat{ }9 CFU/dose. In some cases, at least one strain of a species of the Lactobacillaceae family of a pharmaceutical composition may comprise from about 1×10\hat{ }7 CFU/dose to about 2×10\hat{ }9 CFU/dose. In some cases, at least one strain of Lactobacillus sp. of a pharmaceutical composition may comprise from about 1×10\hat{ }7 CFU/dose to about 1×10\hat{ }9 CFU/dose. In some cases, at least one strain of a species of the Lactobacillaceae family of a pharmaceutical composition may comprise from about 1×10\hat{ }7 CFU/dose to about 1×10\hat{ }9 CFU/dose. In some cases, at least one strain of Lactobacillus sp. of a pharmaceutical composition may comprise from about 1×10\hat{ }7 CFU/dose to about 9×10\hat{ }8 CFU/dose. In some cases, at least one strain of a species of the Lactobacillaceae family of a pharmaceutical composition may comprise from about 1×10\hat{ }7 CFU/dose to about 9×10\hat{ }8 CFU/dose. In some cases, at least one strain of Lactobacillus sp. of a pharmaceutical composition may comprise from about 1×10\hat{ }7 CFU/dose to about 8×10\hat{ }8 CFU/dose. In some cases, at least one strain of a species of the Lactobacillaceae family of a pharmaceutical composition may comprise from about 1×10\hat{ }7 CFU/dose to about 8×10\hat{ }8 CFU/dose. In some cases, at least one strain of Lactobacillus sp. of a pharmaceutical composition may comprise from about 1×10\hat{ }7 CFU/dose to about 7×10\hat{ }8 CFU/dose. In some cases, at least one strain of a species of the Lactobacillaceae family of a pharmaceutical composition may comprise from about 1×10\hat{ }7 CFU/dose to about 7×10\hat{ }8 CFU/dose. In some cases, at least one strain of Lactobacillus sp. of a pharmaceutical composition may comprise from about 1×10\hat{ }7 CFU/dose to about 6×10\hat{ }8 CFU/dose. In some cases, at least one strain of a species of the Lactobacillaceae family of a pharmaceutical composition may comprise from about 1×10\hat{ }7 CFU/dose to about 6×10\hat{ }8 CFU/dose. In some cases, at least one strain of Lactobacillus sp. of a pharmaceutical composition may comprise from about 1×10\hat{ }7 CFU/dose to about 5×10\hat{ }8 CFU/dose. In some cases, at least one strain of a species of the Lactobacillaceae family of a pharmaceutical composition may comprise from about 1×10\hat{ }7 CFU/dose to about 5×10\hat{ }8 CFU/dose. In some cases, at least one strain of Lactobacillus sp. of a pharmaceutical composition may comprise from about 1×10\hat{ }7 CFU/dose to about 4×10\hat{ }8 CFU/dose. In some cases, at least one strain of a species of the Lactobacillaceae family of a pharmaceutical composition may comprise from about 1×10\hat{ }7 CFU/dose to about 4×10\hat{ }8 CFU/dose. In some cases, at least one strain of Lactobacillus sp. of a pharmaceutical composition may comprise from about 1×10\hat{ }7 CFU/dose to about 3×10\hat{ }8 CFU/dose. In some cases, at least one strain of a species of the Lactobacillaceae family of a pharmaceutical composition may comprise from about 1×10\hat{ }7 CFU/dose to about 3×10\hat{ }8 CFU/dose. In some cases, at least one strain of Lactobacillus sp. of a pharmaceutical composition may comprise from about 1×10\hat{ }7 CFU/dose to about 2×10\hat{ }8 CFU/dose. In some cases, at least one strain of a species of the Lactobacillaceae family of a pharmaceutical composition may comprise from about 1×10\hat{ }7 CFU/dose to about 2×10\hat{ }8 CFU/dose. In some cases, at least one strain of Lactobacillus sp. of a pharmaceutical composition may comprise from about 1×10\hat{ }7 CFU/dose to about 1×10\hat{ }8 CFU/dose. In some cases, at least one strain of a species of Lactobacillaceae family of a pharmaceutical composition may comprise from about 1×10\hat{ }7 CFU/dose to about 1×10\hat{ }8 CFU/dose. In some cases, at least one strain of Lactobacillus sp. may comprise about 5×10\hat{ }9 CFU/dose. In some cases, at least one strain of a species of the Lactobacillaceae family may comprise about 5×10\hat{ }9 CFU/dose. In some cases, at least one strain of Lactobacillus sp. may comprise about 4×10\hat{ }9 CFU/dose. In some cases, at least one strain of a species of the Lactobacillaceae family may comprise about 4×10\hat{ }9 CFU/dose. In some cases, at least one strain of Lactobacillus sp. may comprise about 3×10\hat{ }9 CFU/dose. In some cases, at least one strain of a species of the Lactobacillaceae family may comprise about 3×10\hat{ }9 CFU/dose. In some cases, at least one strain of Lactobacillus sp. may comprise about 2×10\hat{ }9 CFU/dose. In some cases, at least one strain of a species of the Lactobacillaceae family may comprise about 2×10\hat{ }9 CFU/dose. In some cases, at least one strain of Lactobacillus sp. may comprise about 1×10\hat{ }9 CFU/dose. In some cases, at least one strain of a species of the Lactobacillaceae family may comprise about 1×10\hat{ }9 CFU/dose. In some cases, at least one strain of Lactobacillus sp. may comprise about 9×10\hat{ }8 CFU/dose. In some cases, at least one strain of a species of the Lactobacillaceae family may comprise about 9×10\hat{ }8 CFU/dose. In some cases, at least one strain of Lactobacillus sp. may comprise about 8×10\hat{ }8 CFU/dose. In some cases, at least one strain of a species of the Lactobacillaceae family may comprise about 8×10\hat{ }8 CFU/dose. In some cases, at least one strain of Lactobacillus sp. may comprise about 7×10\hat{ }8 CFU/dose. In some cases, at least one strain of a species of the Lactobacillaceae family may comprise about 7×10\hat{ }8 CFU/dose. In some cases, at least one strain of Lactobacillus sp. may comprise about 6×10\hat{ }8 CFU/dose. In some cases, at least one strain of a species of the Lactobacillaceae family may comprise about 6×10\hat{ }8 CFU/dose. In some cases, at least one strain of Lactobacillus sp. may comprise about 5×10\hat{ }8 CFU/dose. In some cases, at least one strain of a species of the Lactobacillaceae family may comprise about 5×10\hat{ }8 CFU/dose. In some cases, at least one strain of Lactobacillus sp. may comprise about 4×10\hat{ }8 CFU/dose. In some cases, at least one strain of a species of the Lactobacillaceae family may comprise about 4×10\hat{ }8 CFU/dose. In some cases, at least one strain of Lactobacillus sp. may comprise about 3×10\hat{ }8 CFU/dose. In some cases, at least one strain of a species of the Lactobacillaceae family may comprise about 3×10\hat{ }8 CFU/dose. In some cases, at least one strain of Lactobacillus sp. may comprise about 2×10\hat{ }8 CFU/dose. In some cases, at least one strain of a species of the Lactobacillaceae family may comprise about 2×10\hat{ }8 CFU/dose. In some cases, at least one strain of Lactobacillus sp. may comprise about 1×10\hat{ }8 CFU/dose. In some cases, at least one strain of a species of the Lactobacillaceae family may comprise about 1×10\hat{ }8 CFU/dose. In some cases, at least one strain of Lactobacillus sp. may comprise about 9×10\hat{ }7 CFU/dose. In some cases, at least one strain of a species of the Lactobacillaceae family may comprise about 9×10\hat{ }7 CFU/dose. In some cases, at least one strain of Lactobacillus sp. may comprise about 8×10\hat{ }7 CFU/dose. In some cases, at least one strain of a species of the Lactobacillaceae family may comprise about 8×10\hat{ }7 CFU/dose. In some cases, at least one strain of Lactobacillus sp. may comprise about 7×10\hat{ }7 CFU/dose. In some cases, at least one strain of a species of the Lactobacillaceae family may comprise about 7×10\hat{ }7 CFU/dose. In some cases, at least one strain of Lactobacillus sp. may comprise about 6×10\hat{ }7 CFU/dose. In some cases, at least one strain of a species of the Lactobacillaceae family may comprise about 6×10\hat{ }7 CFU/dose. In some cases, at least one strain of Lactobacillus sp. may comprise about 5×10\hat{ }7 CFU/dose. In some cases, at least one strain of a species of the Lactobacillaceae family may comprise about 5×10\hat{ }7 CFU/dose. In some cases, at least one strain of Lactobacillus sp. may comprise about 4×10\hat{ }7 CFU/dose. In some cases, at least one strain of a species of the Lactobacillaceae family may comprise about 4×10\hat{ }7 CFU/dose. In some cases, at least one strain of Lactobacillus sp. may comprise about 3×10\hat{ }7 CFU/dose. In some cases, at least one strain of a species of the Lactobacillaceae family may comprise about 3×10\hat{ }7 CFU/dose. In some cases, at least one strain of Lactobacillus sp. may comprise about 2×10\hat{ }7 CFU/dose. In some cases, at least one strain of a species of the Lactobacillaceae family may comprise about 2×10\hat{ }7 CFU/dose. In some cases, at least one strain of Lactobacillus sp. may comprise about 1×10\hat{ }7 CFU/dose. In some cases, at least one strain of a species of the Lactobacillaceae family may comprise about 1×10\hat{ }7 CFU/dose.

In some cases, L. crispatus (DSM 33187) of a pharmaceutical composition may comprise from about 1×10\hat{ }7 CFU/dose to about 5×10\hat{ }10 CFU/dose. In some cases, L. crispatus (DSM 33187) of a pharmaceutical composition may comprise from about 1×10\hat{ }7 CFU/dose to about 4×10\hat{ }10 CFU/dose. In some cases, L. crispatus (DSM 33187) of a pharmaceutical composition may comprise from about 1×10\hat{ }7 CFU/dose to about 3×10\hat{ }10 CFU/dose. In some cases, L. crispatus (DSM 33187) of a pharmaceutical composition may comprise from about 1×10\hat{ }7 CFU/dose to about 2×10\hat{ }10 CFU/dose. In some cases, L. crispatus (DSM 33187) of a pharmaceutical composition may comprise from about 1×10\hat{ }7 CFU/dose to about 1×10\hat{ }10 CFU/dose. In some cases, L. crispatus (DSM 33187) of a pharmaceutical composition may comprise from about 1×10\hat{ }7 CFU/dose to about 9×10\hat{ }9 CFU/dose. In some cases, L. crispatus (DSM 33187) of a pharmaceutical composition may comprise from about 1×10\hat{ }7 CFU/dose to about 8×10\hat{ }9 CFU/dose. In some cases, L. crispatus (DSM 33187) of a pharmaceutical composition may comprise from about 1×10\hat{ }7 CFU/dose to about 7×10\hat{ }9 CFU/dose. In some cases, L. crispatus (DSM 33187) of a pharmaceutical composition may comprise from about 1×10\hat{ }7 CFU/dose to about 6×10\hat{ }9 CFU/dose. In some cases, L. crispatus (DSM 33187) of a pharmaceutical composition may comprise from about 1×10\hat{ }7 CFU/dose to about 5×10\hat{ }9 CFU/dose. In some cases, L. crispatus (DSM 33187) of a pharmaceutical composition may comprise from about 1×10\hat{ }7 CFU/dose to about 4×10\hat{ }9 CFU/dose. In some cases, L. crispatus (DSM 33187) of a pharmaceutical composition may comprise from about 1×10\hat{ }7 CFU/dose to about 3×10\hat{ }9 CFU/dose. In some cases, L. crispatus (DSM 33187) of a pharmaceutical composition may comprise from about 1×10\hat{ }7 CFU/dose to about 2×10\hat{ }9 CFU/dose. In some cases, L. crispatus (DSM 33187) of a pharmaceutical composition may comprise from about 1×10\hat{ }7 CFU/dose to about 1×10\hat{ }9 CFU/dose. In some cases, L. crispatus (DSM 33187) of a pharmaceutical composition may comprise from about 1×10\hat{ }7 CFU/dose to about 9×10\hat{ }8 CFU/dose. In some cases, L. crispatus (DSM 33187) of a pharmaceutical composition may comprise from about 1×10\hat{ }7 CFU/dose to about 8×10\hat{ }8 CFU/dose. In some cases, L. crispatus (DSM 33187) of a pharmaceutical composition may comprise from about 1×10\hat{ }7 CFU/dose to about 7×10\hat{ }8 CFU/dose. In some cases, L. crispatus (DSM 33187) of a pharmaceutical composition may comprise from about 1×10\hat{ }7 CFU/dose to about 6×10\hat{ }8 CFU/dose. In some cases, L. crispatus (DSM 33187) of a pharmaceutical composition may comprise from about 1×10\hat{ }7 CFU/dose to about 5×10\hat{ }8 CFU/dose. In some cases, L. crispatus (DSM 33187) of a pharmaceutical composition may comprise from about 1×10\hat{ }7 CFU/dose to about 4×10\hat{ }8 CFU/dose. In some cases, L. crispatus (DSM 33187) of a pharmaceutical composition may comprise from about 1×10\hat{ }7 CFU/dose to about 3×10\hat{ }8 CFU/dose. In some cases, L. crispatus (DSM 33187) of a pharmaceutical composition may comprise from about 1×10\hat{ }7 CFU/dose to about 2×10\hat{ }8 CFU/dose. In some cases, L. crispatus (DSM 33187) of a pharmaceutical composition may comprise from about 1×10\hat{ }7 CFU/dose to about 1×10\hat{ }8 CFU/dose. In some cases, L. crispatus (DSM 33187) may comprise about 5×10\hat{ }9 CFU/dose. In some cases, L. crispatus (DSM 33187) may comprise about 4×10\hat{ }9 CFU/dose. In some cases, L. crispatus (DSM 33187) may comprise about 3×10\hat{ }9 CFU/dose. In some cases, L. crispatus (DSM 33187) may comprise about 2×10\hat{ }9 CFU/dose. In some cases, L. crispatus (DSM 33187) may comprise about 1×10\hat{ }9 CFU/dose. In some cases, L. crispatus (DSM 33187) may comprise about 9×10\hat{ }8 CFU/dose. In some cases, L. crispatus (DSM 33187) may comprise about 8×10\hat{ }8 CFU/dose. In some cases, L. crispatus (DSM 33187) may comprise about 7×10\hat{ }8 CFU/dose. In some cases, L. crispatus (DSM 33187) may comprise about 6×10\hat{ }8 CFU/dose. In some cases, L. crispatus (DSM 33187) may comprise about 5×10\hat{ }8 CFU/dose. In some cases, L. crispatus (DSM 33187) may comprise about 4×10\hat{ }8 CFU/dose. In some cases, L. crispatus (DSM 33187) may comprise about 3×10\hat{ }8 CFU/dose. In some cases, L. crispatus (DSM 33187) may comprise about 2×10\hat{ }8 CFU/dose. In some cases, L. crispatus (DSM 33187) may comprise about 1×10\hat{ }8 CFU/dose. In some cases, L. crispatus (DSM 33187) may comprise about 9×10\hat{ }7 CFU/dose. In some cases, L. crispatus (DSM 33187) may comprise about 8×10\hat{ }7 CFU/dose. In some cases, L. crispatus (DSM 33187) may comprise about 7×10\hat{ }7 CFU/dose. In some cases, L. crispatus (DSM 33187) may comprise about 6×10\hat{ }7 CFU/dose. In some cases, L. crispatus (DSM 33187) may comprise about 5×10\hat{ }7 CFU/dose. In some cases, L. crispatus (DSM 33187) may comprise about 4×10\hat{ }7 CFU/dose. In some cases, L. crispatus (DSM 33187) may comprise about 3×10\hat{ }7 CFU/dose. In some cases, L. crispatus (DSM 33187) may comprise about 2×10\hat{ }7 CFU/dose. In some cases, L. crispatus (DSM 33187) may comprise about 1×10\hat{ }7 CFU/dose.

In some cases herein, a bacterial population for use in a pharmaceutical composition of this disclosure may comprise or consist of about 5×10\hat{ }8 CFU/mL of any of the bacterial strains L. crispatus (DSM 33187), A. muciniphila (DSM 33213), and/or F. prausnitzii (DSM 33185). In such instances, the bacterial population may consist of about 5×10\hat{ }8 CFU/mL of the bacterial strains L. crispatus (DSM 33187), A. muciniphila (DSM 33213), and F. prausnitzii (DSM 33185).

In instances where a pharmaceutical composition is formulated into a unit dose for administration, such CFU/dose values may be per mass unit (e.g., 5×10\hat{ }8 CFU/dose/g) or volume unit (e.g., 5×10\hat{ }8 CFU/dose/mL) of such dosage form. In other cases, the CFU/dose value may be equivalent to CFU/capsule if one dose is administered as one capsule, or if one capsule contains a dose. In some cases, a dose may be in other forms described elsewhere in this disclosure.

### Pharmaceutical Composition Forms and Administrations

In some instances, a pharmaceutical composition may be formulated into a suspension. In some cases, a pharmaceutical composition may be formulated into an oral dosage form. An oral dosage form of a pharmaceutical composition, in some cases, may comprise a capsule, tablet, emulsion, suspension, syrup, gel, gum, paste, herbal tea, drops, dissolving granules, powders, tablets, lyophilizate, a popsicle, foams, or ice cream. In some cases, an oral dosage form of a pharmaceutical composition may comprise a capsule. In some cases, an oral dosage form of a pharmaceutical composition may comprise a dissolving granule. In some cases, an oral dosage form of a pharmaceutical composition may comprise a drop. In some cases, an oral dosage form of a pharmaceutical composition may comprise an emulsion. In some cases, an oral dosage form of a pharmaceutical composition may comprise a foam. In some cases, an oral dosage form of a pharmaceutical composition may comprise a gel. In some cases, an oral dosage form of a pharmaceutical composition may comprise a gum. In some cases, an oral dosage form of a pharmaceutical composition may comprise an herbal tea. In some cases, an oral dosage form of a pharmaceutical composition may comprise an ice cream. In some cases, an oral dosage form of a pharmaceutical composition may comprise a lyophilizate. In some cases, an oral dosage form of a pharmaceutical composition may comprise a paste. In some cases, an oral dosage form of a pharmaceutical composition may comprise a popsicle. In some cases, an oral dosage form of a pharmaceutical composition may comprise a powder. In some cases, an oral dosage form of a pharmaceutical composition may comprise a suspension. In some cases, an oral dosage form of a pharmaceutical composition may comprise a syrup. In some cases, an oral dosage form of a pharmaceutical composition may comprise a tablet. In some cases, an oral dosage form of a pharmaceutical composition may comprise a pill, geltab, sachet, a lozenge, or any other suitable oral dosage form. In some cases, a pharmaceutical composition in an oral dosage or suspension form may be administered alone. In other cases, a pharmaceutical composition in an oral dosage or suspension form may be mixed with a food product for administration to a subject. Such a food product may comprise baby formula, milk, or any derivatives thereof. In some cases, a pharmaceutical composition may be formulated into a parenteral administration form. A parenteral administration form, in some cases, may comprise various non-oral routes, e.g., in the form of a suppository.

In some cases, lyophilization may comprise a water removal process comprising freezing and drying cycles. In some cases, a lyophilized substance may have less than 10%, 9%, 8%, 7%, 6%, 5%, 4%, 3%, 2%, 1% or less of the water content, by weight, in the substance. Lyophilization may comprise a first freezing cycle, followed by subsequent drying cycles. The freezing cycle may freeze a substance such that a sublimation state and not a melt state of the substance will occur in the subsequent drying cycle. The first drying cycle may comprise lowering pressure and increasing temperature to remove the water content from the substance. The first drying cycle may comprise placing the substance in a vacuum. The first drying cycle may comprise slow heating. Optionally, a second drying cycle may be used following the first drying cycle. A second drying cycle may comprise rapid heating in order to break the bonds between the substance and the water molecules.

In some cases, a pharmaceutical composition may be encompassed by a primary container. In some instances, a pharmaceutical composition may be encompassed by a capsule. In some cases, a capsule encompassing a pharmaceutical composition may comprise a plant-based capsule. In some cases, a capsule encompassing a pharmaceutical composition may comprise a vegan capsule. In some cases, a plant-based capsule may comprise a plant-derived material. The plant-derived material, in some cases, may comprise a cellulose-based polymer. In some cases, a plant-based capsule may comprise a hypromellose capsule. In some cases, a plant-based capsule may comprise a hydroxypropyl methylcellulose (HPMC) capsule. In some cases, a plant-based capsule may comprise a starch capsule. In some cases, a plant-based capsule may comprise a hydrolyzed plant-based collagen capsule. In some cases, a plant-based capsule may comprise a pullulan capsule. In some cases, a plant-based capsule may comprise a tapioca capsule. In some cases, a plant-based capsule may comprise the combinations of any plant-based materials described thereof. A primary container, in some cases, may comprise any capsules described herein and thereof and derivatives herein and thereof.

In some cases, a capsule may not be administered to a subject. In some cases, a capsule may not be administered to a subject alongside the pharmaceutical composition. In other cases, a capsule may be administered to a subject.

A capsule may be enteric-coated. An enteric-coated capsule may comprise fatty acids, waxes, shellac, plastics, plant fibers, or any combination thereof. A capsule may have a size of 000, 00, 0, 1, 2, 3, 4, or 5 Empty Pill Capsule Size. In some cases, a capsule may comprise gelatin.

A capsule may be starch-free, gluten-free, and preservative-free. >90% of a capsule dissolves in water, pH=1.2 solution, sodium acetate buffer USP. (pH=4.5), or sodium phosphate buffer (pH=7.2) within 60 minutes, when measured by the dissolution of acetaminophen when the capsule is filled with unformulated acetaminophen. A capsule may have a disintegration endpoint of about 1.6 minutes, as measured at 37° C. with de-ionized water. A capsule may have a disintegration endpoint of about 0.1, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 3.4, 2.5, 2.6, 2.7, 2.8, 2.9, 3, 3.1, 3.2, 3.3, 3.4, 3.5, 3.6, 3.7, 3.8, 3.9, or 4 minutes, as measured at 37° C. with de-ionized water. A capsule may have a disintegration endpoint of 0.1 to 0.5 minutes, 0.51 to 0.6 minutes, 0.61 to 0.7 minutes, 0.71 to 0.8 minutes, 0.81 to 0.9 minutes, 0.91 to 1 minutes, 1.01 to 1.1 minutes, 1.11 to 1.2 minutes, 1.21 to 1.3 minutes, 1.31 to 1.4 minutes, 1.41 to 1.5 minutes, 1.51 to 1.6 minutes, 1.61 to 1.7 minutes, 1.71 to 1.8 minutes, 1.81 to 1.9 minutes, 1.91 to 2 minutes, 2.01 to 2.1 minutes, 2.11 to 2.2 minutes, 2.21 to 2.3 minutes, 2.31 to 2.4 minutes, 2.41 to 2.5 minutes, 2.51 to 2.6 minutes, 2.61 to 2.7 minutes, 2.71 to 2.8 minutes, 2.81 to 2.9 minutes, 2.91 to 3 minutes, 3.01 to 3.1 minutes, 3.11 to 3.2 minutes, 3.21 to 3.3 minutes, 3.31 to 3.4 minutes, 3.41 to 3.5 minutes, 3.51 to 3.6 minutes, 3.61 to 3.7 minutes, 3.71 to 3.8 minutes, 3.81 to 3.9 minutes, or 3.91 to 4 minutes, as measured at 37° C. with de-ionized water. A capsule may have an oxygen permeability (cm³/m²/day) of ≤0.5, as measured by a gas composition in the capsule. A capsule may have an oxygen permeability (cm³/m²/day) of ≤0.0001, ≤0.0005, ≤0.001, ≤0.005, ≤0.01, ≤0.05, ≤0.1, ≤0.5, ≤1, ≤1.5, ≤2, ≤5, or ≤10, as measured by a gas composition in the capsule.

In some cases, a pharmaceutical composition may be lyophilized. In some cases, a pharmaceutical composition may be frozen. Such frozen or lyophilized formulations may be administered in a frozen or lyophilized state to a subject. In some instances, such frozen formulation may be a popsicle, an ice cream, or other frozen formulations.

In some cases, a liquid suspension may be aliquoted into certain volumes to provide a unit dose of such oral dosage form. Such unit dose may have a volume of about 0.25, 0.5, 1, 2, 3, 5, or 10 mL. In some instances, the unit dose of a pharmaceutical composition herein has a volume of about 1 mL. Such pharmaceutical composition may comprise a bacterial population, a cryoprotectant, an antioxidant, an aqueous buffer solution that may from a liquid cell suspension. Such cell suspension may be tested for quality control to ensure it contains a certain number of metabolically active cells per bacterial strain as described herein.

In some instances, pharmaceutical compositions described herein are lyophilized or frozen. Bacterial cells in the lyophilized or frozen pharmaceutical compositions can be stored at −70° C. In some embodiments, the bacterial cells can be stored at 10° C., 4° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In other cases, the bacterial cells can also be stored from −80° C. to −70° C., from −70° C. to −60° C., from −60° C. to −50° C., from −50° C. to −40° C., from −40° C. to −30° C., from −30° C. to −20° C., from −20° C. to −10° C., from −10° C. to 0° C., or from 0° C. to 10° C. In some embodiments, at least 70% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In some cases, at least 75% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In other cases, at least 80% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In some embodiments, at least 85% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In other embodiments, at least 90% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. At least 95% of the stored lyophilized or frozen bacterial cells can also remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In some embodiments, at least 99% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months.

### Cryoprotectant and Antioxidant

Provided herein are pharmaceutical compositions that can comprise one or more cryoprotectant. A cryoprotectant may have the composition described in TABLE 3.

TABLE 3
Recipe for 150 L Cryoprotectant Mix

| | Weight (kg) for 150 L |
|---|---|
| Components | cryoprotectant mix |
| Saccharose | 12 |
| Trehalose | 1.995 |
| Sodium Glutamate | 0.825 |
| L-Cysteine HCI | 0.195 |

Such cryoprotectant can be used to maintain viability of the bacterial cells in a pharmaceutical composition when such composition is frozen or lyophilized, for example, during transport and/or storage prior to use. In some instances, the one or more cryoprotectant can be glycerol, dimethylsulfoxide (DMSO), ethylene glycol, propylene glycol, 2-methyl-2,4-pentanediol, trehalose, sucrose, diethyl glycol, triethylene glycol, polyvinylpyrrolidone (PVP), polyethylene glycol (PEG), saccharose, formamide, glycerol 3-phosphate, proline, methyl alcohol, glucose, bovine serum albumin, polyvinyl alcohol, hydroxyethyl starch, sorbitol, or a combination thereof. Cryoprotectant can comprise an ice blocker. An ice blocker can comprise polyglycerol, polyvinyl alcohol, X-1000 and Z-1000. Such cryoprotectant can be used in a pharmaceutical composition in an amount of about 5, 10, 15, 20, 25, or 30 volume percent (% v/v) or weight percent (% w/w), e.g., depending on whether the pharmaceutical composition is a solid dosage from (e.g., a capsule or tablet) or a liquid dosage from (e.g., a suspension or a gel). Cryoprotectant can also comprise a carbohydrate or an antioxidant. A carbohydrate can comprise trehalose, sucrose, sorbitol, glucose, fructose, saccharose, or a combination thereof.

In some embodiments, pharmaceutical compositions described herein further comprise an antioxidant. A cryoprotectant may comprise an antioxidant. A cryoprotectant may be present in an amount of at least about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, 99% or more of the pharmaceutical composition. A cryoprotectant may be present in an amount of at most about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, or 99% of the pharmaceutical composition. A cryoprotectant may be present in an amount of about 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10% of the pharmaceutical composition. The percentage of the cryoprotectant may be measured by weight or volume of the pharmaceutical composition.

In some embodiments, the antioxidant is L-cysteine. In some embodiments, the L-cysteine is present in an amount of about 0.001%, 0.005%, 0.01%, 0.05%, 0.1%, 0.5%, 1%, 1.5%, 2%, 5%, 10%, 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10% of the pharmaceutical composition. Saccharose can be present in an amount of about 0.1%, 0.5%, 1%, 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 0.1% to 1%, 1% to 5%, 5% to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 51 to 61%, 52 to 62%, 53 to 63%, 54 to 64%, 55 to 65%, 56 to 66%, 57 to 67%, 58 to 68%, or 59 to 69% of the pharmaceutical composition. Trehalose can be present in an amount of about 0.01%, 0.05%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 0.01% to 15%, 0.1% to 20%, 0.01% to 0.1%, 0.11% to 1%, 1 to 11%, 2 to 12%, 3 to 13%, 4 to 14%, 5 to 15%, 6 to 16%, 7 to 17%, 8 to 18%, 9 to 19%, 10 to 20%, 11 to 21%, 12 to 22%, 13 to 23%, 14 to 24%, or 15 to 25% of the pharmaceutical composition. Glycerol can be present in an amount of about 0.1%, 0.5%, 1%, 20%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1 to 21%, 2 to 22%, 3 to 23%, 4 to 24%, 5 to 25%, 6 to 26%, 7 to 27%, 8 to 28%, 9 to 29%, 10 to 30%, 11 to 31%, 12 to 320%, 13 to 33%, 14 to 340%, 15 to 35%, 16 to 360%, 17 to 37%, 18 to 38%, 19 to 390%, or 20 to 40% of the pharmaceutical composition. The percentages thereof may be measured by weight or volume of the pharmaceutical composition.

In some embodiments herein, the cryoprotectant of a pharmaceutical composition herein is glycerol. Such glycerol can be used in an amount of about 20% v/v in a pharmaceutical composition that can comprise a bacterial consortium of one or more, two or more, or three or more bacterial strains selected from TABLE 3. In some embodiments, the bacteria populations can be lyophilized. A lyophilization process can comprise a low temperature dehydration of the bacterial population. In some embodiments, the lyophilization process can comprise subjecting the bacterial population at low temperature and low pressure.

Provided herein are pharmaceutical compositions that can comprise one or more antioxidant. In some instances, such antioxidant can be used to protect anaerobic bacterial species and/or strain(s) that may be present in the pharmaceutical composition. In such instances, the one or more antioxidant can be used to provide anaerobic conditions during storage and/or transport, and/or to protect the bacterial cells from reactive oxygen species. In some embodiments herein, the antioxidant can be ascorbic acid, dithiothreitol, glutathione, phenolic acids (e.g., gallic, protochatechuic, caffeic, and rosmarinic acids), phenolic diterpenes (e.g., carnosol and carnosic acid), flavonoids (e.g., quercetin and catechin), volatile oils (e.g., eugenol, carvacrol, thymol, and menthol), α-Tocopherol (e.g., vitamin E), Trolox, ascorbic acid, vitamin A, vitamin C, coenzyme Q10, manganese, iodide, melatonin, alpha-carotene, astaxanthin, beta-carotene, canthaxanthin, cryptoxanthin, lutein, lycopene, zeaxanthin, flavonoids (e.g., flavones such as apigentin), luteolin, tangeithin, flavonols, isorhamnetin, kaempferol, myricetin, proanthocyanidins, quercetin, eriodictyol, hesperetin, naringenin, catechin, gallocatechin, epicatechin, epigallocatechin, theaflavin, thearubigins, isoflavone phytoestrogens, daidzein, genistein, glycitein, stilbenoids such as resveratrol, pterostilbene, anthocyanins, cyanidin, delphinidin, malvidin, pelargonidin, peonidin, petunidin, chicoric acid, chlorogenic acid, cinnamic acid, ellagic acid, ellagitannins, gallic acid, gallotannins, rosmarinic acid, curcumin, xanthones, capsaicin, bilirubin, citric acid, oxalic acid, phytic acid, N-acetylcysteine, L-cysteine, L-glutamate, L-proline, R-α-lipoic acid, anthocyanins, copper, cryptoxanthins, flavonoids, indoles, isoflavonoids, lignans, selenium, zinc, or a combination thereof. Such one or more antioxidant(s) can be present in a pharmaceutical composition in an amount of at least about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, 99% or more of the pharmaceutical composition. The antioxidant may be present in an amount of at most about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, or 99% of the pharmaceutical composition. The antioxidant may be present in an amount of about 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10% of the pharmaceutical composition. The percentage of the antioxidant may be measured by weight or volume of the pharmaceutical composition. L-glutamate can be present in an amount of about 0.01%, 0.05%, 0.1%, 0.5%, 1%, 1.5%, 2%, 2.1%, 2.2%, 20.3%, 2.4%, 20.5%, 2.6%, 20.7%, 2.8%, 2.9%, 3%, 3.1%, 3.2%, 30.3%, 3.4%, 30.5%, 3.6%, 3.7%, 3.8%, 3.9%, 4%, 4.1%, 4.2%, 40.3%, 4.4%, 40.5%, 4.6%, 4.7%, 40.8%, 4.9%, 50%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 8%, 9%, 10%, 1 to 5%, 1.1 to 5.1%, 1.2 to 5.2%, 1.3 to 5.3%, 1.4 to 5.4%, 1.5 to 5.5%, 1.6 to 5.6%, 1.7 to 5.7%, 1.8 to 5.8%, 1.9 to 5.9%, 2 to 6%, 2.1 to 6.1%, 2.2 to 6.2%, 2.3 to 6.3%, 2.4 to 6.4%, 2.5 to 6.5%, 2.6 to 6.6%, 2.7 to 6.7%, 2.8 to 6.8%, 2.9 to 6.9%, 3 to 7%, 3.1 to 7.1%, 3.2 to 7.2%, 3.3 to 7.3%, 3.4 to 7.4%, 3.5 to 7.5%, 3.6 to 7.6%, 3.7 to 7.7%, 3.8 to 7.8%, 3.9 to 7.9%, or 4 to 8% of the pharmaceutical composition. The percentage of the L-glutamate may be measured by weight or volume of the pharmaceutical composition. In some case, the cryoprotectant can comprise, by weight, about 60% saccharose, about 10% trehalose, about 1% L-cysteine, and about 4% L-glutamate of the pharmaceutical composition.

Provided herein are pharmaceutical compositions that can comprise an aqueous buffer solution. Such aqueous medium can be used as the main storage and transport medium for the bacterial cells. As such, the buffer can contain any one or more of bacterial consortium, cryoprotectant, and antioxidant, either dissolved or suspended, to form a pharmaceutical bacterial populations described herein. In some instances, the aqueous buffer solution can be phosphate buffered saline (PBS), HEPES, or Tris buffer, any other suitable buffer, or any combination thereof. In some embodiments, the buffer is PBS and comprises 137 mM NaCl, 2.7 mM KCl, 10 mM Na₂HPO₄, and 1.8 mM KH₂PO₄. In other cases, the buffer can be PBS and can have a pH of about 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9.

Thus, in some embodiments herein, a pharmaceutical composition comprises a bacterial consortium consisting of about 5×10\hat{ }8 CFU of each of the bacterial strains A. muciniphila (DSM 33213), F. prausnitzii (DSM 33185), and L. crispatus (DSM 33187), about 20% v/v glycerol as cryoprotectant, 0.1% w/w L-cysteine as antioxidant, and PBS buffer containing 137 mM NaCl, 2.7 mM KCl, 10 mM Na₂HPO₄, and 1.8 mM KH₂PO₄. Such pharmaceutical composition can be manufactured and formulated into an orally administrable dosage form using the methods and compositions described herein.

Provided herein are pharmaceutical compositions that can be designed and manufactured to allow storage and/or transport of the pharmaceutical compositions. In some instances, a pharmaceutical composition herein comprising a bacterial consortium can be designed such that the viability of the bacterial cells in the pharmaceutical composition is not or only minimally affected by storage and/or transport. In such instances, the viability of at least about 80%, 85%, 90%, 95%, 97%, or 99% of bacterial cells in the pharmaceutical composition is maintained during storage and/or transport.

In some instances, a pharmaceutical composition herein comprises a cryoprotectant to allow storage at low temperatures at about −70° C. or −80° C. to preserve viability of the bacterial cells. In such instances, the pharmaceutical composition can comprise about 20% v/v glycerol as a cryoprotectant. A pharmaceutical composition herein can further comprise an antioxidant that can preserve an anaerobic environment in the storage or transport vial and can protect the bacterial cells from reactive oxygen species.

In some cases, anaerobic may comprise a state with low oxygen or without oxygen. In some cases, an anaerobic environment may have 21% 20%, 19%, 18% 17%, 16%, 15%, 14%, 13% 12%, 11% 10%, 9%, 8%, 7%, 6% 5%, 4%, 3%, 2%, 1% 1×10\hat{ }−1%, 1×10\hat{ }−2%, 1×10\hat{ }−3%, 1×10\hat{ }−4%, 1×10\hat{ }−5%, 1×10\hat{ }−6%, 1×10\hat{ }−7%, 1×10\hat{ }−8%, 1×10\hat{ }−9%, 1×10\hat{ }−10%, 1×10\hat{ }−11%, or less oxygen, by volume, in the atmosphere of the environment.

In one example, in a pharmaceutical composition herein, live, vegetative bacteria can be preserved frozen in phosphate buffered saline (PBS) with 20% v/v glycerol and 0.1% w/w cysteine to preserve their viability. In such instances, the live bacteria can belong to any one or more of the strains shown in TABLE 1.

In some instances, the cryoprotectants or antioxidants described herein and thereof may be used with the bacterial populations, the pharmaceutical compositions, the methods for producing pharmaceutical compositions, or the methods for large-scale growth of Lactobacillus sp. described in this disclosure. In some instances, the cryoprotectants or antioxidants described herein and thereof may be used with the bacterial populations, the pharmaceutical compositions, the methods for producing pharmaceutical compositions, or the methods for large-scale growth of species of the Lactobacillaceae family described in this disclosure. In some cases, the cryoprotectants or antioxidants described herein and thereof may be used with the growth media or excipients described in this disclosure. In other cases, the cryoprotectants or antioxidants described herein and thereof may be used in any embodiments or examples described in this disclosure.

### Beneficial Properties

A pharmaceutical composition as disclosed herein may have one or more beneficial and/or therapeutic properties. A therapeutic property may comprise anti-inflammatory properties, anti-allergic, anti-infective properties, or anti-cancer properties. A therapeutic property may also comprise a reduction of one or more pro-inflammatory markers and/or metabolites. These may comprise certain immunoglobulins (e.g., IgE), histamines, pro-inflammatory chemokines or cytokines (e.g., IL-4, IL-13, etc), certain T helper cells (e.g., T_{H}17 cells), or certain immune cells such as eosinophils, neutrophils, mast cells, or basophils. The anti-inflammatory compound, marker, and/or metabolite may comprise a cytokine, a microbial lipid, a microbial carbohydrate, a microbial fatty acid, or a microbial amino acid. The anti-inflammatory compound may comprise IL-17. The pro-inflammatory compound may comprise a cytokine, a microbial lipid, a microbial carbohydrate, or a microbial amino acid. The pro-inflammatory compound may comprise IL-4, IL-10, IL-8, IL-13, TNF-a, or MUC5B, or any combination thereof. The microbial lipid may comprise a phospholipid.

A pharmaceutical composition of the present disclosure may comprise one or more bacterial species and/or strains that may produce one or more therapeutically effective compounds. Such compounds may comprise small molecules (e.g., fatty acids, lipids, etc.), peptides, polypeptide, and/or nucleic acids. Such compounds may have beneficial effects in a subject (e.g., a rodent or a human) when administered to the subject (e.g., in an oral formulation). Such beneficial effects may include anti-inflammatory effects. Such anti-inflammatory effects may comprise systemic, i.e., anti-inflammatory effects may comprise elicited in various parts of the body of an organism (e.g., a mammal such as a human). The therapeutic consortia described herein may comprise one or more bacterial cells of one or more bacterial genera, species or strains that may produce small molecule metabolites with beneficial (e.g., therapeutic) properties. The beneficial small molecules may include fatty acids such as produce short-chain fatty acids (SCFAs) and/or lipids such as phospholipids. In particular, the compositions and methods described herein may comprise one or more bacterial cells of one or more bacterial genera, species or strains that produce phospholipids and/or derivatives thereof. Such phospholipid and/or derivative thereof may comprise a phosphatidylcholine, a phosphatidylcholine-like compound and/or a phosphatidylcholine derivative (e.g., a chemically and/or structurally similar molecule to a phosphatidylcholine). The present disclosure provides one or more species and/or strains of Akkermansia sp. that may produce one or more phospholipids and/or derivatives thereof. The Akkermansia strain that produces one or more phospholipids may comprise Akkermansia muciniphila (DSM 33213). At least one of the phospholipids that may comprise produced by Akkermansia muciniphila (DSM 33213) may comprise a phosphatidylcholine and/or a derivative thereof. The production of one or more phosphatidylcholine(s) or derivatives thereof by Akkermansia sp. (e.g., Akkermansia muciniphila (DSM 33213)) may have an anti-inflammatory effect in a subject (e.g., a human).

A bacterial population of the present disclosure, once administered to a subject, may affect the metabolism of one or more compounds in the subject (e.g., a human or non-human animal). Affecting the metabolism of one or more compounds in the subject may comprise beneficial for the subject, e.g., in terms of having preventative and/or therapeutic properties (e.g., anti-inflammatory properties). A bacterial population of the present disclosure may affect and/or alter the metabolism of fatty acids and/or lipids in a subject. A bacterial population may affect and/or alter the metabolism of phospholipids and unsaturated or poly-unsaturated fatty acids. Such fatty acids may include omega-3- and/or omega-6 fatty acids. The metabolic effects that a bacterial population may elicit include the metabolism of linoleic acid (e.g., alpha-linolenic acid), arachidonic acid, and other fatty acids. The metabolism of such fatty acids may comprise affected in a way that results in a reduction of inflammation in a subject. A bacterial population may affect metabolic pathways used to metabolize certain compounds such as fatty acids, lipids, etc. Affecting such metabolic pathways may include at least a partial of full inhibition of certain metabolic pathways, such as pathways that result in the production of pro-inflammatory compounds or metabolites and/or compounds or metabolites that are associated with inflammation. A bacterial population may affect and/or alter a metabolic pathway directly and/or indirectly. For example, a bacterial population of the present disclosure may directly and/or indirectly increase the anti-inflammatory effects of alpha-linolenic acid by reducing the amount of alpha-linolenic acid metabolite(s) that may mediate inflammation and thus counteract the anti-inflammatory effects of that lipid. Thus, a bacterial population may beneficially affect and/or alter a metabolism in a subject. This may result in reducing the incidence of inflammation (e.g., chronic and/or allergic inflammation), a metabolic disease or disorder, an allergy, a dysbiosis, a cancer, or any combination thereof.

A bacterial population of the present disclosure may produce beneficial fatty acids, SCFAs, lipids, and/or phospholipids (e.g., phosphatidylcholine or a derivative thereof). The amount of any of such compounds produced by a bacterial population may comprise at least about 1.1, 1.2, 1.3, 1.4, 1.5, 1.6, 1.7, 1.8, 1.9, 2, 2.2, 2.5, 2.8, 3, 3.5, 4, 5, 6, 7, 8, 9, 10, 20, 50, or 100 times the amount produced by a consortium that does not contain all the bacterial genera, species, and/or strains of the therapeutic consortium.

### Methods of Treatment

In some instances, a pharmaceutical composition may be administered to a subject having or suspected of having a disease. In some cases, a pharmaceutical composition may be administered to a subject having a disease. In other cases, a pharmaceutical composition may be administered to a subject suspected of having a disease. In some cases, a pharmaceutical composition may be administered to a subject having a risk of developing a disease. In some cases, a pharmaceutical composition may be administered to a subject suspected of having a risking of developing a disease. In some cases, a pharmaceutical composition may be administered to a subject to treat a disease in the subject. In some cases, a pharmaceutical composition may be administered to a subject to prevent a disease in the subject. In some cases, when used to prevent a disease, the subject may not have developed the disease before being administered with the pharmaceutical composition.

In some instances, a pharmaceutical composition may be administered to a subject to decrease or prevent an inflammatory activity or inflammation of the subject. In some cases, a pharmaceutical composition may be administered to a subject to decrease or inhibit a pro-inflammatory nucleic acid, protein, metabolite, microbial organism, or any combination thereof; or increase, induce, or activate an anti-inflammatory nucleic acid, protein, metabolite, microbial organism, or any combination thereof of the subject.

In some instances, a disease treated by a pharmaceutical composition may comprise an inflammatory disease. In some cases, an inflammatory disease treated by a pharmaceutical bacterial population may comprise an allergy or dermatitis. In some cases, a disease treated by a pharmaceutical composition may comprise an allergy. In some cases, an inflammatory disease treated by a pharmaceutical composition may comprise dermatitis. In some instances, an inflammatory disease treated by a pharmaceutical composition may comprise atopy, asthma, an autoimmune disease, an autoinflammatory disease, a hypersensitivity, pediatric allergic asthma, allergic asthma, inflammatory bowel disease, Celiac disease, Crohn's disease, colitis, ulcerative colitis, collagenous colitis, lymphocytic colitis, diverticulitis, irritable bowel syndrome, short bowel syndrome, stagnant loop syndrome, chronic persistent diarrhea, intractable diarrhea of infancy, Traveler's diarrhea, immunoproliferative small intestinal disease, chronic prostatitis, postenteritis syndrome, tropical sprue, Whipple's disease, Wolman disease, arthritis, rheumatoid arthritis, Behcet's disease, uveitis, pyoderma gangrenosum, erythema nodosum, traumatic brain injury, psoriatic arthritis, juvenile idiopathic arthritis, multiple sclerosis, systemic lupus erythematosus (SLE), mayasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, bullous pemphigoid, sarcoidosis, ichthyosis, Graves ophthalmopathy, Addison's disease, Vitiligo, acne vulgaris, pelvic inflammatory disease, reperfusion injury, sarcoidosis, transplant rejection, interstitial cystitis, atherosclerosis, food allergy or atopic dermatitis atopic dermatitis. In some instances, an inflammatory disease treated by a pharmaceutical composition may comprise pediatric allergic asthma or inflammatory bowel disease. In some instances, an allergy treated by a pharmaceutical composition may comprise allergic asthma, food allergy, or allergic pediatric asthma. In some cases, an allergy treated by a pharmaceutical composition may comprise allergic asthma. In some cases, an allergy treated by a pharmaceutical composition may comprise food allergy. In some cases, an allergy treated by a pharmaceutical composition may comprise allergic pediatric asthma.

In some instances, a disease treated by a pharmaceutical composition may comprise a metabolic disease. In some cases, a metabolic disease treated by a pharmaceutical composition may comprise obesity, diabetes, or a metabolic syndrome. In some cases, a metabolic disease treated by a pharmaceutical composition may comprise obesity. In some cases, a metabolic disease treated by a pharmaceutical composition may comprise diabetes. In some cases, a metabolic disease treated by a pharmaceutical composition may comprise a metabolic syndrome. In some cases, a metabolic disease treated by a pharmaceutical composition may also comprise pre-diabetes, type 1 diabetes, type 2 diabetes, diabetes complications, prediabetes, non-alcoholic fatty liver disease (NAFLD), weight loss, insulin-deficiency or insulin-resistance related disorders, glucose intolerance, abnormal lipid metabolism, atherosclerosis, hypertension, cardiac pathology, stroke, hyperglycemia, hepatic steatosis, dyslipidemia, dysfunction of the immune system associated with overweight and obesity, cardiovascular diseases, high cholesterol, elevated triglycerides, asthma, sleep apnea, osteoarthritis, neuro-degeneration, gallbladder disease, syndrome X, inflammatory and immune disorders, atherogenic dyslipidemia or cancer.

In some instances, a subject may have a disease. In some cases, a subject may be suspected of having a disease. In some cases, a subject may have a risk of developing a disease. In other cases, a subject may be suspected of having a risking of developing a disease.

In some cases, a subject administered with the pharmaceutical compositions described herein and thereof may comprise a human subject. A human subject may be an infant, a child, an adolescent, or an adult. A human subject may be an infant. A human subject may be a child. A human subject may be an adolescent. A human subject may be an adult. In some cases, a second subject may be a different individual from a subject.

A human subject that is administered with the pharmaceutical compositions described herein and thereof, in some cases, have an age of at least about 1 day old, at least about 2 days old, at least about 3 days old, at least about 4 days old, at least about 5 days old, at least about 6 days old, at least about 1 week old, at least about 2 weeks old, at least about 3 weeks old, at least about 4 weeks old, at least about 1 month old, at least about 2 months old, at least about 3 months old, at least about 4 months old, at least about 5 months old, at least about 6 months old, at least about 7 months old, at least about 8 months old, at least about 9 months old, at least about 10 months old, at least about 11 months old, at least about 1 year old, at least about 2 years old, at least about 3 years old, at least about 4 years old, at least about 5 years old, at least about 6 years old, at least about 7 years old, at least about 8 years old, at least about 9 years old, at least about 10 years old, at least about 11 years old, at least about 12 years old, at least about 13 years old, at least about 14 years old, at least about 15 years old, at least about 16 years old, at least about 17 years old, at least about 18 years old, at least about 19 years old, at least about 20 years old, at least about 21 years old, at least about 22 years old, at least about 23 years old, at least about 24 years old, at least about 25 years old, at least about 26 years old, at least about 27 years old, at least about 28 years old, at least about 29 years old, at least about 30 years old, at least about 31 years old, at least about 32 years old, at least about 33 years old, at least about 34 years old, at least about 35 years old, at least about 36 years old, at least about 37 years old, at least about 38 years old, at least about 39 years old, at least about 40 years old, at least about 41 years old, at least about 42 years old, at least about 43 years old, at least about 44 years old, at least about 45 years old, at least about 46 years old, at least about 47 years old, at least about 48 years old, at least about 49 years old, at least about 50 years old, at least about 51 years old, at least about 52 years old, at least about 53 years old, at least about 54 years old, at least about 55 years old, at least about 56 years old, at least about 57 years old, at least about 58 years old, at least about 59 years old, at least about 60 years old, at least about 61 years old, at least about 62 years old, at least about 63 years old, at least about 64 years old, at least about 65 years old, at least about 66 years old, at least about 67 years old, at least about 68 years old, at least about 69 years old, at least about 70 years old, at least about 71 years old, at least about 72 years old, at least about 73 years old, at least about 74 years old, at least about 75 years old, at least about 76 years old, at least about 77 years old, at least about 78 years old, at least about 79 years old, at least about 80 years old, at least about 81 years old, at least about 82 years old, at least about 83 years old, at least about 84 years old, at least about 85 years old, at least about 86 years old, at least about 87 years old, at least about 88 years old, at least about 89 years old, at least about 90 years old, at least about 91 years old, at least about 92 years old, at least about 93 years old, at least about 94 years old, at least about 95 years old, at least about 96 years old, at least about 97 years old, at least about 98 years old, at least about 99 years old, or at least about 100 years old.

In some cases, an infant may have an age of at most about 1 day old, at least about 1 day old, at least about 2 days old, at least about 3 days old, at least about 4 days old, at least about 5 days old, at least about 6 days old, at least about 1 week old, at least about 2 weeks old, at least about 3 weeks old, at least about 4 weeks old, at least about 1 month old, at least about 2 months old, at least about 3 months old, at least about 4 months old, at least about 5 months old, at least about 6 months old, at least about 7 months old, at least about 8 months old, at least about 9 months old, at least about 10 months old, at least about 11 months old, or at most about 1 year old.

In some cases, an infant may have an age of at most about 1 year old.

In some cases, a child may have an age of at least about 1 day old, at least about 2 days old, at least about 3 days old, at least about 4 days old, at least about 5 days old, at least about 6 days old, at least about 1 week old, at least about 2 weeks old, at least about 3 weeks old, at least about 4 weeks old, at least about 1 month old, at least about 2 months old, at least about 3 months old, at least about 4 months old, at least about 5 months old, at least about 6 months old, at least about 7 months old, at least about 8 months old, at least about 9 months old, at least about 10 months old, at least about 11 months old, at least about 1 year old, at least about 2 years old, at least about 3 years old, at least about 4 years old, at least about 5 years old, at least about 6 years old, at least about 7 years old, at least about 8 years old, at least about 9 years old, at least about 10 years old, at least about 11 years old, at least about 12 years old, at least about 13 years old, at least about 14 years old, at least about 15 years old, at least about 16 years old, at least about 17 years old, or at most about 18 years old. In some cases, a child may have an age of at most about 18 years old.

In some cases, an adolescent may have an age of about at least about 10 months old, at least about 11 months old, at least about 1 year old, at least about 2 years old, at least about 3 years old, at least about 4 years old, at least about 5 years old, at least about 6 years old, at least about 7 years old, at least about 8 years old, at least about 9 years old, at least about 10 years old, at least about 11 years old, at least about 12 years old, at least about 13 years old, at least about 14 years old, at least about 15 years old, at least about 16 years old, at least about 17 years old, at least about 18 years old, or at most about 19 years old. In some cases, an adolescent may have an age from about 10 years old to about 19 years old.

In some cases, an adult may have an age of at least about 18 years old.

In some instances, a pharmaceutical composition may be administered to a subject for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, or at least about 5 years. In some cases, a pharmaceutical composition may be administered to a subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some cases, a pharmaceutical composition may be administered to a subject for at least about 1 day. In some cases, a pharmaceutical composition may be administered to a subject for at least about 2 days. In some cases, a pharmaceutical composition may be administered to a subject for at least about 3 days. In some cases, a pharmaceutical composition may be administered to a subject for at least about 4 days. In some cases, a pharmaceutical composition may be administered to a subject for at least about 5 days. In some cases, a pharmaceutical composition may be administered to a subject for at least about 6 days. In some cases, a pharmaceutical composition may be administered to a subject for at least about 7 days. In some cases, a pharmaceutical composition may be administered to a subject for at least about 8 days. In some cases, a pharmaceutical composition may be administered to a subject for at least about 9 days. In some cases, a pharmaceutical composition may be administered to a subject for at least about 10 days. In some cases, a pharmaceutical composition may be administered to a subject for at least about 11 days. In some cases, a pharmaceutical composition may be administered to a subject for at least about 12 days. In some cases, a pharmaceutical composition may be administered to a subject for at least about 13 days. In some cases, a pharmaceutical composition may be administered to a subject for at least about 14 days.

A pharmaceutical composition herein can be administered for various periods of time according to different administration schedules. A treatment period may vary between subjects and individuals and can depend on various factors as described herein, e.g., disease state, age, etc. In some instances, a subject can be treated for one day to at least about one week, for about a week to about one month, or for about one month to about one year. In such instances, the subject can be treated for about one month, two months, or three months. In some cases, treatment can be performed on consecutive days, consecutive weeks, and/or consecutive months. In some embodiments, a pharmaceutical composition is administered for about 28, 29, or 30 consecutive days.

In some instances, a time interval between two administrations of two pharmaceutical compositions may comprise at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 1 day, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year subsequent to the subject is administered with a pharmaceutical composition. In some cases, a time interval between two administrations of two pharmaceutical compositions may comprise at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some cases, a time interval between two administrations of two pharmaceutical compositions may comprise at least about 1 day. In some cases, a time interval between two administrations of two pharmaceutical compositions may comprise at least about 2 days. In some cases, a time interval between two administrations of two pharmaceutical compositions may comprise at least about 3 days. In some cases, a time interval between two administrations of two pharmaceutical compositions may comprise at least about 4 days. In some cases, a time interval between two administrations of two pharmaceutical compositions may comprise at least about 5 days. In some cases, a time interval between two administrations of two pharmaceutical compositions may comprise at least about 6 days. In some cases, a time interval between two administrations of two pharmaceutical compositions may comprise at least about 7 days. In some cases, a time interval between two administrations of two pharmaceutical compositions may comprise at least about 8 days. In some cases, a time interval between two administrations of two pharmaceutical compositions may comprise at least about 9 days. In some cases, a time interval between two administrations of two pharmaceutical compositions may comprise at least about 10 days. In some cases, a time interval between two administrations of two pharmaceutical compositions may comprise at least about 11 days. In some cases, a time interval between two administrations of two pharmaceutical compositions may comprise at least about 12 days. In some cases, a time interval between two administrations of two pharmaceutical compositions may comprise at least about 13 days. In some cases, a time interval between two administrations of two pharmaceutical compositions may comprise at least about 14 days.

Methods of treatment herein can include administering a pharmaceutical composition of this disclosure once, two, three, four, five, six, seven, eight, nine, ten, eleven, or twelve times daily. In various instances, a pharmaceutical composition of this disclosure is administered twice daily. Such twice daily administration can be performed in the morning and in the evening. In such cases, there can be a period of about 8, 12, or 16 hours between the first and the second administration of a given day.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

These examples are provided for illustrative purposes only and not to limit the scope of the claims provided herein.

### Example 1: Clinical Protocol for Administering a Pharmaceutical Composition

Provided herein are the protocols for orally administering to a human subject a pharmaceutical composition A and monitoring the safety and biological/clinical effects of the composition.

### Administration and Assessment of the Pharmaceutical Composition

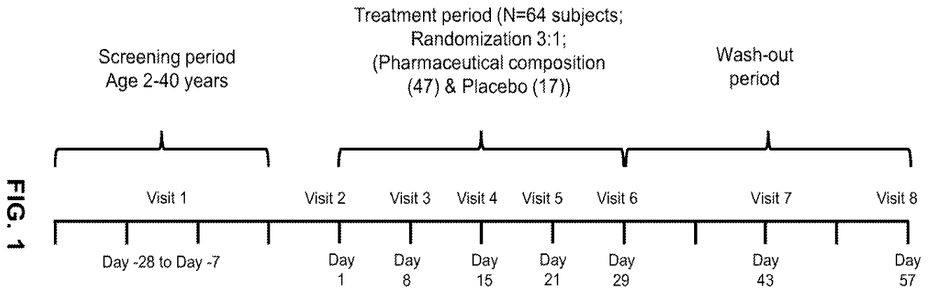

Pharmaceutical composition A comprising the bacterial population consisting of the bacterial strains Akkermansia muciniphila (DSM 33213), Faecalibacterium prausnitzii (DSM 33185), and Lactobacillus crispatus (DSM 33187) was evaluated in a Phase 1b, multi-centered, randomized, double-blind, placebo-controlled, parallel group, three sequential part study of the bacterial population in multi-sensitized (to two or more allergens) human subjects who were otherwise healthy, as illustrated in FIG. 1.

Subjects were separated into three categories (cohorts). The first category (cohort 1) contained 23 subjects, 18-40 years of age, multi-sensitized to two or more allergens, who were otherwise healthy (male:female; approximately 1:1) (randomized 17:6 test:placebo); the second category (cohort 2) contained 23 subjects, 12-17 years of age, multi-sensitized to two or more allergens, who were otherwise healthy (male:female; approximately 1:1) (randomized 17:6 test:placebo); and the third category (cohort 3) contained 18 subjects 2-11 years of age, multi-sensitized to two or more allergens, who are otherwise healthy (male:female; approximately 1:1) (randomized 13:5 test:placebo). Subjects were randomized 3:1 with regards to the pharmaceutical composition vs. placebo.

Treatment of human subjects consisted of twice daily oral administration (approximately every 12 hours+/−4 hours by mixing with food or milk) of pharmaceutical composition A for 28 days. Each 1 mL dose of pharmaceutical composition A contained three live bacterial strains, each present at 5×10\hat{ }8 CFU per dose: Lactobacillus crispatus (DSM 33187), Faecalibacterium prausnitzii (DSM 33185), and Akkermansia muciniphila (DSM 33213). The pharmaceutical composition was supplied as a single frozen glycerol stock containing all three bacterial species. Each dose of the pharmaceutical composition was provided in a 2 mL polypropylene screw cap vial with a silicone washer seal. The vial contained all three live bacterial strains suspended in a buffered glycerol solution. The buffered glycerol solution was composed of standard phosphate buffered saline (PBS, 137 mM NaCl, 2.7 mM KCl, 10 mM Na₂HPO₄, and 1.8 mM KH₂PO₄), 20% v/v glycerol, and 0.1% w/w cysteine as an antioxidant. The volume of the pharmaceutical composition dose was approximately 1 mL.

For the purpose of this example, the pharmaceutical composition was stored at −70° C., and a temperature log was maintained. Once provided to the human subjects, the pharmaceutical composition was stored in a freezer at or below −18° C. The pharmaceutical composition doses were sealed in its cryovial (e.g., 2 mL cryovial) containers to maintain the potency and purity of the product prior to thawing for consumption. The frozen stocks of pharmaceutical composition were thawed at room temperature for 5-10 minutes and immediately consumed as mentioned in dosing instructions.

The placebo treatment in this example consisted of twice daily oral administration (approximately every 12±4 hours by mixing with food or milk) of excipients (placebo: phosphate buffered saline (PBS) as described for product formulation with 20% v/v glycerol and 0.1% w/w cysteine) for 28 days. Each 1 mL dose was identical in volume to the test product. The frozen stock was thawed at room temperature for 5-10 minutes, and the contents of the vial were immediately mixed in adequate quantity (1-2 ounces) of cold or room temperature milk (including breast milk, liquid infant formulas, cow's milk, almond milk, and soy milk) or foods such as applesauce and yogurt.

As illustrated in FIG. 1, screening visits were conducted from 28 days up to 7 days prior to day 1 (baseline visit). Dosing began on Day 1. Subjects were treated for 28 days with subsequent visits on days 8, 15, 22, and 29. Follow-up visits were conducted on days 43 and 57. Each visit after baseline had a window of ±1 day.

Fecal and plasma samples of the subjects were collected on Day 1/Visit 2 (baseline), Days 8, 15, 21, and 29/Visits 3-6 (treatment), and Days 43 and 57/Visits 7-8 (washout).

Assessments on the safety of the pharmaceutical compositions were performed during the 28-day treatment period and up to 28 days (Day 57) of washout thereafter by (i) reporting adverse events; (ii) reporting concomitant medications; (iii) performing physical examinations and vital signs; and (iv) performing laboratory tests including serum chemistry, liver and renal function panels, hematology, complete blood count with differential, and urinalysis. In the third part of this study (i.e., the washout period), blood and urine samples were not collected for safety assessments. But all other assessments, except for those requiring blood and urine sampling, were performed.

Assessments on the biological/clinical effects of pharmaceutical composition A were performed by evaluating change in the following parameters: (i) pharmacokinetics; (ii) stool microbiome analysis; (iii) stool and plasma metabolic profiling; and (iv) stool and plasma inflammatory markers. Questionnaire(s) and adverse effect (AE) and medication use reporting in diary.

Various laboratory variables were also determined in this study: (i) Hematological variables including hematocrit, hemoglobin, mean corpuscular hemoglobin, mean corpuscular hemoglobin concentration, mean corpuscular volume, platelet count, red blood cell distribution width, red blood cell count, and white blood cell count with differential; (ii) urinalysis parameters including appearance (e.g., color and character), bilirubin, urobilinogen, protein content, glucose levels, ketones, leukocyte esterase, urine blood, nitrite, pH, and specific gravity; (iii) biochemical parameters including glucose levels, uric acid, BUN (blood urea nitrogen), creatinine, BUN/creatinine ratio, eGFR (estimated glomerular filtration rate), sodium, potassium, chloride, bicarbonate, calcium, albumin, total bilirubin, alkaline phosphatase levels, AST (aspartate aminotransferase) levels, ALT (alanine transaminase), gamma-glutamyltransferase (GGT), total cholesterol levels, and triglyceride levels.

The protocol for administering pharmaceutical composition A and assessments on the biological/clinical effects of pharmaceutical composition A are applicable to any pharmaceutical compositions described in this disclosure. The biological/clinical may comprise any microbial community, microbial diversity, bacterial population, steroid compound, microbially-derived metabolite, membrane lipid, or pro-inflammatory immune marker described in this disclosure.

### Safe and Well-Tolerated Pharmaceutical Composition

Pharmaceutical composition A was safe and well-tolerated. The safety data of the population composition comprising the bacterial population is shown in TABLE 4.

TABLE 4
Safety Profile of the Pharmaceutical Composition
Comprising the Bacterial Population

| | Placebo | Bacterial Population |
|---|---|---|
| Number (#) of participants | 17 | 47 |
| Total # TEAEs*, n (%) | 74 (4.6%) | 207 (4.4%) |
| # of subjects | 10 (58.8%) | 32 (68.1%) |
| SAEs** | 0 | 0 |
| TEAEs leading to | 0 | 0 |
| withdrawals | | |

*TEAE: Treatment Emergent Adverse Events
**SAE: Serious adverse events

The pharmaceutical composition showed high dose adherence (˜ 97%) and low participant drop-out rate (fewer than 5% of the participants dropped out; none of them was related to the treatment). No SAE had been observed. Adverse events (TEAE) potentially associated with the treatment of the pharmaceutical composition was minimal. All the events were diary related (e.g., burping and flatulence) and similarly impacted both the placebo and treatment groups.

### Example 2: Pharmacokinetics of a Pharmaceutical Composition

Provided herein are the pharmacokinetic analyses of pharmaceutical composition A. Co-association of Faecalibacterium sp. and Akkermansia sp. in the Stool of Subjects Administered with Pharmaceutical Composition A

The strains of Faecalibacterium sp. and Akkermansia sp. exhibited co-association in the gastrointestinal tract when administered orally.

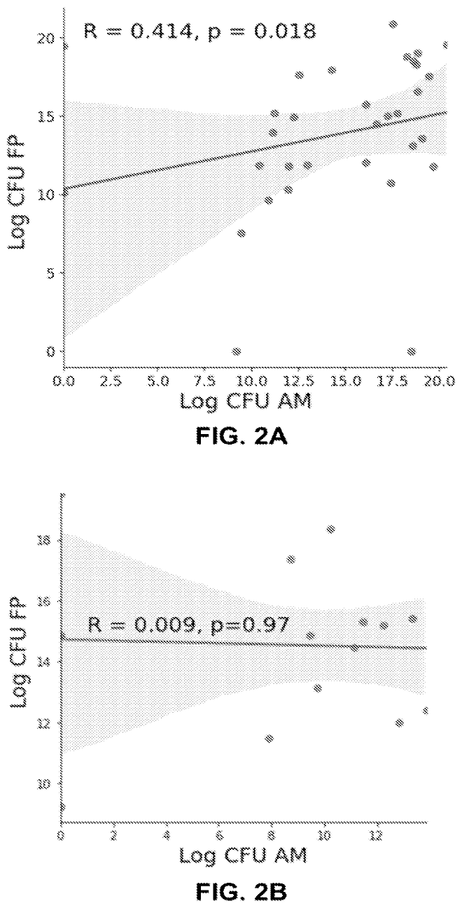

As shown in FIG. 2A, Faecalibacterium prausnitzii (DSM 33185) and Akkermansia muciniphila (DSM 33213) were positively and statistically significantly correlated in the stool sample of the subject administered with pharmaceutical composition A in the treatment and washout periods. In contrast, as shown in FIG. 2B, the two bacterial strains did not exhibit any correlation in the stool samples of the subjects treated with the placebo control.

Therefore, administering the pharmaceutical composition positively regulated the abundance of the strains of the bacterial population of the composition in the gastrointestinal tract.

### Engraftment of the Administered Bacterial Population

Multiple bacterial strains of the pharmaceutical composition engrafted in the gastrointestinal tract when administered.

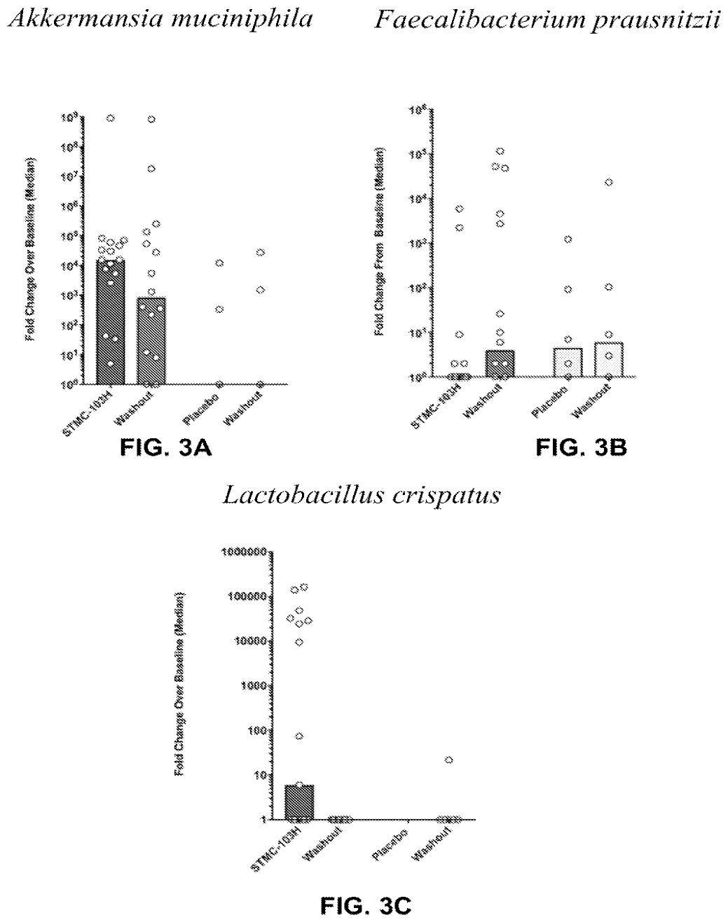

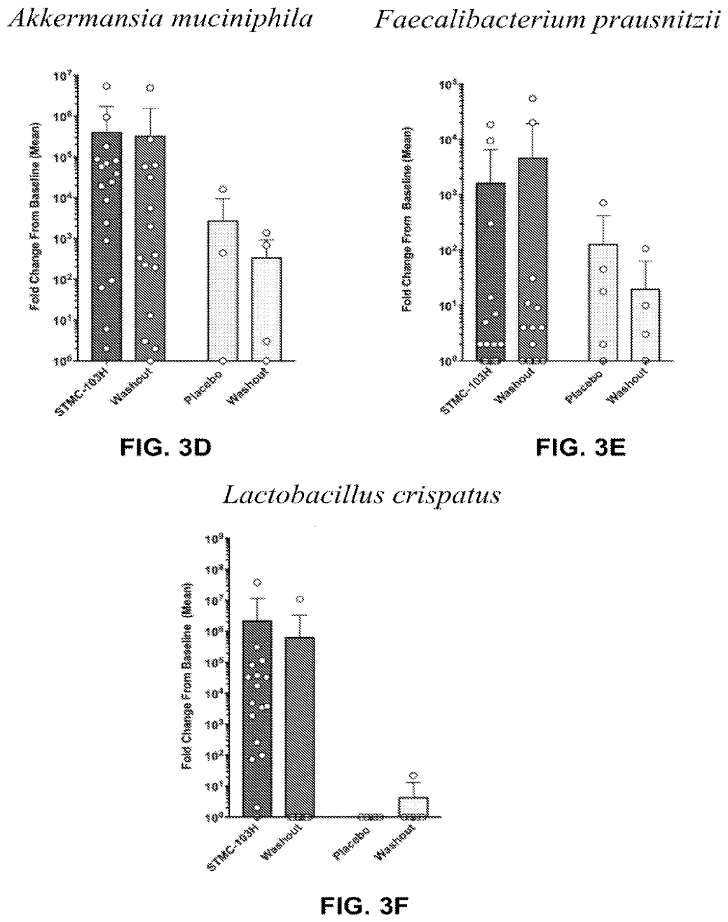

The median numbers of Akkermansia muciniphila (DSM 33213) exhibited statistically significant increases over the baseline level during both the treatment period and after the 1-month washout period, showing partial engraftment in the gastrointestinal tract of the subject administered with the pharmaceutical composition, as shown in FIG. 3A.

Moreover, as shown in FIG. 3C, the median number of Lactobacillus crispatus (DSM 33187) also exhibited a significant increase over the baseline abundance during the treatment period. As shown in FIG. 3B, Faecalibacterium prausnitzii (DSM 33185) displayed a statistically significant increase over the baseline abundance in the 1-month washout period. Akkermansia muciniphila (DSM 33213) and Lactobacillus crispatus (DSM 33187) did not display any statistically significant increase over the baseline abundance in the placebo group in any period, as illustrated in FIGS. 3A-3C. Further, the mean numbers of Akkermansia muciniphila (DSM 33213), Faecalibacterium prausnitzii (DSM 33185), and Lactobacillus crispatus (DSM 33187) in the treatment and washout period also exhibited significant increase above those of the baseline and placebo, as shown in FIGS. 3D-F.

Collectively, these results suggested that each of the bacterial population in the pharmaceutical composition engrafted in the subject being treated.

### Example 3: Microbiome Changes in Response to a Pharmaceutical Composition

Provided herein are the analyses of the microbiomes in the subjects administered with the pharmaceutical composition.

Pharmaceutical composition A not only increased the abundance of the bacterial population but increased and decreased various species of the microbiome of the subject.

### Microbiome Profiling by Sequencing

To profile the impact of pharmaceutical composition A on the microbial community, the sequences of the prokaryotic 16S ribosomal RNA gene of the subject administered with the composition were analyzed. The bacterial 16S rRNA gene is approximately 1500 bp long and contains nine variable regions interspersed between conserved regions. The DNA sequence in these variable regions corresponds to phylogenetic classification of bacteria, such as family, genus, and species. The analysis of the subject administered with pharmaceutical composition A was derived from Illumina MiSeq sequencing of 16S gene amplicons containing the V3-V4 hypervariable regions (spanning base pairs 341 to 805).

The pipeline of the sequencing comprised: (i) PCR amplification of the 16S rRNA gene V3-V4 region with Illumina overhand primers using high quality stool DNA template; (ii) amplicon validation using gel electrophoresis; (iii) amplicon indexing by the addition of adapters in another PCR reaction; (iv) individual library quantification using Qubit quantification; (v) amplicon normalization; and (vi) library sequencing using Illumina MiSeq sequencing platform with extend read lengths 2×301 bp.

The pipeline of the sequence analysis comprised: (i) importing demuxed fastq sequence files into Qline2; (ii) DADA2 quality filtering and denoising; (iii) filtering and rarefying amplicon sequence variant (ASV) table; (iv) assigning taxonomy to sequence reads; (v) generating phylogenetic trees; (vi) generating alpha rarefaction curves; (vii) generating alpha-diversity indexes and testing for differences; (viii) testing the relationship between beta-diversity and variables of interest using LME modeling; (ix) differential abundance analysis ANCOM (Qlime2); and (x) importing tables into R for differential abundance analysis using the MaAsLin2 package.

The sequence analysis of the three cohorts are summarized in TABLE 6.

TABLE 6
Sequencing analysis summary of all three cohorts

| | Cohort 1 | Cohort 2 | Cohort 3 |
|---|---|---|---|
| Number of clinical | 136 | 148 | 127 |
| samples sequenced | | | |
| Average number of | 5.87 | 6.43 | 6.67 |
| unique samples | | | |
| per subject sequenced | | | |
| Average number of raw | 73743 | 119482 | 151509 |
| reads per sample | | | |
| Average number of | 46480 | 78140 | 100652 |
| reads per | | | |
| sample passing DADA2 | | | |
| Number of ASVs in | 1867 | 1698 | 1593 |
| filtered dataset | | | |

Samples from baseline (V2), four treatment periods (V3-V6), and two post-treatment washouts (V7 & V8) were collected from the three cohorts for the sequencing analysis. V7 sample was collected two weeks post-cessation of treatment. V8 sample was collected four weeks post-cessation of treatment. In cohort 1, V7 sample was not collected.

Subjects of cohort 1 displayed the highest number of distinct ASVs at 1,867, whereas those of cohort 3 displayed the lowest number of ASVs at 1,593 despite deeper sequencing depth. This finding was consistent with previously described lower microbiota diversity in infants and children.

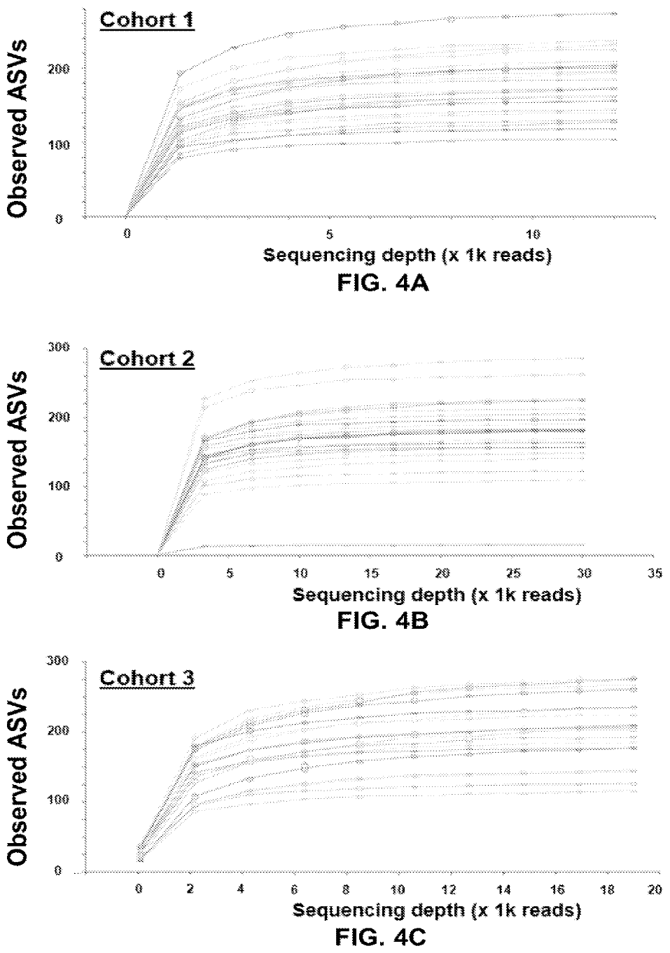

Rarefaction curve analysis of the three cohorts showed that the increase in new bacterial taxa in the observed OTUs approached saturation at a sequencing depth of about 4000 reads per sample, as shown in FIGS. 4A-C. Hence, the sequencing depth (i.e., 12000 reads per sample for cohort 1; 30000 reads per sample for cohort 2, and 19000 reads per sample for cohort 3) employed covered most bacterial taxa in the subjects of all cohorts. Similar analysis using Observed OTUs, Faith's, and Shannon supported a similar conclusion (data not shown).

### Subjects in Different Cohorts Have Unique Gastrointestinal Microbiomes

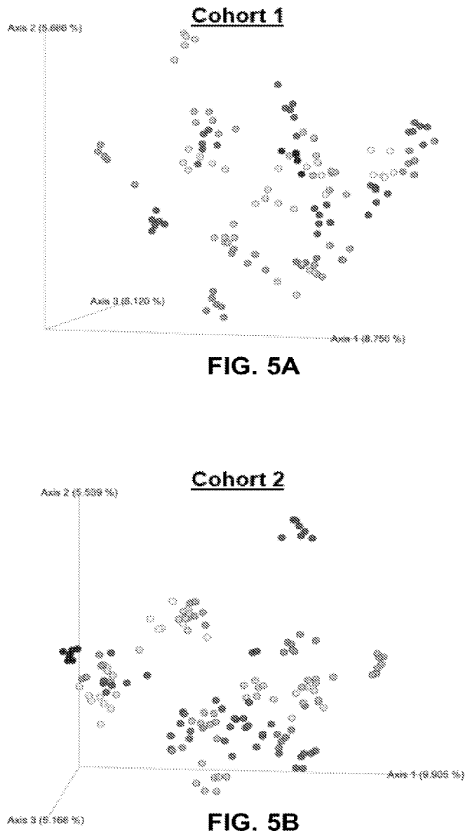

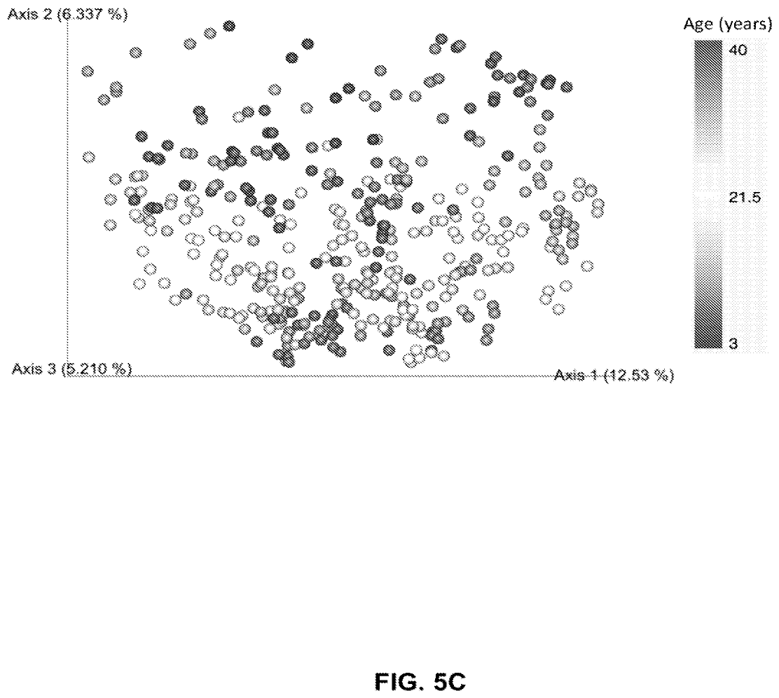

To analyze the microbiomes of the subjects in different cohorts (age groups), the (dis)similarity of the microbiomes based on the Jaccard distance matrix was calculated. As shown in FIGS. 5A-B, the clustering of the samples of each subject across various time points suggested that the overall stability of microbial communities within an individual. The separation of individuals (i.e., each distinct color) supported the uniqueness of the gastrointestinal community with the subjects.

When the Unweighted UniFrac Distance of the microbiome of every subject in all three cohorts were visualized together in a beta-diversity emperor plot, as shown in FIG. 5C, the microbiomes showed stratification by age groups. The microbiomes of each cohort clustered with each other, suggesting that each cohort (i.e., age group) had a unique microbiome composition. The results suggested that the gastrointestinal microbiome continued to evolve even into adulthood (i.e., cohort 2 versus cohort 3), further complementing the previous suggestion that the gut microbiota can achieve an “adult-like” configuration by about three years of age.

### Global Microbiome Diversity Shift

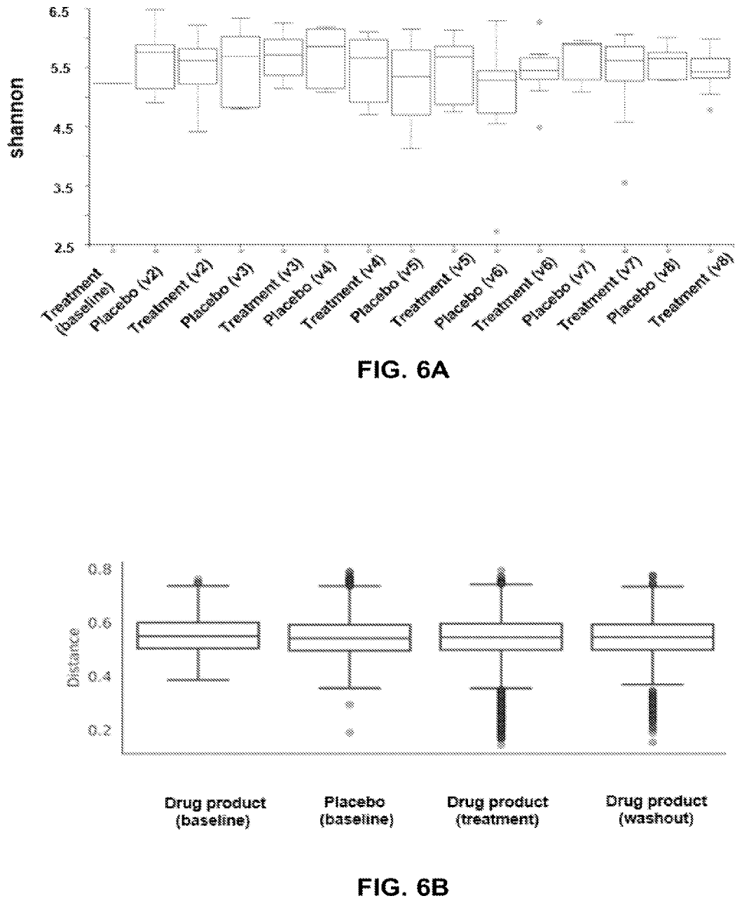

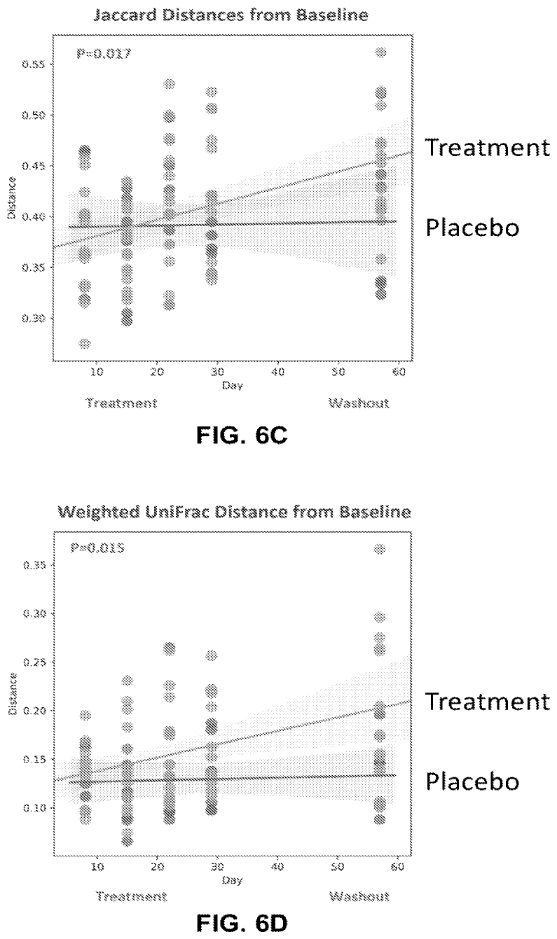

Alpha-diversity of the OTUs of each time point for each cohort was calculated to determine if the total number of bacterial taxa varied between different time points after the subjects were administered with pharmaceutical composition A. As shown in FIG. 6A, comparisons of alpha-diversity distance of the microbiome of the baseline and the treated sample in each cohort showed no significant difference (Kruskal-Wallis test) at all time points (visit or V denoted in FIG. 6A), suggesting that the total number of species of a microbiome was not impacted by pharmaceutical composition A. Analyses using Faith's supported a similar conclusion.

Alpha-diversity distance may not capture the changes of the microbiome because it only measures the total number of microbial taxa in a sample. The make-up of the microbiome may undergo significant changes without a corresponding change in the total number of taxa. To measure the evolution of the microbiome, beta-diversity distance was analyzed. It used co-variate data (e.g., treatment group, time point, age, body mass index) to allow for identification of significant changes to the gut microbiota associated with the variables.

Jaccard, unweighted UniFrac, weighted UniFrac, and Bray-Curtis distance matrices were generated for comparisons, as each described a different characteristic of the dataset (e.g., presence/absence or abundance of a taxon). As shown in FIG. 6B, no significant changes to overall microbial community were observed in the comparisons of placebo versus the pharmaceutical composition A (drug product) treatment at a single time point or the drug product baseline to the end of the treatment. However, standard statistical approach of FIG. 6B used PERMANOA, which may not be appropriate for repeated measures, such as repeated temporal data from the same individuals obtained from EXAMPLE 2. An appropriate analysis approach may consider the repeated measured performed. It may be able to determine if the differences between treatment groups are significant if individual differences are incorporated.

To this end, the beta-diversity distance was first calculated between the samples from a single individual. The distance of the sample was calculated in two ways: (i) the distance of two consecutive time points, or (ii) the distance of any time points to the baseline. A linear mixed effects (LME) model was then used to test the relationship between these distances (response variable) and one or more independent variables, such as treatment, incorporating the repeated-measures sampling of individuals.

Using such methods, the gastrointestinal microbiome diverged significantly from baseline during and after the pharmaceutical composition A treatment. As shown in FIGS. 6C-D, the microbial community in the stool sample of the subject administered with the pharmaceutical composition exhibited statistically significant divergence from the basement level through the end of treatment (Day 28) and well into the washout period (Day 56) after the treatment. In contrast, the microbial community in the stool sample of the subject receiving the placebo did not show any changes. The differences in Jaccard and weighted UniFrac distances suggested differences in the presence/absence and abundance of taxa over time.

These results suggested that the pharmaceutical composition impacted and regulated the whole microbiome community in the gastrointestinal tract of the treated subject.

### Specific Taxa Changes in Response to Pharmaceutical Composition A

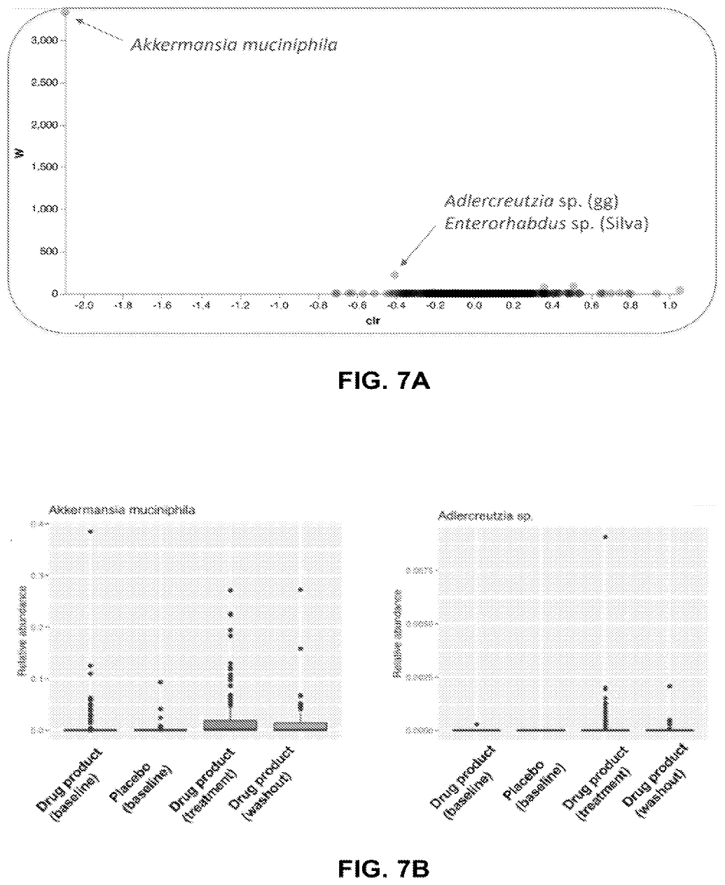

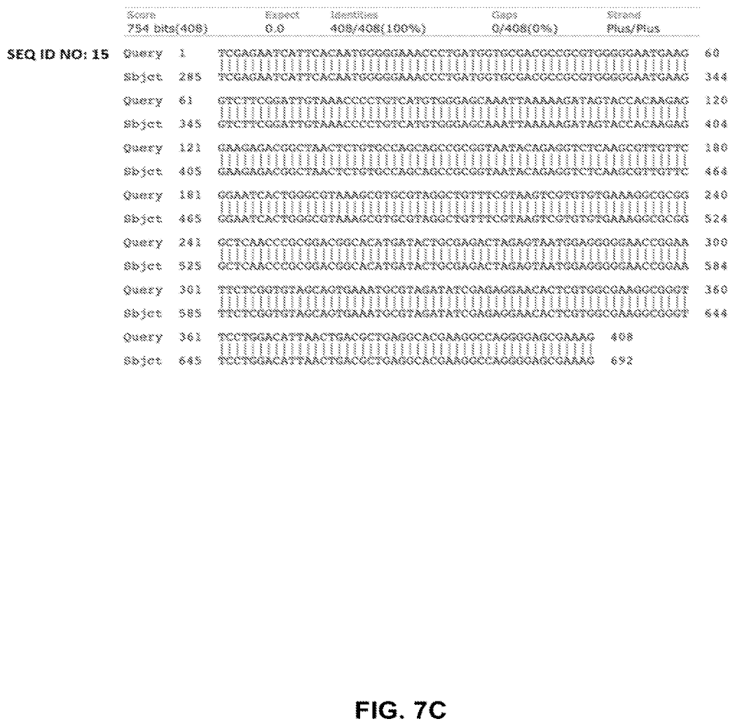

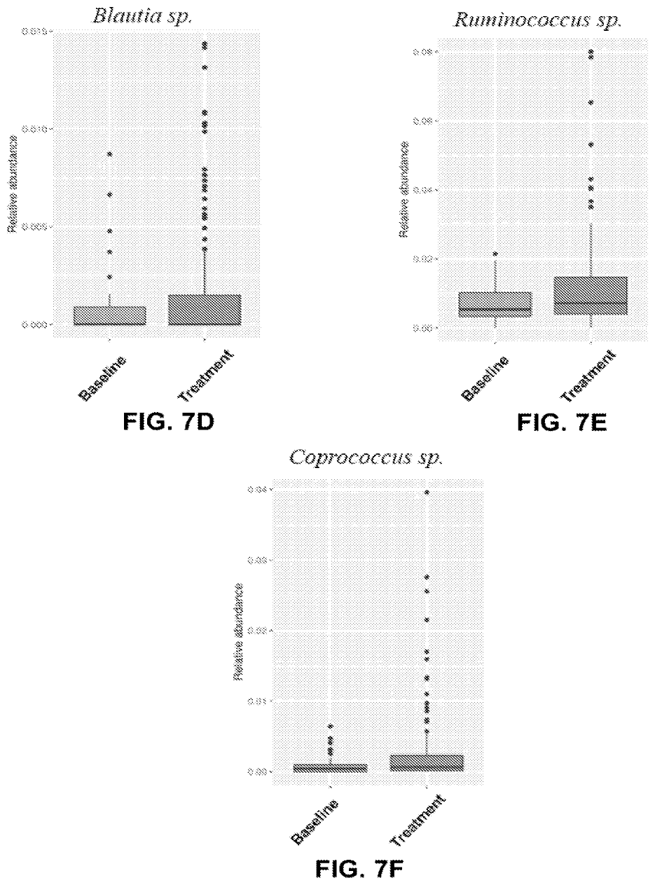

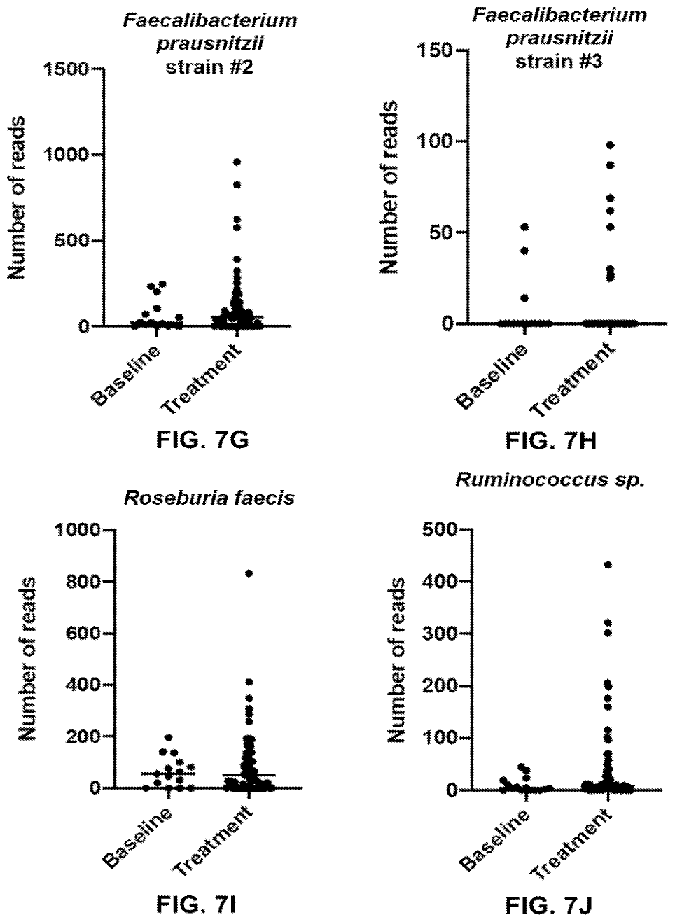

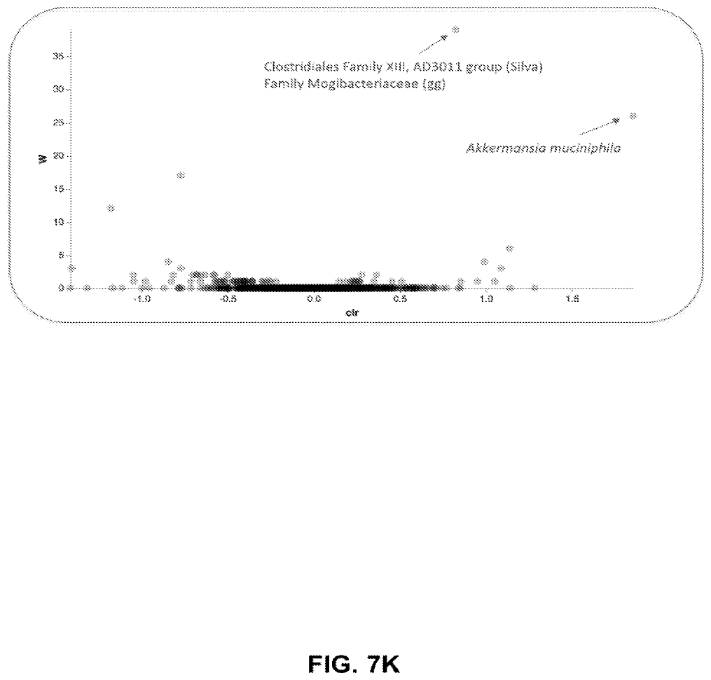

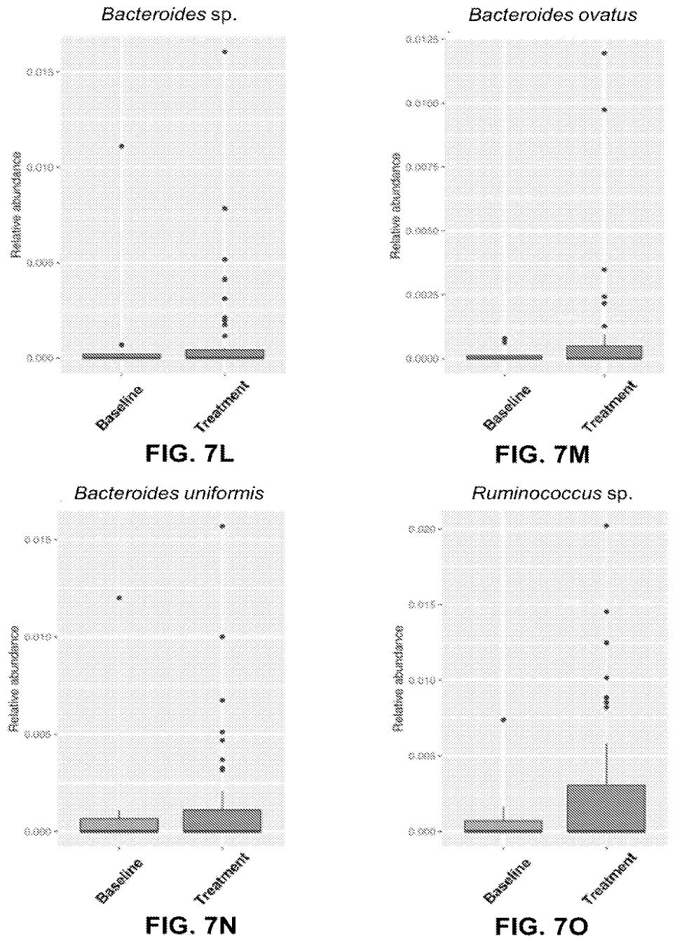

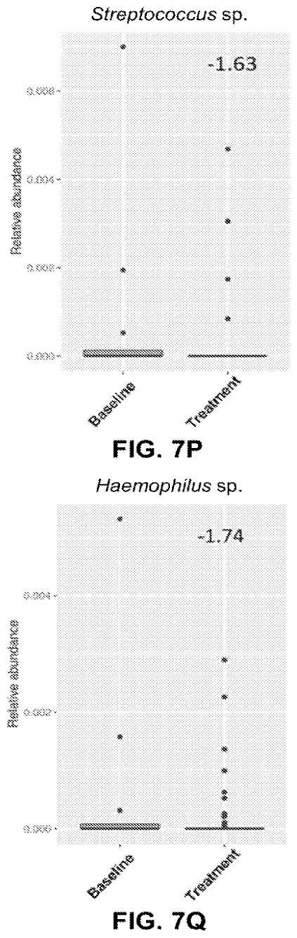

To identify the specific taxa that diverged from the baseline after the pharmaceutical composition A treatment, enrichment analysis of specific taxa using Statistical Framework Analysis of Composition of Microbiomes (ANCOM) was carried out. Two metrices were calculated: (i) W statistic that represented the number of times of the null hypothesis (e.g., the average abundance of a given species of ASV is equal to that in the other group) was rejected for a given species or ASV; and (ii) Centered Log Ratio that represented the log fold change of each taxon compared to the average. As shown in FIGS. 7A-B, Akkermansia muciniphila, Adlercreutzia sp. and Enteroehabdus sp. were enriched after the subjects were administered with pharmaceutical composition A, relative to the placebo control. The 100% sequence identity between the enriched and reference Akkermansia muciniphila showed that the enriched Akkermansia sp. was A. muciniphila (DSM 33213), as illustrated in FIG. 7C.

To identify additional taxa in the subject administered with pharmaceutical composition A, the microbiomes were analyzed using Applies Generalized Additive Models for Location, Scale and Shape (GAMLSS) with a zero-inflated beta (BEZI) family for analysis of the MetamicrobiomeR package in R. Estimates were log (odds ratio) of relative abundances between sample groups. Such approach was advantageous because the results were compared across studies and repeated measure. TABLE 7 shows a summary of the most enriched and depleted taxa identified.

TABLE 7
MetamicrobiomeR of all three cohorts

| | Cohort 1 | Cohort 2 | Cohort 3 |
|---|---|---|---|
| Total number of | 22 | 14 | 14 |
| taxa significantly | | | |
| enriched (ASVs) | | | |
| Most significantly | Bacteroidaceae | Streptococcus | Bacteroides ovatus |
| enriched taxa | Blautia producta | Coprococcus eutactus | Bacteroides plebeius |
| | Bacterioides eggerthii | Christensenellaceae | |
| | Clostridiaceae | | |
| | SMB53 | | |
| Total number of | 8 | 10 | 17 |
| taxa significantly | | | |
| depleted (ASVs) | | | |
| Most significantly | Enterococcus | Peptostreptococcaceae | Eggerthella lenta |
| depleted taxa | Turicibacter | Bacteroides ovatus | Coprococcus |
| | Oscillospira | Clostriaiaceae | Faecalibacterium |
| | Clostridiales | Slackia | prausnitzii * |
| | Coprococcus eutactus | | Haemophilus |
| | | | parainfluenzae |
| | | | Veillonella dispar |

* A different Faecalibacterium prausnitzii strain from that of pharmaceutical composition A

In the subjects of all cohorts, various beneficial butyrate-producing bacterial taxa, such as Blautia sp., Ruminococcus sp., and Coprococcus sp. were enriched in the gastrointestinal microbiota after the pharmaceutical composition A treatment (FIGS. 7D-F). Multiple butyrate-producing F. prausnitzii strains—different from the one in pharmaceutical composition A-Roseburia faecis, and Ruminococcus sp. were also enriched in the subjects of cohort 1 during the composition treatment compared to that of the baseline, as illustrated in FIGS. 7 G-J. Therefore, pharmaceutical composition A elevated the abundance of the beneficial bacterial taxa in the gastrointestinal tract of the subject.

In the subjects of cohort 3, the ANCOM analysis suggested that Closrtridales family XIII and family Mogibacteriaceae were enriched by pharmaceutical composition A (FIG. 7K). MetamicrobiomeR showed that Bacteroides sp., such as Bacteroides ovatus and Bacteroides uniformis, and Ruminococcus sp. were enriched in the treatment period of pharmaceutical composition A (FIGS. 7L-0). The Bacteroidetes colonization can be associated with a more “mature gut microbiota profile” and protection from opportunistic pathogens in infants. Early Bacteroidetes colonization in infants has been linked with natural birth and protection from allergy development, while cesarean birth can be associated with decreased Bacteroidetes. These results suggested that the pharmaceutical composition shifted the microbiome in young subjects into an adult-like one. Pathogenic species associated with asthma exacerbation in children, such as Streptococcus sp. and Haemophilus sp., were depleted by pharmaceutical composition A in the subjects of cohort 3 (FIGS. 7P-Q).

These results supported that pharmaceutical composition A shifted a microbiome to one with the makeup of that of a healthy subject, enriching beneficial microbial taxa while depleting the harmful ones.

### Example 4: Metabolic and Immune Marker Changes Associated with Decreased Inflammatory Activity

The pharmaceutical composition decreased the inflammatory activity of a subject, as illustrated by the metabolic and immune markers associated with the activity.

### Metabolic Change Induced by Pharmaceutical Composition A

Untargeted metabolomes of the subjects from the treatment and placebo groups were analyzed using untargeted mass spectrometry (Metabolon). TABLE 8 lists the summary of the analysis in the subjects of cohort 1 and cohort 2.

TABLE 8
Summary of metabolites with significant fold changes in the subjects
of cohorts ½

| | | | Number of | Number of | Number of | Number of |
|---|---|---|---|---|---|---|
| | | | metabolites | metabolites | metabolites | metabolites |
| | | | with a | with a | with a | with a |
| | First | Second | significant | significant | significant | significant |
| Cohort | condition | condition | increase * | decrease ** | increase *** | decrease **** |
| 1 | PBO BL | PBO E/T | 49 | 26 | 36 | 41 |
| | PBO BL | PBO W/O | 19 | 44 | 22 | 44 |
| | PBO E/T | PBO W/O | 6 | 33 | 8 | 37 |
| | PCA BL | PCA E/T | 41 | 43 | 51 | 39 |
| | PCA BL | PCA W/O | 35 | 72 | 30 | 40 |
| | PCA E/T | PCA W/O | 25 | 36 | 41 | 45 |
| | PBO BL | PCA BL | 39 | 10 | 29 | 13 |
| | PBO E/T | PCA E/T | 62 | 34 | 56 | 20 |
| | PBO W/O | PCA W/O | 49 | 5 | 40 | 13 |
| 2 | PBO BL | PBO E/T | 3 | 52 | 7 | 41 |
| | PBO BL | PBO W/O | 13 | 13 | 19 | 22 |
| | PBO E/T | PBO W/O | 51 | 6 | 49 | 7 |
| | PCA BL | PCA E/T | 17 | 27 | 14 | 25 |
| | PCA BL | PCA W/O | 53 | 21 | 38 | 14 |
| | PCA E/T | PCA W/O | 47 | 14 | 40 | 8 |
| | PBO BL | PCA BL | 3 | 42 | 7 | 39 |
| | PBO E/T | PCA E/T | 11 | 32 | 19 | 15 |
| | PBO W/O | PCA W/O | 10 | 19 | 11 | 21 |

PBO: placebo
PCA: pharmaceutical composition A
BL: baseline
E/T: End of Treatment (after 4 weeks of daily treatment with placebo or pharmaceutical composition A)
W/O: washout (4 weeks after cessation of treatment)
* with a significant increase in the second condition compared to the first condition with p ≤ 0 .05
** with a significant decrease in the second condition compared to the first condition with ≤ 0.05
*** with a significant increase in the second condition compared to the first condition with 0.5 < p < 0.1
**** with a significant decrease in the second condition compared to the first condition with 0.5 < p < 0.1

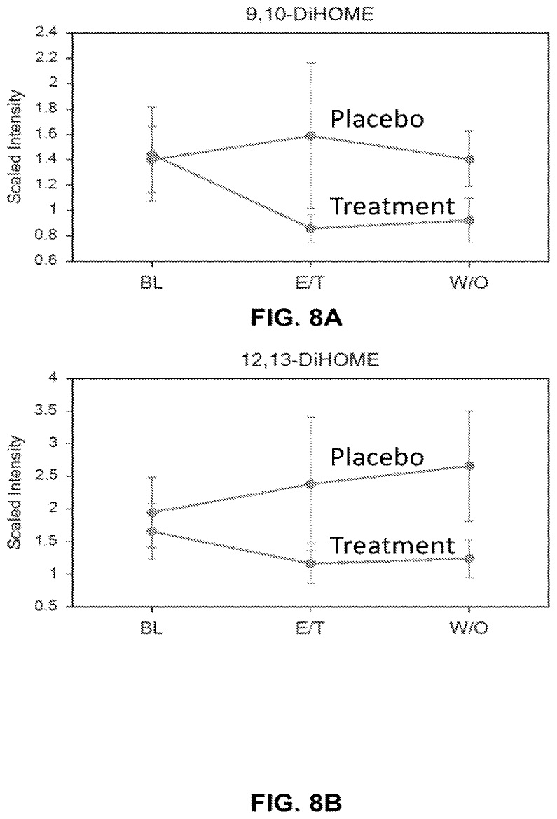

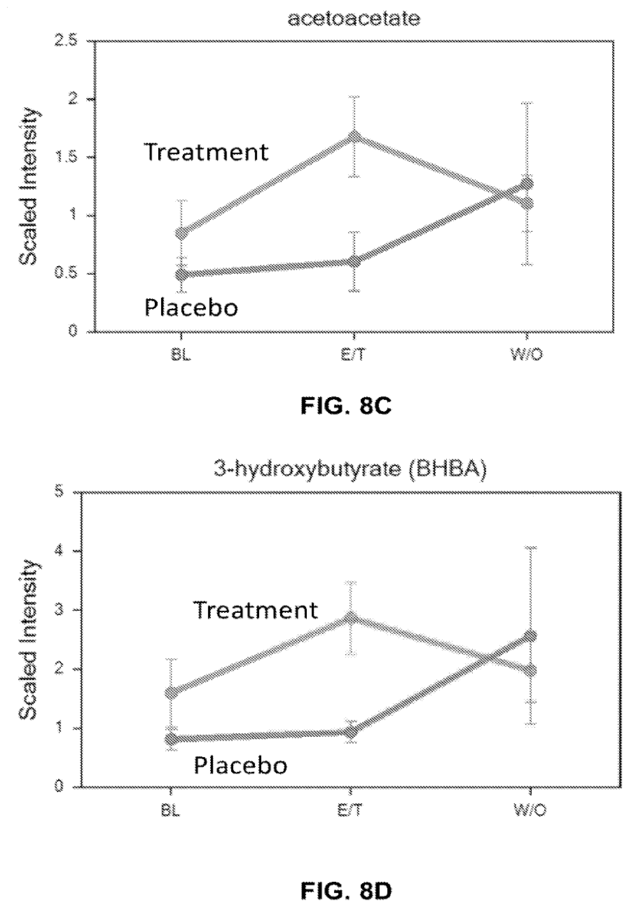

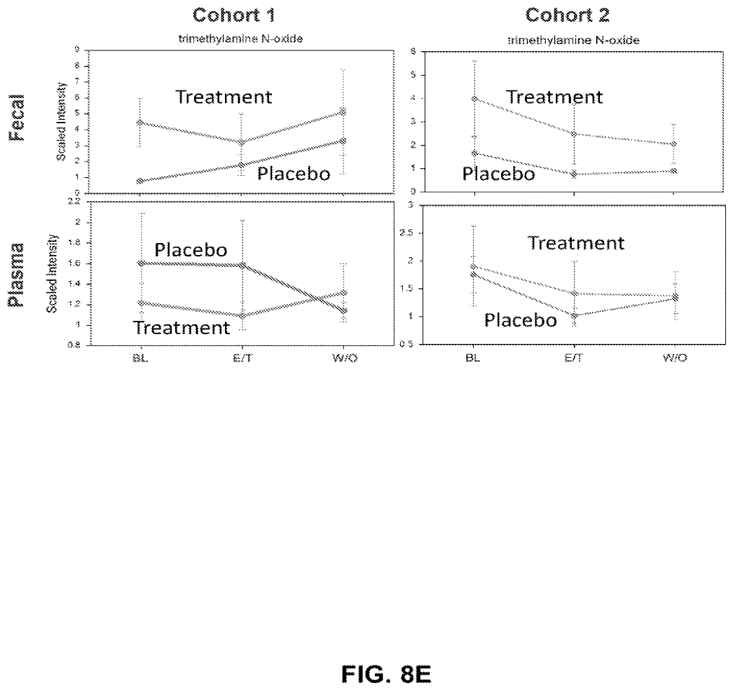

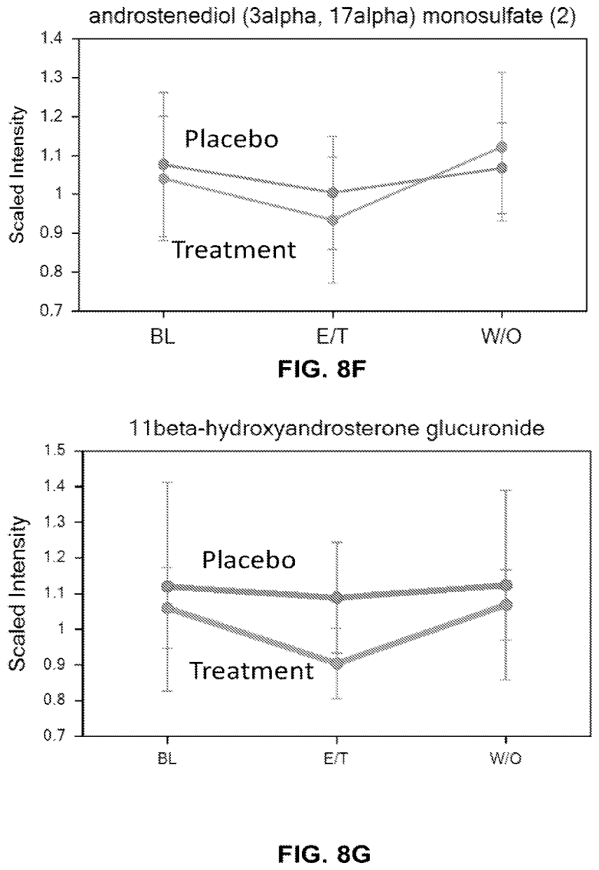

As shown in FIGS. 8A-81B and TABLE 9, in cohorts 1 and 2, the pharmaceutical composition decreased the level of 9-,10-DiHOME and 12, 13-DiHOME, compounds positively correlated with the inflammation, in the subject through the end of treatment and well into the washout period after the treatment, as compared to those of the placebo group.

TABLE 9
Summary of DiHOME lipid level in subjects administered
with pharmaceutical composition A

| | | Fold-change of | Fold-change of | |
|---|---|---|---|---|
| | | pharmaceutical | pharmaceutical | P-value |
| | | composition A | composition A | (Welch |
| | | treatment over | treatment over | two- |
| Sample | | baseline in | baseline in | sample |
| Type | Compound | cohort 1 | cohort 2 | t-test) |
| Fecal | 12,13-DiHOME | 0.808 | 0.702 | 0.083 |
| Fecal | 12,13-DiHOME | 0.645 | 0.595 | 0.093 |
| Plasma | 9,10-DiHOME | 0.667 | 0.914 | 0.352 |
| Plasma | 9,10-DiHOME | 1.044 | 0.606 | 0.404 |

As shown in FIGS. 8C-8D and TABLE 10, pharmaceutical composition A increased the level of acetoacetate and 3-hydroxybutyrate (BHBA) in the subjects during the treatment period, as compared to those of the placebo group.

TABLE 10
Summary of circulating plasma ketone level in subjects
administered with pharmaceutical composition A

| | Fold-change of | Fold-change of | |
|---|---|---|---|
| | pharmaceutical | pharmaceutical | |
| | composition A | composition A | P-value |
| | treatment over | treatment over | (Welch |
| | baseline in | baseline in | two-sample |
| Compound | cohort 1 | cohort 2 | t-test) |
| Acetoacetate | 1.976 | 1.397 | 0.015 |
| BHBA | 1.793 | 1.365 | 0.079 |

The result was consistent with an increase of butyrate-producing bacterial taxa induced by pharmaceutical composition A, as illustrated in EXAMPLE 4. Like other ketones, acetoacetate and BHBA can have cytoprotective and neuroprotective effects, some of which can occur through the inhibition of histone deacetylation. BHBA can also block inflammasome-mediated inflammatory disease.

As shown in FIG. 8E and TABLE 11, pharmaceutical composition A increased the level of trimethylamine N-oxide (TMAO)—a compound that can contribute to inflammation, altered bile acids and cholesterol transport, platelet hyperactivity, and foam cell formation—in the subject during the treatment period, as compared to those of the placebo group.

TABLE 11
Summary of TMAO level in subjects administered
with pharmaceutical composition A

| | Fold-change of | Fold-change of | |
|---|---|---|---|
| | pharmaceutical | pharmaceutical | |
| | composition A | composition A | P-value |
| | treatment over | treatment over | (Welch |
| Sample | baseline in | baseline in | two-sample |
| type | cohort 1 | cohort 2 | t-test) |
| Fecal | 0.723 | 0.622 | 0.078 |
| Plasma | 10.897 | 0.581 | 0.040 |

As shown in FIGS. 8F-8G and TABLE 12, pharmaceutical composition A increased the level of androgenic plasma steroids in the subject during the treatment period, as compared to those of the placebo group.

TABLE 12
Summary of circulating plasma ketone level in subjects
administered with pharmaceutical composition A

| | Fold-change of | Fold-change of | |
|---|---|---|---|
| | pharmaceutical | pharmaceutical | |
| | composition A | composition A | P-value |
| | treatment over | treatment over | (Welch |
| | baseline in | baseline in | two-sample |
| Compound | cohort 1 | cohort 2 | t-test) |
| Androstenediol | 0.844 | 0.933 | 0.045 |
| (3alpha,17alpha) | | | |
| monosulfate | | | |
| 11beta- | 0.853 | 0.840 | 0.066 |
| hydroxyandrosterone | | | |
| glucuronide | | | |
| Androsterone | 0.840 | 0.893 | 0.090 |
| glucuronide | | | |

Steroid sex hormones can be involved in the development and severity of asthma. Further, there can be disparities in asthma occurrence and severity between sexes.

Multiple membrane lipid species were also downregulated by pharmaceutical composition A, as shown in TABLE 13.

TABLE 13
Summary of Circulating Blood Membrane Lipid Level in Subjects
Administered with Pharmaceutical composition A

| | | Fold-change of | Fold-change of | |
|---|---|---|---|---|
| | | pharmaceutical | pharmaceutical | |
| | | composition A | composition A | P-value |
| | | treatment over | treatment over | (Welch |
| | | baseline in | baseline in | two-sample |
| Compound | Species | cohort 1 | cohort 2 | t-test) |
| 1,2- | Phosphatidylcholine (PC) | 1.024 | 1.092 | 0.047 |
| dipalmitoyl- | | | | |
| GPC | | | | |
| (16:0/16:0) | | | | |
| 1-palmitoyl-2- | PC | 1.175 | 1.305 | 0.060 |
| palmitoleoyl- | | | | |
| GPC | | | | |
| (16:0/16:1) | | | | |
| 1-myristoy1-2- | PC | 1.101 | 1.731 | 0.091 |
| palmitoyl- | | | | |
| GPC | | | | |
| (14:0/16:0) | | | | |
| 1-palmitoyl-2- | PC | 1.071 | 1.068 | 0.099 |
| oleoyl-GPC | | | | |
| (16:0/18:1) | | | | |
| 1-palmitoyl-2- | Phosphatidylethanolamine | 1.161 | 1.203 | 0.045 |
| arachidonoyl- | (PE) | | | |
| GPE | | | | |
| (16:0/20:4) | | | | |
| 1-palmitoyl-2- | PE | 1.140 | 1.268 | 0.089 |
| linoleoyl-GPE | | | | |
| (16:0/18:2) | | | | |
| 1-palmitoyl-2- | Phosphatidylinositol (PI) | 1.035 | 1.241 | 0.241 |
| linoleoyl-GPI | | | | |
| (16:0/18:2) | | | | |

### Decreased Inflammatory Immune Marker Expression

The pharmaceutical composition decreased the expression of inflammatory immune markers.

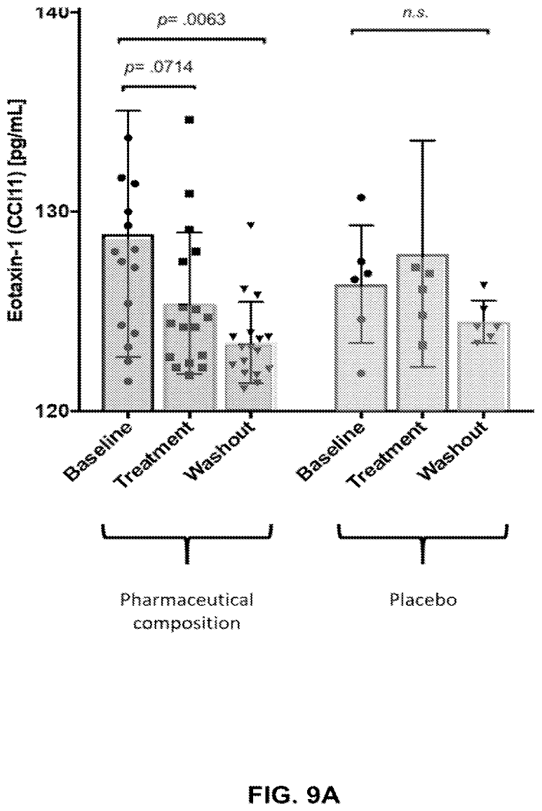

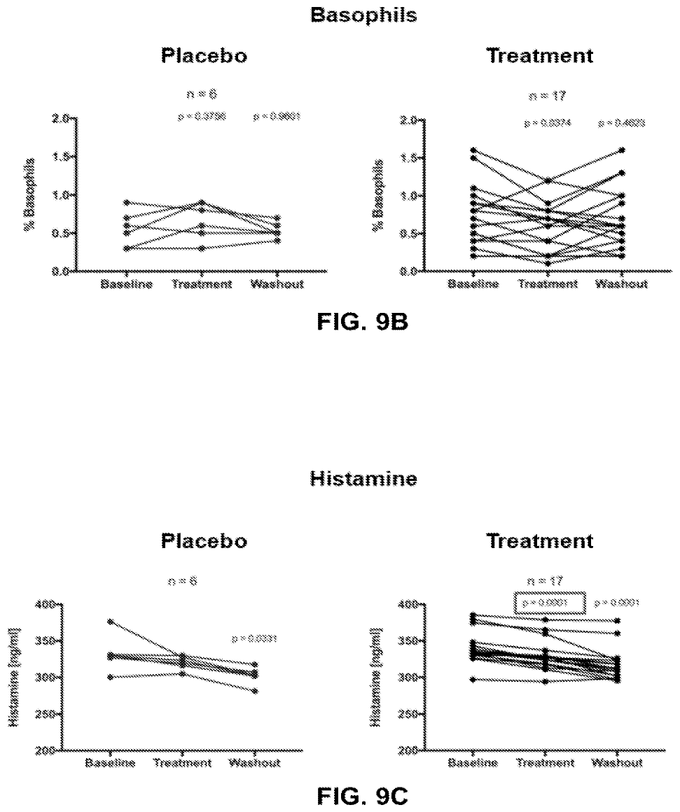

To measure the immunological impact of the pharmaceutical composition, the expression level of eotaxin-1 protein in the blood, encoded by the CCL11 gene, was measured using ELISA. As shown in FIG. 9A, the pharmaceutical composition statistically significantly decreased the level of eotaxin-1 in the subjects well into the washout period after the treatment. The difference of the level of eotaxin-1 of the subjects treated with the pharmaceutical bacterial population between the baseline level and washout period was statistically significant (p=0.063). There was also a difference in the level of eotaxin-1 of the subjects treated with the pharmaceutical bacterial population between the baseline level and the treatment period. Similar changes were not observed in the subjects treated with placebo control. Therefore, the bacterial population reduced the expression of the expression of inflammatory markers, indicating that a downregulation of the inflammatory activity in the subject administered with the pharmaceutical composition. Other inflammatory markers, such as histamine and basophils, also showed statistically significant decrease in the treatment or washout period (FIGS. 9B-C).

Altogether, the results suggested that the pharmaceutical composition comprising the bacterial population comprising Lactobacillus sp. (or species of the Lactobacillaceae family), Faecalibacterium sp., and Akkermansia sp. decreased inflammatory activity.

### Example 5: Growth, Isolation, and Characterization of Bacterial Strains

Provided herein are methods for the growth, isolation, and characterization of bacterial strains isolated from a human sample. Such strains may be used as part of a bacterial consortium described herein.

### 1. Growth of Bacteria

Generally, the procedures for growing bacteria as described herein can be used for culturing obligatory and facultative anaerobic bacterial strains. The bacterial strains in this example were derived from a human fecal sample and were grown on selective media. Upon subculturing, the colonies were transferred to a liquid medium and subsequently prepared for PCR and sequencing. Colonies were also preserved as glycerol stocks.

First, human fecal samples were collected in anaerobic transport media (Anaerobic Systems As-915) or in fecal collection vials sealed within a plastic bag containing an anaerobic atmosphere generating system (e.g., AnaeroPouch Thermo Fisher R686001). All samples were immediately transferred into an anaerobic chamber to minimize transit time and potential oxygen exposure to ensure viability of the anaerobic strains.

Serial dilution tubes were prepared by aliquoting 0.9 mL of PBS+Cys (1×PBS+0.1% w/w L-cysteine) into 13 tubes (with a volume of 1.5 mL). Using a disposable spatula or loop, 20-30 mg of sample was transferred into a first 1.5 mL tube containing 0.9 mL of PBS+Cys. The resulting mixture was vortexed for approximately 30 seconds and 0.1 mL of the resulting, homogenous solution was transferred into the second 1.5 mL tube containing 0.9 mL of PBS+Cys. This step was then repeated until all 13 tubes contained serial dilutions (e.g., 1-10\hat{ }−12 dilutions in vials 1-13) of the sample.

Using a disposable hockey stick spreader, −0.1 mL from the sample tubes containing the dilutions 10\hat{ }−5 to 10\hat{ }−12 (vials 6-13) were added to separate agar plates containing the selective agar growth media. The agar plates were then sealed with parafilm to prevent evaporation and placed into an incubator for 72 hours at 37° C. Colonies that fit a colony's (e.g., L. crispatus (DSM 33187), A. muciniphila (DSM 33213), F. prausnitzii (DSM 33185), etc.) specific morphology were identified, and placed on a new, pre-reduced agar plate for isolation. The agar plates were sealed with parafilm and placed into an anaerobic incubator for another 72 hours at 37° C.

The isolated colonies were transferred into liquid media by picking specifically isolated colonies from culture plates and resuspending the colonies in 1 mL of pre-reduced liquid broth. Positive and negative controls of selected organisms were inoculated in parallel to compare for growth and monitor for contamination, respectively. All liquid colony samples were then incubated for 72 hours at 37° C.

Using the positive and negative controls, positive match broth cultures were identified. Glycerol stocks of positive match broth cultures were prepared by transferring 0.75 mL of the broth culture solution into a 2 mL cryotube containing 0.75 mL of 50% v/v glycerol in PBS. Sealed cryotube samples were then removed from the anaerobic chamber and stored at −80° C. The remaining broth culture samples were used for isolate identification using 16S-based PCR as described below.

### 2. 16S-based PCR for Isolate Identification

Broth culture samples were centrifuged to form cell pellets and the resulting supernatant was carefully removed to leave the formed cell pellet intact. The cell pellet was then resuspended in 0.5-1 mL ultrapure water.

The PCR Mastermix for a final reaction volume of 50 μL was prepared using the following PCR components (NEB E5000S) and volumes: 10× Buffer (5 μL), 10 mM dNTPs (1 μL), 10 μM 27F Forward Primer (1 μL), 10 μM 1492R Reverse Primer (1 μL), Tag Polymerase (0.25 μL), and sterile water (40.25 μL). The PCR Mastermix (48.5 μL) and 1.5 μL of resuspended bacterial cells were placed into a 0.2 mL PCR strip tube and vortexed before the PCR reaction samples were exposed to the following thermocycler protocol (TABLE 14):

TABLE 14
PCR Thermocycler Protocol.

| Step | Temp | Time | |
|---|---|---|---|
| 1 | 95° C. | 2 | min |
| 2 | 95° C. | 30 | sec |
| 3 | 50° C. | 30 | sec |
| 4 | 68° C. | 1 min 30 sec (30 | |
| | | repeats of steps 2-4) | |
| 5 | 72° C. | 5 | min |

| | | |
|---|---|---|
| 6 | 4° C. | HOLD |

Upon completion of the PCR reactions, samples were submitted or Sanger sequencing using GENEWIZ or a comparable vendor.

### Example 6: Quantification of Bacterial Cells in Fecal DNA Using qPCR

The use of quantitative polymerase chain reaction (qPCR) is described for quantification of the bacterial strains Akkermansia muciniphila (DSM 33213), Faecalibacterium prausnitzii (DSM 33185), and Lactobacillus crispatus (DSM 33187) in a sample. This example describes strain quantification using specific primers, qPCR and fecal DNA as a template.

### 1. Materials

TABLE 15 below shows exemplary strain-specific primer sequences used for strain quantification:

TABLE 15
Strain-specific Primer Sequences.

| | | | | GC |
|---|---|---|---|---|
| Primer | SEQ ID | | | Content |
| Sequence (5′ to 3′) | NO | Strain | Tm | (%) |
| TATCCGGACTCCTCCATCTG | 1 | A. muciniphila (DSM 33213) | 55 | 62.4 |
| TGTTCGTGCGTTCTTACCTG | 2 | A. muciniphila (DSM 33213) | 50 | 60.4 |
| ATTCCTGAGAAGGCCAGGAT | 3 | A. muciniphila (DSM 33213) | 60.4 | 50 |
| CTGCCGACAAGCATTCCTAT | 4 | A. muciniphila (DSM 33213) | 60.4 | 50 |
| ACCAAGGTTAGCCGCTTTTT | 5 | A. muciniphila (DSM 33213) | 45 | 58.4 |
| CTTGCCCAACAAAATGACCT | 6 | A. muciniphila (DSM 33213) | 45 | 58.4 |
| GTAAGCTCTGTTTCGGCAGCAC | 7 | F. prausnitzii (DSM 33185) | 62.3 | 54.5 |
| ACATTGCACGCTTTGCCGAC | 8 | F. prausnitzii (DSM 33185) | 62.7 | 55 |
| AATTCAGGTTCGGCTGCTGT | 9 | F. prausnitzii (DSM 33185) | 60.4 | 50 |
| CAGGCAGACGTTCTGCTACT | 10 | F. prausnitzii (DSM 33185) | 62.4 | 55 |
| ACGCGTCTCTTTTTGAGCAC | 11 | L. crispatus (DSM 33187) | 60.4 | 50 |
| CCAAATTCAAAGGACTTGGGCT | 12 | L. crispatus (DSM 33187) | 60.8 | 45.45 |
| CGTAGTCCACTTAAGAAGGCCG | 13 | L. crispatus (DSM 33187) | 60.73 | 54.55 |
| GGGCTTTCTTCAAACCTGGC | 14 | L. crispatus (DSM 33187) | 59.68 | 55 |

PacBio sequencing was performed on genomic DNA extracted from L. crispatus (DSM 33187), F. prausnitzii (DSM 33185), A. muciniphila (DSM 33213). Using these data, a comparative genomic analysis of all bacterial strains was performed to identify unique regions within the genomes of L. crispatus (DSM 33187), F. prausnitzii (DSM 33185), A. muciniphila (DSM 33213). After identification of unique regions, qPCR primer pairs (see TABLE 15 above) were designed to target the unique regions present within these strains.

Additional materials used for this experiment included: (i) A. muciniphila (DSM 33213), F. prausnitzii (DSM 33185), L. crispatus (DSM 33187) DNA; (ii) Human Fecal DNA Sample Control (did not contain strains); (iii) extracted DNA from Clinical Samples; (iv) metal 384 qPCR plate holder; (v) DNA/RNAse free, sterile Eppendorf tubes; and (vi) QuantStudio 6 qPCR Thermocycler.

### 2. Procedure

Clinical sample names and DNA concentrations were calculated electronically, and all samples were normalized to 10 ng/μL with a final volume of 100 μL. Each run utilized a positive control standard curve of 7 points generated with pure bacterial DNA diluted in a human fecal DNA background. These standards (abbreviated as “std”) were premade and aliquoted for ease of use. For the standard curve, the following serial dilutions of target strain DNA were included in the run (TABLE 16):

TABLE 16
Standard Strain DNA Preparations.

| | | |
|---|---|---|
| | Final Amount | Final DNA |
| | of Strain- | Amount |
| | specific | per Well |
| | DNA per | (including |
| | Reaction | Control |
| Standard | well | Fecal DNA) |

| | | | |
|---|---|---|---|
| Std 1 | 10 | ng | 50 ng |
| Std 2 | 1 | ng | 50 ng |
| Std 3 | 0.1 | ng | 50 ng |
| Std 4 | 0.01 | ng | 50 ng |
| Std 5 | 0.001 | ng | 50 ng |
| Std 6 | 0.0001 | ng | 50 ng |
| Std 7 | 0.00001 | ng | 50 ng |

The following items were used for strain DNA quantification experiments:

TABLE 17
Materials for DNA Quantification Experiments.

| Item | Quantity |
|---|---|
| Fecal DNA from Clinical | 161 samples |
| Samples (store on ice) | (max) |
| A. muciniphila (DSM 33213), | ST Strain DNA |
| F. prausnitzii (DSM 33185) or | (Plate Specific) |
| L. crispatus (DSM 33187) | |
| Standard DNA | |
| 384 well plate (sterile) | 1 |
| 96 well plate (sterile) | 2 |
| Eppendorf Tubes (sterile) | 3 |
| 15 mL Falcon Tube (sterile) | 1 |
| Pipette | 1000 μL, 200 μL, 10 μL, |
| | multichannel pipette |
| Pipette tips (sterile) | 1 box of 1000 μL, |
| | 200 μL, 10 μL, |
| USP WFI (sterile) | 50 mL |
| Tube Rack | 1 |
| 384 Metal Cooling Plate | 1 |
| Styrofoam cooler full of ice | 1 |
| Sealing Film | 2 |
| Primers (forward and reverse) | 1 stock of each strain |
| SYBR Select Master Mix (store on ice) | One (1) 5 mL bottle |

The calculated amount of water and DNA solution was added to the appropriate wells. The qPCR plate was sealed, vortexed (5-10 seconds) and centrifuged for 2 minutes at 1000 rpm.

### 3. Preparation of Primer Stocks and Master Mix

The forward and reverse strain specific primers were completely thawed, and primer stocks were maintained at 100 μM in 1×TE buffer (see, for example, TABLE 15). In two Eppendorf tubes (one for the forward primer and one for the reverse primer) 360 μL of sterile USP grade WFI was added. Then, 40 μL of forward primer solution was added and homogenized, and the same step was repeated for the reverse primer solution. The resulting 10 μM primers solutions were combined with SYBR Select Master Mix (2× Stock) and sterile water and homogenized.

Using the qPCR 384 Well Plate, 20 μL of qPCR Master Mix were aliquoted into wells A-N 1-24 and O 1-14. After master mix had been aliquoted into all 350 wells, using the DNA normalization plate, 5 μL of DNA of all wells in row A were taken up and inoculated into the odd wells in row A in the reaction plate. Then, 5 μL of DNA from all wells in row A was transferred and inoculated into the even wells in row A in the reaction plate. The last 2 steps were repeated for all wells until the DNA was added to the entire reaction plate, except row P. Subsequently, the standard curve stock DNA plate of the appropriate strain was transferred to the Biosafety Cabinet, followed by pipetting the standards into the correct wells of row P per the reaction plate setup. Following mixing and centrifugation, the samples were placed in the Quantstudio qPCR machine.

The following cycling conditions were used as shown below in TABLE 18:

TABLE 18
Cycling Conditions.

Cycling Conditions

| Step | Temp. ° C. | Time | | |
|---|---|---|---|---|
| Activation | 50 | 2 | min | |
| Initial Denaturation | 95 | 2 | min | |
| Denaturation | 95 | 15 | seconds | 40 Cycles |
| Anneal/Extend | 60 | 1 | min | |

TABLE 19 below shows standard curve control cycle threshold (CT) and primer melting temperature (TM) values. TABLE 19 further shows, for each of the selected strains F. prausntzii (DSM 33185), L. crispatus (DSM 33187), and A. muciniphila (DSM 33213), the estimated number of strain cells in human fecal DNA with standard amounts of strain cell DNA (e.g., 1 ng, 0.1 ng, 0.01 ng, and 0.001 ng) used for generating a standard curve that may be used to quantify the amount of strain cell DNA (using a human fecal DNA background), and the same of amounts of strain cell DNA (e.g., 1 ng, 0.1 ng, 0.01 ng, and 0.001 ng) in water (e.g., without fecal DNA background):

TABLE 19
Standard Curve Control Values and Primer TM Values.

| | Estimated | Strain and Primers | Average | Average |
|---|---|---|---|---|
| Sample Name | No. of Cells | Name | CT | TM 1 |
| 50 ng Human Fecal DNA | NA | F. prausnitzii Primer 03 | 39.32 | 83.24 |
| 50 ng HF DNA + 1 ng Fp DNA | 290000 | F. prausnitzii Primer 03 | 21.51 | 83.44 |
| 50 ng HF DNA + 0.1 ng Fp DNA | 29000 | F. prausnitzii Primer 03 | 23.14 | 83.34 |
| 50 ng HF DNA + 0.01 ng Fp DNA | 2900 | F. prausnitzii Primer 03 | 26.04 | 83.39 |
| 50 ng HF DNA + 0.001 ng Fp DNA | 290 | F. prausnitzii Primer 03 | 29.67 | 83.34 |
| Water | 0 | F. prausnitzii Primer 03 | NA | 61.27 |
| 1 ng Fp DNA | 290000 | F. prausnitzii Primer 03 | 21.29 | 83.64 |
| 0.1 ng Fp DNA | 29000 | F. prausnitzii Primer 03 | 23.79 | 83.59 |
| 0.01 ng Fp DNA | 2900 | F. prausnitzii Primer 03 | 26.77 | 83.54 |
| 0.001 ng Fp DNA | 290 | F. prausnitzii Primer 03 | 30.63 | 88.32 |
| 50 ng Human Fecal DNA | NA | L. crispatus Primer 02 | 34.54 | 73.69 |
| 50 ng HF DNA + 1 ng Lc DNA | 279000 | L. crispatus Primer 02 | 19.63 | 73.98 |
| 50 ng HF DNA + 0.1 ng Lc DNA | 27900 | L. crispatus Primer 02 | 22.98 | 74.03 |
| 50 ng HF DNA + 0.01 ng Lc DNA | 2790 | L. crispatus Primer 02 | 26.15 | 73.93 |
| 50 ng HF DNA + 0.001 ng Lc DNA | 279 | L. crispatus Primer 02 | 30.43 | 73.98 |
| Water | 0 | L. crispatus Primer 02 | 36.00 | 73.93 |
| 1 ng Lc DNA | 279000 | L. crispatus Primer 02 | 20.47 | 74.28 |
| 0.1 ng Lc DNA | 27900 | L. crispatus Primer 02 | 24.01 | 74.28 |
| 0.01 ng Lc DNA | 2790 | L. crispatus Primer 02 | 26.95 | 74.23 |
| 0.001 ng Lc DNA | 279 | L. crispatus Primer 02 | 30.16 | 74.08 |
| 50 ng Human Fecal DNA | NA | A. muciniphila Primer 03 | 35.29 | 77.54 |
| 50 ng HF DNA + 1 ng Am DNA | 337000 | A. muciniphila Primer 03 | 18.63 | 77.59 |
| 50 ng HF DNA + 0.1 ng Am DNA | 33700 | A. muciniphila Primer 03 | 22.01 | 77.59 |
| 50 ng HF DNA + 0.01 ng Am DNA | 3370 | A. muciniphila Primer 03 | 25.07 | 77.59 |
| 50 ng HF DNA + 0.001 ng Am DNA | 337 | A. muciniphila Primer 03 | 27.82 | 77.49 |
| Water | 0 | A. muciniphila Primer 03 | 37.85 | 67.33 |
| 1 ng Am DNA | 337000 | A. muciniphila Primer 03 | 20.29 | 77.78 |
| 0.1 ng Am DNA | 33700 | A. muciniphila Primer 03 | 23.26 | 77.73 |
| 0.01 ng Am DNA | 3370 | A. muciniphila Primer 03 | 26.13 | 77.78 |

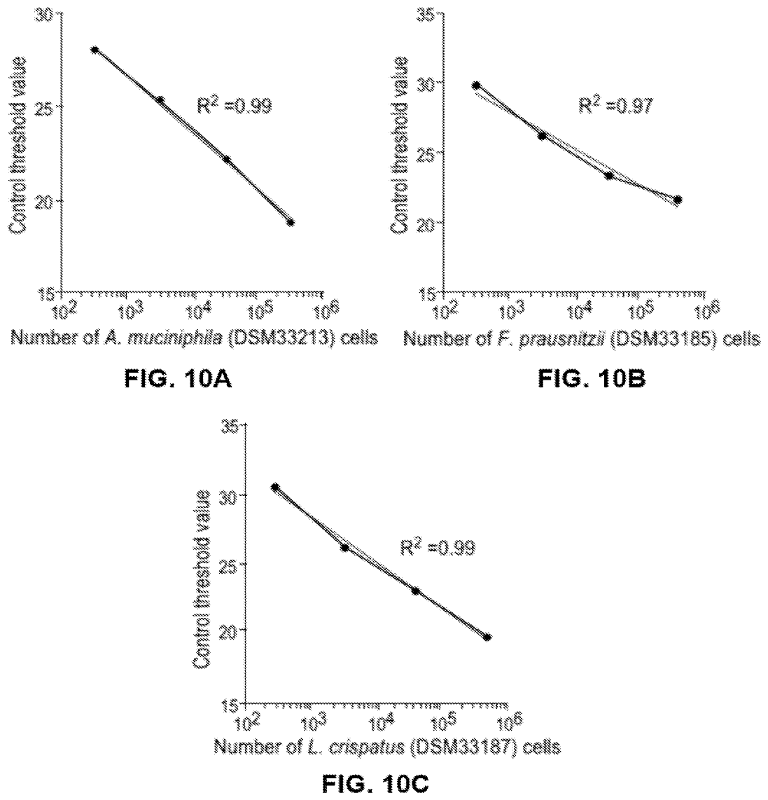

FIGS. 10A-C show limit of detection curves for the three selected strains A. muciniphila (DSM 33213), F. prausnitzii (DSM 33185), and L. crispatus (DSM 33187) cells, respectively, that were generated by plotting the measured CT values against the number of estimated strain cells as shown in TABLE 19 above.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

1. A method comprising:
(a) administering to a subject a pharmaceutical composition comprising a bacterial population comprising at least two bacterial species selected from the group consisting of Akkermansia sp., Faecalibacterium sp., and Lactobacillus sp.;
(b) assaying a microbial community in said subject relative to a control microbial community.
   2. A method comprising:
(a) obtaining a sample comprising a microbial community from a subject;
(b) assaying said microbial community relative to a control microbial community, wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp.
   3. The method of embodiment 1 or 2, wherein said assaying of (b) comprises repeated measures of said microbial community.
   4. The method of any one of embodiments 1-3, wherein said assaying of (b) comprises a linear mixed effect (LME) model.
   5. The method of any one of embodiments 1-4, wherein said assaying of (b) measures a microbial diversity of said microbial community.
   6. The method of embodiment 5, wherein said microbial diversity comprises an alpha-diversity or a beta-diversity.
   7. The method of embodiment 6, wherein said microbial diversity comprises said alpha-diversity.
   8. The method of embodiment 6 or 7, wherein said microbial diversity comprises said beta-diversity.
   9. The method of embodiment 8, wherein said beta-diversity is measured by a weighted UniFrac distance or a Jaccard distance.
   10. The method of embodiment 9, wherein said beta-diversity is measured by a weighted UniFrac distance.
   11. The method of embodiment 10, wherein said beta-diversity is measured by a Jaccard distance.
   12. The method of any one of embodiments 1-11, wherein said assaying of (b) identifies an enrichment or a depletion of at least one bacterial species in said microbial community.
   13. The method of embodiment 12, wherein said assaying of (b) identifies said enrichment of at least one bacterial species in said microbial community.
   14. The method of embodiment 13, wherein said at least one enriched bacterial species in said microbial community synthesizes short chain fatty acids (SCFAs).
   15. The method of any one of embodiments 12-14, wherein said assaying of (b) identifies said depletion of at least one bacterial species in said microbial community.
   16. The method of embodiment 15, wherein said at least one depleted bacterial species in said microbial community is associated with an inflammatory disease.
   17. The method of embodiment 16, wherein said inflammatory disease comprises an allergy or a dermatitis.
   18. The method of any one of embodiments 12-17, wherein said assaying of (b) identifies said enrichment or said depletion of at least one bacterial species in said microbial community using Statistical Framework Analysis of Composition of Microbiomes (ANCOM) or Applies Generalized Additive Models for Location, Scale and Shape (GAMLSS).
   19. The method of any one of embodiments 12-18, wherein said enriched bacterial species in said microbial community comprises one strain selected from the group consisting of family Bacteroidaceae and family Christensenellaceae.
   20. The method of any one of embodiments 12-19, wherein said enriched bacterial species in said microbial community comprises one strain selected from the group consisting of genus Bacteroides and genus Clostridiaceae SMB53.
   21. The method of any one of embodiments 12-20, wherein said enriched bacterial species in said microbial community comprises one strain selected from the group consisting of Ruminococcus sp. and Streptococcus sp.
   22. The method of any one of embodiments 12-21, wherein said enriched bacterial species in said microbial community comprises one strain selected from the group consisting of Roseburia faecis, Blautia producta, Bacteroides eggerthii, Bacteroides plebeius, and Coprococcus eutactus.
   23. The method of any one of embodiments 12-22, wherein said depleted bacterial species in said microbial community comprises one strain selected from the group consisting of family Clostridiales, family Peptostreptococcaceae, and family Clostridiaceae.
   24. The method of any one of embodiments 12-23, wherein said depleted bacterial species in said microbial community comprises one strain selected from the group consisting of genus Turicibacter, genus Oscillospira, genus Slackia, and genus Coprococcus.
   25. The method of any one of embodiments 12-24, wherein said depleted bacterial species in said microbial community comprises one strain selected from the group consisting of Streptococcus sp., Haemophilus sp., and Enterococcus sp.
   26. The method of any one of embodiments 12-25, wherein said depleted bacterial species in said microbial community comprises one strain selected from the group consisting of Bacteroides ovatus, Eggerthella lenta, Haemophilus parainfluenzae, and Veillonella dispar.
   27. The method of any one of embodiments 1-26, wherein said control microbial community comprises a microbial community in said subject without being administered to said pharmaceutical composition.
   28. The method of any one of embodiments 1-27, wherein said control microbial community comprises a microbial community in said subject prior to being administered to said pharmaceutical composition.
   29. The method of any one of embodiments 1-28, wherein said subject is an adult, an adolescent, or an infant.
   30. The method of any one of embodiments 1-29, wherein said subject has or is at risk of having an inflammatory disease, and wherein said inflammatory disease comprises an allergy or a dermatitis.
   31. The method of embodiment 30, wherein said allergy is allergic asthma, allergic pediatric asthma, or food allergy.
   32. The method of any one of embodiments 1-31, wherein said pharmaceutical composition is stored in a plant-based capsule prior to being administered to said subject.
   33. The method of any one of embodiments 1-32, wherein said bacterial population comprises said Akkermansia sp., said Faecalibacterium sp., and said Lactobacillus sp.
   34. The method of any one of embodiments 1-33, wherein said bacterial species is selected from the strains listed in Table 1.
   35. The method of embodiment 34, wherein said bacterial population comprises A. muciniphila (DSM 33213), F. prausnitzii (DSM 33185), or L. crispatus (DSM 33187).
   36. The method of embodiment 35, wherein said bacterial population comprises A. muciniphila (DSM 33213), F. prausnitzii (DSM 33185), and L. crispatus (DSM 33187).
   37. The method of any one of embodiments 1-36, wherein each bacterial species is present in an amount from about 10\hat{ }3 CFU/dose to about 10\hat{ }12 CFU/dose.
   38. The method of any one of embodiments 1-37, wherein each bacterial species is present in an amount from about 10\hat{ }7 CFU/dose to about 10\hat{ }10 CFU/dose.
   39. The method of any one of embodiments 1-38, wherein said Akkermansia sp. is present in an amount of about 10\hat{ }6 CFU/dose to about 2×10\hat{ }9 CFU/dose.
   40. The method of any one of embodiments 1-39, wherein said Faecalibacterium sp. is present in an amount of about 10\hat{ }6 CFU/dose to about 2×10\hat{ }9 CFU/dose.
   41. The method of any one of embodiments 1-40, wherein said Lactobacillus sp. is present in an amount of about 10\hat{ }7 CFU/dose to about 5×10\hat{ }9 CFU/dose.
   42. The method of any one of embodiments 1-41, wherein said bacterial population is present in a total amount of about 10\hat{ }3 CFU/dose to about 10\hat{ }12 CFU/dose.
   43. The method of any one of embodiments 1-42, wherein said bacterial population is present in a total amount of about 10\hat{ }7 CFU/dose to about 10\hat{ }10 CFU/dose.
   44. A method comprising:
(a) administering to a subject a pharmaceutical composition comprising a bacterial population comprising at least two bacterial species selected from the group consisting of Akkermansia sp., Faecalibacterium sp., and Lactobacillus sp.;
(b) assaying at least one bacterial species of said bacterial population in said subject relative to said at least one species of a control bacterial population.
   45. A method comprising:
(a) obtaining a sample from a subject;
(b) assaying at least one bacterial species of a bacterial population of said sample relative to said at least one species of a control bacterial population, wherein said sample is obtained from said subject subsequent to being administered with a pharmaceutical composition comprising said bacterial population comprising at least two bacterial species selected from the group consisting of Akkermansia sp., Faecalibacterium sp., and Lactobacillus sp.
   46. The method of embodiment 44 or 45, wherein said assaying of (b) comprises taking a mean abundance or a median abundance of said at least one bacterial species of said bacterial population and said control bacterial population.
   47. The method of any one of embodiments 44-46, wherein said control bacterial population comprises said bacterial population in said subject without being administered to said pharmaceutical composition.
   48. The method of any one of embodiments 44-47, wherein said control bacterial population comprises said bacterial population in said subject prior to being administered to said pharmaceutical composition.
   49. The method of any one of embodiments 44-48, wherein said at least one bacterial species of said bacterial population is present in said sample during said subject is administered to said pharmaceutical composition.
   50. The method of any one of embodiments 44-49, wherein said at least one bacterial species of said bacterial population is present in said sample in a washout period subsequent to said subject is administered to said pharmaceutical composition.
   51. The method of any one of embodiments 44-50, wherein said assaying of (b) comprises a quantitative PCR.
   52. The method of any one of embodiments 44-51, wherein said subject is an adult, an adolescent, or an infant.
   53. The method of any one of embodiments 44-52, wherein said subject has or is at risk of having an inflammatory disease, and wherein said inflammatory disease comprises an allergy or a dermatitis.
   54. The method of embodiment 53, wherein said allergy is allergic asthma, allergic pediatric asthma, or food allergy.
   55. The method of any one of embodiments 44-54, wherein said pharmaceutical composition is stored in a plant-based capsule prior to being administered to said subject.
   56. The method of any one of embodiments 44-55, wherein said bacterial population comprises said Akkermansia sp., said Faecalibacterium sp., and said Lactobacillus sp.
   57. The method of any one of embodiments 44-56, wherein said bacterial species is selected from the strains listed in Table 1.
   58. The method of embodiment 57, wherein said bacterial population comprises A. muciniphila (DSM 33213), F. prausnitzii (DSM 33185), or L. crispatus (DSM 33187).
   59. The method of embodiment 58, wherein said bacterial population comprises A. muciniphila (DSM 33213), F. prausnitzii (DSM 33185), and L. crispatus (DSM 33187).
   60. The method of any one of embodiments 44-59, wherein each bacterial species is present in an amount from about 10\hat{ }3 CFU/dose to about 10\hat{ }12 CFU/dose.
   61. The method of any one of embodiments 44-60, wherein each bacterial species is present in an amount from about 10\hat{ }7 CFU/dose to about 10\hat{ }10 CFU/dose.
   62. The method of any one of embodiments 44-61, wherein said Akkermansia sp. is present in an amount of about 10\hat{ }6 CFU/dose to about 2×10\hat{ }9 CFU/dose.
   63. The method of any one of embodiments 44-62, wherein said Faecalibacterium sp. is present in an amount of about 10\hat{ }6 CFU/dose to about 2×10\hat{ }9 CFU/dose.
   64. The method of any one of embodiments 44-63, wherein said Lactobacillus sp. is present in an amount of about 10\hat{ }7 CFU/dose to about 5×10\hat{ }9 CFU/dose.
   65. The method of any one of embodiments 44-64, wherein said bacterial population is present in a total amount of about 10\hat{ }3 CFU/dose to about 10\hat{ }12 CFU/dose.
   66. The method of any one of embodiments 44-65, wherein said bacterial population is present in a total amount of about 10\hat{ }7 CFU/dose to about 10\hat{ }10 CFU/dose.
   67. A method comprising:
(a) administering to a subject a pharmaceutical composition comprising a bacterial population comprising at least one strain of Akkermansia sp., at least one strain of Faecalibacterium sp., or at least one strain of Lactobacillus sp.;
(b) assaying a steroid compound in said subject relative to said steroid compound in a control sample.
   68. A method comprising:
(a) obtaining a sample comprising a steroid compound from a subject;
(b) assaying said steroid compound relative to said steroid compound in a control sample,
wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp.
   69. The method of embodiment 67 or 68, wherein said steroid compound comprises an androgenic steroid compound.
   70. The method of embodiment 69, wherein said androgenic steroid compound is selected from the group consisting of androstenediol (3alpha, 17alpha) monosulfate, 11beta-hydroxyandrosterone glucuronide, and androsterone glucuronide.
   71. The method of any one of embodiments 67-70, wherein said steroid compound in a control sample comprises said steroid compound in a subject without being administered to said pharmaceutical composition.
   72. The method of any one of embodiments 67-71, wherein said steroid compound in a control sample comprises said steroid compound in said subject prior to being administered to said pharmaceutical composition.
   73. The method of any one of embodiments 67-72, wherein said steroid compound is present in said subject during said subject is administered to said pharmaceutical composition.
   74. The method of any one of embodiments 67-73, wherein said steroid compound is present in said subject in a washout period subsequent to said subject is administered to said pharmaceutical composition.
   75. The method of any one of embodiments 67-74, wherein said assaying of (b) comprises a liquid chromatography mass spectrometry (LC-MS).
   76. The method of any one of embodiments 67-75, wherein said subject is an adult, an adolescent, or an infant.
   77. The method of any one of embodiments 67-76, wherein said subject has or is at risk of having an inflammatory disease, and wherein said inflammatory disease comprises an allergy or a dermatitis.
   78. The method of embodiment 77, wherein said allergy is allergic asthma, allergic pediatric asthma, or food allergy.
   79. The method of any one of embodiments 67-78, wherein said pharmaceutical composition is stored in a plant-based capsule prior to being administered to said subject.
   80. The method of any one of embodiments 67-79, wherein said bacterial population comprises said Akkermansia sp., said Faecalibacterium sp., and said Lactobacillus sp.
   81. The method of any one of embodiments 67-80, wherein said bacterial species is selected from the strains listed in Table 1.
   82. The method of embodiment 81, wherein said bacterial population comprises A. muciniphila (DSM 33213), F. prausnitzii (DSM 33185), or L. crispatus (DSM 33187).
   83. The method of embodiment 82, wherein said bacterial population comprises A. muciniphila (DSM 33213), F. prausnitzii (DSM 33185), and L. crispatus (DSM 33187).
   84. The method of any one of embodiments 67-83, wherein each bacterial species is present in an amount from about 10\hat{ }3 CFU/dose to about 10\hat{ }12 CFU/dose.
   85. The method of any one of embodiments 67-84, wherein each bacterial species is present in an amount from about 10\hat{ }7 CFU/dose to about 10\hat{ }10 CFU/dose.
   86. The method of any one of embodiments 67-85, wherein said Akkermansia sp. is present in an amount of about 10\hat{ }6 CFU/dose to about 2×10\hat{ }9 CFU/dose.
   87. The method of any one of embodiments 67-86, wherein said Faecalibacterium sp. is present in an amount of about 10\hat{ }6 CFU/dose to about 2×10\hat{ }9 CFU/dose.
   88. The method of any one of embodiments 67-87, wherein said Lactobacillus sp. is present in an amount of about 10\hat{ }7 CFU/dose to about 5×10\hat{ }9 CFU/dose.
   89. The method of any one of embodiments 67-88, wherein said bacterial population is present in a total amount of about 10\hat{ }3 CFU/dose to about 10\hat{ }12 CFU/dose.
   90. The method of any one of embodiments 67-89, wherein said bacterial population is present in a total amount of about 10\hat{ }7 CFU/dose to about 10\hat{ }10 CFU/dose.
   91. A method comprising:
(a) administering to a subject a pharmaceutical composition comprising a bacterial population comprising at least one strain of Akkermansia sp., at least one strain of Faecalibacterium sp., or at least one strain of Lactobacillus sp.;
(b) assaying a microbially-derived metabolite in said subject relative to said microbially-derived metabolite in a control sample, wherein said microbially-derived metabolite is an amine oxide.
   92. A method comprising:
(a) obtaining a sample comprising a microbially-derived metabolite from a subject;
(b) assaying said microbially-derived metabolite relative to said microbially-derived metabolite in a control sample,
wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp.,
wherein said microbially-derived metabolite is an amine oxide.
   93. The method of embodiment 91 or 92, wherein said microbially-derived metabolite comprises trimethylamine N-oxide.
   94. The method of any one of embodiments 91-93, wherein said microbially-derived metabolite in a control sample comprises said microbially-derived metabolite in a subject without being administered to said pharmaceutical composition.
   95. The method of any one of embodiments 91-94, wherein said microbially-derived metabolite in a control sample comprises said microbially-derived metabolite in said subject prior to being administered to said pharmaceutical composition.
   96. The method of any one of embodiments 91-95, wherein said microbially-derived metabolite is present in said subject during said subject is administered to said pharmaceutical composition.
   97. The method of any one of embodiments 91-96, wherein said microbially-derived metabolite is present in said subject in a washout period subsequent to said subject is administered to said pharmaceutical composition.
   98. The method of any one of embodiments 91-97, wherein said assaying of (b) comprises a liquid chromatography mass spectrometry (LC-MS).
   99. The method of any one of embodiments 91-98, wherein said subject is an adult, an adolescent, or an infant.
   100. The method of any one of embodiments 91-99, wherein said subject has or is at risk of having an inflammatory disease, and wherein said inflammatory disease comprises an allergy or a dermatitis.
   101. The method of embodiment 100, wherein said allergy is allergic asthma, allergic pediatric asthma, or food allergy.
   102. The method of any one of embodiments 91-101, wherein said pharmaceutical composition is stored in a plant-based capsule prior to being administered to said subject.
   103. The method of any one of embodiments 91-102, wherein said bacterial population comprises said Akkermansia sp., said Faecalibacterium sp., and said Lactobacillus sp.
   104. The method of any one of embodiments 91-103, wherein said bacterial species is selected from the strains listed in Table 1.
   105. The method of embodiment 104, wherein said bacterial population comprises A. muciniphila (DSM 33213), F. prausnitzii (DSM 33185), or L. crispatus (DSM 33187).
   106. The method of embodiment 105, wherein said bacterial population comprises A. muciniphila (DSM 33213), F. prausnitzii (DSM 33185), and L. crispatus (DSM 33187).
   107. The method of any one of embodiments 91-106, wherein each bacterial species is present in an amount from about 10\hat{ }3 CFU/dose to about 10\hat{ }12 CFU/dose.
   108. The method of any one of embodiments 91-107, wherein each bacterial species is present in an amount from about 10\hat{ }7 CFU/dose to about 10\hat{ }10 CFU/dose.
   109. The method of any one of embodiments 91-108, wherein said Akkermansia sp. is present in an amount of about 10\hat{ }6 CFU/dose to about 2×10\hat{ }9 CFU/dose.
   110. The method of any one of embodiments 91-109, wherein said Faecalibacterium sp. is present in an amount of about 10\hat{ }6 CFU/dose to about 2×10\hat{ }9 CFU/dose.
   111. The method of any one of embodiments 91-110, wherein said Lactobacillus sp. is present in an amount of about 10\hat{ }7 CFU/dose to about 5×10\hat{ }9 CFU/dose.
   112. The method of any one of embodiments 91-111, wherein said bacterial population is present in a total amount of about 10\hat{ }3 CFU/dose to about 10\hat{ }12 CFU/dose.
   113. The method of any one of embodiments 91-112, wherein said bacterial population is present in a total amount of about 10\hat{ }7 CFU/dose to about 10\hat{ }10 CFU/dose.
   114. A method comprising:
(a) administering to a subject a pharmaceutical composition comprising a bacterial population comprising at least one strain of Akkermansia sp., at least one strain of Faecalibacterium sp., or at least one strain of Lactobacillus sp.;
(b) assaying a membrane lipid in said subject relative to said membrane lipid in a control sample,
wherein said membrane lipid comprises phosphatidylinositol (PI) or phosphatidylethanolamine (PE).
   115. A method comprising:
(a) obtaining a sample comprising a membrane lipid from a subject;
(b) assaying said membrane lipid relative to said membrane lipid in a control sample,
wherein said membrane lipid comprises phosphatidylinositol (PI) or phosphatidylethanolamine (PE), and
wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp.
   116. The method of embodiment 114 or 115, wherein said membrane lipid in a control sample comprises said membrane lipid in a subject without being administered to said pharmaceutical composition.
   117. The method of any one of embodiments 114-116, wherein said membrane lipid in a control sample comprises said membrane lipid in said subject prior to being administered to said pharmaceutical composition.
   118. The method of any one of embodiments 114-117, wherein said membrane lipid is present in said subject during said subject is administered to said pharmaceutical composition.
   119. The method of any one of embodiments 114-118, wherein said membrane lipid is present in said subject in a washout period subsequent to said subject is administered to said pharmaceutical composition.
   120. The method of any one of embodiments 114-119, wherein said assaying of (b) comprises a liquid chromatography mass spectrometry (LC-MS).
   121. The method of any one of embodiments 114-120, wherein said subject is an adult, an adolescent, or an infant.
   122. The method of any one of embodiments 114-121, wherein said subject has or is at risk of having an inflammatory disease, and wherein said inflammatory disease comprises an allergy or a dermatitis.
   123. The method of embodiment 122, wherein said allergy is allergic asthma, allergic pediatric asthma, or food allergy.
   124. The method of any one of embodiments 114-123, wherein said pharmaceutical composition is stored in a plant-based capsule prior to being administered to said subject.
   125. The method of any one of embodiments 114-124, wherein said bacterial population comprises said Akkermansia sp., said Faecalibacterium sp., and said Lactobacillus sp.
   126. The method of any one of embodiments 114-125, wherein said bacterial species is selected from the strains listed in Table 1.
   127. The method of embodiment 126, wherein said bacterial population comprises A. muciniphila (DSM 33213), F. prausnitzii (DSM 33185), or L. crispatus (DSM 33187).
   128. The method of embodiment 127, wherein said bacterial population comprises A. muciniphila (DSM 33213), F. prausnitzii (DSM 33185), and L. crispatus (DSM 33187).
   129. The method of any one of embodiments 114-128, wherein each bacterial species is present in an amount from about 10\hat{ }3 CFU/dose to about 10\hat{ }12 CFU/dose.
   130. The method of any one of embodiments 114-129, wherein each bacterial species is present in an amount from about 10\hat{ }7 CFU/dose to about 10\hat{ }10 CFU/dose.
   131. The method of any one of embodiments 114-130, wherein said Akkermansia sp. is present in an amount of about 10\hat{ }6 CFU/dose to about 2×10\hat{ }9 CFU/dose.
   132. The method of any one of embodiments 114-131, wherein said Faecalibacterium sp. is present in an amount of about 10\hat{ }6 CFU/dose to about 2×10\hat{ }9 CFU/dose.
   133. The method of any one of embodiments 114-132, wherein said Lactobacillus sp. is present in an amount of about 10\hat{ }7 CFU/dose to about 5×10\hat{ }9 CFU/dose.
   134. The method of any one of embodiments 114-133, wherein said bacterial population is present in a total amount of about 10\hat{ }3 CFU/dose to about 10\hat{ }12 CFU/dose.
   135. The method of any one of embodiments 114-134, wherein said bacterial population is present in a total amount of about 10\hat{ }7 CFU/dose to about 10\hat{ }10 CFU/dose.
   136. A method comprising:
(a) administering to a subject a pharmaceutical composition comprising a bacterial population comprising at least one strain of Akkermansia sp., at least one strain of Faecalibacterium sp., or at least one strain of Lactobacillus sp.;
(b) assaying a pro-inflammatory immune marker relative to said pro-inflammatory immune marker in a control sample,
wherein said pro-inflammatory immune marker in present in a washout period subsequent to said administering of (a).
   137. A method comprising:
(a) obtaining a sample comprising a pro-inflammatory immune marker from a subject;
(b) assaying said pro-inflammatory immune marker relative to said pro-inflammatory immune marker in a control sample,
wherein said sample is obtained from said subject subsequent to being administered with a pharmaceutical composition comprising a bacterial population comprising at least two bacterial species selected from the group consisting of Akkermansia sp., Faecalibacterium sp., and Lactobacillus sp., and
wherein said pro-inflammatory immune marker in present in a washout period subsequent to said subject is administered with said pharmaceutical composition.
   138. The method of embodiment 136 or 137, wherein said pro-inflammatory immune marker comprises basophil, eotaxin, histamine, IL-4, or a combination thereof.
   139. The method of any one of embodiments 136-138, wherein said control pro-inflammatory immune marker comprises said pro-inflammatory immune marker in a subject without being administered to said pharmaceutical composition.
   140. The method of any one of embodiments 136-139, wherein said control pro-inflammatory immune marker comprises said pro-inflammatory immune marker in said subject prior to being administered to said pharmaceutical composition.
   141. The method of any one of embodiments 136-140, wherein said assaying of (b) comprises an enzyme-linked immunosorbent assay (ELISA) or cytometry.
   142. The method of any one of embodiments 136-141, wherein said subject is an adult, an adolescent, or an infant.
   143. The method of any one of embodiments 136-142, wherein said subject has or is at risk of having an inflammatory disease, and wherein said inflammatory disease comprises an allergy or a dermatitis.
   144. The method of embodiment 143, wherein said allergy is allergic asthma, allergic pediatric asthma, or food allergy.
   145. The method of any one of embodiments 136-144, wherein said pharmaceutical composition is stored in a plant-based capsule prior to being administered to said subject.
   146. The method of any one of embodiments 136-145, wherein said bacterial population comprises said Akkermansia sp., said Faecalibacterium sp., and said Lactobacillus sp.
   147. The method of any one of embodiments 136-146, wherein said bacterial species is selected from the strains listed in Table 1.
   148. The method of embodiment 147, wherein said bacterial population comprises A. muciniphila (DSM 33213), F. prausnitzii (DSM 33185), or L. crispatus (DSM 33187).
   149. The method of embodiment 148, wherein said bacterial population comprises A. muciniphila (DSM 33213), F. prausnitzii (DSM 33185), and L. crispatus (DSM 33187).
   150. The method of any one of embodiments 136-149, wherein each bacterial species is present in an amount from about 10\hat{ }3 CFU/dose to about 10\hat{ }12 CFU/dose.
   151. The method of any one of embodiments 136-150, wherein each bacterial species is present in an amount from about 10\hat{ }7 CFU/dose to about 10\hat{ }10 CFU/dose.
   152. The method of any one of embodiments 136-151, wherein said Akkermansia sp. is present in an amount of about 10\hat{ }6 CFU/dose to about 2×10\hat{ }9 CFU/dose.
   153. The method of any one of embodiments 136-152, wherein said Faecalibacterium sp. is present in an amount of about 10\hat{ }6 CFU/dose to about 2×10\hat{ }9 CFU/dose.
   154. The method of any one of embodiments 136-153, wherein said Lactobacillus sp. is present in an amount of about 10\hat{ }7 CFU/dose to about 5×10\hat{ }9 CFU/dose.
   155. The method of any one of embodiments 136-154, wherein said bacterial population is present in a total amount of about 10\hat{ }3 CFU/dose to about 10\hat{ }12 CFU/dose.
   156. The method of any one of embodiments 136-155, wherein said bacterial population is present in a total amount of about 10\hat{ }7 CFU/dose to about 10\hat{ }10 CFU/dose.

1.-156. (canceled)

157. A method comprising:
(a) obtaining a sample comprising a microbial community from a subject;
(b) assaying said microbial community relative to a control microbial community, wherein said sample is obtained from said subject subsequent to being administered with a pharmaceutical composition comprising a bacterial population comprising at least one strain of Akkermansia glycaniphila, at least one strain of Bacteroides thetaiotaomicron, and at least one strain of Lactobacillus aviarius.

158. The method of claim 157, wherein said assaying of (b) measures a microbial diversity of said microbial community, and wherein said microbial diversity comprises an alpha-diversity or a beta-diversity.

159. The method of claim 158, wherein said microbial diversity comprises said beta-diversity, and wherein said beta-diversity is measured by a weighted UniFrac distance or a Jaccard distance.

160. The method of claim 159, wherein said assaying of (b) identifies an enrichment or a depletion of at least one bacterial species in said microbial community.

161. The method of claim 160, wherein said assaying of (b) identifies said enrichment or said depletion of said at least one bacterial species in said microbial community using Statistical Framework Analysis of Composition of Microbiomes (ANCOM) or Applies Generalized Additive Models for Location, Scale and Shape (GAMLSS).

162. The method of claim 161, wherein said at least one depleted bacterial species in said microbial community is associated with an inflammatory disease or a metabolic disease.

163. The method of claim 162, wherein said at least one depleted bacterial species in said microbial community is associated with said inflammatory disease comprising atopy, asthma, an autoimmune disease, an autoinflammatory disease, a hypersensitivity, pediatric allergic asthma, allergic asthma, inflammatory bowel disease, Celiac disease, Crohn's disease, colitis, ulcerative colitis, collagenous colitis, lymphocytic colitis, diverticulitis, irritable bowel syndrome, short bowel syndrome, stagnant loop syndrome, chronic persistent diarrhea, intractable diarrhea of infancy, Traveler's diarrhea, immunoproliferative small intestinal disease, chronic prostatitis, postenteritis syndrome, tropical sprue, Whipple's disease, Wolman disease, arthritis, rheumatoid arthritis, Behcet's disease, uveitis, pyoderma gangrenosum, erythema nodosum, traumatic brain injury, psoriatic arthritis, juvenile idiopathic arthritis, multiple sclerosis, systemic lupus erythematosus (SLE), mayasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, bullous pemphigoid, sarcoidosis, ichthyosis, Graves ophthalmopathy, Addison's disease, Vitiligo, acne vulgaris, pelvic inflammatory disease, reperfusion injury, sarcoidosis, transplant rejection, interstitial cystitis, atherosclerosis, food allergy, or atopic dermatitis.

164. The method of claim 162, wherein said at least one depleted bacterial species in said microbial community is associated with said metabolic disease comprising pre-diabetes, type 1 diabetes, type 2 diabetes, diabetes complications, prediabetes, non-alcoholic fatty liver disease (NAFLD), weight loss, insulin-deficiency or insulin-resistance related disorders, glucose intolerance, abnormal lipid metabolism, atherosclerosis, hypertension, cardiac pathology, stroke, hyperglycemia, hepatic steatosis, dyslipidemia, dysfunction of said immune system associated with overweight and obesity, cardiovascular diseases, high cholesterol, elevated triglycerides, asthma, sleep apnea, osteoarthritis, neuro-degeneration, gallbladder disease, syndrome X, inflammatory and immune disorders, atherogenic dyslipidemia, or cancer.

165. A method comprising:
(a) administering to a subject a pharmaceutical composition comprising a bacterial population comprising at least one strain of Akkermansia glycaniphila, at least one strain of Bacteroides thetaiotaomicron, and at least one strain of Lactobacillus vaccinostercus;
(b) assaying a steroid compound or a microbially-derived metabolite in said subject relative to said steroid compound or said microbially-derived metabolite, respectively, in a control sample.

166. The method of claim 165, wherein (b) comprises assaying said microbially-derived metabolite in said subject relative to said microbially-derived metabolite in said control sample, and wherein said microbially-derived metabolite comprises a bile acid, a short chain fatty acid (SCFA), an amino acid, a branched-chain of amino acids, a linear-chain of amino acids, a trimethylamine N-oxide, an indole, a derivative thereof, or a combination thereof.

167. The method of claim 165, wherein (b) comprises assaying said steroid compound in said subject relative to said steroid compound in said control sample, and wherein said steroid compound comprises a progestogenic steroid compound, a corticosteroid steroid compound, an androgenic steroid compound, an estrogenic steroid compound, or a combination thereof.

168. The method of claim 167, wherein said steroid compound comprises said androgenic steroid compound comprising androstenediol (3alpha, 17alpha) monosulfate, 11beta-hydroxyandrosterone glucuronide, androsterone glucuronide, a derivative thereof, or a combination thereof.

169. A method comprising:
(a) administering to a subject a pharmaceutical composition comprising a bacterial population comprising at least one strain of Akkermansia glycaniphila, at least one strain of Bacteroides thetaiotaomicron, and at least one strain of Lactobacillus vinii;
(b) assaying a membrane lipid or a pro-inflammatory immune marker in said subject relative to said membrane lipid or said pro-inflammatory immune marker respectively, in a control sample.

170. The method of claim 169, wherein (b) comprises assaying said membrane lipid in said subject relative to said membrane lipid in said control sample, and wherein said membrane lipid comprises a phospholipid, a glycolipid, a cholesterol, or a combination thereof.

171. The method of claim 170, wherein said membrane lipid comprises said phospholipid comprising a phosphatidylcholine (PC), a phosphatidylethanolamine (PE), a phosphatidylinositol (PI) or a combination thereof.

172. The method of claim 171, wherein phospholipid comprises said PC comprising 1,2-dipalmitoyl-GPC (16:0/16:0), 1-palmitoyl-2-palmitoleoyl-GPC (16:0/16:1), 1-myristoyl-2-palmitoyl-GPC (14:0/16:0), 1-palmitoyl-2-oleoyl-GPC (16:0/18:1) or a combination thereof.

173. The method of claim 171, wherein phospholipid comprises said PE comprising 1-palmitoyl-2-arachidonoyl-GPE (16:0/20:4), 1-palmitoyl-2-linoleoyl-GPE (16:0/18:2), or a combination thereof.

174. The method of claim 171, wherein phospholipid comprises said PI comprising 1-palmitoyl-2-linoleoyl-GPI (16:0/18:2).

175. The method of claim 169, wherein (b) comprises assaying said pro-inflammatory immune marker in said subject relative to said pro-inflammatory immune marker in said control sample, and wherein said pro-inflammatory immune marker comprises a nucleotide, a polynucleotide, a protein, an amino acid, a peptide, a lipid, a cell, a chemical compound, a saccharide, or a polysaccharide.

176. The method of claim 169, wherein (b) comprises assaying said pro-inflammatory immune marker in said subject relative to said pro-inflammatory immune marker in said control sample, and wherein said pro-inflammatory immune marker comprises a basophil, an eotaxin, a histamine, an interleukin-4 (IL-4), or a combination thereof
